(19) United States
(12) Patent Application Publication
(10) Pub. No.: US 2025/0043291 A1
(43) Pub. Date: Feb. 6, 2025

(54) TRANSMISSIBLE ELEMENTS COMPRISING PATHWAY MODIFICATION SYSTEMS AND EXOGENOUS NUCLEIC ACIDS FOR THE PRODUCTION OF MOLECULES OF INTEREST

(71) Applicant: SNIPR Biome ApS, Copenhagen (DK)

(72) Inventors: Jennifer LE MIÈRE, Ely (GB); Emilie SØNDBERG, Copenhagen (DK); Szabolcs SEMSEY, Copenhagen (DK); Ioannis MOUGIAKOS, Copenhagen (DK)

(21) Appl. No.: 18/763,680

(22) Filed: Jul. 3, 2024

(30) Foreign Application Priority Data
Jul. 4, 2023 (GB) 2310227.0

(51) Int. Cl. C12N 15/70; A61K 35/747; C12N 9/22; C12N 15/113; C12N 15/74

(52) U.S. Cl. CPC C12N 15/70; A61K 35/747; C12N 9/22; C12N 15/113; C12N 15/74; C12N 2310/14; C12N 2310/20

(57) ABSTRACT

The present invention relates to transmissible elements (e.g. conjugative plasmids, phage and phagemid) which are engineered to express molecules of interest (MOIs) and a pathway modification system (PMS) which is able to introduce mutations in target endogenous genes in a recipient bacterium, or is able to produce nucleic acids which inhibit or increase expression of target endogenous genes in a recipient bacterium, or is able to express peptide inhibitor molecules which inhibit a target endogenous protein in a recipient bacterium. The invention relates to host cells containing the transmissible elements, to pharmaceutical compositions containing such transmissible elements or host cells, and their use in the treatment of various diseases.

### CROSS REFERENCE TO RELATED APPLICATIONS

This application claims priority to and benefit of GB Patent Application No. 2310227.0 filed on Jul. 4, 2023, the disclosures of which are herein incorporated by reference in their entirety.

### REFERENCE TO AN ELECTRONIC SEQUENCE LISTING

The content of the electronic sequence listing (786212002200seglist.xml; Size: 112,792 bytes; and Date of Creation: Jun. 27, 2024) is herein incorporated by reference in its entirety.

### BACKGROUND TO INVENTION

There have been many attempts to engineer bacteria to produce molecules of interest (MOIs) in situ in local environments, such as the gut microbiome of animals, such as humans, or in soil, water and other natural microbiomes (see for example, Omer et al., Frontiers Bioengineering & Biotechnology, 10, 870675, 2022, doi:10.3389/fbioe.2022.870675, and Bai et al., Nat. Rev. Bioeng., 1, 665-679, 2023, doi:https://doi.org/10.1038/s44222-023-00072-2, for human medical uses; Li et al., AIChE 1., 69(12), e18228, 2023, doi:10.1002/aic.18228 and Zhang et al., Environmental Science: Water Research & Technology, 6, 1967-1985, 2020, doi https://doi.org/10.1039/DOEW00393J for water treatment; and Ribello et al., Bioengineered, 12(2), 12839-12853, 2021, doi: 10.1080/21655979.2021.2004645 for a review on bacteria-based soil treatments). For example, it has been proposed that engineered bacteria can be used to deliver several different categories of molecule for therapeutic uses. For example, Lactococcus lactis has been engineered to deliver anti-tumor necrosis factor (TNF)-α antibody for the treatment of chronic inflammatory bowel disease (IBD), see Vandenbroucke et al., Mucosal Immunol., 3, 2010, 49-56; and Bacteroides ovatus has been engineered to produce human keratinocyte growth factor-2 (KGF-2) to treat IBD, see Hamady et al, Gut, 59, 2010, 461-469. In other fields, Bacillus subtilis has been engineered to express a S-adenosylmethionine methyltransferase gene (CmarsM) which converts most of the inorganic arsenic contained in organic manure into dimethylarsenate and trimethylarsine oxide, as a bioremediation strategy, see Huang et al., Appl. Environ. Microbiol., 81 (19), 2015, 6718-6724. In water treatment applications, Synechococcus elongatus has been engineered to produce an oxidative laccase enzyme which can degrade a number of different water contaminants, such as dyes and other chemical pollutants, see Datta et al., Nature Communications, 14, 4742, 2023.

One ongoing problem which is yet to be addressed is how to effectively introduce such modified bacteria in a way that they reach a sufficient colonisation level to allow them to produce quantities of the MOI to produce the desired effect. In human applications, often the administration of antibiotics precedes the administration of the modified bacteria, killing the existing bacteria in the microbiome to create a niche, into which the modified bacteria can grow. However, there are a number of problems associated with this approach:
1. The use of antibiotics tends to remove bacteria indiscriminately, and may remove helpful and useful bacteria from the microbiome of interest;
2. The remaining bacteria may comprise a higher amount of antibiotic resistant bacteria, which may subsequently be more difficult to remove;
2. The engineered bacteria must compete for colonisation of the niche with the bacteria remaining in the microbiome of interest. This may result in fewer of the engineered bacteria than desired and/or an increase in undesirable (e.g. antimicrobial resistant) bacteria filling the niche if those undesirable bacteria have a competitive advantage over the engineered bacteria.

It has been proposed to use conjugative plasmids (see, for example, WO2020/010452A1 (Société de Commercialisation des Produits de la Recherche Appliquée Socpra Sciences Santé et Humaines S.E.C.), WO2021/037732A1 (SNIPR Biome, Aps), WO2020/229372A1 (Folium Food Science, Limited), WO2015/069682A2 (President and Fellows of Harvard College), and Hamilton et al., Nature Comms, 10:4544, 2019, doi: https://doi.org/10.1038/s41467-019-12448-3), or phage (see, for example, WO2021/250284A1 (Eligo Bioscience), WO2021/092210A1 and WO2022/098916A1 (Locus Biosciences, Inc) and WO2016/205276A1 (North Carolina State University)) to deliver exogenous genes to target recipient bacteria. The main purpose of these disclosures is killing of the recipient bacteria, re-sensitisation to antibiotics or delivery of genetic cargo providing a competitive advantage to the recipient bacteria. Some disclose the addition of the exogenous genes and circuitry for destroying the delivered plasmids (known as kill switches), which target and cut the plasmids comprised by the recipient bacteria.

WO2021/204967A1 (Eligo Biosciences) proposes to use phage to deliver a base editor to a target recipient cell which edits a target sequence in the recipient bacteria

The present invention aims to solve some of the above-mentioned problems.

### SUMMARY

The present invention, instead of trying to introduce a modified bacterium directly into the microbiome of interest uses transmissible elements, which may be comprised within a donor bacterium, a phage or as part of a packaged phagemid. These transmissible elements provide the recipient bacteria, i.e. those which already exist within the microbiome of interest with exogen

The present invention therefore provides the following:

### In a First Configuration:

There is provided a transmissible element (e.g. a plasmid, conjugative plasmid, phage or phagemid) for transmission to a recipient bacterium, or a bacterium comprising said transmissible element, wherein the transmissible element comprises:
A) at least one exogenous nucleic acid sequence to the recipient bacterium for the production of a first molecule of interest (MOI) in the recipient bacterium; and
B) a Pathway Modulation System (PMS) which encodes a nucleic acid modifier or a peptide molecule to modulate a target endogenous nucleic acid or protein for the production or consumption (or degradation) of the first MOI in the recipient bacterium.

### In a Second Configuration:

There is provided a pharmaceutical composition comprising (i) a donor bacterium as described herein, or (ii) a transmissible element as described herein, and a pharmaceutically acceptable excipient or carrier.

### In a Third Configuration:

There is provided a method of modifying recipient bacteria in the gut of a subject, comprising administering to said subject (i) a donor bacterium as described herein, or (ii) a transmissible element as described herein or (iii) a pharmaceutical composition as described herein.

### In a Fourth Configuration:

There is provided a method of treating or preventing a disease or condition in a subject in need thereof, said method comprising administering to a subject in need thereof (i) a donor bacterium as described herein, or (ii) a transmissible element as described herein, or (iii) a pharmaceutical composition as described herein, and wherein the subject comprises one or more strains of bacteria which are capable of the transmissible element being transmitted to said one or more strains of bacteria.

### In a Fifth Configuration:

There is provided a method of treating a metabolic disease, such as a cardiovascular metabolic disease, optionally selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase 1/3 deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome (in particular selected from metabolic syndrome, type 2 diabetes (including complications of type 2 diabetes, e.g. insulin sensitivity in type 2 diabetes), and non-alcoholic fatty liver disease), said method comprising administering to a subject in need thereof (i) a donor bacterium as described herein, or (ii) a transmissible element as described herein, or (iii) a pharmaceutical composition as described herein.

### BRIEF DESCRIPTION OF THE FIGURES

FIG. 1: Schematic diagram of a recipient bacterial cell which is engineered to produce a first MOI. Dashed lines represent the recipient bacterial cell. EXP1 is an endogenous exporter capable of exporting the first MOI out of the cell. D1 is an endogenous enzyme which is capable of degrading or destroying the first MOI. G1 (square box) is an exogenous nucleic acid, e.g. a gene, for production of the first MOI.

FIG. 2: Schematic diagram of a recipient bacterial cell which is engineered to convert a second MOI to a first MOI. Dashed lines represent the recipient bacterial cell. In this example, the cell comprises two related endogenous pathways. The first comprises multiple enzymes (E1, E2, E3, E4 . . . etc) which convert substrates (S1, S2, S3 . . . etc) into the second MOI, or into other metabolites (M1 . . . etc). In the second endogenous pathway, other substrates (P, X, X1, X2 . . . etc) are converted by other endogenous enzymes, one of which (in this example, E1) is common between the first and second pathways. Substrates may be imported into the cell by endogenous importers (IMP1, IMP2 . . . etc). Any of the substrates (in this example, S1) may also be exported out of the cell by endogenous exporters (EXP2). Any of the substrates may be converted to an undesirable product (in this example, M1) by an endogenous enzyme (in this example, E4). EXP1 is an exporter capable of exporting the first MOI out of the cell. D1 is an endogenous enzyme which is capable of degrading or destroying the first MOI. G1, G2 . . . etc (square box) are exogenous nucleic acids, e.g. one or more genes, for conversion of the second MOI to the first MOI.

FIG. 3: Schematic diagram showing the biosynthetic pathway for the conversion of indole to indole-3-acetic acid (IAA). Note that in the absence of a specific gene for the conversion of IAA to IBA, it is believed (without being bound by theory) to be an equilibrium reaction which spontaneously converts IAA to IBA.

FIG. 4: Graphical representation of the plasmids used in Examples 1 and 2. The Cas-S variant that each plasmid expresses is noted at the left side of the figure. The name of each plasmid is noted at the bottom left side of each plasmid. The Cas-S genes are presented as boxes with solid fills and the corresponding gene names are noted above each box. Dotted lines correspond to deleted Cas-S genes. The expression of the Cas-S genes was driven by the pBolA promoter, which is presented as an arrow that points to the direction of the transcription. The expression of the crRNA module was driven by the ‘Leader’ sequence, presented as a box filled with diagonal stripes and is located right upstream (5′) of the crRNA expressing module. The CRISPR repeats (SEQ ID No:6) of the crRNA expressing module are presented as triangles and the spacer is presented as a circle. The locations of the BsmBI recognition sites are indicated with asterisk (*) signs. The tetracycline resistance marker gene, the SC101 origin of replication and the repA101 replication protein gene are presented as boxes with checker, brick and grid fills respectively.

FIG. 5: Schematic representation of the positions of the protospacers employed for the Cas-S CRISPRi assays for downregulation of the GFP expression from strain b5815 that contains chromosomally integrated gfp gene. Each protospacer position is indicated with a triangle and the ID of each protospacer is noted above the triangle.

FIGS. 6A-6C: Graphical representation of the chromosomal CRISPRi activity of the wild type Cas-S system on the GFP expression of strain b5815 over a 24-hour period. The targeted protospacers were located (FIG. 6A) at the beginning of the gfp orf and in the p70a promoter region (Protospacers indicated in brackets: S1F, S1R, S2F, S2R, S3F, S3R), (FIG. 6B) at the end of the gfp orf and the 200 bp regions upstream and downstream of gfp (Protospacers indicated in brackets S4F, S4R, S5F, S6F, S6R) (FIG. 6C) 1 kb upstream (Protospacers indicated in brackets: U1kF, U1kR) and 2 kb upstream (Protospacers indicated in brackets: U2kF, U2kR) of the gfp orf.

FIGS. 7A-7B: Graphical representation of the chromosomal CRISPRi activity of the ΔcasS3 Type-S system on the GFP expression of strain b5815 over a 24-hour period. The targeted protospacers were located (FIG. 7A) at the beginning of the gfp orf and in the p70a promoter region (Protospacers S1F, S1R, S2F, S2R, S3F, S3R), (FIG. 7B) at the end of the gfp orf and the 200 bp regions upstream and downstream of gfp (Protospacers S4F, S4R, S5F, S6F, S6R).

FIGS. 8A-8C: Graphical representation of the chromosomal CRISPRi activity of the (FIG. 8A) ΔcasS3 Cas-S system, (FIG. 8B) ΔcasS1ΔcasS3 Cas-S system and (FIG. 8C) ΔcasS1 Cas-S system on the GFP expression of strain b5815 over a 24-hour period. The targeted protospacers were located at the beginning of the gfp orf and in the p70a promoter region (Protospacers S1F, S1R, S2F, S2R, S3F, S3R). The plasmids employed for the transformation of b5815 strain is denoted with (:).

FIGS. 9A-9D: Graphical representation of on-plasmid CRISPRi activity of the (FIG. 9A) ΔcasS3 Cas-S system, (FIG. 9B) ΔcasS4 Cas-S system, (FIG. 9C) ΔcasS4 Cas-S system and (FIG. 9D) ΔcasS1ΔcasS3 Cas-S system on the GFP expression of strain b6386 over a 24-hour period. The targeted protospacers were located at the beginning of the gfp orf and in the p70a promoter region (Protospacers S1F, S1R, S2F, S2R, S3F, S3R).

FIG. 10A-10D: Schematic representation of the plasmid combinations employed for the (FIG. 10A) CasS1, (FIG. 10B) CasS1 (ΔCasS3), (FIG. 10C) CasS3 and (FIG. 10D) CasS4 base editing assays. The asterix (*) indicates that the spacer is directed against one of: a non-targeting control, S1F, S1R, S2F, S2R, S3F or S3R.

FIG. 11A-11D: Heatmap based visualization of representative sequencing results from the Type-S CRISPR/Cas base editing assays. The PmCDA1 cytidine deaminase is fused to the C-terminus of (FIG. 11A) the Cas-S1 subunit of the wild type Cas-S CRISPR/Cas system, (FIG. 11B) the Cas-S1 subunit of the ΔCas-S3 Cas-S CRISPR/Cas system, (FIG. 11C) the Cas-S3 subunit of the wild type Cas-S CRISPR/Cas system, (FIG. 11D) the Cas-S2 subunit of the wild type Cas-S CRISPR/Cas system. On the top side of each box are noted i) the code of the tested protospacer and ii) the nucleotide sequence of the protospacer (highlighted with a boxy arrow that additionally shows the direction of the protospacer) together with the sequence of the immediately neighboring genomic regions where base-editing modifications were detected. On the bottom side of each box are noted i) the codes of unique Sanger sequencing results (each from a distinct colony) and ii) all the C-to-T modifications, or G-to-A modifications when the reverse strand was targeted and modified, that were detected in the sequencing results. The color intensity of each heatmap cell represents the relative C to T (or G to A) abundance for each position, according to the height ratio of the corresponding C and T peaks from the Sanger sequencing results. For example, black boxes represent colonies with 100% C to T mutated genotype for the corresponding positions, whereas off-white boxes correspond to 95% to 5% (reliable limit of detection for Sanger sequencing) C to T mixed mutant genotypes for the corresponding positions.

FIG. 12: Schematic representation of the modifications introduced into the conjugative 810 plasmid. The DNA inserted to p2464 contains a chloramphenicol marker. The DNA inserted to p2806 and p3024 contains genes necessary to produce (G1-63) and export (G4) a molecule of interest (MOI), as well as a chloramphenicol resistance marker for selection. The DNA inserted to p3024 and p2986 have a CasS-based base-editing system. The system consists of 7 genes, where some are fused to produce a single protein. The PmCDA1-UGI base-editor is connected to the C-terminus of CasS4 via a linker and is directed to the position given by the spacer, targeting an endogenous gene, where premature stop codons are introduced. Uracil DNA glycosylase inhibitor (UGI), cytidine deaminase (PmCDA1).

FIG. 13: Bar chart showing the total number of recipients (R) and transconjugants (T) after conjugation of plasmids p2464 (ß10, control), p2806 (ß10 with MOI pathway only), p2986 (ß10 with Base-editor) and p3024 (ß10 with both MOI pathway and Base-editor) from b8524 (donor) into b5652 (recipient).

FIG. 14: B5652 containing p2464 (empty vector), p2806 (MOI pathway only), p2986 (base-editor) or p3024 (pathway and base-editor) is grown in M9 (supplemented with 1 mM substrate for MOI production) overnight and then measured for MOI production. The production pathway is necessary for high levels of MOI production, which are further increased by the introduction of a premature stop codon in the target gene by the base-editing system. The graph is the result of 4 biological replicates +/−1 st dev. p2806 and p3024 are significantly different using a T-test with p=0.009.

### DETAILED DESCRIPTION

As previously mentioned, one ongoing problem with the delivery of genetically modified bacterial cells which is yet to be addressed is how to effectively introduce such modified bacteria in a way that they reach a sufficient colonisation level in a native microbiome to allow them to produce quantities of the MOI to produce the desired effect. In human applications, often the administration of antibiotics precedes the administration of the modified bacteria, killing the existing bacteria in the microbiome to create a niche, into which the modified bacteria can grow. However, there are a number of problems associated with this approach:
1. The use of antibiotics tends to remove bacteria indiscriminately, and may remove helpful and useful bacteria from the microbiome of interest;
2. The remaining bacteria may comprise a higher amount of antibiotic resistant bacteria, which may subsequently be more difficult to remove;
2. The engineered bacteria must compete for colonisation of the niche with the bacteria remaining in the microbiome of interest. This may result in fewer of the engineered bacteria than desired and/or an increase in undesirable (e.g. antimicrobial resistant) bacteria filling the niche if those undesirable bacteria have a competitive advantage over the engineered bacteria.

Whereas a genetically modified bacterium can comprise genetic modifications in both endogenous genes and in the addition of exogenous nucleic acids, when using a conjugative plasmid, there has not been much progress in using transmissible elements to introduced targeted and precise modifications in native nucleic acids in the recipient bacteria with the aim of enhancing the production of a first MOI. Most applications seek to employ an engineered bacterium in which native nucleic acids have been modified through genetic engineering to increase or decrease expression in combination with the addition of exogenous genes for the expression of desired products, optionally with the addition of exporters and/or importers in the bacterium.

The present inventors have realised that engineered transmissible elements, such as conjugative plasmids may not only be used to deliver genes and nucleic acids for producing a first MOI, but that also, coupled with a Pathway Modification System (PMS), these conjugative plasmids are able to efficiently modify endogenous genes in the recipient bacteria, which can enhance the production of the first MOI, or increase degradation of a second, undesirable MOI.

Thus, there is provided a transmissible element for transmission to a recipient bacterium, or a bacterium comprising said transmissible element, wherein the transmissible element comprises A) at least one exogenous nucleic acid sequence to the recipient bacterium for the production of a first molecule of interest (MOI) in the recipient bacterium; and B) a Pathway Modulation System (PMS) which encodes a nucleic acid modifier to modulate a target endogenous nucleic acid for the production or consumption (or degradation) of the first MOI in the recipient bacterium.

There is also provided a transmissible element for transmission to a recipient bacterium, or a bacterium comprising said transmissible element, wherein the transmissible element comprises A) at least one exogenous nucleic acid sequence to the recipient bacterium for the production of a first molecule of interest (MOI) in the recipient bacterium; and B) a Pathway Modulation System (PMS) which encodes a a peptide molecule to modulate a target endogenous protein for the production or consumption (or degradation) of the first MOI in the recipient bacterium.

The transmissible elements described herein, and donor (host) cells comprising the transmissible elements (e.g. conjugative plasmids) described herein are useful as medicaments. Formulations are described elsewhere herein.

### Definitions

A “beneficial metabolite” refers to a molecule that is beneficial to the local environment of the recipient or donor cell, or to the microbiome as a whole, or indeed causes downstream beneficial effects to the organism which may comprise the cell or microbiome. Beneficial metabolites may not be present, or may be present in too low a concentration in the recipient bacterium which results in a detrimental effect in the recipient (or microbiome or organism), optionally as compared to other bacteria in a microbiome. Beneficial metabolites may be molecules which provide a competitive advantage to the recipient or donor bacterium. In some embodiments, a beneficial metabolite may be a food source, such as a sugar which can be used by the recipient or donor bacterium, thus providing a competitive advantage to the bacteria which are able to use that food source compared to other bacteria in a microbiome which are not able to use that food source.

As used herein, a “conjugative plasmid” is a particular transmissible element. A conjugative plasmid is a plasmid which, when comprised within a bacterial cell (“host” or “donor” cell, used interchangeably herein) is able to be transferred to another bacterium (“recipient” cell) through the mechanism of bacterial conjugation. Bacterial conjugation is the unidirectional and horizontal transmission of genetic information from one bacterium to another. Conjugative plasmids generally fall into two classes: mobilizable plasmids and self-transmissible plasmids. Mobilizable plasmids comprise at least an origin of transfer (oriT), a relaxase and other genetic information on the plasmid which is transferred to the recipient cell, and require helper functions provided by e.g. a second plasmid or the chromosome of the donor cell to effect the plasmid transfer. On the other hand, self-transmissible plasmids, in addition to the genetic information on the plasmid which is transferred to the recipient cell, also contain all the molecular machinery needed for self-transfer (e.g. for pilus formation and initiation of gene transfer) on the same plasmid. Both mobilizable plasmids and self-transmissible plasmids comprise relaxase genes which recognises the origin of transfer (oriT) and catalyses both the initial cleavage of oriT in the donor, to produce the DNA strand from the plasmid that will be transferred, as well as the final ligation of the transported DNA in the recipient cell that reconstitutes the conjugated plasmid. Thus, in one embodiment, the conjugative plasmid is a mobilizable plasmid. In another embodiment, the conjugative plasmid is a self-transmissible plasmid. In another embodiment, the conjugative plasmid includes an origin of transfer (oriT). Plasmid mobility, mechanisms and structures are described in more detail in Smillie et al., Microbiol. Mol. Biol. Rev., 74(3): 434-452, 2010 doi: 10.1128/MMBR.00020-10, which is incorporated herein in its entirety.

Bacterial cells possessing a conjugative self-transmissible plasmid contain a surface structure (pilus) encoded by the conjugative machinery on the plasmid that is involved in the coupling of donor and recipient cells, and the transfer of the genetic information contained within the plasmid. Conjugation involves contact between cells, and the transfer of genetic traits can be mediated by many plasmids. Among all natural transfer mechanisms, conjugation is the most efficient. For example, F plasmid of E. coli pCFlO plasmid of Enterococcus faecalis and pXO16 plasmid of Bacillus thuringiensis employ different mechanisms for the establishment of mating pairs, the sizes of mating aggregates are different, and they have different host ranges within gram-negative (F) as well as gram-positive (pCFlO and pXO16) bacteria. Their plasmid sizes are also different; 54, 100 and 200 kb, respectively. Remarkably, however despite differences in origin and size, those conjugation systems are able to sustain efficient conjugative transfer in liquid medium. The conjugative process permits the protection of plasmid DNA against environmental nucleases, and thus efficient delivery of plasmid DNA into a recipient cell can be obtained. Conjugation functions are naturally plasmid encoded. Numerous conjugative plasmids (and transposons) are known, which can transfer associated genes within one species (narrow host range) or between many species (broad host range). Transmissible plasmids are widespread across the domain of bacteria and similar systems of horizontal gene transfer through pilus structures have recently been described for Archaea. Engineered conjugative plasmids are described in more detail in WO2021/037732 (SNIPR Biome ApS), which is incorporated herein in its entirety. The features of such conjugative plasmids and bacterial cells comprising them as described in the claims as filed in WO2021/037732 are also incorporated herein by reference.

“Constitutive promoter” refers to a promoter that is capable of facilitating continuous transcription of a coding sequence or gene under its control and/or to which it is operably linked. Constitutive promoters and variants are well known in the art and are described elsewhere herein.

A “detrimental metabolite” refers to a molecule that is harmful to the local environment of the recipient or donor cell, or to the microbiome as a whole, or indeed causes downstream harmful effects to the organism which may comprise the cell or microbiome. Detrimental metabolites, whilst needed and/or required to a certain degree, may be present in too high a concentration in the recipient bacterium which results in a detrimental effect in the recipient (or microbiome or organism), optionally as compared to other bacteria in a microbiome. Detrimental metabolites may be molecules which cause a fitness cost to the recipient or donor bacterium. In embodiments where the second MOI is a detrimental metabolite, the first MOI is a molecule which either has a neutral effect of the recipient or donor bacterium (or microbiome or organism), or is easily excreted by the cell and/or organism, or is a beneficial metabolite.

“Exogenous” or “heterologous” are used interchangeably and refer to a nucleotide sequence that is not normally found in a given cell in nature. The given cell is usually the donor or recipient bacterium, in particular the recipient bacterium. Thus, an exogenous or heterologous nucleic acid may be one which is not naturally found in the recipient bacterium, i.e. the nucleic acid is exogenous to the recipient bacterium. As used herein, a heterologous sequence encompasses a nucleic acid sequence (or amino acid sequence) that is exogenously introduced into a given cell. A heterologous gene includes a native gene, or fragment thereof, that has been introduced into the cell. For example, a heterologous gene may include a native coding sequence that is a portion of a chimeric gene to include a native coding sequence that is a portion of a chimeric gene to include non-native regulatory regions that is reintroduced into the cell. A heterologous gene may also include a native gene, or fragment thereof, introduced into a non-native cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. For plasmids, such as conjugative plasmid, a heterologous gene is one which has been engineered to be included on such plasmid (e.g. conjugative plasmid). However in alternative embodiments relating to plasmids, such as conjugative plasmids, a heterologous or exogenous gene is heterologous to the recipient bacterium.

As used herein an “exporter” refers to a membrane-integrated protein which is capable of exporting a molecule (such as a naturally occurring cellular substrate or metabolite, an MOI, or any other peptide, protein or small molecule) out of the bacterial cell. As discussed elsewhere herein, in some bacteria there are no endogenous exporters of MOIs, and thus the cell must rely on diffusion of the MOIs to remove the MOIs from the cell. Without being bound by theory, the export of MOIs in gram-negative bacteria may be particularly problematic, due to the presence of both an inner and outer membrane. Thus, the addition of a heterologous exporter capable of exporting the MOI in the transmissible element may provide further benefits in this type of bacterial cell. In other bacteria, there may be endogenous exporters which export substrates which are used in the production of the second MOI. When these substrates are exported from the cell, they are no longer available to be used in the production of the second MOI, thus potentially decreasing the amount of second MOI available in the cell to be converted into the first MOI. It may be desirable to deactivate or reduce the ability of these exporters to export the endogenous substrates from the cell. The ability of any given protein to act as an exporter can be easily measured by those skilled in the art. Identification of putative exporters can be achieved through literature searches as well as by using protein and genetic databases, such as GenBank, which are well-known to those skilled in the art.

As used herein an “importer” refers to a membrane-integrated protein which is capable of importing a molecule (such as a naturally occurring cellular substrate or metabolite, a second MOI or any other peptide, protein or small molecule) into the bacterial cell. As discussed elsewhere herein, in some bacteria there are no endogenous importers of second MOIs and/or of substrates which are used in the production of the second MOI, and thus the cell must rely on diffusion of the second and/or such substrates MOIs to import them into the cell. Thus, the addition of a heterologous importer capable of importing the second MOI and/or substrates which are used in the production of the second MOI in the transmissible element may provide further benefits in this type of bacterial cell. The ability of any given protein to act as an importer can be easily measured by those skilled in the art. Identification of putative importers can be achieved through literature searches as well as by using protein and genetic databases, such as GenBank, which are well-known to those skilled in the art.

An “inducible promoter” refers to a promoter which transcribes a coding sequence or gene under its control and/or to which it is operably linked in the presence of an inducer of said promoter. The inducer may be one or more environmental condition(s) and/or one or more inducing molecule(s).

A “kill switch” refers to a biocontainment system which is included in the transmissible element (e.g. the conjugative plasmid) and is designed to destroy the recipient bacterium, or in the case of a plasmid (e.g. a conjugative plasmid), either the plasmid itself only, or the plasmid and the bacterium comprising the plasmid together, when no longer contained within its desired environment (e.g. within a microbiome, such as a gut microbiome, within a subject). Such means are well-known in the art, and are regulatable, for example by the addition of non-naturally occurring substances (e.g. synthetic amino acids), temperature and the like.

A “microbiome,” as used herein, refers to the totality of microbes in a particular environment (e.g. in/on an organism, in a marine environment (e.g. ocean), and/or in a terrestrial environment (e.g. soil)). In some embodiments, a microbiome may refer to the totality of microbes that reside, or are stably maintained, for example, on the surface and in deep layers of the skin, in the saliva and oral mucosa, in the conjunctiva, and in the gastrointestinal tracts of an organism. The microbiome may exist within any of the organs described elsewhere herein.

“Peptide” and “protein” are used interchangeably herein.

“Phage” or “bacteriophage” are used interchangeably and refer to obligate intracellular parasites that multiply inside bacteria by co-opting some or all of the host biosynthetic machinery.

Phage genomes come in a variety of sizes and shapes. Most phages range in size from 24-200 nm in diameter. Phages contain nucleic acid (i.e., genome) and proteins, and may be enveloped by a lipid membrane. Depending upon the phage, the genome can be either DNA or RNA, and can exist in either circular or linear forms. The size of the phage genome varies depending upon the phage. The simplest phages have genomes that are only a few thousand nucleotides in size, while the more complex phages may contain more than 100,000 nucleotides in their genome, and in rare instances more than 1,000,000. The number and amount of individual types of protein in phage particles will vary depending upon the phage.

A “phagemid” refers to a bacteriophage-derived vector containing the replication origin of a plasmid and the packaging site of a bacteriophage. Examples of phagemids that may be used in accordance with the present disclosure include, without limitation, M13-derived phagemids containing the f1 origin for filamentous bacteriophage packaging such as, for example, pBluescript II SK (+/−) and KS (+/−) phagemids, pBC SK and KS phagemids, pADL and P1-based phagemids (see, e.g. Westwater C A et al., Microbiology 148, 943-50 (2002); Kittleson J T et al., ACS Synthetic Biology 1, 583-89 (2012); Mead D A et al., Biotechnology 10, 85-102 (1988)). Other phagemids may be used and, for example, can be made to work with packaging systems from natural, engineered or evolved bacteriophage.

“Preventing” as used herein in relation to transcription or expression of a gene or protein refers to a complete ablation of transcription or expression.

“Reducing” as used herein in relation to transcription or expression of a gene or protein refers to a reduction in gene transcription or expression, such as at least a 50% reduction of transcription or expression. In one embodiment, the reduction is at least a 60%, at least a 70%, at least an 80% reduction. In one embodiment, the reduction is at least an 85% reduction. In one embodiment, the reduction is at least a 90% reduction. In one embodiment, the reduction is at least a 95% reduction. In one embodiment, the reduction is at least a 97% reduction. In one embodiment, the reduction is at least a 99% reduction. In one embodiment, the reduction is a 100% reduction.

As used herein, with respect to treatment methods, “prevention” includes a reducing of the risk of contracting the disease. The “treatment or prevention” may be complete or partial treatment or prevention, i.e. a reduction, but not complete reduction of the disease/condition or symptoms thereof; or a reducing of the risk but not total prevention of the disease/condition or a symptom thereof. Similarly, the methods treat or prevent (i.e. reduces the risk of) an undesirable symptom of the disease or condition or the therapy.

A “therapeutic metabolite” refers to a molecule that is able to act in the local environment of the donor or recipient cell to provide a therapeutic effect. This may be by, for example, counteracting the presence of another detrimental molecule or causing a downstream reaction which provides the therapeutic effect.

A “transmissible element” as used herein refers to any means which can be transferred to a recipient bacterium, including, but not limited to a plasmid, a conjugative plasmid, a phage or a phagemid. In some embodiments, a transmissible element may include other mobile genetic elements, such as transposons and the like. In a particular embodiment, the transmissible element is selected from a conjugative plasmid, and phage and a phagemid. In another particular embodiment, the transmissible element is a conjugative plasmid.

Any percentage identity herein may be at least (about) 70%. For example, any percentage identity herein may be at least (about) 80%. For example, any percentage identity herein may be at least (about) 90%. For example, any percentage identity herein may be at least (about) 95%. For example, any percentage identity herein may be at least (about) 96%. For example, any percentage identity herein may be at least (about) 97%. For example, any percentage identity may be at least (about) 98%. For example, any percentage identity herein may be at least (about) 99%. Percent identity for amino acid sequences are determined using the blastP algorithm with the following parameters:—

The default parameters are adjusted for short input sequences, the expect threshold is set at 0.05 and the length of the seed sequence that initiates an alignment is set at 6. Regions of low compositional complexity are masked. The employed scoring matrix is ‘BLOSUM62’, with scoring costs to create and extend a gap being 11 and 1 respectively. Conditional compositional score matrix adjustment is employed to compensate for amino acid composition of compared sequences.

Percent identity for nucleotide sequences are determined using the blastn algorithm with the following parameters:—

The default parameters are automatically adjusted for short input sequences, the expect threshold is set at 0.05 and the length of the seed sequence that initiates an alignment is set at 28. Regions of low compositional complexity are masked. The query sequence is masked while producing seed sequences used to scan databases, but not masked for extensions. Matches are scored as +1 and mismatches are scored as −2.

For example, an amino acid sequence described herein is identical to the reference SEQ ID No, except for 1, 2, 3, 4, 5, 6, 7, 8, 9 or 10 amino acid changes, in particular 1 to 5, for example 1 to 3, such as 1 or 2, e.g. 1 amino acid change. For example, a nucleic acid sequence described herein is identical to the reference SEQ ID No, except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide changes.

For example, an amino acid sequence described herein is identical to the reference SEQ ID No except for a total number of amino acid changes wherein the total number is no more than (about) 5, 10, 15, 20, 25 or 30% of the number of amino acids in the reference sequence. For example, a nucleotide sequence described herein is identical to the reference SEQ ID No except for a total number of nucleotide changes wherein the total number is no more than (about) 5, 10, 15, 20, 25 or 30% of the number of nucleotides in the reference sequence.

### Conjugative Plasmids

In particular embodiments, the transmissible element is a conjugative plasmid. A conjugative plasmid is a plasmid which, when comprised within a bacterial cell (“donor” or “host” cell) is able to be transferred to another bacterium (“recipient” cell) through the mechanism of bacterial conjugation. Bacterial conjugation is the unidirectional and horizontal transmission of genetic information (“horizontal gene transfer”) from one bacterium to another. Conjugative plasmids generally fall into two classes: mobilizable plasmids and self-transmissible plasmids.

In any embodiment, the conjugative plasmid is capable of being transferred to a recipient bacterial cell. In any embodiment, the conjugative plasmid is transferred to a bacterial cell (i.e. a recipient cell). The recipient cell may be any bacterial cell described elsewhere herein (e.g. a gram-negative bacterial cell).

There is provided a conjugative plasmid for transmission to a recipient bacterium which comprises A) at least one exogenous nucleic acid sequence for the production of a first molecule of interest (MOI) (as described elsewhere herein) in the recipient bacterium, and B) a PMS (as described elsewhere herein) which encodes a nucleic acid modifier or a peptide molecule to modulate a target endogenous nucleic acid or protein for the production or consumption (or degradation) of the first MOI in the recipient bacterium.

The conjugative plasmids described herein and donor (host) cells comprising such conjugative plasmids are useful as medicaments.

There is provided a donor (host) cell comprising a conjugative plasmid as described herein. The donor (host) cell may be any bacterial cell described elsewhere herein (e.g. a gram-negative bacterial cell).

Mobilizable plasmids comprise at least an origin of transfer (oriT), a relaxase and other genetic information on the plasmid which is transferred to the recipient cell. They require helper functions provided by e.g. a second plasmid or the chromosome of the donor cell to effect the plasmid transfer. In one embodiment, the conjugative plasmid is a mobilizable plasmid. In one embodiment, the conjugative plasmid is a mobilizable plasmid comprising an origin of transfer (oriT) and a relaxase.

A self-transmissible plasmid, in addition to the genetic information on the plasmid which is transferred to the recipient cell, also contain all the molecular machinery needed for self-transfer (e.g. for pilus formation and initiation of gene transfer) on the same plasmid. In one embodiment, the conjugative plasmid is a self-transmissible plasmid. In one embodiment, the conjugative plasmid is a self-transmissible plasmid which comprises all of the molecular machinery necessary for self-transfer. In one embodiment, the conjugative plasmid comprises an oriT and encodes all proteins required to mobilise the plasmid for conjugative transfer between cells.

Engineered conjugative plasmids are described in more detail in WO2021/037732 (SNIPR Biome ApS), which is incorporated herein in its entirety. The features of such conjugative plasmids and bacterial cells comprising them as described in the claims as filed in WO2021/037732 are also incorporated herein by reference.

Thus, in one embodiment, the conjugative plasmid is devoid of a hypC2 nucleotide sequence, or a homologue thereof, for example a hypC2 nucleotide sequence of Seq ID No:78. The conjugative plasmid may comprise an OriT of an IncX plasmid. The conjugative plasmid may be an IncX plasmid. The conjugative plasmid may be a β10 plasmid.

The conjugative plasmid (or any other plasmid described herein) may be a plasmid based on any plasmid found in a bacterium disclosed herein. For example, the plasmid may be an Enterobacteriaceae plasmid. In one embodiment, the plasmid is an E. coli, Klebsiella, Saknonella, Erwinia, Shigella, Pantoea, Proteus or Citrobacter plasmid. In one embodiment, the plasmid may be from a genera selected from Bifidobacterium, Bacteroides, Lactobacillus, Lacticaseibacillus, Lactiplantbacillus, Levilactobacillus, Ligilactobacillus, Limosilactobacillus and Lactococcus. In one embodiment, the plasmid may be from a strain belonging to a genera selected from a Bifidobacterium genus or a Bacteroides genus. In one embodiment, the genera is a Bacteroides genus. In one embodiment, the genera is a Bifidobacterium genus. In one embodiment, the plasmid may be from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus brevis, Ligilactobacillus salivarius, Limosilactobacillus reuteri and Lactococcus lactis. In one embodiment, the plasmid may be from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the plasmid may be from a genus or species disclosed in Table 2.

In an example, the conjugative plasmid is capable of replicating in a bacterial cell from any bacterial genus or species described herein. For example, the conjugative plasmid is capable of replicating in an E. coli, Klebsiella, Salmonella, Erwinia, Shigella, Pantoea, Proteus or Citrobacter host (donor) cell. In one embodiment, the conjugative plasmid is capable of replicating in a bacterial cell from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the conjugative plasmid is capable of replicating in a bacterial cell from a strain belonging to a genera selected from a Bifidobacterium genus or a Bacteroides genus. In one embodiment, the genera is a Bacteroides genus. In one embodiment, the genera is a Bifidobacterium genus. In one embodiment, the conjugative plasmid is capable of replicating in a bacterial cell from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus breves, Ligilactobacillus salivarius, Limosilactobacillus reuteri and Lactococcus lactis. In one embodiment, the conjugative plasmid is capable of replicating in a bacterial cell from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the conjugative plasmid is capable of replicating in a bacterial cell from a genus or species disclosed in Table 2Table 2.

In one embodiment the conjugative plasmid (or any other plasmid described herein) is capable of being hosted in an Enterobacteriaceae cell. In one embodiment, the plasmid is capable of being hosted in an E. coli, Klebsiella, Salmonella, Erwinia, Shigella, Pantoea, Proteus or Citrobacter cell. In one embodiment, the plasmid is capable of being hosted in a bacterial cell from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the plasmid is capable of being hosted in a bacterial cell from a strain belonging to a genera selected from a Bifidobacterium genus or a Bacteroides genus. In one embodiment, the genera is a Bacteroides genus. In one embodiment, the genera is a Bifidobacterium genus. In one embodiment, the plasmid is capable of replicating in a bacterial cell from a species which is selected from Bifidobacterium longum, Bifidobacterium breve, Bifidobacterium, adolescentis, Bacteroides uniformis, Bacteroides vulgatus, Bacteroides thetaiotaomicron, Lactobacillus gasseri, Lacticaseibacillus paracasei, Lactoplantibacillus plantarum, Levilactobacillus brevis, Ligilactobacillus salivarius, Limosilactobacillus reuteri and Lactococcus lactis. In one embodiment, the plasmid is capable of being hosted in a bacterial cell from a species which is selected from Bifidobacterium longum, Bifidobacterium breve, Bifidobacterium adolescentis, Bacteroides uniforms, Bacteroides vulgatus and Bacteroides thetaiotaomicron. In one embodiment, the plasmid is capable of being hosted in a bacterial cell from a genus or species disclosed in Table 2Table 2.

In one embodiment, the conjugative plasmid is capable of being conjugatively transferred to an Enterobacteriaceae cell. In one embodiment, the plasmid is capable of being conjugatively transferred to an E. coli, Klebsiella, Salmonella, Erwinia, Shigella, Pantoea, Proteus or Citrobacter cell. In one embodiment, the conjugative plasmid is capable of being conjugatively transferred to a bacterial cell from a genera selected from Bifidobacterium, Bacteroides, Lactobacillus, Lacticaseibacillus, Lactiplantibacillus, Levilactobacillus, Ligilactobacillus, Limosilactobacillus and Lactococcus. In one embodiment, the conjugative plasmid is capable of being conjugatively transferred to in a bacterial cell from a strain belonging to a genera selected from a Bifidobacterium genus or a Bacteroides genus. In one embodiment, the genera is a Bacteroides genus. In one embodiment, the genera is a Bifidobacterium genus. In one embodiment, the conjugative plasmid is capable of being conjugatively transferred to a bacterial cell from a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus breves, Ligilactobacillus salivarius, Limosilactobacillus reuteri and Lactococcus lactis. In one embodiment, the conjugative plasmid is capable of being conjugatively transferred to a bacterial cell from a species which is selected from Bifidobacterium longum, Bifidobacterium breve, Bifidobacterium adolescentis, Bacteroides uniformis, Bacteroides vulgatus and Bacteroides thetaiotaomicron. In one embodiment, the conjugative plasmid is capable of being conjugatively transferred to a bacterial cell from a genus or species disclosed in Table 2Table 2.

The conjugative plasmid may be based on a bacterial conjugative plasmid selected from one of the following bacterial conjugative plasmid families: IncA, IncB/O (Inc10), IncC, IncD, IncE, IncF1, IncF2, IncG, IncHI1, IncHI2, Incl1, Incl2, IncJ, 10 IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and Inc18.

In one embodiment, the conjugative plasmid is a conjugative plasmid isolated from E. coli. In another embodiment, the conjugative plasmid is a β10 conjugative plasmid from E. coli (e.g. as shown in Example 3 herein).

In one embodiment, the donor (host) cell comprising a conjugative plasmid as described herein is an E. coli (such as a symbioflor E. coli, e.g. G6/7) host cell (e.g. as shown in Example 3 herein). In another embodiment, the donor (host) cell comprising a conjugative plasmid as described herein is an E. hormaechei host cell (e.g. as shown in Example 3 herein).

In one embodiment, the recipient cell is an E. coli recipient strain comprising a conjugative plasmid as described herein. The plasmid is introduced via conjugation from a donor (host) cell described herein (e.g. as shown in Example 3 herein). The E. coli recipient strain may be comprised by a native microbiome, e.g. in the gut of a subject. In another embodiment, the recipient cell is a Klebsiella recipient strain comprising a conjugative plasmid as described herein. The plasmid is introduced via conjugation from a donor (host) cell described herein (e.g. as shown in Example 3 herein). The Klebsiella recipient strain may be comprised by a native microbiome, e.g. in the gut of a subject.

In any embodiment described herein, the transmissible element may be capable of being introduced into the recipient bacterium without heat stock. In any embodiment described herein, the transmissible element may be capable of being introduced into a recipient bacterium without electroporation. In any embodiment described herein, the transmissible element may be capable of being introduced into a recipient bacterium without in vitro transformation techniques, including chemical induction (e.g. using calcium).

In one particular embodiment, there is provided a donor (host) bacterium comprising a conjugative plasmid as described elsewhere herein. In another particular embodiment, there is provided a recipient bacterium comprising a conjugative plasmid as described elsewhere herein.

### Alternative Transmissible Elements

As an alternative, the transmissible element is delivered by a phage particle. In one embodiment, the transmissible element is a phage or a phagemid (e.g. a phagemid which is packaged in a phage particle). Thus, there is provided a phage particle comprising a transmissible element as described elsewhere herein. In one embodiment, the phage chromosome comprises a transmissible element as described elsewhere herein. In another embodiment, the phage particle comprises a plasmid (e.g. a phagemid) which comprises a transmissible element as described elsewhere herein.

The at least one exogenous nucleic acid sequence to the recipient bacterium for the production of a first MOI and the PMS which encodes a nucleic acid modifier or a peptide molecule may be delivered by a phage particle. Thus, there is a provided a phage particle for transmission to a recipient bacterium comprising A) at least one exogenous nucleic acid sequence for the production of a first molecule of interest (MOI) (as described elsewhere herein) in the recipient bacterium, and B) a PMS (as described elsewhere herein) which encodes a nucleic acid modifier or a peptide molecule to modulate a target endogenous nucleic acid or protein for the production or consumption (or degradation) of the first MOI in the recipient bacterium.

In one embodiment, the at least one exogenous nucleic acid sequence for the production of a first molecule of interest (MOI) in the recipient bacterium and the PMS which encodes a nucleic acid modifier or a peptide molecule to modulate a target endogenous nucleic acid or protein for the production or consumption (or degradation) of the first MOI in the recipient bacterium may be comprised by a phagemid within a phage particle. Thus, there is provided a phagemid comprised within a phage particle for transmission to a recipient bacterium, wherein the phagemid comprises A) at least one exogenous nucleic acid sequence for the production of a first molecule of interest (MOI) (as described elsewhere herein) in the recipient bacterium, and B) a PMS (as described elsewhere herein) which encodes a nucleic acid modifier or a peptide molecule to modulate a target endogenous nucleic acid or protein for the production or consumption (or degradation) of the first MOI in the recipient bacterium. There is provided a recipient bacterium which comprises a transmissible element which is a plasmid which has been introduced by a packaged phagemid (i.e. a phagemid packaged within a phage particle). There is also provided a host bacterium comprising a transmissible element which is a phagemid and is used for the production of a packaged phagemid (i.e. a phagemid packaged within a phage particle).

There is also provided a plasmid comprised within a phage particle for transmission to a recipient bacterium, wherein the plasmid comprises A) at least one exogenous nucleic acid sequence for the production of a first molecule of interest (MOI) (as described elsewhere herein) in the recipient bacterium, and B) a PMS (as described elsewhere herein) which encodes a nucleic acid modifier or a peptide molecule to modulate a target endogenous nucleic acid or protein for the production or consumption (or degradation) of the first MOI in the recipient bacterium. There is provided a recipient bacterium which comprises a transmissible element which is a plasmid which has been introduced by a phage. There is also provided a host bacterium comprising a transmissible element which is a plasmid comprised within a phage particle which is used for the production of said phage.

In another embodiment, the at least one exogenous nucleic acid sequence for the production of a first molecule of interest (MOI) in the recipient bacterium and the PMS which encodes a nucleic acid modifier or a peptide molecule to modulate a target endogenous nucleic acid or protein for the production or consumption (or degradation) of the first MOI in the recipient bacterium may be comprised in the chromosome of the phage particle. Thus, there is provided a phage for transmission to a recipient bacterium, wherein the phage chromosome comprises A) at least one exogenous nucleic acid sequence for the production of a first molecule of interest (MOI) (as described elsewhere herein) in the recipient bacterium, and B) a PMS (as described elsewhere herein) which encodes a nucleic acid modifier or a peptide molecule to modulate a target endogenous nucleic acid or protein for the production or consumption (or degradation) of the first MOI in the recipient bacterium. There is provided a recipient bacterium which comprises a transmissible element (as described elsewhere herein) which is a prophage which has been introduced by a phage. There is also provided a host bacterium comprising a transmissible element (as described elsewhere herein) comprised by the chromosome of a phage which is used for the production of said phage.

The phage chromosome, plasmid or the phagemid may further comprise a kill switch as described elsewhere herein.

Phages that may be used as transmissible elements herein may be as described below.

The phage may be a tail phage.

The tail phage may be a Faecalibacterium tail phage. Examples of Faecalibacterium tail phages include Faecalibacterium phage FP_Brigit, Faecalibacterium phage FP_Epona, Faecalibacterium phage FP_Lagaffe, Faecalibacterium phage FP_Lugh, Faecalibacterium phage FP_Mushu, Faecalibacterium phage FP_Oengus, Faecalibacterium phage FP_Taranis and Faecalibacterium phage FP_Toutatis.

The tail phage may be a Bacteriodes tail phage. Examples of Bacteroides tail phages include Bacteroides phage crAss002, Bacteroides phage crAss001, Bacteroides phage DAC15 and Azobacteroides phage ProJPt-Bp1.

The tail phage may be a Bifidobacterium tail phage. Examples of Bifidobacterium tail phages include Bifidobacterium phage BadAargau2 and Bifidobacterium phage BadAztec1.

The tail phage may be a Clostridium tail phage. Examples of Clostridium tail phages include Clostridium phage CpV1, Clostridium phage CPD2, Clostridium phage CPS1, Clostridium phage phi24R, Clostridium phage phiCP7R, Clostridium phage CPS2, Clostridium phage phiCPV4, Clostridium phage phiZP2, Clostridium phage susfortuna, Clostridium phage phiCD505, Clostridium phage CDKM9, Clostridium phage CDKM15, Clostridium phage phiMMPO2, Clostridium phage phiCD111, Clostridium phage phiCD146, Clostridium phage phiCD38-2, Clostridium phage phi CD119, Clostridium phage phiCDHM19, Clostridium phage phiCD506, Clostridium phage phiCD481-1, Clostridium phage phiCDHM11, Clostridium phage phiCDHM13, Clostridium phage phiCDHM14, Clostridium phage phiMMPO4, Clostridium phage CDMH1, Clostridium phage phiMMP01, and Clostridium phage phiMMP03.

The tail phage may be a Enterococcus tail phage. Examples of Enterococcus tail phages include Enterococcus phage ECP3, Enterococcus phage EF24C, Enterococcus phage phiEF24C-P2, Enterococcus phage EFLK1, Enterococcus phage EFDG1, Enterococcus phage EFP01, Enterococcus phage EN12-phi1, Enterococcus phage AE4_17, Enterococcus phage vB_EfaP_Ef6.2, Enterococcus phage vB_EfaP_Ef6.3, Enterococcus phage vB_EfaP_Ef7.2, Enterococcus phage vB_EfaP_Ef7.3, Enterococcus phage vB_EfaP_Ef7.4, Enterococcus phage vB_Efae230P-4, Enterococcus phage vB_EfaP_Efmus1, Enterococcus phage vB_EfaP_Efmus3, Enterococcus phage vB_EfaP_Efmus4, Enterococcus phage Idefix, Enterococcus phage vB_EfaP_IME195, Enterococcus phage vB_EfaP_IME199, Enterococcus phage vB_EfaP_Zip, Enterococcus phage 9183, Enterococcus phage nattely, Enterococcus phage vipetofem, Enterococcus phage vB_EfaS_140, Enterococcus phage vB_EfaS_AL2, Enterococcus phage vB_EfaS_AL3, Enterococcus phage AUEF3, Enterococcus phage Ec-ZZ2, Enterococcus phage IME_EF3, Enterococcus phage IME-EF4, Enterococcus phage EfaCPT1, Enterococcus phage vB_EfaS_IME196, Enterococcus phage LY0322, Enterococcus phage PMBT2, Enterococcus phage SANTORI, Enterococcus phage phiSHEF2, Enterococcus phage phiSHEF4, Enterococcus phage phiSHEF5, Enterococcus phage phiFL1A, Enterococcus phage phiFL2A, Enterococcus phage phiFL3A, Enterococcus phage BC611, Enterococcus phage IME-EF1 and Enterococcus phage SAP6.

The tail phage may be a Klebsiella tail phage. Examples of Klebsiella tail phages include Klebsiella phage 0507-KN2-1, Klebsiella phage 13, Klebsiella phage 1513, Klebsiella phage 2044-307w, Klebsiella phage 3LV2017, Klebsiella phage 4LV2017, Klebsiella phage AltoGao, Klebsiella phage F19, Klebsiella phage GH-K3, Klebsiella phage GML-KpCol1, Klebsiella phage Henu1, Klebsiella phage JD001, Klebsiella phage JD18, Klebsiella phage JY917, Klebsiella phage K11, Klebsiella phage K5, Klebsiella phage K5-2, Klebsiella phage K5-4, Klebsiella phage K64-1, Klebsiella phage KL, Klebsiella phage KLPN1, Klebsiella phage KN1-1, Klebsiella phage KN3-1, Klebsiella phage KN4-1, Klebsiella phage KNP2, Klebsiella phage KOX1, Klebsiella phage KP-Rio/2015, Klebsiella phage KP15, Klebsiella phage KP179, Klebsiella phage KP1801, Klebsiella phage Kp2, Klebsiella phage KP27, Klebsiella phage KP32, Klebsiella phage KP32_isolate 192, Klebsiella phage KP32_isolate 194, Klebsiella phage KP32_isolate 195, Klebsiella phage KP32_isolate 196, Klebsiella phage KP34, Klebsiella phage KP36, Klebsiella phage KP8, Klebsiella phage KpCHEMY26, Klebsiella phage KpKT21phi1, Klebsiella phage KPN N137, Klebsiella phage KPN N141, Klebsiella phage KpS8, Klebsiella phage kpssk3, Klebsiella phage KPV15, Klebsiella phage KpV41, Klebsiella phage KpV475, Klebsiella phage KpV71, Klebsiella phage KPV811, Klebsiella phage LASTA, Klebsiella phage Magnus, Klebsiella phage Marfa, Klebsiella phage Matisse, Klebsiella phage May, Klebsiella phage Menlow, Klebsiella phage MezzoGao, Klebsiella phage Mineola, Klebsiella phage myPSH1235, Klebsiella phage NJS1, Klebsiella phage NTUH-K2044-K1-1, Klebsiella phage Pharr, Klebsiella phage phiBO1E, Klebsiella phage PhiKpNIH-2, Klebsiella phage phiKpS2, Klebsiella phage PKO111, Klebsiella phage PKP126, Klebsiella phage Pylas, Klebsiella phage Seifer, Klebsiella phage SH-Kp 152234, Klebsiella phage SH-Kp 152410, Klebsiella phage Shelby, Klebsiella phage Sin4, Klebsiella phage Skenny, Klebsiella phage Soft, Klebsiella phage SopranoGao, Klebsiella phage ST13-OXA48phi12.1, Klebsiella phage ST147-VIM1phi7.1, Klebsiella phage ST15-OXA48phi14.1, Klebsiella phage ST16-OXA48phi5.4, Klebsiella phage ST437-OXA245phi4.1, Klebsiella phage ST437-OXA245phi4.2, Klebsiella phage ST512-KPC3phi13.2, Klebsiella phage Sugarland, Klebsiella phage Sushi, Klebsiella phage Sweeny, Klebsiella phage TAH8, Klebsiella phage TSK1, Klebsiella phage UPM 2146, Klebsiella phage vB_KIeM_RaK2, Klebsiella phage vB_Kp1, Klebsiella phage vB_Kpn_F48, Klebsiella phage vB_Kpn_IME260, Klebsiella phage vB_KpnM_BIS47, Klebsiella phage vB_KpnM_KB57, Klebsiella phage vB_KpnM_KpS110, Klebsiella phage vB_KpnM_KpV477, Klebsiella phage vB_KpnM_KpV52, Klebsiella phage vB_KpnM_KpV79, Klebsiella phage vB_KpnP_BIS33, Klebsiella phage vB_KpnP_IL33, Klebsiella phage vB_KpnP_IME205, Klebsiella phage vB_KpnP_KpV289, Klebsiella phage vB_KpnP_KpV48, Klebsiella phage vB_KpnP_KpV74, Klebsiella phage vB_KpnP_KpV763, Klebsiella phage vB_KpnP_KpV766, Klebsiella phage vB_KpnP_KpV767, Klebsiella phage vB_KpnP_P184, Klebsiella phage vB_KpnP_PRA33, Klebsiella phage vB_KpnP_SU503, Klebsiella phage vB_KpnP_SU552A, Klebsiella phage vB_KpnS_15-38_KLPPOU149, Klebsiella phage vB_KpnS_Alina, Klebsiella phage vB_KpnS_Call, Klebsiella phage vB_KpnS_Domnhall, Klebsiella phage vB_KpnS_FZ10, Klebsiella phage vB_KpnS_IME279, Klebsiella phage vB_KpnS_IMGroot, Klebsiella phage vB_KpnS_KpV522, Klebsiella phage vB_KpnS_SegesCirculi, Klebsiella phage vB_KpP_FBKp27, Klebsiella phage YMC16/01/N133_KPN_BP, Klebsiella phage ZCKP1, Klebsiella phage_vB_KpnP_IME321 and Klebsiella pneumoniae strain E16KP0115 plasmid unnamed.

The tail phage may be a Fusabacterium tail phage. An example of a Fusobacterium tail phage is Fusobacterium phage FNU1.

The tail phage may be a Lactobacillus phage. Examples of Lactobacillus tail phages include Lactobacillus phage 3-521, Lactobacillus phage 521B, Lactobacillus phage A2, Lactobacillus phage ATCC 8014-B1, Lactobacillus phage ATCC 8014-B2, Lactobacillus phage Bacchae, Lactobacillus phage BH1, Lactobacillus phage Bromius, Lactobacillus phage c2, Lactobacillus phage c5, Lactobacillus phage CL1, Lactobacillus phage CL2, Lactobacillus phage iA2, Lactobacillus phage Iacchus, Lactobacillus phage ilp1308, Lactobacillus phage iLp84, Lactobacillus phage J-1, Lactobacillus phage Lb, Lactobacillus phage Lb338-1, Lactobacillus phage LBR48, Lactobacillus phage Lc-Nu, Lactobacillus phage Ld17, Lactobacillus phage Ld25A, Lactobacillus phage Ld3, Lactobacillus phage Ldl1, Lactobacillus phage Lenus, Lactobacillus phage LF1, Lactobacillus phage LfeInf, Lactobacillus phage U, Lactobacillus phage LL-Ku, Lactobacillus phage LP65, Lactobacillus phage Lpa804, Lactobacillus phage LpeD, Lactobacillus phage Lrm1, Lactobacillus phage Maenad, Lactobacillus phage Nyseid, Lactobacillus phage P1, Lactobacillus phage PhiAT3, Lactobacillus phage phiJL-1, Lactobacillus phage phiLdb, Lactobacillus phage PL-1, Lactobacillus phage PLE2, Lactobacillus phage PLE3, Lactobacillus phage SA-C12, Lactobacillus phage SAC12B, Lactobacillus phage Satyr, Lactobacillus phage Semele, Lactobacillus phage T25 and Lactobacillus phage ViSo-2018a.

The tail phage may be a Escherichia tail phage. Examples of Escherichia tail phages include Escherichia coli 0157 typing phage 3, Escherichia coli strain 13P477T plasmid p13P477T-2, Escherichia phage 121Q, Escherichia phage 13a, Escherichia phage 172-1, Escherichia phage 186, Escherichia phage 1H12, Escherichia phage 285P, Escherichia phage 2B8, Escherichia phage 2G7b, Escherichia phage 2H10, Escherichia phage 4A7, Escherichia phage 4MG, Escherichia phage 500465-1, Escherichia phage 500465-2, Escherichia phage 503458, Escherichia phage 520873, Escherichia phage 64795_ec1, Escherichia phage 933W, Escherichia phage aalborv, Escherichia phage AAPEc6, Escherichia phage aaroes, Escherichia phage ADB-2, Escherichia phage alia, Escherichia phage alpha3, Escherichia phage anhysbys, Escherichia phage AnYang, Escherichia phage APCEc01, Escherichia phage APECc02, Escherichia phage AR1, Escherichia phage ArgO145, Escherichia phage atuna, Escherichia phage Av-05, Escherichia phage BA14, Escherichia phage BF23, Escherichia phage Bp4, Escherichia phage Bp7, Escherichia phage C1, Escherichia phage C119, Escherichia phage C130_2, Escherichia phage C5, Escherichia phage CAjan, Escherichia phage CBA120, Escherichia phage CF2, Escherichia phage chee24, Escherichia phage CICC 80001, Escherichia phage damhaus, Escherichia phage DE3, Escherichia phage DT571/2, Escherichia phage DT57C, Escherichia phage DTL, Escherichia phage E21, Escherichia phage e4/1c, Escherichia phage Ebrios, Escherichia phage EC1-UPM, Escherichia phage EC121, Escherichia phage EC6, Escherichia phage EC6098, Escherichia phage ECA2, Escherichia phage ECBP1, Escherichia phage ECBP2, Escherichia phage ECBP5, Escherichia phage ECD7, Escherichia phage ECML-117, Escherichia phage ECML-134, Escherichia phage ECML-4, Escherichia phage EcNP1, Escherichia phage Eco_BIFF, Escherichia phage ECO4, Escherichia phage EcoDS1, Escherichia phage ECP1, Escherichia phage EcS1, Escherichia phage EG1, Escherichia phage egaa, Escherichia phage EK99P-1, Escherichia phage EP75, Escherichia phage EPS7, Escherichia phage ESCO13, Escherichia phage ESC05, Escherichia phage ESSI2_ev015, Escherichia phage ESSI2_ev040, Escherichia phage ESSI2_ev129, Escherichia phage ESSI2_ev239, Escherichia phage ev017, Escherichia phage ev099, Escherichia phage ev207, Escherichia phage ev243, Escherichia phage F2, Escherichia phage FEC14, Escherichia phage FEC19, Escherichia phage FFH2, Escherichia phage flopper, Escherichia phage fp01, Escherichia phage FV3, Escherichia phage G4, Escherichia phage GA2A, Escherichia phage Gostya9, Escherichia phage grams, Escherichia phage H8, Escherichia phage haarsle, Escherichia phage Henu7, Escherichia phage Henu8, Escherichia phage herni, Escherichia phage HK022, Escherichia phage HK106, Escherichia phage HK446, Escherichia phage HK542, Escherichia phage HK544, Escherichia phage HK578, Escherichia phage HK629, Escherichia phage HK630, Escherichia phage HK633, Escherichia phage HK75, Escherichia phage HK97, Escherichia phage HP3, Escherichia phage HX01, Escherichia phage HY01, Escherichia phage HYO2, Escherichia phage HY03, Escherichia phage HZ2R8, Escherichia phage HZP2, Escherichia phage 122, Escherichia phage ID2 Moscow/ID/2001, Escherichia phage ID21, Escherichia phage ID32, Escherichia phage ID52, Escherichia phage ID62, Escherichia phage If1, Escherichia phage IME08, Escherichia phage ime09, Escherichia phage IME11, Escherichia phage IMM-002, Escherichia phage 38-65, Escherichia phage JES2013, Escherichia phage JH2, Escherichia phage JK06, Escherichia phage JL1, Escherichia phage JLK-2012, Escherichia phage JMPW1, Escherichia phage JMPW2, Escherichia phage JS10, Escherichia phage JS98, Escherichia phage JSE, Escherichia phage K1-5, Escherichia phage K1F, Escherichia phage K1G, Escherichia phage K1H, Escherichia phage K1ind1, Escherichia phage K1ind2, Escherichia phage K30, Escherichia phage KBNP1711, Escherichia phage KrT03, Escherichia phage Lambda, Escherichia phage LL11, Escherichia phage LL2, Escherichia phage LL5, Escherichia phage LM33_P1, Escherichia phage Lw1, Escherichia phage Lyzl2581Vzw, Escherichia phage M13, Escherichia phage Mangalitsa, Escherichia phage mEp234, Escherichia phage mEpX1, Escherichia phage mEpX2, Escherichia phage Min27, Escherichia phage Minoma, Escherichia phage MLF4, Escherichia phage Mt1B1_P17, Escherichia phage Mu, Escherichia phage Murica, Escherichia phage mutPK1A2, Escherichia phage muut, Escherichia phage MX01, Escherichia phage N15, Escherichia phage N30, Escherichia phage N4, Escherichia phage NC-A, Escherichia phage NC28, Escherichia phage NC29, Escherichia phage NC35, Escherichia phage nepoznato, Escherichia phage nieznany, Escherichia phage NJ01, Escherichia phage OSYSP, Escherichia phage p000v, Escherichia phage P1, Escherichia phage P2, Escherichia phage P2_2H1, Escherichia phage P2_2H4, Escherichia phage P2_4A7b, Escherichia phage P2_482, Escherichia phage P2_4C9, Escherichia phage P2_4E6b, Escherichia phage P483, Escherichia phage P694, Escherichia phage P88, Escherichia phage P88_4B11, Escherichia phage PA2, Escherichia phage PA28, Escherichia phage PBECO4, Escherichia phage PE3-1, Escherichia phage PE37, Escherichia phage PGN590, Escherichia phage PGT2, Escherichia phage phAPEC8, Escherichia phage PhaxI, Escherichia phage Phi1, Escherichia phage phi191, Escherichia phage phi92, Escherichia phage phiAPCEc03, Escherichia phage phiC120, Escherichia phage phiE142, Escherichia phage phiEB49, Escherichia phage phiEco32, Escherichia phage phiEcoM-GJ1, Escherichia phage phiK, Escherichia phage phiKP26, Escherichia phage phiKT, Escherichia phage phiLLS, Escherichia phage phiSUSP1, Escherichia phage phiSUSP2, Escherichia phage phiV10, Escherichia phage phiX174, Escherichia phage phT4A, Escherichia phage Pollock, Escherichia phage PP01, Escherichia phage prol47, Escherichia phage pro483, Escherichia phage PTXUO4, Escherichia phage Qbeta, Escherichia phage QL01, Escherichia phage RB14, Escherichia phage RB16, Escherichia phage RB3, Escherichia phage RB32, Escherichia phage RB43, Escherichia phage RB49, Escherichia phage RB69, Escherichia phage RCS47, Escherichia phage Ro451w, Escherichia phage Roguel, Escherichia phage Rtp, Escherichia phage saus132, Escherichia phage SECphi27, Escherichia phage Seurat, Escherichia phage SF, Escherichia phage SH2026Stx1, Escherichia phage Skarpretter, Escherichia phage slur02, Escherichia phage slur03, Escherichia phage slur04, Escherichia phage slur07, Escherichia phage slur09, Escherichia phage slurl6, Escherichia phage Sortsne, Escherichia phage SP15, Escherichia phage SRT7, Escherichia phage SRT8, Escherichia phage SSL-2009a, Escherichia phage St-1, Escherichia phage ST31, Escherichia phage ST32, Escherichia phage Stx2 II, Escherichia phage T1, Escherichia phage T2, Escherichia phage T4, Escherichia phage T3, Escherichia phage T7, Escherichia phage teqdroes, Escherichia phage teqhad, Escherichia phage teqskov, Escherichia phage tiwna, Escherichia phage TL-2011c, Escherichia phage Tls, Escherichia phage tonijn, Escherichia phage tonn, Escherichia phage tonnikala, Escherichia phage tuntematon, Escherichia phage tunus, Escherichia phage UFV-AREG1, Escherichia phage ukendt, Escherichia phage V18, Escherichia phage V5, Escherichia phage vB_Eco_mar001J1, Escherichia phage vB_Eco_mar003J3, Escherichia phage vB_Eco_mar004NP2, Escherichia phage vB_Eco_SLUR29, Escherichia phage vB_Eco_swan01, Escherichia phage vB_EcoM_005, Escherichia phage vB_EcoM_112, Escherichia phage vB_EcoM_ACG-C40, Escherichia phage vB_EcoM_Aff5, Escherichia phage vB_EcoM_AYO145A, Escherichia phage vB_EcoM_DalCa, Escherichia phage vB_EcoM_EC01230-10, Escherichia phage vB_EcoM_EC0078, Escherichia phage vB_EcoM_F1, Escherichia phage vB_EcoM_G4498, Escherichia phage vB_EcoM_G4507, Escherichia phage vB_EcoM_G50, Escherichia phage vB_EcoM_G8, Escherichia phage vB_EcoM_G9062, Escherichia phage vB_EcoM_Goslar, Escherichia phage vB_EcoM_IME537, Escherichia phage vB_EcoM_JS19, Escherichia phage vB_EcoM_KAW1E185, Escherichia phage vB_EcoM_KWBSE43-6, Escherichia phage vB_EcoM_Lutter, Escherichia phage vB_EcoM_NBG2, Escherichia phage vB_EcoM_OE5505, Escherichia phage vB_EcoM_Ozark, Escherichia phage vB_EcoM_PhAPEC2, Escherichia phage vB_EcoM_PHB05, Escherichia phage vB_EcoM_Schickermooser, Escherichia phage vB_EcoM_VR20, Escherichia phage vB_EcoM_VR25, Escherichia phage vB_EcoM_VR26, Escherichia phage vB_EcoM_VR7, Escherichia phage vB_EcoM_WFC, Escherichia phage vB_EcoM_WFH, Escherichia phage vB_EcoM-12474III, Escherichia phage vB_EcoM-4HA13, Escherichia phage vB_EcoM-ep3, Escherichia phage vB_EcoM-fFiEco06, Escherichia phage vB_EcoM-G28, Escherichia phage vB_EcoM-Ro121c4YLVW, Escherichia phage vB_EcoM-UFV13, Escherichia phage vB_EcoM-VpaEl, Escherichia phage vb_EcoM-VR5, Escherichia phage vB_EcoP_248, Escherichia phage vB_EcoP_ACG-C91, Escherichia phage vB_EcoP_B, Escherichia phage vB_EcoP_C, Escherichia phage vB_EcoP_F, Escherichia phage vB_EcoP_G7C, Escherichia phage vB_EcoP_K, Escherichia phage vB_EcoP_PhAPEC5, Escherichia phage vB_EcoP_PhAPEC7, Escherichia phage vB_EcoP_S523, Escherichia phage vB_EcoP_SU10, Escherichia phage vB_EcoS Sa1791w, Escherichia phage vB_EcoS_ACG-M12, Escherichia phage vB_EcoS_AHP42, Escherichia phage vB_EcoS_AHS24, Escherichia phage vB_EcoS_AKFV33, Escherichia phage vB_EcoS_AKS96, Escherichia phage vB_Ecos_CEB_EC3a, Escherichia phage vB_EcoS_ESCO41, Escherichia phage vB_EcoS_FFH_1, Escherichia phage vB_EcoS_G29-2, Escherichia phage vB_EcoS_HdH2, Escherichia phage vB_EcoS_IME347, Escherichia phage vB_EcoS_IME542, Escherichia phage vB_EcoS_NBD2, Escherichia phage vB_EcoS_PHB17, Escheichia phage vB_EcoS_SH2, Escherichia phage vB_EcoS_swi2, Escherichia phage vB_EcoS_W011D, Escherichia phage vB_EcoS-12210I, Escherichia phage vB_EcoS-95, Escherichia phage vB_EcoS-DELF2, Escherichia phage vB_EcoS-Golestan, Escherichia phage vB_EcoS-IME253, Escherichia phage vB_vPM_PD06, Escherichia phage vB_vPM_PD112, Escherichia phage VEc3, Escherichia phage VEc33, Escherichia phage VEcB, Escherichia phage vojen, Escherichia phage WA45, Escherichia phage WG01, Escherichia phage Wphi, Escherichia phage wV7, Escherichia phage wV8, Escherichia phage YD-2008.s, Escherichia phage YUEEL01, Escherichia phage YZ1, Escherichia phage ZG49, Escherichia phage_idtsur, Escherichia phage_Vec13, Escherichia typing phage 1 and Escherichia virus KFS-EC.

The tail phage may be a Streptococcus tail phage. Examples of Streptococcus tail phages include Streptococcus phage 01205, Streptococcus phage 2972, Streptococcus phage 7201, Streptococcus phage 858, Streptococcus phage Abc2, Streptococcus phage ALQ13.2, Streptococcus phage C1, Streptococcus phage Cp-7, Streptococcus phage Cp1, Streptococcus phage DT1, Streptococcus phage Sfi11, Streptococcus phage Sfi19, Streptococcus phage Sfi21 and Streptococcus phage SP-QS1.

The tail phage may be a Providencia tail phage. Examples of Providencia tail phages include Providencia phage Kokobel1, Providencia phage PSTCR5, Providencia phage Redjac, Providencia phage vB_PreS_PR1, Providencia phage vB_PreS-PibeRecoleta, Providencia phage vB_PreS-Stilesk and Providencia phage vB_PstP_PS3.

The tail phage may be a Salmonella tail phage. Examples of Salmonella tail phages include

The tail phage may be a Helicobacter tail phage. Examples of Helicobacter tail phages include Helicobacter phage 1961P, Helicobacter phage KHP30 and Helicobacter phage KHP40.

The tail phage may be a Shigella tail phage. Examples of Shigella tail phages include Shigella phage 2019SD1, Shigella phage 75/02 Stx, Shigella phage CM8, Shigella phage DS8, Shigella phage EP23, Shigella phage JK16, Shigella phage JK45, Shigella phage KNP5, Shigella phage MK-13, Shigella phage phi25-307, Shigella phage phiSboM-AG3, Shigella phage POCJ13, Shigella phage pSb-1, Shigella phage pSf-1, Shigella phage pSf-2, Shigella phage pSs-1, Shigella phage Sd1, Shigella phage Sf11 SMD-2017, Shigella phage Sf12, Shigella phage Sf13, Shigella phage Sf14, Shigella phage Sf17, Shigella phage Sf21, Shigella phage Sf22, Shigella phage Sf23, Shigella phage Sf24, Shigella phage Sf6, Shigella phage Sfin-1, Shigella phage Sfin-3, Shigella phage SfMu, Shigella phage SFN6B, Shigella phage SFPH2, Shigella phage SH6, Shigella phage SH7, Shigella phage SHBML-50-1, Shigella phage SHBML-50-1, Shigella phage Shf125875, Shigella phage Shfl1, Shigella phage Shfl2, Shigella phage SHFML-11, Shigella phage SHFML-26, Shigella phage SHSML-45, Shigella phage SHSML-52-1, Shigella phage SP18, Shigella phage Ss-VASD, Shigella phage SSP1, Shigella phage vB_SflS-ISF001, Shigella phage vB_ShiP_A7, Shigella phage vB_SsoS_008, Shigella phage vB_SsoS-ISF002 and Shigella phage_Buco.

The tail phage may be a Pseudomonas tail phage. Examples of Pseudomonas tail phages include Pseudomonas aeruginosa PS75 adTyT-supercont1.7, Pseudomonas phage 119X, Pseudomonas phage 14-1, Pseudomonas phage 17A, Pseudomonas phage 22PfluR64PP, Pseudomonas phage 73, Pseudomonas phage Achelous, Pseudomonas phage Alpheus, Pseudomonas phage Andromeda, Pseudomonas phage antinowhere, Pseudomonas phage B3, Pseudomonas phage Bf7, Pseudomonas phage Bjorn, Pseudomonas phage BrSP1, Pseudomonas phage crassa, Pseudomonas phage D3, Pseudomonas phage D3112, Pseudomonas phage datas, Pseudomonas phage DL60, Pseudomonas phage DL62, Pseudomonas phage DL68, Pseudomonas phage DMS3, Pseudomonas phage Dobby, Pseudomonas phage EL, Pseudomonas phage Epa14, Pseudomonas phage Epa5, Pseudomonas phage EPa61, Pseudomonas phage Epa7, Pseudomonas phage F_HA0480sp/Pa1651, Pseudomonas phage F116, Pseudomonas phage F8, Pseudomonas phage gh-1, Pseudomonas phage H66, Pseudomonas phage Henninger, Pseudomonas phage inbricus, Pseudomonas phage JBD67, Pseudomonas phage JD18, Pseudomonas phage JG004, Pseudomonas phage JG024, Pseudomonas phage KNP, Pseudomonas phage KPP10, Pseudomonas phage KPP12, Pseudomonas phage KPP21, Pseudomonas phage KPP25, Pseudomonas phage Lana, Pseudomonas phage LBL3, Pseudomonas phage LIT1, Pseudomonas phage Littlefix, Pseudomonas phage LKA1, Pseudomonas phage LKD16, Pseudomonas phage LKO4, Pseudomonas phage LMA2, Pseudomonas phage LPB1, Pseudomonas phage LUZ19, Pseudomonas phage LUZ24, Pseudomonas phage LUZ7, Pseudomonas phage M6, Pseudomonas phage MP1412, Pseudomonas phage MP22, Pseudomonas phage MP29, Pseudomonas phage MP38, Pseudomonas phage MPK6, Pseudomonas phage MPK7Pseudomonas phage Nerthus, Pseudomonas phage NH-4, Pseudomonas phage nickie, Pseudomonas phage Njord, Pseudomonas phage Noxifer, Pseudomonas phage NP1, Pseudomonas phage NV1, Pseudomonas phage PA01, Pseudomonas phage PA1/KOR/2010, Pseudomonas phage PA10, Pseudomonas phage PA11, Pseudomonas phage PA26, Pseudomonas phage PA5, Pseudomonas phage PA7, Pseudomonas phage PA8P1, Pseudomonas phage PaBG, Pseudomonas phage PAE1, Pseudomonas phage PaGU11, Pseudomonas phage PAK_P1, Pseudomonas phage PAK_P2, Pseudomonas phage PAK_P3, Pseudomonas phage PAK_P4, Pseudomonas phage PaMx11, Pseudomonas phage PaMx25, Pseudomonas phage PaMx28, Pseudomonas phage PaMx41, Pseudomonas phage PaMx74, Pseudomonas phage PaP1, Pseudomonas phage PaP3, Pseudomonas phage PaP4, Pseudomonas phage PAXYB1, Pseudomonas phage PB1, Pseudomonas phage Persinger, Pseudomonas phage Pf-10, Pseudomonas phage Pf1, Pseudomonas phage Pf1 ERZ-2017, Pseudomonas phage pf16, Pseudomonas phage Pf3, Pseudomonas phage Pf3, Pseudomonas phage pf8_ST274-AUS411, Pseudomonas phage PFP1, Pseudomonas phage phCDa, Pseudomonas phage phi-2, Pseudomonas phage Phi-S1, Pseudomonas phage phi12, Pseudomonas phage phi13, Pseudomonas phage phi15, Pseudomonas phage phi2954, Pseudomonas phage phi3, Pseudomonas phage phi6, Pseudomonas phage phi8, Pseudomonas phage PhiCHU, Pseudomonas phage phiCTX, Pseudomonas phage phiIBB-PAA2, Pseudomonas phage phiIBB-PF7A, Pseudomonas phage phikF77, Pseudomonas phage phiKMV, Pseudomonas phage phiKTN6, Pseudomonas phage phiKZ, Pseudomonas phage phiMK, Pseudomonas phage phiNFS, Pseudomonas phage phiNN, Pseudomonas phage phiNV3, Pseudomonas phage phiPMW, Pseudomonas phage phiPsa17, Pseudomonas phage phiPSA2, Pseudomonas phage phiPsa374, Pseudomonas phage PhiR18, Pseudomonas phage phiYY, Pseudomonas phage PMBT14, Pseudomonas phage PMBT3, Pseudomonas phage PMG1, Pseudomonas phage PollyC, Pseudomonas phage PP7, Pseudomonas phage PPPL-1, Pseudomonas phage PPpW-4, Pseudomonas phage PRR1, Pseudomonas phage PspYZU01, Pseudomonas phage PspYZU05, Pseudomonas phage PspYZU08, Pseudomonas phage PT2, Pseudomonas phage PT5, Pseudomonas phage R12, Pseudomonas phage R26, Pseudomonas phage RLP, Pseudomonas phage sh12, Pseudomonas phage SL1, Pseudomonas phage SL2, Pseudomonas phage SM1, Pseudomonas phage SN, Pseudomonas phage tabemarius, Pseudomonas phage tf, Pseudomonas phage TL, Pseudomonas phage UFV-P2, Pseudomonas phage uligo, Pseudomonas phage UNO-SLW1, Pseudomonas phage vB_Pae_BR319a, Pseudomonas phage vB_Pae_PS44, Pseudomonas phage vB_Pae-Kakheti25, Pseudomonas phage vB_Pae-SS2019X1, Pseudomonas phage vB_PaeM_C1-14_Ab28, Pseudomonas phage vB_PaeM_C2-10_Ab02, Pseudomonas phage vB_PaeM_C2-10_Abl, Pseudomonas phage vB_PaeM_CEB_DP1, Pseudomonas phage vB_PaeM_E215, Pseudomonas phage vB_PaeM_E217, Pseudomonas phage vB_PaeM_G1, Pseudomonas phage vB_PaeM_LS1, Pseudomonas phage vB_PaeM_MAG1, Pseudomonas phage vB_PaeM_PAO1_Ab03, Pseudomonas phage vB_PaeM_PS24, Pseudomonas phage vB_PaeM_SCUT-S1, Pseudomonas phage vB_PaeM_SCUT-S1, Pseudomonas phage vB_PaeM_USP_1, Pseudomonas phage v8_PaeP_130_113, Pseudomonas phage vB_PaeP_C2-10_Ab09, Pseudomonas phage vB_PaeP_C2-10_Ab22, Pseudomonas phage vB_PaeP_PA01_1-15pyo, Pseudomonas phage vB_PaeP_PA01_Ab05, Pseudomonas phage vB_PaeP_PPA-ABTNL, Pseudomonas phage vB_PaeS_PA01_Ab18, Pseudomonas phage vB_PaeS_PA01_Ab19, Pseudomonas phage vB_PaeS_PM105, Pseudomonas phage vB_PaeS_SCH_Ab26, Pseudomonas phage vB_PsyM_KIL1, Pseudomonas phage vB_PsyM_KIL4, Pseudomonas phage vB_PsyP_3MF5, Pseudomonas phage VCM, Pseudomonas phage VSW-3, Pseudomonas phage WRT, Pseudomonas phage YMC11/06/C171_PPU_BP, Pseudomonas phage YuA, Pseudomonas phage ZC01, Pseudomonas phage ZC03, Pseudomonas phage ZC08, Pseudomonas phage Zigelbrucke, Pseudomonas phage Zuri, Pseudomonas sp. S4_EA_1b YA0848_38 and Pseudomonas virus Pa193.

The tail phage may be a Staphylocuccus tail phage. Examples of Staphylocuccus tail phages include Staphylococcus phage 187, Staphylococcus phage 23MRA, Staphylococcus phage 2638A, Staphylococcus phage 29, Staphylococcus phage 3 AJ-2017, Staphylococcus phage 37, Staphylococcus phage 3A, Staphylococcus phage 3MRA, Staphylococcus phage 42e, Staphylococcus phage 47, Staphylococcus phage 52A, Staphylococcus phage 53, Staphylococcus phage 55, Staphylococcus phage 676Z, Staphylococcus phage 69, Staphylococcus phage 6ec, Staphylococcus phage 71, Staphylococcus phage 77, Staphylococcus phage 80, Staphylococcus phage 80alpha, Staphylococcus phage 85, Staphylococcus phage 88, Staphylococcus phage 92, Staphylococcus phage 96, Staphylococcus phage A3R, Staphylococcus phage A5W, Staphylococcus phage Andhra, Staphylococcus phage B122, Staphylococcus phage B166, Staphylococcus phage B236, Staphylococcus phage Baq_Sau1, Staphylococcus phage BP39, Staphylococcus phage CNPH82, Staphylococcus phage CNPx, Staphylococcus phage CSA13, Staphylococcus phage EW, Staphylococcus phage Fi200W, Staphylococcus phage G1, Staphylococcus phage G15, Staphylococcus phage GRCS, Staphylococcus phage Henu2, Staphylococcus phage HSA84, Staphylococcus phage IME-SA1, Staphylococcus phage IME-SA118, Staphylococcus phage IME-SA119, Staphylococcus phage IME-SA2, Staphylococcus phage IME1348_01, Staphylococcus phage IME1361_01, Staphylococcus phage ISP, Staphylococcus phage JD007, Staphylococcus phage JS01, Staphylococcus phage K, Staphylococcus phage LH1, Staphylococcus phage LSA2366, Staphylococcus phage MCE-2014, Staphylococcus phage MSA6, Staphylococcus phage P108, Staphylococcus phage P1105, Staphylococcus phage P240, Staphylococcus phage P282, Staphylococcus phage P4W, Staphylococcus phage P630, Staphylococcus phage P954, Staphylococcus phage Pabna, Staphylococcus phage PH15, Staphylococcus phage phi 11, Staphylococcus phage phi 12, Staphylococcus phage phi 13, Staphylococcus phage phi2958PVL, Staphylococcus phage phi44AHJD, Staphylococcus phage phi7247PVL, Staphylococcus phage phiBU01, Staphylococcus phage phiETA, Staphylococcus phage phiETA2, Staphylococcus phage phiETA3, Staphylococcus phage phiIBB-SEP1, Staphylococcus phage phiIPLA-RODI, Staphylococcus phage phiJB, Staphylococcus phage phiMR11, Staphylococcus phage phiMR25, Staphylococcus phage phiNM1, Staphylococcus phage phiNM2, Staphylococcus phage phiNM3, Staphylococcus phage phiNM4, Staphylococcus phage phiPVL-CN125, Staphylococcus phage phiPVL108, Staphylococcus phage phiSA_BS1, Staphylococcus phage phiSA_BS2, Staphylococcus phage phiSA12, Staphylococcus phage phiSa2wa_1st, Staphylococcus phage phiSa2wa_st121mssa, Staphylococcus phage phiSa2wa_st22, Staphylococcus phage phiSa2wa_st30, Staphylococcus phage phiSa2wa_st5, Staphylococcus phage phiSa2wa_st78, Staphylococcus phage phiSauS-IPLA35, Staphylococcus phage phiSLT, Staphylococcus phage phiSP44-1, Staphylococcus phage Portland, Staphylococcus phage Portland, Staphylococcus phage PSa3, Staphylococcus phage PVL, Staphylococcus phage ROSA, Staphylococcus phage S24-1 DNA, Staphylococcus phage S25-3, Staphylococcus phage S25-4, Staphylococcus phage SA1014ruMSSAST7, Staphylococcus phage SA11, Staphylococcus phage SA13, Staphylococcus phage SA137ruMSSAST121PVL, Staphylococcus phage SA345ruMSSAST8, Staphylococcus phage SA46-CL1, Staphylococcus phage SAS, Staphylococcus phage SA7, Staphylococcus phage SA75, Staphylococcus phage SA780ruMSSAST101, Staphylococcus phage SA97, Staphylococcus phage SAP-2, Staphylococcus phage SAP-26, Staphylococcus phage SAPli, Staphylococcus phage SAP33, Staphylococcus phage Sb-1, Staphylococcus phage SCH1, Staphylococcus phage Sebago, Staphylococcus phage SH-St 15644, Staphylococcus phage SLPW, Staphylococcus phage SN8, Staphylococcus phage SP120, Staphylococcus phage SP197, Staphylococcus phage SP276, Staphylococcus phage SP5, Staphylococcus phage SpT152, Staphylococcus phage St 134, Staphylococcus phage Staph1N, Staphylococcus phage Stau2, Staphylococcus phage StauST398-1, Staphylococcus phage StauST398-2, Staphylococcus phage StauST398-3, Staphylococcus phage StauST398-4, Staphylococcus phage StauST398-5, Staphylococcus phage Teami, Staphylococcus phage TEM126, Staphylococcus phage tp310-1, Staphylococcus phage tp310-2, Staphylococcus phage Twort, Staphylococcus phage UPMK_2, Staphylococcus phage vB_SauM_Remus, Staphylococcus phage vB_SauM_Romulus, Staphylococcus phage vB_SauP_phiAGO1.3, Staphylococcus phage vB_SauS_fPfSau02, Staphylococcus phage vB_SauS_JS02, Staphylococcus phage vB_SauS_phi2, Staphylococcus phage vB_SauS-phiIPLA88, Staphylococcus phage vB_SauS-SAP27, Staphylococcus phage vB_SepiS-phiIPLA5, Staphylococcus phage vB_SepiS-phiIPLA7, Staphylococcus phage vB_SepM_philPLA-CiC, Staphylococcus phage vB_SepS_SEP9, Staphylococcus phage vB_SpsM_WIS42, Staphylococcus phage vB_SpsS_QT1, Staphylococcus phage vB_SscM-1, Staphylococcus phage X2 and Staphylococcus phage YMC/09/04/R1988.

The tail phage may be a Clostridioides tail phage. Examples of Clostridioides tail phages include Clostridioides phage phiC2 and Clostridioides phage phiCD27.

The tail phage may be a Acinetobacter tail phage. Examples of Acinetobacter tail phages include Acinetobacter phage 133, Acinetobacter phage AB1, Acinetobacter phage AB3, Acinetobacter phage Abpl, Acinetobacter phage AbP2, Acinetobacter phage AbTZA1, Acinetobacter phage Acj61, Acinetobacter phage Acj9, Acinetobacter phage AM101, Acinetobacter phage AP205, Acinetobacter phage AP22, Acinetobacter phage Fril, Acinetobacter phage IME-200, Acinetobacter phage IMEAB3, Acinetobacter phage KARL-1, Acinetobacter phage Loki, Acinetobacter phage LZ35, Acinetobacter phage Petty, Acinetobacter phage phiAB1, Acinetobacter phage phiAb6, Acinetobacter phage Presley, Acinetobacter phage SH-Ab 15519, Acinetobacter phage SWH-Ab-1, Acinetobacter phage SWH-Ab-3, Acinetobacter phage vB_AbaM_Apostate, Acinetobacter phage vB_AbaM_B09_Aci01-1, Acinetobacter phage vBAbaM_B09_Aci02-2, Acinetobacter phage vB_AbaM_B09_Aci05, Acinetobacter phage vB_AbaM_Berthold, Acinetobacter phage vB_AbaM_Kimel, Acinetobacter phage vB_AbaM_Konradin, Acinetobacter phage vB_AbaM_Lazarus, Acinetobacter phage vB_AbaM_ME3, Acinetobacter phage vB_AbaM_PhT2, Acinetobacter phage vB_AbaM-IME-AB2, Acinetobacter phage vB_AbaP_Acibel007, Acinetobacter phage vB_AbaP_ASh1, Acinetobacter phage vB_AbaP_AS12, Acinetobacter phage vB_AbaP_B1, Acinetobacter phage vB_AbaP_B3, Acinetobacter phage vB_AbaP_B5, Acinetobacter phage vB_AbaP_D2, Acinetobacter phage vB_AbaP_PD-6A3, Acinetobacter phage vB_AbaP_PD-AB9, AcinetDbacter phage vB_ApiM_fHyAci03, Acinetobacter phage vB_ApiP_P1, Acinetobacter phage vB_ApiP_P2, Acinetobacter phage VB_ApiP_XC38, Acinetobacter phage WCHABP1, Acinetobacter phage WCHABP12, Acinetobacter phage WCHABP5, Acinetobacter phage YMC-13-01-C62, Acinetobacter phage YMC/09/02/B1251, Acinetobacter phage YMC11/11/R3177, Acinetobacter phage ZZ1, Acinetobacter phage_AbKT21phiIII, Acinetobacter phage_vB_AbaP_46-62_Aci07 and Acinetobacter phage_vB_AbaP_B09_Aci08.

Any of the tail phages described herein may be used to deliver the vector to the first cells. Where a tail phage is used to deliver the vector to the first cells, the first cells are of a species that is capable of being infected by the phage.

The phage may be a filamentous phage.

The filamentous phage may be a Vibrio filamentous phage. Examples of Vibrio filamentous phages include Vibrio phage CTXphi, Vibrio phage pre-CTX, Vibrio phage KSF1, Vibrio phage fs1, Vibrio phage ND1-fs1, Vibrio phage VEJ, Vibrio phage VGJ, Vibrio phage VP24-2_Ke, Vibrio phage VSK, Vibrio phage VSKK, Vibrio phage fs2, Vibrio phage VFJ, Vibrio phage VAI1, Vibrio phage VALG_phi6, Vibrio phage Vf03K6, Vibrio phage VfO4K68, Vibrio phage VCY, Vibrio phage VALG_phi8, Vibrio phage Vf12, Vibrio phage Vf33 and Vibrio phage XacF13.

The filamentous phage may be a Xanthomonas filamentous phage. Examples of Xanthomonas filamentous phages include Xanthomonas phage Cf1c, Xanthomonas phage XacF1, Xanthomonas phage Cf2, Xanthomonas phage phi Lf2, Xanthomonas phage phiLf UK, Xanthomonas phage phiXv2, Xanthomonas phage Xf109 and Xanthomonas phage Xf409.

The filamentous phage may be a Ralstonia filamentous phage. Examples of Ralstonia filamentous phages include Ralstonia phage Rs551, Ralstonia phage RS603, Ralstonia phage RSIBR3, Ralstonia phage RSM1, Ralstonia phage RSM3, Ralstonia phage RSMSuper, Ralstonia phage PE226, Ralstonia phage p12J, Ralstonia phage RS611, Ralstonia phage RSBg, Ralstonia phage RSS-TH1, Ralstonia phage RSS0 and Ralstonia phage RSS1.

The filamentous phage may be an Escherichia filamentous phage. Examples of Escherichia filamentous phages include Escherichia phage If1, Escherichia phage fd and Escherichia phage 122.

The filamentous phage may be an Enterobacteria filamentous phage. Examples of Enterobacteia filamentous phages include Enterobacteria phage f1 and Enterobacteria phage M13.

The filamentous phage may be an Erwinia filamentous phage. An example of an Erwinia filamentous phage includes Erwinia phage PEar6.

The filamentous phage may be a Pseudomonas filamentous phage. Examples of Pseudomonas filamentous phages include Pseudomonas phage Pf1, Pseudomonas phage pf8_ST274-AUS411 and Pseudomonas phage Pf3

The filamentous phage may be a Salmonella filamentous phage. An example of an Salmonella filamentous phage includes Salmonella phage IKe.

The filamentous phage may be a Stenotrophomonas filamentous phage. Examples of Stenotrophomonas filamentous phages include Stenotrophomonas phage PSH1, Stenotrophomonas phage SMA6, Stenotrophomonas phage phi SHP2, Stenotrophomonas phage SMA9 and Stenotrophomonas phage SMA7.

In any embodiment described herein, the transmissible element may be capable of being introduced into the recipient bacterium without heat stock. In any embodiment described herein, the transmissible element may be capable of being introduced into a recipient bacterium without electroporation. In any embodiment described herein, the transmissible element may be capable of being introduced into a recipient bacterium without in vitro transformation techniques, including chemical induction (e.g. using calcium).

### Pathway Modification Systems (PMS)

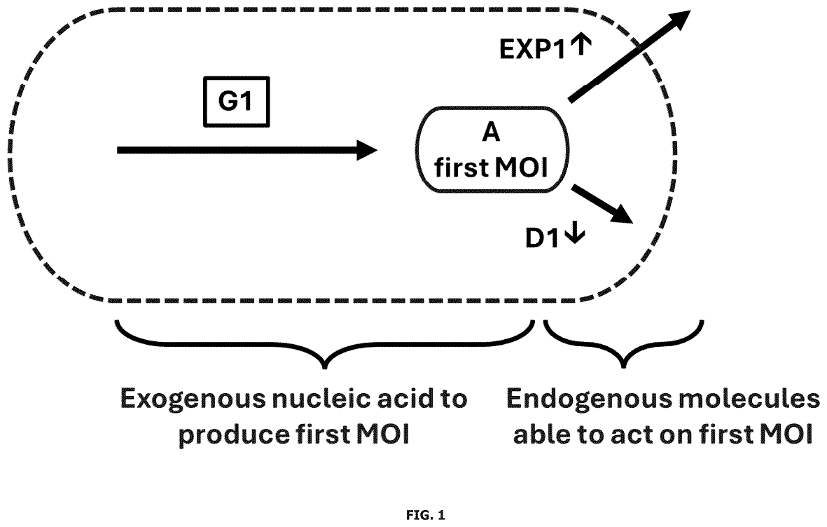

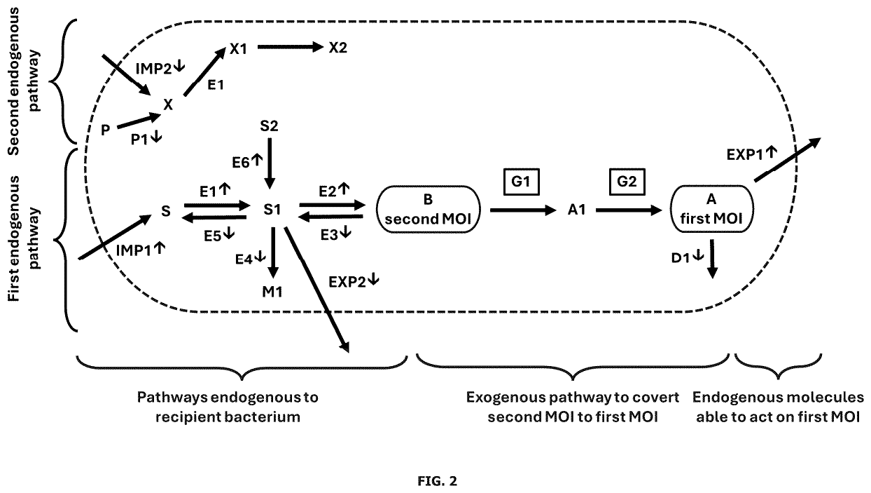

The transmissible elements, such as plasmids, in particular the conjugative plasmids described herein comprise a Pathway Modification System (PMS). PMSs are designed to target endogenous genes of a recipient bacterium which genes will aid the production of or increase the amount of the first MOI. Examples of endogenous targets are shown in FIGS. 1 and 2.

As shown in FIG. 1, at least one exogenous gene G1 (square box) has been transferred to the recipient cell via a transmissible element (such as a conjugative plasmid, a phage or phagemid packaged in a phage particle). The at least one exogenous gene G1 produces a first molecule of interest (MOI), A (solid oval). The first MOI (A) may be degraded by endogenous enzymes of the cell, represented by D1. Thus, downregulation or blocking of such enzymes (D1) will result in an increase of the amount of the first MOI (A) to provide the desired therapeutic or beneficial effect. Equally, the recipient cell may comprise an endogenous exporter, EXP1 which is capable of exporting the first MOI out of the cell. Thus, upregulation or increasing the activity of such exporters will result in an increase of the amount of the first MOI to provide the desired therapeutic effect in the local microbiome (or organism), whilst preventing accumulation of the first MOI in the cell which could cause a fitness disadvantage to the recipient (or donor) bacterium.

As shown in FIG. 2, an exogenous metabolic pathway comprising genes encoding one or more enzymes, in this example, two enzymes, G1 and G2 (square boxes) has been transferred to the recipient cell via a transmissible element (such as a conjugative plasmid, a phage or phagemid packaged in a phage particle). This exogenous pathway produces a first molecule of interest (MOI), A (solid oval) from a second MOI, B (solid oval), which is already present in the cell. The first exogenous enzyme G1 converts the second MOI (B) to an intermediate molecule (A1), which itself is then converted by the enzyme G2 into the desired first MOI (A). The second MOI is an endogenous molecule or may be exogenously added to the cell (for example in the case of a rare carbohydrate food source which is converted to a sugar to be used for growth by the donor or recipient cell). It will be appreciated that any number of exogenous enzymes may be comprised by the transmissible element, according to the desired manipulations of the second MOI to produce the first MOI.

The recipient cell may directly import the second MOI (B) via an importer (not shown in FIG. 2). If such an importer exists, increasing its expression and/or activity may result in more uptake of the second MOI (B) into the cell, which in turn may result in more second MOI being produced by increasing the amount of second MOI being processed by the exogenous genes (G1 and G2) in the pathway to make the first MOI (A).

When the second MOI (B) is endogenous to the recipient cell, the recipient cell may comprise an endogenous pathway for the production of the second MOI (B), shown in FIG. 2 on the left hand side. This may comprise one or more precursor substrates (shown here as S and S1, but it will be appreciated that many more precursor substrates may be present, depending on the complexity of the endogenous pathway for producing the second MOI). Increasing the amounts of these precursor substrates and encouraging the forward reaction (represented in FIG. 2 as S to S1, and S1 to the second MOI (B)) will increase the amount of the second MOI (B) available to be converted into the first MOI (A). This may be achieved in a number of ways, each of which may be affected by the PMS (or any combination thereof) by encoding different nucleic acid modifiers (e.g. different spacer or gRNA sequences) and/or different peptide molecules in the PMS.

Firstly, increasing the import of any of the precursor substrates (shown here as import of S with IMP1, but may be applicable to any of S, S1, S2 . . . etc) by upregulating the amount or expression and/or activity of the relevant importer (IMP1) will increase the amount of that substrate in the cell which is available to be converted in the forward reaction.

Secondly, the forward reaction may be encouraged by increasing the amount and/or expression and/or activity of the forward enzymes (E1 and E2). Increasing the amount, expression or activity of the forward enzymes (E1 and E2) may be achieved, for example, by upregulation of expression, de-repression of expression or by making expression constitutive.

Thirdly, the reverse reaction may be reduced or discouraged by decreasing the amount and/or expression and/or activity of the reverse enzymes (E3 and E5). Decreasing the amount, expression or activity of the reverse reaction enzymes (E3 and E5) may be achieved by downregulation, repression or prevention of gene expression or by blocking the active site of the enzyme with a peptide inhibitor.

Fourthly, some substrates may be able to be produced by more than one enzymatic reaction, as shown here for S1, which can be produced from both precursor substrate S (by enzyme E1) and precursor substrate S2 (by enzyme E6). Thus, increasing the amount and/or expression and/or activity of E6 will also increase the amount of S1 which is available in the cell for conversion to the second MOI (B). Increasing the amount, expression or activity of the common enzyme (S1) may be achieved, for example, by upregulation of expression, de-repression of expression or by making expression constitutive.

Fifthly, the desired endogenous pathway for the production of the second MOI (B) may branch to remove a particular substrate (shown here with S1 being converted by enzyme E4 into the undesired metabolite M1) into an alternative pathway. Thus, to keep the amount of that substrate available for conversion to the second MOI (B), downregulation and/or blocking of the competing branch of the pathway may be achieved by downregulation or prevention of gene expression of E4 or by blocking the active site of the enzyme E4 with a peptide inhibitor, to prevent the formation of metabolite M1. Alternatively, a desired precursor substrate (shown here as S1) may be removed by export from the cell (shown here by exporter EXP2). Thus, to keep the amount of that substrate available for conversion to the second MOI (B), downregulation and/or blocking of the exporter of the desired substrate (S1) may be achieved by downregulation or prevention of gene expression of the exporter (EXP2) or by blocking the active site of the exporter (EXP2) with a peptide inhibitor.

Sixthly, enzymes which form part of the desired endogenous pathway may also be active in alternative and unrelated pathways, such as the pathway represented here by the import of X (shown on the top left side of FIG. 2), and its conversion to X1 by the same enzyme (E1) which converts S to S1 in the desired pathway. Thus, it is desired to reduce the amount of X which is present in the cell, which makes more of the enzyme (E1) available for carrying out the desired forward reaction to covert S to S1 (to then be converted to make the second MOI (B)). This may be achieved by downregulation and/or blocking any importer of X (IMP2), or by blocking the active site of the importer (IMP2) with a peptide inhibitor. Additionally or alternatively, reduction of the amount of X in the cell may be achieved by downregulating and/or blocking any enzyme which can produce X (shown in FIG. 2 as enzyme P1, which converts a precursor substrate P into X).

Finally, the first MOI (A) may be degraded by endogenous enzymes of the cell, represented by D1. Thus, downregulation or blocking of such enzymes (D1) will result in an increase of the amount of the first MOI (A) to provide the desired therapeutic or beneficial effect. Equally, the recipient cell may comprise an endogenous exporter, EXP1 which is capable of exporting the first MOI out of the cell. Thus, upregulation or increasing the activity of such exporters will result in an increase of the amount of the first MOI to provide the desired therapeutic effect in the local microbiome, whilst preventing accumulation of the first MOI in the cell and potentially causing a fitness disadvantage.

In any of the modifications discussed above, the PMS may be programmed to introduce specific point mutations in the expressed protein (e.g. the enzyme, importer or exporter) which results in a change in activity (i.e. an increase or decrease) of that protein. This may be in the active site, or, in the case of decreasing activity, a mutation in a protein-protein interface or one or more amino acid residues required for correct folding or assembly of an active protein.

A skilled person will appreciate that regulation of some endogenous genes in bacteria may be complex, involving regulatory regions which may be spaced apart from the target nucleic acid whose expression is modified by the PMS. It is envisaged that any nucleic acid modifier of the PMS may increase or decrease expression of a target nucleic acid by modifying nucleic acids (such as repressors, activators, RNA and other regulatory regions) which are spaced apart from the coding regions of the expressed target peptide (e.g. enzyme, importer or exporter).

Thus, in one embodiment, the PMS is programmed to reduce or prevent expression of a target endogenous nucleic acid in the recipient bacterium. The PMS may encode a nucleic acid modifier. In a particular embodiment, the PMS prevents expression of the target endogenous nucleic acid.

Thus, there is provided a transmissible element as described herein, or a recipient bacterium or donor bacterium as described herein comprising a transmissible element, wherein the nucleic acid modifier expressed by the PMS of B) reduces expression (or prevents expression) of the target endogenous nucleic acid, and wherein the reduction in expression of the target endogenous nucleic acid increases or maintains the production of the first MOI. There is also provided a transmissible element as described herein, or a recipient bacterium or donor bacterium as described herein comprising a transmissible element, wherein the nucleic acid modifier expressed by the PMS of B) reduces expression (or prevents expression) of the target endogenous nucleic acid, and wherein the reduction in expression of the target endogenous nucleic acid reduces consumption or degradation of the first MOI. In particular embodiments, the expression is prevented by the nucleic acid modifier.

In one embodiment, the target endogenous nucleic acid encodes an enzyme (for example as shown in FIG. 2 as D1) which degrades the first MOI (A), and the reduction of expression of the enzyme increases or maintains production of the first MOI by reducing consumption or degradation of the first MOI. In a particular embodiment, the reduction of expression of the enzyme increases production of the first MOI.

In one embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an enzyme (E3) in the endogenous pathway which converts the second MOI (B) into a substrate (S1) which is earlier in the endogenous pathway,

and the reduction of expression of the enzyme (E3) increases or maintains production of the first MOI by reducing degradation of said second MOI (B) to said substrate (S1) in the recipient bacterium. In a particular embodiment, the reduction of expression of the enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an enzyme (E4) in the endogenous pathway which converts a first substrate (S1) used in the production of the second MOI (B) into a second substrate (M1) which is not used in the production of said second MOI,

and the reduction of expression of the enzyme (E4) increases or maintains production of the first MOI by increasing the amount of said first substrate (S1) in the recipient bacterium. In a particular embodiment, the reduction of expression of the enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an enzyme (E5) in the endogenous pathway which converts a first substrate (S1) of the endogenous pathway into a second substrate (S) which is earlier in the endogenous pathway,

and the reduction of expression of the enzyme (E5) increases or maintains production of the first MOI by reducing degradation of said first substrate (S1) in the recipient bacterium. In a particular embodiment, the reduction of expression of the enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an exporter (EXP2) of a substrate in the endogenous pathway (S1),

and the reduction of expression of the exporter (EXP2) increases or maintains production of the first MOI by increasing the amount of said substrate (S1) in the recipient bacterium. In a particular embodiment, the reduction of expression of the exporter increases production of the first MOI.

In one embodiment, the exogenous nucleic acid of A) encodes one or more protein(s) (G1, G2 . . . GX) for the conversion of a second MOI (B) to the first MOI (A); and

wherein the recipient bacterium comprises:

a first endogenous pathway comprising at least one enzyme for the conversion of one or more substrates (S, S1, S2 . . . etc) into the second MOI (B), and

a second endogenous pathway comprising at least one enzyme (E1) which is common to the first and second endogenous pathways,

and the at least one common enzyme (E1) can convert a first substrate of the first endogenous pathway (S) and a second, different substrate of the second endogenous pathway (X); and

wherein the target endogenous nucleic acid encodes an importer (IMP2) which imports the second, different substrate of the second endogenous pathway (X),

and the reduction of expression of the importer (IMP2) increases or maintains production of the first MOI by increasing the availability and/or activity of said common enzyme (E1) to convert said first substrate (S) in the first endogenous pathway in the recipient bacterium. In a particular embodiment, the reduction of expression of the importer increases production of the first MOI.

In another embodiment, the exogenous nucleic acid of A) encodes one or more protein(s) (G1, G2 . . . GX) for the conversion of a second MOI (B) to the first MOI (A); and

wherein the recipient bacterium comprises:

a first endogenous pathway comprising at least one enzyme for the conversion of one or more substrates (S, S1, S2 . . . etc) into the second MOI (B), and

a second endogenous pathway comprising at least one enzyme (E1) which is common to the first and second endogenous pathways,

and the at least one common enzyme (E1) can convert a first substrate of the first endogenous pathway (S) and a second, different substrate of the second endogenous pathway (X); and

wherein the target endogenous nucleic acid encodes an enzyme which is part of the second endogenous pathway and is for the production of the second, different substrate of the second endogenous pathway (X), for example is an endogenous enzyme (P1) which is part of the second endogenous pathway and is for the direct production of the second, different substrate of the second endogenous pathway (X),

and the reduction of expression of the enzyme (P1) increases or maintains production of the first MOI by reducing the amount of the second, different substrate of the second endogenous pathway (X), thereby increasing the availability and/or activity of said common enzyme (E1) to convert said first substrate (S) in the first endogenous pathway in the recipient bacterium. In a particular embodiment, the reduction of expression of the enzyme increases production of the first MOI.

In any embodiment relating to reducing or preventing expression of a target endogenous nucleic acid in the recipient bacterium with a nucleic acid modifier expressed by the PMS of B), the nucleic acid modifier may reduce expression (or prevent expression) of the target endogenous nucleic acid by introducing a modification to one or more nucleotides in the endogenous DNA (e.g. comprised by the chromosome or a plasmid) of the recipient bacterium. The modification may be one or more point mutations in one or more codons of the target endogenous nucleic acid, such as introduction of a premature stop codon (e.g. as shown in Example 3 herein). When the target endogenous nucleic acid encodes a protein (such as an enzyme, importer or exporter), the modification may introduce a stop codon in the target endogenous nucleic acid, thereby preventing expression of said protein. Alternatively, the modification may truncate expression of said protein, such that any expressed protein is non-functional. When the target endogenous nucleic acid comprises a ribosome binding site to control expression of a protein expressed from the target endogenous nucleic acid (such as an enzyme, importer or exporter), the modification may modify the ribosome binding site, thereby reducing or preventing expression of said protein. Alternatively, the target endogenous nucleic acid comprises a translational start site to control expression of a protein expressed from the target endogenous nucleic acid (such as an enzyme, importer or exporter), the modification may modify the translational start site, thereby reducing or preventing expression of said protein. In a particular embodiment, the nucleic acid modifier prevents expression of the target endogenous nucleic acid. The modification may be a modification to increase expression of a repressor of the target endogenous nucleic acid. The modification may be a modification to decrease expression of an activator of the target endogenous nucleic acid.

Alternatively, the nucleic acid modifier may reduce expression (or prevent expression) of the target endogenous nucleic acid by producing a nucleic acid inhibitor molecule, for example a small regulatory RNA (sRNA). The small regulatory RNA may bind to a promoter or coding sequence of the target endogenous nucleic acid, or to mRNA expressed from the nucleic acid sequence, thereby blocking transcription machinery from producing the encoded protein. When the target endogenous nucleic acid comprises a promoter to control expression of a protein expressed from the target endogenous nucleic acid (such as an enzyme, importer or exporter) and the modification may modify the promoter, thereby reducing or preventing expression of said protein. In one embodiment, the target endogenous nucleic acid encodes a protein (such as an enzyme, importer or exporter) and the nucleic acid inhibitor molecule is a small regulatory RNA (sRNA) which binds to the transcribed mRNA encoding said protein, thereby preventing expression of said protein, or truncating expression such that any expressed protein is non-functional. In another embodiment, the nucleic acid inhibitor molecule is a small regulatory RNA (sRNA) which binds to and causes degradation of an mRNA molecule expressed from the target endogenous nucleic acid encoding the protein (e.g. a or the enzyme, or a or the importer, or a or the exporter), thereby reducing or preventing the translation of said target endogenous protein. In a particular embodiment, the nucleic acid modifier reduces expression of the target endogenous nucleic acid.

In one embodiment, the PMS is programmed increases expression of the target endogenous nucleic acid. The PMS may encode a nucleic acid modifier.

Thus, there is provided a transmissible element as described herein, or a recipient bacterium or donor bacterium as described herein comprising a transmissible element, wherein the nucleic acid modifier expressed by the PMS of B) increases expression of the target endogenous nucleic acid, and wherein the increase in expression of the target endogenous nucleic acid increases or maintains the production of the first MOI.

In one embodiment, the target endogenous nucleic acid encodes an exporter (for example, as shown in FIG. 2 as EXP1) which is capable of exporting the first MOI (A) to the periplasm of the recipient bacterium, to the cell surface of the recipient bacterium, or to the extracellular space outside of the recipient bacterium, and the increase of expression of the exporter increases or maintains production of the first MOI by increasing export of said first MOI to the periplasm of the recipient bacterium, to the cell surface of the recipient bacterium, or to the extracellular space outside of the recipient bacterium. In a particular embodiment, the increase in expression of the exporter increases production of the first MOI.

In one embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an enzyme (E1) in the endogenous pathway which converts a first substrate (S) into a second substrate (S1) in the endogenous pathway,

and the increase of expression of the enzyme increases or maintains production of the first MOI by increasing the amount of said second substrate (S1) in the recipient bacterium. In a particular embodiment, the increase in expression of the enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an enzyme (E2) in the endogenous pathway which converts a first substrate (S) into the second MOI (B) in the endogenous pathway,

and the increase of expression of the enzyme increases or maintains production of the first MOI by increasing the amount of said second MOI (B) in the recipient bacterium. In a particular embodiment, the increase in expression of the enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous nucleic acid encodes an importer (IMP1) in the endogenous pathway which imports a first substrate (S) into the recipient bacterium for use in the endogenous pathway,

and the increase of expression of the importer (IMP1) increases or maintains production of the first MOI by increasing the amount of said first substrate (S) in the recipient bacterium. In a particular embodiment, the increase in expression of the importer increases production of the first MOI.

In one embodiment, the exogenous nucleic acid of A) encodes one or more protein(s) (G1, G2 . . . GX) for the conversion of a second MOI (B) to the first MOI (A); and

wherein the recipient bacterium comprises:

a first endogenous pathway comprising at least one enzyme for the conversion of one or more substrates (S, S1, S2 . . . etc) into the second MOI (B), and

a second endogenous pathway, which is a branch of the first endogenous pathway and comprises at least one substrate is a substrate (S1) which is common to the first and second endogenous pathways; and

wherein the target endogenous nucleic acid encodes an enzyme (E6) which converts a second substrate of the second endogenous pathway (S2) to the common substrate (S1),

and the increase in expression of the enzyme (E6) increases or maintains production of the first MOI by increasing the amount of the common substrate (S1) in the recipient bacterium. In a particular embodiment, the increase in expression of the enzyme increases production of the first MOI.

In any embodiment relating to increasing expression of a target endogenous nucleic acid in the recipient bacterium with a nucleic acid modifier expressed by the PMS of B), the nucleic acid modifier may increase expression of the target endogenous nucleic acid by introducing a modification to one or more nucleotides in the endogenous DNA (e.g. comprised by the chromosome or a plasmid) of the recipient bacterium. When the target endogenous nucleic acid encodes a gene encoding a protein (e.g. a or the enzyme, or a or the importer, or a or the exporter), and the modification may modify a repressor of said gene, thereby reducing or preventing expression of the repressor, which de-represses expression of said gene and increases expression of the target endogenous nucleic acid. When the target endogenous nucleic acid encodes a gene encoding a protein (e.g. a or the enzyme, or a or the importer, or a or the exporter) preceded by a leader sequence, the modification may modify a nucleic acid encoding the leader sequence in said gene, thereby increasing expression of said protein. When the target endogenous nucleic acid encodes a gene encoding a protein (e.g. a or the enzyme, or a or the importer, or a or the exporter), the modification may modify the promoter of said gene, thereby increasing expression of said protein. For example, the modification may introduce a mutation which increases the strength of the promoter of the target gene. When the target endogenous nucleic acid comprises a ribosome binding site to control expression of a protein expressed from the target endogenous nucleic acid (such as an enzyme, importer or exporter), the modification may modify the ribosome binding site, thereby increasing expression of said protein. The modification may introduce one or more mutations to optimize the sequence of the ribosome binding site. When the target endogenous nucleic acid comprises a translational start site to control expression of a protein expressed from the target endogenous nucleic acid (such as an enzyme, importer or exporter), the modification may modify the translational start site, thereby increasing expression of said protein. The modification may introduce one or more mutations to optimize the sequence of the translational start site. The modification may be a modification to decrease expression of a repressor of the target endogenous nucleic acid. The modification may be a modification to increase expression of an activator of the target endogenous nucleic acid.

Alternatively, the nucleic acid modifier may increase expression of the target endogenous nucleic acid by producing a nucleic acid activator molecule, for example a small regulatory RNA (sRNA). When the target endogenous nucleic acid encodes a protein (e.g. a or the enzyme, or a or the importer, or a or the exporter), the nucleic acid activator molecule may bind a repressor of said gene, thereby reducing or preventing expression of the repressor, which de-represses expression of said gene and increases expression of the target endogenous nucleic acid. When the target endogenous nucleic acid encodes a translation activator mRNA, the nucleic acid activator molecule may be a small regulatory RNA (sRNA) which binds the mRNA and activates translation of said translation activator. Kim et al., Mol. Cells, 42(5), 426-439, 2019, doi:10.14348/molcells.2019.0040 (incorporated herein by reference in its entirety) describes Hfq-independent activation of rpoS by DsrA (a sRNA) in E. coli.

Thus, in one embodiment, the PMS is programmed to reduce or prevent activity or function of a target endogenous protein in the recipient bacterium. The PMS may encode a peptide molecule. In a particular embodiment, the PMS encodes a peptide molecule which blocks the function of a target endogenous protein.

Thus, there is provided a transmissible element as described herein, or a recipient bacterium or donor bacterium as described herein comprising a transmissible element, wherein the peptide molecule expressed by the PMS of B) is an inhibitor to reduce or prevent (e.g. block) activity or function of the target endogenous protein in the recipient bacterium, wherein the reduction or prevention in the activity or function of said protein increases or maintains the production of the first MOI. There is also provided a transmissible element as described herein, or a recipient bacterium or donor bacterium as described herein comprising a transmissible element, wherein the peptide molecule expressed by the PMS of B) is an inhibitor to reduce or prevent (e.g. block) activity or function of the target endogenous protein in the recipient bacterium, wherein the reduction or prevention in the activity or function of said protein prevents the consumption of the first MOI.

In one embodiment, the target endogenous protein is an enzyme (for example as shown in FIG. 2 as D1) which degrades the first MOI (A), and the reduction or prevention of activity or function of said protein increases or maintains (e.g. increases) production of the first MOI by reducing consumption or degradation of the first MOI.

In one embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous protein is:
(a) an enzyme (E3) in the endogenous pathway which converts the second MOI (B) into a substrate (S1) which is earlier in the endogenous pathway,

and the reduction or prevention of the activity or function of said enzyme (E3) increases or maintains (e.g. increases) production of the first MOI by reducing degradation of said second MOI (B) to said substrate (S1) in the recipient bacterium. In a particular embodiment, the PMS encodes a peptide molecule which blocks the function of the enzyme. In a particular embodiment, the reduction or prevention of the activity or function of said enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous protein is an enzyme (E4) in the endogenous pathway which converts a first substrate (S1) used in the production of the second MOI (B) into a second substrate (M1) which is not used in the production of the second MOI,

and the reduction or prevention of the activity or function of said enzyme (E4) increases or maintains (e.g. increases) production of the first MOI by increasing the amount of said first substrate (S1) in the recipient bacterium. In a particular embodiment, the PMS encodes a peptide molecule which blocks the function of the enzyme. In a particular embodiment, the reduction or prevention of the activity or function of said enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous protein is an enzyme (E5) in the endogenous pathway which converts a first substrate (S1) of the endogenous pathway into a second substrate (S) which is earlier in the endogenous pathway,

and the reduction or prevention of the activity or function of said enzyme (E5) increases or maintains (e.g. increases) production of the first MOI by reducing degradation of said first substrate (S1) in the recipient bacterium. In a particular embodiment, the PMS encodes a peptide molecule which blocks the function of the enzyme. In a particular embodiment, the reduction or prevention of the activity or function of said enzyme increases production of the first MOI.

In another embodiment, the exogenous nucleic acid of A) encodes one or more protein(s) (G1, G2 . . . GX) for the conversion of a second MOI (B) to the first MOI (A), and

wherein the recipient bacterium comprises an endogenous pathway for the conversion of one or more substrates (S, S1, S2 . . . etc) into the second MOI (B), and

wherein the target endogenous protein is an exporter (EXP2) of a substrate in the endogenous pathway (S1),

and the reduction or prevention of the activity or function of said exporter (EXP2) increases or maintains (e.g. increases) production of the first MOI by increasing the amount of said substrate (S1) in the recipient bacterium. In a particular embodiment, the PMS encodes a peptide molecule which blocks the function of the exporter. In a particular embodiment, the reduction or prevention of the activity or function of said exporter increases production of the first MOI.

In one embodiment, the exogenous nucleic acid of A) encodes one or more protein(s) (G1, G2 . . . GX) for the conversion of a second MOI (B) to the first MOI (A); and

wherein the recipient bacterium comprises:

a first endogenous pathway comprising at least one enzyme for the conversion of one or more substrates (S, S1, S2 . . . etc) into the second MOI (B), and

a second endogenous pathway comprising at least one enzyme (E1) which is common to the first and second endogenous pathways,

and the at least one common enzyme (E1) can convert a first substrate of the first endogenous pathway (S) and a second, different substrate of the second endogenous pathway (X); and

wherein the target endogenous protein is an importer (IMP2) which imports the second, different substrate of the second endogenous pathway (X),

and the reduction or prevention of the activity or function of said importer (IMP2) increases or maintains (e.g. increases) production of the first MOI by increasing the availability and/or activity of said common enzyme (E1) to convert said first substrate (S) in the first endogenous pathway in the recipient bacterium. In a particular embodiment, the PMS encodes a peptide molecule which blocks the function of the importer. In a particular embodiment, the reduction or prevention of the activity or function of said importer increases production of the first MOI.

In another embodiment, the exogenous nucleic acid of A) encodes one or more protein(s) (G1, G2 . . . GX) for the conversion of a second MOI (B) to the first MOI (A); and

wherein the recipient bacterium comprises:

a first endogenous pathway comprising at least one enzyme for the conversion of one or more substrates (S, S1, S2 . . . etc) into the second MOI (B), and

a second endogenous pathway comprising at least one enzyme (E1) which is common to the first and second endogenous pathways,

and the at least one common enzyme (E1) can convert a first substrate of the first endogenous pathway (S) and a second, different substrate of the second endogenous pathway (X); and

wherein the target endogenous protein is an enzyme which is part of the second endogenous pathway and is for the production of the second, different substrate of the second endogenous pathway (X), for example is an endogenous enzyme (P1) which is part of the second endogenous pathway and is for the direct production of the second, different substrate of the second endogenous pathway (X),

and the reduction or prevention of the activity or function of the enzyme (P1) increases or maintains (e.g. increases) production of the first MOI by increasing the availability and/or activity of said common enzyme (E1) to convert said first substrate (S) in the first endogenous pathway in the recipient bacterium. In a particular embodiment, the PMS encodes a peptide molecule which blocks the function of the enzyme. In a particular embodiment, the reduction or prevention of the activity or function of said enzyme increases production of the first MOI.

In any embodiment relating to reducing or preventing (e.g. blocking) activity or function of the target endogenous protein which is an enzyme, the peptide molecule inhibitor may bind to the active site of the target endogenous enzyme but cannot be converted by the enzyme, thereby preventing a new substrate from being bound and converted by said enzyme. Alternatively, when the target endogenous protein is an importer or exporter, the peptide molecule inhibitor may bind in a channel of the target endogenous importer or exporter, but does not get transported through the channel of said importer or exporter, thereby preventing transport of any other substrate through said channel. Alternatively, the peptide molecule inhibitor may bind to the target endogenous protein (e.g. an enzyme, exporter or importer) and change the conformation of said protein, thereby reducing or preventing the endogenous function of said target endogenous protein. For example the peptide inhibitor may prevent correct folding of said protein or correct assembly of said protein as part of a multi-unit protein.

The target may be a protein, a transcription factor, DNA (e.g. an exon, an intron, a promoter and/or a leader sequence) or RNA. In one embodiment, the PMS targets an expressed protein. In one embodiment, the PMS targets a transcription factor. In one embodiment, the PMS targets DNA, in particular a coding sequence and/or a promoter. In one embodiment, the PMS targets RNA.

In one embodiment, the PMS provides means to block enzymatic activity of a target protein. In one embodiment, the PMS provides means to reduce or prevent transcription or expression of a target gene. In one embodiment, the PMS expresses a targeting molecule which is specific for the target sequence, and results in a reduction or ablation of activity of the target (e.g. by blocking enzymatic activity and/or by reducing or preventing expression of the target protein or gene).

In any embodiment described herein, optionally the PMS does not modulate the target endogenous nucleic acid sequence in the recipient bacterium which is a virulence gene, antibiotic resistance gene, or an essential gene. Optionally, the PMS does not modulate the target endogenous nucleic acid in the recipient bacterium which is a toxin gene. Optionally, the PMS does not modulate the target endogenous nucleic acid in the recipient bacterium by introducing a double stranded break in DNA or RNA comprised by the recipient bacterium. Optionally, the PMS does not modulate the target endogenous nucleic acid in the recipient bacterium by removing a (second) plasmid comprised by the recipient bacterium, wherein the (second) plasmid is not the transmissible element. Optionally, the PMS does not modulate the target endogenous nucleic acid in the recipient bacterium by killing the recipient bacterium. In any embodiment described herein, the PMS optionally does not modulate any combination of the aforementioned target nucleic acid methods.

In one embodiment, the PMS is under the control of an inducible promoter. In another embodiment, the PMS is under the control of a constitutive promoter.

In one embodiment, the PMS encodes a system selected from the group consisting of:
(a) a base editor;
(b) a prime editor;
(c) an RNAi system;
(d) a CRISPRi system; and
(e) a nuclease (e.g. a CRISPR/Cas system, a TALEN or a zinc finger).

Many of the systems below rely on the use of a CRISPR/Cas protein fused to an editing moiety. CRISPR/Cas proteins are well-known to those skilled in the art. In any embodiment described herein which may use a CRISPR/Cas protein, which does not rely on the ability of the CRISPR/Cas protein per se to be able to introduce a single or double stranded nucleic acid break, a CasS system (e.g. comprising one or more of CasS1, CasS2, CasS3, CasS4 and CasS5 proteins) as described elsewhere herein may be used.

### Base Editors

In a particular embodiment, the PMS encodes a nucleic acid modifier which is a base editor.

Base editors that may be used as a PMS are well known in the art. The PMS may encode a nucleic acid modifier which is a base editor which is a modified nuclease that is modified to be unable to perform DNA double strand breaks, while retaining its DNA binding capacity and that also is fused to a domain to perform base editing. Base editors are described in general, for example, in Rees t al., Nature Genetics Reviews, 19, 2018, 770-788, which is incorporated herein by reference in its entirety.

Thus, base editors include systems where CRISPR/Cas protein(s) (such as dead-Cas9 (dCas9) or nickase Cas9 (nCas9)) is/are fused to a cytosine or adenosine deaminase domain and directed to the target sequence to make the desired modification. Thus, in one embodiment, the PMS encodes a dCas9 or nCas9 fused to a cytosine or adenosine deaminase domain and a guide RNA (or crRNA).

In one embodiment, the base editor is selected from:
A. Cytosine Base Editors (CBEs) that convert C:G into T:A (Komor et al, Nature, 533:420-4, 2016, incorporated herein by reference in its entirety)
CBEs rely on ssDNA cytidine deaminase among which: APOBEC1, rAPOBEC1, APOBEC1 mutant or evolved version (evoAPOBEC1), and APOBEC homologs (APOBEC3A (eA3A), Anc689), Cytidine deaminase 1 (CDA 1), evoCDA 1, FERNY, evoFERNY.
B. Adenine Base Editors (ABEs) that convert A:T into G:C (Gaudelli et al., Nature, 551 (7681), 464-471, 2017, incorporated herein by reference in its entirety)
ABEs rely on deoxyadenosine deaminase activity of a tandem fusion TadA-TadA* where TadA* is an evolved version of TadA, an E. coli tRNA adenosine deaminase enzyme, able to convert adenosine into inosine on ssDNA. TadA* include TadA-8a-e and TadA-7.10.
C. Cytosine Guanine Base Editors (CGBEs) that convert C:G into G:C (Chen et al, Biorxiv, 2020; Kurt et al., Nature Biotechnology, 2020, both incorporated herein by reference in its entirety)
CGBEs generally consist of a nickase CRISPR/Cas protein fused to a cytosine deaminase (rAPOBEC) and base excision repair proteins, such as rXRCC1, or of a nickase CRISPR/Cas protein fused to a rat APOBEC1 variant (R33A) protein and an E. coli derived uracil DNA N-glycosylase (eUNG).
D. Cytosine Adenine Base Editors (CABEs) that convert C:G into A:T (Zhao et al., Nature Biotechnology, 2020, incorporated herein by reference in its entirety)
CABEs generally consist of a nickase CRISPR/Cas protein (e.g. a Cas9 nickase), a cytidine deaminase (e.g. AID), and a uracil-DNA glycosylase (Ung).
E. Adenine Cytosine Base Editors (ACBEs) that convert A:T into C:G (WO2020/181180, incorporated herein by reference in its entirety)
ACBEs generally include a nucleic acid programmable DNA-binding protein and an adenine oxidase.
F. Adenine Thymine Base Editors (ATBEs) that convert A:T into T:A (WO2020/181202, incorporated herein by reference in its entirety)
ATBEs generally consist of a CRISPR/Cas nickase protein (e.g. a Cas9 nickase) and one or more adenosine deaminase or an oxidase domain.
G. Thymine Adenine Base Editor (TABE) that convert T:A into A:T (WO2020/181193; WO2020/181178; WO2020/181195, each of which is incorporated herein by reference in its entirety).

TABEs generally consist of a CRISPR/Cas nickase protein (e.g. a Cas9 nickase) and an adenosine methyltransferase, a thymine alkyltransferase, or an adenosine deaminase domain.

Further additional modules can be added to base editors to increase precision, modularity and editing efficiency. These include the addition of one or two uracil DNA glycosylase inhibitor domain(s) (UGI) to prevent base excision repair mechanism to revert base edition, which is used in the CasS-BE described hereinbelow in the Examples. Another module that can be added is Mu-GAM that decreases the insertion-deletion rate by inhibiting DNA repair by the non-homologous end joining mechanism (NHEJ) in the cell. For CRISPR/Cas based base editing systems, the CRISPR/Cas system may rely on nickase activity (for example by mutating Cas9 to form the well-known nCas9 D10A). The use of a nickase cuts the non-edited strand, and favours its repair and thus the fixing of the edited base.

In an example, the base editor is selected from the group consisting of: POBEC1, rAPOBEC1, APOBEC1 mutant or evolved version (evoAPOBEC1), APOBEC homologs (APOBEC3A (eA3A), Anc689), Cytidine deaminase 1 (CDA1), evoCDA 1, FERNY, evoFERNY, BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Casl2a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRYCBE, SpGCBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3 and AncBE4. Examples of DNA-based editor proteins include, but are not limited to BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Casl2a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRYCBE, SpG-CBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3 and AncBE4.

A particular example of a base editor is shown in Example 2 below. Unlike many CRISPR/Cas-based base editing tools, which use single-subunit effector modules from class 2 CRISPR/Cas systems, the CasS-base editing system described herein uses a multi-subunit effector module from a new class 1 CRISPR/Cas system. In this example, a UGI domain is added.

In particular embodiments, the PMS encodes a fusion protein comprising a polypeptide (Px), wherein Px comprises
I. an amino acid sequence that is at least 90% identical to a sequence selected from SEQ ID Nos:1-5; and is fused to
II. a base editor selected from an adenine base editor (ABE, e.g. an adenosine deaminase), a cytosine base editor (CBE, e.g. a cytidine deaminase or an apolipoprotein B mRNA-editing complex (APOBEC) family deaminase), a cytosine guanine base editor (CGBE), a cytosine adenine base editor (CABE), an adenine cytosine base editor (ACBE), an adenine thymine base editor (ATBE), a thymine adenine base editor (TABE), and optionally a uracil DNA glycosylase inhibitor (UGI) protein; and
the PMS further encodes a guide RNA or crRNA.

In particular, the polypeptide comprises in part I. an amino acid sequence that is at least 90% identical to SEQ ID No:4.

In particular embodiments, the base editor of part II. is a CBE. In further particular embodiments, the CBE is a PmCDA1 cytidine deaminase (optionally in combination with a UGI protein).

The fusion may be direct or indirect. Indirect fusion may use any of the linkers described elsewhere herein.

In another embodiment, the PMS encodes a fusion protein-ribonucleic acid complex comprising:
(i) a fusion protein polypeptide (Px), wherein Px comprises
   I. a Cas-S1, Cas-S3 or Cas-S4 protein (in particular a Cas-S4 protein); and is fused to
   II. a base editor selected from an adenine base editor (ABE, e.g. an adenosine deaminase), a cytosine base editor (CBE, e.g. a cytidine deaminase or an apolipoprotein B mRNA-editing complex (APOBEC) family deaminase), a cytosine guanine base editor (CGBE), a cytosine adenine base editor (CABE), an adenine cytosine base editor (ACBE), an adenine thymine base editor (ATBE), a thymine adenine base editor (TABE), and optionally a uracil DNA glycosylase inhibitor (UGI) protein;
(ii) a Cas-S2 protein;
(iii) a Cas-S5 protein;
(iv) a guide RNA or crRNA comprising a spacer sequence that is cognate to a first protospacer in a target sequence; and
(v) optionally a protein which is selected from a Cas-S1, Cas-S3 and Cas-S4 protein, and is of a different CasS protein class to the protein recited in part (i)a) (in particular a Cas-S1 protein); and
(vi) optionally a protein which is selected from a Cas-S1, Cas-S3 and Cas-S4 protein, and is of a different CasS protein class to the protein recited in part (i)a), and, if present, is of a different CasS protein class to the protein recited in part (v) (in particular a Cas-S3 protein).

In another embodiment, the PMS encodes a fusion protein complex comprising a fusion protein in a complex with further proteins, and a crRNA or gRNA, wherein the fusion protein comprises a protein of the formula A-B-C-D-E (in 5′ to 3′ orientation), wherein:
A is a CasS protein selected from a Cas-S1, Cas-S3 or Cas-S4 (in particular, Cas-S1);
B is optionally present, and when present comprises a linker (for example any of the linkers as described herein, in particular an XTEN linker, for example having the amino acid sequence of SEQ ID No:17);
C is a base editor, such as a cytidine deaminase (e.g. a PmCDA1 cytidine deaminase as described elsewhere herein, for example having the amino acid sequence of SEQ ID No:19);
D is optionally present and when present comprises a linker (for example any of the linkers as described herein, in particular the 10 amino acid linker of SEQ ID No:23);
E is a uracil DNA glycosylase inhibitor (UGI) protein (as described in more detail elsewhere herein, for example having the amino acid sequence of SEQ ID No:21); and
the further proteins in complex with the fusion protein comprise a Cas-S2 and a Cas-S5 protein and a Cas-S1 and/or a Cas-S4 protein as required such that the fusion protein complex comprises at least a Cas-S1, Cas-S2, Cas-S4 and a Cas-S5 protein.

In any embodiment relating to CasS-based base editors, in particular, the base editor of II. is a CBE. In one particular embodiment, the base editor of II. is a PmCDA1 cytidine deaminase (as described elsewhere herein). In one embodiment, the base editor of II. is a PmCDA1 cytidine deaminase in combination with a UGI protein. In one embodiment, the base editor of II. is a CBE linked via a linker (as described elsewhere herein) to a UGI protein. In one embodiment, the base editor of II. is a PmCDA1 cytidine deaminase linked via a linker (as described elsewhere herein) to a UGI protein. In one embodiment, the base editor of II. is a CBE linked via a linker (as described elsewhere herein) to a UGI protein, and the CBE is linked to a CasS protein described herein via a linker (as described elsewhere herein). In one embodiment, the base editor of II. is a PmCDA1 cytidine deaminase linked via a linker (as described elsewhere herein) to a UGI protein, and the PmCDA1 is linked to a CasS protein described herein via a linker (as described elsewhere herein). In one embodiment, the base editor of II. is a CBE linked via a linker (as described elsewhere herein) to a UGI protein, and the UGI protein is linked to a CasS protein described herein via a linker (as described elsewhere herein). In one embodiment, the base editor of II. is a PmCDA1 cytidine deaminase linked via a linker (as described elsewhere herein) to a UGI protein, and the UGI protein is linked to a CasS protein described herein via a linker (as described elsewhere herein). In one embodiment, the CasS protein is selected from a CasS1 protein, a CasS3 protein and a CasS4 protein.

The base editor may be a sea lamprey base editor. In one embodiment, the base editor is a PmCDA1 cytidine deaminase having the amino acid sequence encoded by SEQ ID No:19. In one embodiment, the base editor is from sea lamprey.

In one embodiment, the base editor is fused to the C-terminus of the amino acid sequence of part I. In an embodiment, the base editor (e.g. PmCDA1 cytidine deaminase) is fused via a peptide linker to the amino acid sequence of Part I. The peptide linker may be as described elsewhere herein. The base editor (e.g. PmCDA1 cytidine deaminase) may be fused viaa peptide linker to a CasS protein. The linker may from (about) 10 to 20 amino acids in length. The linker may be from (about) 10 to 22, from (about) 11 to 21, from (about) 12 to 20, from (about) 13 to 19, from (about) 14 to 18, from (about) 15 to 17 amino acids in length. The linker may be (about) 16 amino acids in length. The linker may be a linker having the amino acid sequence of SEQ ID No:17. The linker may be any linker described herein. In an embodiment, the UGI protein is fused to the base editor. The UGI protein may have an amino acid sequence of SEQ ID No:21. The UGI protein may be as described elsewhere herein. The UGI protein may be fused directly to the base editor. The UGI protein may be fused to the base editor via a linker, e.g. a peptide linker as described elsewhere herein.

In one embodiment, the UGI protein is fused to the base editor viaa peptide linker. The UGI protein may be fused to the base editor via a peptide linker of from (about) 8 to 12 amino acids in length. The peptide linker may be from (about) 9 to 11 amino acids in length. The linker may be (about) 10 amino acids in length. The linker may have the amino acid sequence of SEQ ID No:23. The linker may be fused to the C-terminus of the base editor, and the UGI protein is fused to the C-terminus of the linker. The linker may be fused to the N-terminus of the base editor, and the UGI protein is fused to the N-terminus of the linker. The linker may be as described elsewhere herein.

In an embodiment, the fusion protein may be in the form of a fusion protein complex which comprises at least 3 further polypeptides,

wherein the three further polypeptides each have an amino acid sequence that is at least 90% identical to a sequence selected from the sequences of SEQ ID No:1, SEQ ID No:2, SEQ ID No:4 and SEQ ID No:5,

and wherein the fusion protein complex comprises amino acid sequences that are at least 90% identical to each of the sequences of SEQ ID No:1, SEQ ID No:2, SEQ ID No:4 and SEQ ID No:5.

In another embodiment, the fusion protein may be in the form of a fusion protein complex which comprises at least 4 further polypeptides, and wherein the fusion protein complex comprises amino acid sequences that are at least 90% identical to each of the sequences of SEQ ID No:1, SEQ ID No:2, SEQ ID No:3, SEQ ID No:4 and SEQ ID No:5.

For example, an amino acid sequence of the polypeptide is identical to SEQ ID No:1 (Cas-S1) except for 1, 2, 3, 4, 5, 6, 7, 8, 9 or 10 amino acid changes, in particular 1 to 5, for example 1 to 3, such as 1 or 2, e.g. 1 amino acid change. For example, an amino acid sequence of the polypeptide is identical to SEQ ID No:2 (Cas-S2) except for 1, 2, 3, 4, 5, 6, 7, 8, 9 or 10 amino acid changes, in particular 1 to 5, for example 1 to 3, such as 1 or 2, e.g. 1 amino acid change. For example, an amino acid sequence of the polypeptide is identical to SEQ ID No:3 (Cas-S3) except for 1, 2, 3, 4, 5, 6, 7, 8, 9 or 10 amino acid changes, in particular 1 to 5, for example 1 to 3, such as 1 or 2, e.g. 1 amino acid change. For example, an amino acid sequence of the polypeptide is identical to SEQ ID No:4 (Cas-S4) except for 1, 2, 3, 4, 5, 6, 7, 8, 9 or 10 amino acid changes, in particular 1 to 5, for example 1 to 3, such as 1 or 2, e.g. 1 amino acid change. For example, an amino acid sequence of the polypeptide is identical to SEQ ID No:5 (Cas-S5) except for 1, 2, 3, 4, 5, 6, 7, 8, 9 or 10 amino acid changes, in particular 1 to 5, for example 1 to 3, such as 1 or 2, e.g. 1 amino acid change.

In one embodiment, the fusion protein complex forms a ribonucleoprotein complex with the gRNA or crRNA which comprises a spacer that is cognate to a first protospacer in the target sequence. In one embodiment, the fusion protein forms a ribonucleoprotein complex with a gRNA or crRNA which comprises a spacer that is cognate to a first protospacer in a target sequence. In one embodiment, the gRNA or crRNA comprises two repeat sequences (e.g. two repeat sequences). The gRNA or crRNA may comprise at least one repeat sequence comprising the nucleotide sequence of SEQ ID No:6. The gRNA or crRNA may comprise two repeat sequences comprising the nucleotide sequence of SEQ ID No:6. The gRNA or crRNA may comprise at least one repeat sequence comprising the nucleotide sequence of SEQ ID No:102. The gRNA or crRNA may comprise two repeat sequences comprising the nucleotide sequence of SEQ ID No:102.

The gRNA or crRNA may comprise a spacer that is cognate to a first protospacer in the target sequence, wherein the protospacer is immediately adjacent to a Protospacer Adjacent Motif (PAM) sequence in the target sequence selected from 5′-AAC-3′, 5′-ATG-3′, 5′-AAA-3′, 5′-AAG-3′, 5′-ACG-3′, 5′-AAT-3 5′-ACA-3′, 5′-ACT-3′, 5′-ATC-3′, 5′-ATA-3′, 5′-GAG-3′, 5′-TAG-3′, 5′-ACC-3′, 5′-AGG-3′, 5′-ATT-3′, 5′-GAC-3′ and 5′-GTG-3′.

The protospacer may be immediately adjacent to a Protospacer Adjacent Motif (PAM) sequence in the target sequence selected from 5′-AAC-3′, 5′-ATG-3′, 5′-AAA-3′, 5′-AAG-3′, 5′-ACG-3′, 5′-AAT-3′, 5′-ACA-3′, 5′-ACT-3′, 5′-ATC-3, 5′-ATA-3′, 5′-GAG-3′ and 5′-TAG-3′. The protospacer may be immediately adjacent to a PAM sequence in the target sequence selected from 5′-AAC-3′, 5′-ATG-3′, 5′-AAA-3′, 5′-AAG-3′, 5′-ACG-3′, 5′-AAT-3′ and 5′-ACA-3′. The protospacer may be immediately adjacent to a PAM sequence in the target sequence selected from 5′-AAC-3′ and 5′-ATG-3′. The protospacer may be immediately adjacent to a PAM sequence in the target sequence which is 5′-AAC-3′.

The gRNA or crRNA may comprise a spacer that is cognate to a first protospacer in a target sequence, wherein the spacer sequence is from (about) 25 to 39 nucleotides in length. The spacer sequence may be from (about) 28 to 32 nucleotides in length. The spacer sequence may be about 32 nucleotides in length. The spacer sequence may be 32 nucleotides in length. The spacer may be (about) 70% (such as (about) 80%, or (about) 90%, or (about) 95%) complementary to the protospacer sequence in the target sequence. The spacer may be 100% complementary to the protospacer sequence in the target sequence.

Nucleotides 1 to 28 of the spacer may be identical to the complement of nucleotides 1 to 28 of the protospacer sequence which is immediately 5′ of the PAM sequence in the target sequence. The spacer may be identical to the complement of the protospacer in the target sequence. The spacer may be identical to the complement of the protospacer in the target sequence across its entire length. The spacer may be (about) 80% (for example (about) 90%) identical to the complement of the protospacer in the target sequence across its entire length. The spacer may be identical to the complement of the protospacer in the target sequence across the first 1 to 28 nucleotides which are immediately 5′ of the PAM sequence in the target sequence, and (about) 80% (e.g. (about) 90%) identical across the rest of the nucleotides in the protospacer.

Dual- or multi-base editors may also be used in the present disclosure, where two or more of the above mentioned base editors are fused together, thus enabling the editing of two different bases at the same time. For example a CBE may be fused with an ABE to enable both the conversion of C:G into T:A, and the conversion of A:T into G:C.

### Prime Editors

In one embodiment, the PMS encodes a nucleic acid modifier which is a prime editor.

Prime editors that may be used as a PMS are well known in the art. Prime editors include systems where Cas9 (dead-Cas9) or nCas9 (nickase Cas9) is fused to a reverse transcriptase domain and directed to the target sequence to make the desired modification with the help of a pegRNA (prime editing guide RNA, which is a guide RNA that includes a template region for reverse transcription). Thus, in one embodiment, the PMS encodes a Cas9 (dead-Cas9) or nCas9 (nickase Cas9) fused to a reverse transcriptase domain and a pegRNA (prime editing guide RNA) which directs the prime editor to the target endogenous nucleic acid to modify said target nucleic acid.

Prime Editing allows introduction of insertions, deletions (indels), and 12 base-to-base conversions. Prime editing relies on the ability of a reverse transcriptase, fused to a Cas nickase variant, to convert RNA sequence brought by a pegRNA into DNA at the nick site generated by the Cas protein. The DNA flap generated from this process is then included or not in the targeted DNA sequence.

Prime editors, as described in Anzalone et al., Nature, 576, 149-157, 2019, which is hereby incorporated by reference in its entirety, generally consist of a nickase CRISPR/Cas protein (e.g. nCas9) fused to a reverse transcriptase used in combination with a pegRNA. These systems include systems such as a Cas9-H840A (as Cas9 nickase variant) fused to a reverse transcriptase domain such as M-MLV RT or its mutant version (for example selected from M-MLV RT(D200N), M-MLV RT(D200N/L603W) and M-MLV RT(D200N/L603WfT330P/T306K/W313F)) in combination with a pegRNA.

Examples of prime editing systems include, but are not limited to PE1, PE1-M1, PE1-M2, PE1-M3, PE1-M6, PE1-M15, PE1-M3inv, PE2, PE3 and PE3b.

### RNA Interference Systems

In one embodiment, the PMS encodes a nucleic acid modifier which is an RNAi system.

For bacterial uses, the RNAi system comprises small regulatory RNAs (sRNAs). These may be naturally occurring, or synthetic. In a particular embodiment, the sRNA is an artificial sRNA. These sRNAs are sometimes referred to as non-coding RNAs. sRNAs function by base pairing between an sRNA and a messenger RNA (mRNA), which can regulate gene expression by changing the accessibility of the ribosome-binding site or altering the RNA-tumover rate. Such systems are described for example, in Noro et al., RNA Biol. 2017; 14(2), 206-218, 2016, doi:10.1080/15476286.2016.1270001, which is incorporated herein by reference in its entirety.

RNAi systems are also commercially available (e.g. from ThermoFischer etc).

### CRISPR Interference Systems (CRISPRi)

In one embodiment, the PMS encodes a nucleic acid modifier which is a CRISPRi system.

CRISPRi systems that may be used in the PMS are well-known in the art, and are commercially available (e.g. from VectorBuikier, Horizon Discovery, etc). CRISPRi systems include systems where dCas9 (dead-Cas9) is fused to one or more transcriptional repressor(s). When coupled with a gRNA (or crRNA) which is complementary to the target sequence, dCas9 targets a specific DNA location and the repressor reduces or prevents expression of the target gene. Usually, the gRNA (or crRNA) targets a region downstream of (e.g. 0-300 base pairs) the target gene's transcriptional start site (TSS) to result in interference. Thus, in one embodiment, the PMS encodes a nucleic acid modifier which is a CRISPRi system which encodes a dCas9 (dead-Cas9) fused to one or more transcriptional repressor(s) and a gRNA (or crRNA) which directs the CRISPRi system to the target endogenous nucleic acid to modify said target nucleic acid.

Examples of fusions include dCas9-KRAB (see, for example, Gilbert, et al., Cell, 154(2), 442-51, 2013; Moghadam, et al., Nat. Cell Biol., 22(9), 1143-1154, 2020; and Alerasool, er al., Nat. Methods, 17(11), 1093-1096, 2020; each of which is incorporated herein by reference in its entirety), dCas9-KRAB-MeCP2, dCas9-SALL1-SDS3 (see for example Alland, et al., Mol. & Cell Bio., 22(8), 2743-2750, 2002, incorporated herein by reference in its entirety). Thus, the CRISPRi system encodes a system selected from: dCas9-KRAB, dCas9-KRAB-MeCP2 and dCas9-SALL1-SDS3.

In a particular embodiment, the CRISPRi system is a CasS system, as described elsewhere herein, in combination with a gRNA or crRNA also as described elsewhere herein. In one embodiment, PMS encodes a nucleic acid modifier which is a CRISPRi system comprising:
a) a nucleotide sequence that encodes a polypeptide which comprises an amino acid sequence that is at least 90% identical to SEQ ID NO:2;
b) a nucleotide sequence that encodes a polypeptide which comprises an amino acid sequence that is at least 90% identical to SEQ ID NO:4; and
c) a nucleotide sequence that encodes a polypeptide which comprises an amino acid sequence that is at least 90% identical to SEQ ID NO:5; and
d) a guide RNA or crRNA.

The CRISPRi system may optionally further comprise a nucleotide sequence that encodes a polypeptide which comprises an amino acid sequence that is at least 90% identical to SEQ ID NO:1. The CRISPRi system may further comprises a nucleotide sequence that encodes a polypeptide which comprises an amino acid sequence that is at least 90% identical to SEQ ID NO:3.

For the CasS CRISPRi systems described herein, the crRNA or gRNA may have any of the features described herein in the Base Editor section.

### Nuclease Systems

In one embodiment, the PMS encodes a nucleic acid modifier which is a nuclease. In one embodiment, the PMS encodes a nucleic acid modifier which is a nuclease and a gRNA or crRNA.

Nuclease systems that may be used in the PMS are well known in the art. In particular, nucleases which are mutated such that they are no longer able to cleave DNA can be used to silence genes. Suitable nuclease systems include, but are not limited to CRISPR/Cas systems, such as dCas9, type IV CRIPSR/Cas systems or type I CRISPR/Cas systems in which the nuclease (Cas3) has been removed, a TALEN or a zinc finger protein. Thus, in one embodiment, the PMS encodes a nucleic acid modifier which is a nuclease which cannot introduce double stranded breaks in the target endogenous nucleic acid, and the gRNA (or crRNA) directs the nuclease to the target endogenous nucleic acid to modify said target nucleic acid.

In one embodiment, the nuclease is selected from a meganuclease, a zinc finger, a TALEN or a restriction enzyme which has been modified to prevent cutting of DNA or RNA.

### Other Gene Targeting Systems for Use as the PMS

Other systems for gene editing are being developed. Thus, further examples of systems which may be employed in the PMS described herein include the following systems:
1) Cas9 Retron precISe Parallel Editing via homologY (“CRISPEY”), which is a retron RNA fused to the sgRNA and expressed together with Cas9 and the retron proteins including at least the reverse transcriptase, as described in Sharon eal., Cell, 175, 2018, 544-557.e16, which is incorporated herein by reference in its entirety.
2) The SCRIBE strategy, which uses a retron system expressed in combination with a recombinase, promoting the recombination of single stranded DNA, also known as single stranded annealing proteins (SSAPs), see for example Farzadfard & Lu, Science, 346, 2014, 1256272, which is incorporated by reference herein in its entirety. Such recombinases include but are not limited to phage recombinases such as lambda red, recET, Sak, Sak4, and SSAPs described in Wannier et al, Biorxiv, 2020.01.14.906594, 2020, doi: 10.1101/2020.01.14.906594, which is hereby incorporated by reference in its entirety.
3) The targetron system, which is based on group II introns described in Karberg et al., Nat Biotechnol 19, 2001, 1162-7, which is incorporated herein by reference in its entirety, and which has been adapted to many bacterial species.
4) Other retron based gene targeting approaches are described in Simon et al., Nucleic Acids Res, 47, 2019, 11007-11019, which is incorporated herein by reference in its entirety.

### PMS Encoding Peptide Molecules

In one embodiment, the PMS encodes peptide inhibitor molecules. These are usually short peptide mimetics, which have structural similarity to the native substrate of the target endogenous protein, i.e. an enzyme, exporter or importer (e.g. as illustrated in FIG. 1 or FIG. 2). The peptide inhibitors, unlike the native substrates, are unable to undergo the catalytic reaction of the enzyme, and thus remain bound within the active site of the target endogenous protein, i.e. an enzyme, exporter or importer, thus preventing the undesirable reaction from taking place.

Alternatively, the peptide inhibitor molecules may bind on the surface of the target endogenous protein, and thereby prevent the correct assembly of a multi-subunit protein, which multi-subunit protein cannot perform its native function without all the subunits, or cannot perform its natice function with the bound peptide molecule on its surface.

Alternatively, the peptide inhibitor molecules may bind to a nascent endogenous target protein as it is being transcribed, and thereby prevent the correct folding of the endogenous target protein.

Some peptide mimetics are known and can be used for the described applications.

Various methods exist for identifying and testing potential peptide inhibitors. For example, Mukhija et al., Eur. J. Biochem., 254(2):433-8, 1998, doi:10.1046/j.1432-1327.1998.2540433.x (incorporated herein by reference in its entirety) discloses methods for preparing inhibitor peptides of enzyme I of the bacterial phosphotransferase system using immobilized combinatorial peptide libraries and phosphorimaging. Fu et al., PLoS ONE, 12(8), e0182847, 2017, doi: https://doi.org/10.1371/joumal.pone.0182847 (incorporated herein by reference in its entirety) discloses methodology that uses molecular dynamics and point-variant screening to identify short peptide motifs that are critical for inhibiting a particular enzyme, β-galactosidase. Chen et al, Nat. Commun. 15, 1611, 2024, doi:https://doi.org/10.1038/s41467-024-45766-2 (incorporated herein by reference in its entirety) discloses a method which integrates a Gated Recurrent Unit-based Variational Autoencoder (using generative deep learning) with Rosetta FlexPepDock for peptide sequence generation and binding affinity assessment followed by molecular dynamics simulations to design peptides for experimental binding studies.

Crystal structures of many proteins, such as enzymes, importers and exporters are known or can be predicted, and software exists (for example Alphafold, see https://alphafold.ebi.ac.uk/) which may be able to predict the structure of the 3D structure of the protein, to further enable design of such peptide inhibitor molecules.

### Exogenous Genes for the Production of a First MOI

The transmissible elements described herein comprise at least one exogenous nucleic acid sequence to the recipient bacterium for the production of a first molecule of interest (MOI) in the recipient bacterium. The exogenous nucleic acid sequence may encode the first MOI. The exogenous nucleic acid may be an exogenous gene for the production of the first MOI.

The at least one exogenous nucleic acid for the production of a first MOI may encode an MOI which is a therapeutic molecule.

In one embodiment, the first MOI is a therapeutic peptide molecule, e.g. a therapeutic protein. Therapeutic proteins may be used to, for example, replace a protein that is deficient or abnormal, augment an existing biological pathway, provide a novel function or activity, interfere with a molecule or organism, and/or deliver other compounds or proteins, such as a radionuclide, cytotoxic drug, or effector proteins.

Therapeutic proteins include antibody fragments, hormones, interleukins, cytokines, chemokines, eukaryotic growth factors and/or enzymes.

When the first MOI is a protein molecule (e.g. a therapeutic protein molecule), a bacterial transport signal peptide (SP) may be needed so that the bacteria can secrete the active substances out of the bacteria. SPs are short peptides with 16-30 amino acids and are N-terminal extensions of secreted proteins. They act as a target and recognition signal for signal peptidases which remove SPs from the translocated protein across the cytoplasmic membrane and cell wall. This process will result in the extracellular release of the mature protein or peptide.

Thus, a signal peptide may also be required to aid secretion of the first MOI (e.g. therapeutic protein molecule) to the periplasm of the recipient bacterium, to the cell surface of the recipient bacterium, or to the extracellular space outside of the recipient bacterium. In any embodiment herein, the transmissible element may further comprise a (exogenous) nucleic acid encoding a signal peptide for secretion of the first MOI (e.g. therapeutic protein molecule) to the periplasm of the recipient bacterium. In any embodiment herein, the transmissible element may further comprise a (exogenous) nucleic acid encoding a signal peptide for secretion of the first MOI (e.g. therapeutic protein molecule) to the cell surface of the recipient bacterium. In any embodiment herein, the transmissible element may further comprise a (exogenous) nucleic acid encoding a signal peptide for secretion of the first MOI (e.g. therapeutic protein molecule) to the extracellular space outside of the recipient bacterium.

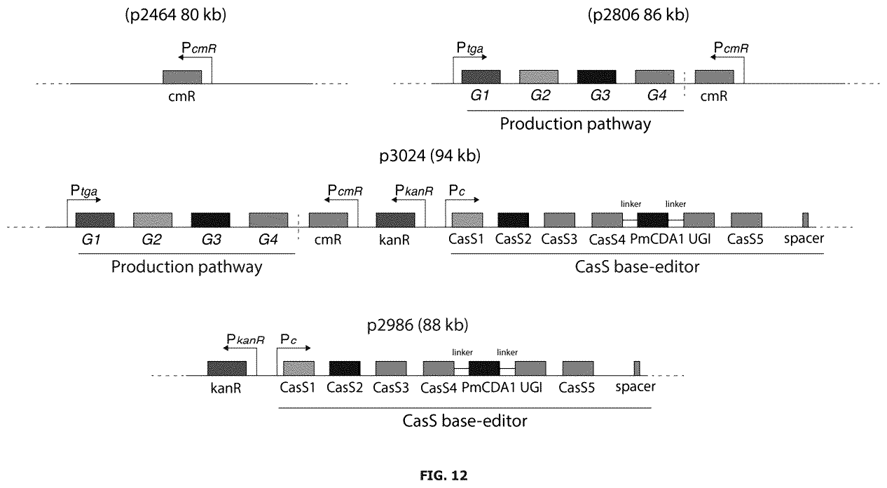

Further, as is known in the art, diffusion of molecules through bacterial membrane(s) (both gram-negative and gram-positive bacteria) can limit the amount of a particular molecule which diffuses to the local environment. Without being bound by theory, in general, bacterial membrane(s) are hydrophobic, so the more hydrophilic the first MOI is, the less likely it is to be able to cross the membrane. However, certain molecules which are detrimental to the cell (for example toxins, bacteriocins, etc) are exported more often, along with certain molecules which provide specific extra-cellular functions (such as molecules associated with quorum sensing, iron acquisition, etc). In addition, some molecules are actively transported through the membrane(s) by dedicated exporters (for example, excess amino acids may be exported from the cell to maintain homeostasis, such as the alaE exporter of alanine, and the leuE exporter of leucine both found in E. coli), or exporters which export a certain class of molecule (for example, the setA transporter found in E. coli exports various sugar molecules). Thus, in many bacteria, secretion of MOIs may be limited by the rate of diffusion. The inventors have engineered conjugative plasmids to express heterologous exporters which are capable of exporting an first MOI across the membrane(s) of the bacteria. Unexpectedly, heterologous exporters are able to form and function within the bacterial membrane (G4 in FIG. 12 is an exporter of the first MOI). This leads to an increased secretion of the first MOI into the local environment, and contributes to the reduced fitness disadvantage of the expression of the first MOI within the bacteria, because (without being bound by theory) the first MOI is removed from the cytoplasm and/or periplasm into the local environment.

Thus, in any embodiment herein, the transmissible element may further comprise a nucleic acid encoding one or more exporter(s) of the first MOI (e.g. the therapeutic protein molecule or metabolite) from the recipient bacterium. In any embodiment herein, the transmissible element may further comprise an exogenous nucleic acid encoding one or more exporter(s) of the first MOI (e.g. the therapeutic protein molecule or metabolite) from the recipient bacterium. In a particular embodiment, the nucleic acid encodes an exporter of the first MOI.

In one embodiment, the exogenous nucleic acid of A) encodes, in 5′ to 3′ direction, a promoter, a signal peptide, at least one nucleic acid for the expression of the first MOI (e.g. therapeutic protein) and optionally a further nucleic acid sequence encoding one or more exporter(s) of the first MOI (e.g. therapeutic protein) from the recipient bacterium. In a particular embodiment, the nucleic acid encodes an exporter of the first MOI.

The therapeutic molecule may be an antibody therapy. In particular, the therapeutic molecule is an antibody fragment. The therapeutic molecule may comprise an antibody (or in particular an antibody fragment) comprising the binding domains of any of the specific antibody molecules described herein. The therapeutic molecule may comprise an antibody fragment which binds to any of the targets of any of the antibody molecules described herein. Fc fusion proteins are also contemplated.

The antibody therapy may be an immune checkpoint inhibitor antibody or fragment thereof. The antibody or fragment thereof may be selected from an anti-PD-L1, anti-PD-1, anti-CTLA4, anti-TIM3, anti-TNFa superfamily member (such as an anti-TNFa, TNFR1 or BAFF), anti-IL6R, anti-IL-4Ra, or anti-PCSK9.

Examples of antibodies, antibody fragments, and/or Fc fusion proteins include, without limitation, Abagovomab, Abciximab, Actoxumab, Adalimumab, Adecatumumab, Afelimomab, Afutuzumab, Alacizumab pegol, ALD, Alemtuzumab, Alirocumab, Altumomab pentetate, Amatuximab, Anatumomab mafenatox, Anifrolumab, Anrukinzumab, Apolizumab, Arcitumomab, Aselizumab, Atinumab, Atlizumab (or tocilizumab), Atorolimumab, Bapineuzumab, Basiliximab, Bavituximab, Bectumomab, Belimumab, Benralizumab, Bertilimumab, Besilesomab, Bevacizumab, Bezotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, Cedelizumab, Certolizumab, pegol, Cetuximab, Citatuzumab bogatox, Cixutumumab, Clazakizumab, Clenoliximab, Clivatuzumab tetraxetan, Conatumumab, Concizumab, Crenezumab, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizu mab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Namatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN, Ticilimumab (or tremelimumab), Tildrakizumab, Tigatuzumab, TNX-, Tocilizumab (or atlizumab), Toralizumab, Tositumomab, Tovetumab, Tralokinumab, Trastuzumab, TRBS, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and Zolimomab aritox.

Therapeutic hormones include glucagon-like peptide-1 (GLP-1) or GLP-t and insulin. Other examples of peptide hormones include, without limitation, Amylin (or Islet Amyloid Polypeptide), Antimullerian hormone (or MOllerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Brain natriuretic peptide, Calcitonin, Cholecystokinin, Corticotropin-releasing hormone, Enkephalin, Endothelin, Erythropoietin, Follicle-stimulating hormone, Galanin, Gastrin, Ghrelin, Glucagon, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Lipotropin, Luteinizing hormone, Melanocyte stimulating hormone, Motilin, Orexin, Oxytocin, Pancreatic polypeptide, Parathyroid hormone, Prolactin, Prolactin releasing hormone, Relaxin, Renin, Secretin, Somatosta tin, Thrombopoietin, Thyroid-stimulating hormone (or thyrotropin), and Thyrotropin-releasing hormone.

Examples of interleukins include, without limitation, interleukin 1, 2, 3, 4, 5, 6, 7, 8, 9, 10, 11, 12, 13, 14, 15, 16 or 17. The interleukin may be interleukin-2, interleukin-4, interleukin-6, interleukin-7, interleukin-10, interleukin-11 or interleukin-13. The interleukin may in particular be interleukin 1-17.

Examples of cytokines include, without limitation pegfilgratim, anakinra, emfilermin, denileukin diftitox, interferon-α, erythropoietin, granulocyte-macrophage colony stimulating factor, interleukin-1 receptor antagonist, granulocyte colony stimulating factor, Cintredekin besudotox, Romiplostim, Regramostim, Albinterferon Alfa-2B, Maxy-G34, Thrombopoietin, stem cell factors, erythropoiesis stimulating agents, Avotermin, Nagrestipen, Binetrakin, Dulanermin, Muplestim, Moigramostim, Balugrastim, Lipegfilgrastim, CD40-ligand, Leridistim, Bempegaldesleukin, Pegilodecakin, ALT-801, Teceleukin, Lorukafusp-α, edodekin-α, viral macrophage inflammatory protein-II (vMIP), interferon-κ, Efineptakin-α and Tengonermin. Examples of interferons (IFNs) include, without limitation, IFN-α, IFN-β, IFN-ω and IFN-γ.

Examples of chemokines include, without limitation, CC chemokines (e.g. CCR1, CCR2, CCR3, CCR4, CCR5, CCR6, CCR7, CCR8, CCR9 and CCR10), CXC chemokines (e.g. CXCR1, XCR2, CXCR3, CXCR4, CXCR5, CXC6 and CXCR7), C chemokines or CX3C chemokines (e.g. CX3CR1).

Examples of growth factors include, without limitation, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-0), Tumor necrosis factor-alpha (TNF-α), Vascular endothelial growth factor (VEGF), placental growth factor (PIGF), Foetal Bovine Somatotrophin (FBS) and IL-1-IL7.

Examples of enzymes include, without limitation, any of the enzymes assigned an Enzyme Commission Number (EC) number (e.g. EC1-EC6) by the International Union of Biochemistry and Molecular Biology (IUBMB) (Webb, Edwin C. Enzyme nomenclature 1992: recommendations of the Nomenclature Committee of the Intemational Union of Biochemistry and Molecular Biology on the nomenclature and classification of enzymes. San Diego: Published for the Intemational Union of Biochemistry and Molecular Biology by Academic Press. ISBN 0-12-227164-5 (1992), incorporated herein by reference). Other examples include: styrene monooxygenase (StyAB), toluene dioxygenase (TODC1C2AB), luciferase and lactase. In some embodiments, the enzyme is toluene dioxygenase. In some embodiments, the enzyme is styrene monoxygenase.

In any embodiment, the first MOI is a therapeutic molecule described in Table 1 below.

TABLE 1
MOIs produced by genetically modified bacteria

| | | | Therapeutic | | |
|---|---|---|---|---|---|
| Engineered strain | Disease | Study | Molecule | Applications | References |
| Lactococcus lactis | Type2 Diabetes | In vitro | rExd4 | Enhances glucose-dependent insulin secretion and | Zeng et al. |
| | | | | activates the PI3-K/AKT signal pathway | (2017) |
| Lactococcus lactis | Mucositis | In vitro | Pancreatitis- | It reduces intestinal inflammation by preventing 5- | Carvalho et al. |
| | | | associated protein | fluoracil | (2017) |
| Lactobacillus | Colorectal cancer | In vitro | Gamma- | Activates anti-proliferative, anti-migration, and anti- | An et al. (2021) |
| plantarum | | | aminobutyric acid | invasion effects against 5-FU-resistant HT-29 cells | |
| Lactococcus lactis | Cancer | In vivo | Anti-tumor-genic | Capable of inducing effective elimination of human | Ciaćma et al. |
| | | | human TRAIL | colon cancer cells in vivo | (2018) |
| Lactobacillus | Colorectal cancer | In vivo and | YYC-3 | It reduces colon cancer metastasis | Yue et al. |
| plantarum | | In vitro | | | (2020a) |
| Lactococcus lactis | Rheumatoid | In vivo | Hsp65-Lac | Prevent the induction of chronic and acute models of | Gusmao-Silva |
| | arthritis | | | arthritis | et al., (2020) |
| Lactococcus lactis | H. pylori | In vivo | HpaA | It provides anti-H. pylori vaccination with potent | Zhang et al. |
| | infection | | | immunogenicity | (2017) |
| Lactococcus lactis | H. pylori | In vivo | napA | Produces and delivers the oral vaccine against H. | Peng et al., |
| | infection | | | pylori | (2018) |
| Lactococcus lactis | Vibrio cholera | In vivo | β-lactamase | Hinder cholera progression and improve disease | Mao et al. |
| | infection | | | surveillance in populations at risk of cholera | (2018) |
| | | | | outbreaks | |
| Bifidobacterium | Breast cancer | In vitro | Trastuzumab scFv | Provide in situ delivery to kill cancer cells | Kikuchi et al. |
| longum | | | | | (2017) |
| Bifidobacterium | Irritable bowel | In vitro and | LL-37 | Provide therapeutic effect against bacterial diarrhea | Guo et al. |
| longum | syndrome | In vivo | | by reducing the population of E. coli and S. aureus | (2017) |
| Bifidobacterium | Inflammatory | | MnSOD | Reduced DSS-induced ulcerative colitis in mice | Liu et al. |
| longum | bowel disease | | | | (2018) |
| E. coli Nissle 1917 | VRE infection | In vivo | Enterocin A, | Specifically targeted and kill Enterococcus, potent | Geldart et al. |
| | | | Enterocin B, and | activity against both Enterococcus | (2018) |
| | | | Hiracin JM79 | faecium and Enterococcus faecali | |
| E. coli Nissle | IBD | In vitro and | TFF | Treat inflammation and help to rebuild the intestinal | Praveschotinunt |
| | | in vivo | | epithelium | et al. (2019) |
| Salmonella | Melanoma Cancer | In vivo | Interferon-gamma | S. typhimurium and IFN-g showed therapeutic | Yoon et al. |
| typhimurium | | | (IFN-γ) | potential for the treatment | (2017) |
| E. coli Nissle 1917 | Phenylketonuria | In vivo | phenylalanine | Degradation of accumulated phenylalanine (Phe) by | Puurunen et al. |
| | | | ammonia lyase | deamination of Phe to the non-toxic products trans- | (2021) |
| | | | and L-amino acid | cinnamate (TCA) and phenylpyruvate | |
| | | | deaminase | | |
| E. coli | Cancer | In vivo | nanobody | Lysis within tumour to release nanobody | Chowdhury et |
| | | | antagonist of | | al. (2019) |
| | | | CD47 | | |
| L. lactis | Diabetes | In vitro | exendin-4 | Expressing bioactive exendin-4 to promote insulin | Zeng et al |
| | | | | secretion and beta-cell proliferation | (2017) |
| L. lactis | Inflammation | In vivo | Anti-TNF scFv | Anti-TNF scFv expression | Chiabal et al |
| | | | | | (2019) |

Table produced from Omer et al., supra and Zhou et al., Engineering Microbiol., 2(3), 100034, 2022, doi: https://doi.org/10.1016/j.engmic.2022.100034
Zeng et al., Appl. Microbiol. Biotechnol., 101, 7177-7186, 2017, doi: 10.1007/s00253-017-8410-6
Carvalho et al., Microb. Cel. Fact., 16, 27-0624, 2017, doi: 10.1186/s12934-017-0624-x
An et al. J. Microbiol., 59, 202-21, 2021, doi: 10.1007/s12275-021-0562-5
Claćma et al., Microb, Cel. Fact., 17, 018-1028, 2018, doi :10.1186/s12934-018-1028-2
Yue et al., Microb. Cel. Fact., 19, 213, 2020, doi: 10.1186/s12934-020-01466-2
Gusmao-Silva et al., Front. Immunol., 11, 562905, 2020, doi: 10.3389/fimmu.2020.562905
Zhang et al., PLOS One, 12, e0188960, 2017, doi: 10.1371/journal.pone.0188960
Peng et al., Sci. Rep., 8, 6435, 2018, doi: 10.1038/s41598-018-24879-x
Mao et al., Sci. Transl, Med., 10, eaao2586, 2018, doi: 10.1126/scitranslmed.aao2586
Kikuchi et al., Biochem. Biophysical Res. Commun., 493(1), 306-312, 2017, doi: 10.1016/j.bbrc.2017.09.026
Guo et al., J. Pharm. Biomed. Sci., 7(6), 2017, doi: 10.20936/JPBMS/170602
Liu et al., Int. Immunopharmacology, 57, 25-32, 2018, doi: 10.1016/j.intimp.2018.02.004
Geldart et al., Bioeng, Translational Med., 3, 197-208, 2018, doi: 10.1002/btm2.10107
Praveschotinunt et al., Nat. Commun., 10, 5580, 2019, doi: 10.1038/s41467-019-13336-6
Yoon et al., Eur. J. Cancer, 70, 48-61, 2017, doi: 10.1016/j.ejca.2016.10.010
Puurunen et al., Nat. Metab., 3, 1125-1132, 2021, https://doi.org/10.1038/s42255-021-00430-7
Chowdhury et al., Nat. Med., 25, 1057-1063, 2019, https://doi.org/10.1038/s41591-019-0498-z
Zeng et al., Appl. Microbiol. Biotechnol., 101, 7177-7186, 2017, https://doi.org/10.1007/s00253-017-8410-6
Chiabai et al., BMC Biotechnol., 19, 38, 2019, https://doi.org/10.1186/s12896-019-0518-6

Other therapeutic molecules include, without limitation, anticoagulants, blood factors, bone morphogenetic proteins, engineered protein scaffolds, and thrombolytics. Other examples include those that bind non-covalently to target (e.g. monoclonal antibodies or fragments thereof), those that affect covalent bonds (e.g. enzymes), and those that exert activity without specific interactions (e.g. serum albumin).

Therapeutic molecules include recombinant therapeutic proteins, used to treat, for example, cancers, immune disorders, infections and/or other diseases.

In one embodiment, the therapeutic protein is selected from Etanercept, Bevacizumab, Rituximab, Adalimumab, Infliximab, Trastuzumab, Insulin glargine, Epoetin alfa, Pegfilgrastim, Ranibizumab, Darbepoetin alfa, Interferon beta-1α, Insulin aspart, Rhu insulin, Octocog alfa, Insulin lispro, Cetuximab, Peginterferon alfa-2a, Interferon beta-1b, Eptacog alfa, Insulin aspart, OnabotulinumtoxinA, Epoetin beta, Rec antihemophilic factor, Filgrastin, Insulin detemir, Natalizumab, Insulin (humulin) and Palivizumab.

Examples of Fc fusion proteins include, without limitation, Etanercept, Alefacept, Abatacept, Rilonacept, Romiplostim, Belatacept and Aflibercept.

Examples of anticoagulants and/or blood factors include, without limitation, Protein C, Protein S, and antithrombin, Factors I-VIII, prothrombinase, prothrombin, thrombin von Willebrand Factor (vWF), fibrinogen, fibrin and fibrinopeptides.

Examples of bone morphogenetic proteins (BMPs) include, without limitation, BMP1-BMP7, BMP8a, BMP8b, BMP10, and BMP15.

Other examples of therapeutic proteins include, without limitation, Insulin (blood glucose regulator), Pramlintide acetate (glucose control), Growth hormone GH (growth failure), Pegvisoman (growth hormone receptor antagonist), Mecasermin (IGF1, growth failure), Factor VIII (coagulation factor), Factor IX (coagulation factor, Protein C concentrate (anti-coagulation), α1-proteinase inhibitor (anti-trypsin inhibitor), Erythropoietin (stimulates erythropoiesis), Filgrastim (granulocyte colony-stimulating factor, G-CSF; stimulates neutrophil proliferation), Sargramostim 36, 37 (granulocyternacrophage colony-stimulating factor, GM-CSF), Oprelvekin (interleukin-11, IL-11), Human follicle-stimulating hormone (FSH), Human chorionic gonadotropin (HCG), Lutropin-α (human luteinizing hormone), Interleukin 2 (IL-2), Interleukin-1 Receptor Agonist, Denileukin diftitox (fusion of IL-2 and Diphtheria toxin), Interferon alfacon 1 (consensus interferon), Interferon-α2a (IFN-α2a), Interferon-α2b (IFN-α2b), Interferon-αn3 (IFN-αn3), Interferon-β1a (rIFN-β), Interferon-plb (rIFN-β), Interferon-γb (IFNγ), Salnon calcitonin (32-amino acid linear polypeptide hormone), Teriparatide (part of human parathyroid hormone 1-34 residues), Exenatide (Incretin mimetic with actions similar to glucagon-like peptide 1), Octreotide (octapeptide that mimics natural somatostatin), Dibotermin-α (recombinant human bone morphogenic protein 2), Recombinant human bone morphogenic protein 7, Histrelin acetate (gonadotropin-releasing hormone; GnRH), Palifermin (Keratinocyte growth factor, KGF), Becaplermin (platelet-derived growth factor, PDGF), Nesiritide (recombinant human B-type natriuretic peptide), Lepirudin (recombinant variant of hirudin, another variant is Bivalirudin), Anakinra (interleukin 1 (IL-1) receptor antagonist), Enfuviritide (an HIV-1 gp41-derived peptide), β-Glucocerebrosidase (hydrolyzes to glucose and ceramide), Alglucosidase-α (degrades glycogen), Laronidase (digests glycosaminoglycans within lysosomes), Idursulfase (cleaves O-sulfate preventing GAGs accumulation), Galsulfase (cleave terminal sulphage from GAGs), Agalsidase-β (human α-galactosidase A, hydrolyzes glycosphingolipids), Lactase (digest lactose), Pancreatic enzymes (lipase, amylase, protease; digest food), Adenosine deaminase (metabolizes adenosine), Tissue plasminogen activator (tPA, serine protease involved in the breakdown of blood clots), Factor VIIa (serine protease, causes blood to clot), Drotrecogin-α (serine protease, human activated protein C), Trypsin (serine protease, hydrolyzes proteins), Botulinum toxin type A (protease, inactivates SNAP-25 which is involved in synaptic vesicle fusion), Botulinum toxin type B (protease that inactivates SNAP-25 which is involved in synaptic vesicle fusion), Collagenase (endopeptidase, digest native collagen), Human deoxyribonuclease I (endonuclease, DNase I, cleaves DNA), Hyaluronidase (hydrolyzes hyaluronan), Papain (cysteine protease, hydrolyzes proteins), L-Asparaginase (catalyzes the conversion of L-asparagine to aspartic acid and ammonia), Rasburicase (urate oxidase, catalyzes the conversion of uric acid to allantoin), Streptokinase (Anistreplase is anisoylated plasminogen streptokinase activator complex (APSAC)), and Antithrombin III (serine protease inhibitor).

Examples of small molecule therapeutics include beneficial metabolites. Therapeutics include GLP1, GLP2, IL10, IL22, TNF-α, a STING agonist such as ci-di-AMP and human epidermal growth factor. Beneficial metabolites include, without limitation, L-DOPA, butyrate, propionate, acetate, GLP1, GLP2, IL10, IL22, indole, tryptophan, indole-3-acetic acid (IAA), arginine, tryptophan, kynurenine and kynurenic acid.

Production of TNF-α (first MOI) is described in WO2022/072636A1 (Synlogic Operating Company, Inc.), incorporated herein by reference in its entirety.

Production of the STING agonist, ci-di-AMP (first MOI), using the dacA gene from Lisetria monocytogenes is disclosed in WO2022/150779A1 and WO2020/097424A1 (both Synlogic Operating Company, Inc.), each of which is incorporated herein by reference in its entirety. This MOI is useful in treating cancers.

Production of human epidermal growth factor (hEGF, first MOI), which uses the human EGF cDNA sequence (NCBI accession number:gq214314, codon-optimised for expression in E. coli), with N-terminal see secretion signal sequences of either PhoA (21 amino acids), PcIB (22 amuio acids), or OmpA (21 amino acids) is disclosed in WO2022/221273A1 (Synlogic Operating Company, Inc.), incorporated herein by reference in its entirety. Other expression constructs which could be used in the present disclosure for expression of a first MOI are disclosed in Example 1 therein.

In one embodiment, the at least one exogenous nucleic acid sequence of A) does not comprise any reporter genes. In one embodiment, the at least one exogenous nucleic acid sequence of A) does not comprise any antibiotic resistance genes.

### Exogenous Genes for the Conversion of a Second MOI to a First MOI

The at least one exogenous nucleic acid for the production of a first MOI of A) may encode one or more protein(s) (G1, G2 . . . GX) for the conversion of a second MOI (B) to the first MOI (A).

Where a second MOI is converted to a first MOI, the first MOI may be selected from a therapeutic molecule (e.g. as described elsewhere herein) and a beneficial or therapeutic metabolite.

For example, the therapeutic molecule may be a prodrug, and the protein encoded by the at least one exogenous nucleic acid is an enzyme which converts the prodrug to the active therapeutic drug. In another example, the therapeutic molecule is a peptide or fusion protein, and the protein encoded by the at least one exogenous nucleic acid cleaves the peptide or fusion protein to release a desired sub-unit of the original peptide or fusion protein.

Examples of small molecule therapeutics include beneficial metabolites. Beneficial metabolites include, without limitation, L-3,4-dihydroxyphenylalanine (L-DOPA), butyrate, propionate, acetate, GLP1, GLP2, I10, IL22, indole, tryptophan, indole-3-acetic acid (IAA), arginine, tryptophan, kynurenine and kynurenic acid.

Alternatively, the second MOI may be a detrimental metabolite. In this embodiment, the first MOI is either neutral or does not cause the detrimental effect of the second MOI to the cell, organism or microbiome. Thus, reducing levels of detrimental metabolites may be achieved by the transmissible elements herein.

Such detrimental metabolites include leucine, isoleucine, valine, chenodeoxycholic acid (CDCA), oxalate, uric acid, propionate and methionine.

In a particular embodiment, the first MOI is a beneficial metabolite or a therapeutic metabolite.

L-DOPA (a first MOI), for example, can be made from tyrosine (second MOI) using tyrosine 3-hydroxylase (EC 1.14.16.2); or from L-phenylalanine (second MOI) using two enzymes, phenylalanine 4-hydroxylase (EC 1.14.16.1) and tyrosine 3-hydroxylase. Methods for gene insertion into bacteria are provided in WO2022/013407A1 (Danmarks Tekniske Universitet), which is incorporated herein by reference in its entirety.

Genes for producing butyrate (first MOI) from crotonyl-CoA (second MOI) are described for example in WO2017/136792A2 and WO2017/074566A1 (Synlogic Operating Company, Inc.), which are both incorporated herein by reference in their entirety. Genes may include one or more (for example all) of the genes from butyrate production pathway from Peptoclostridium difficile 630 (bcd2, etfB3, elfA3, thiA1, hbd, crt2, pbt, and buk); or trans-2-enoynl-CoA reductase (ter) from Treponema denticola, in combination with thiA1, hbd, crt2, pbt, and buk from Peptoclostridium difficile 630; or a thioesterase (tesb) from E. Coli in combination with bcd2, ebfB3, el^{f}A3, thiA1, hbd, crt2 from Peptoclostridium difficile 630; or ter from Treponema denticola, in combination with thiA1, hbd, crt2, pbt and buk from Peptoclostridium difficile 630 and tesB from E. coli. Any of the intermediate substrates of these reactions could be the second MOI, and the number of exogenous genes could be reduced if the recipient bacterium is able to produce the intermediate substrate itself.

WO2017/074566A1 (Synlogic Operating Company, Inc.), incorporated herein by reference in its entirety, discloses genes for the production of gut barrier enhancer molecules, such as propionate, acetate, GLP2, IL-22, IL-10, indole and tryptophan (each first MOIs).

WO2017/123418A1, WO2021/242897A1 and WO2023/225667A2 (all Synlogic Operating Company, Inc.), each of which is incorporated herein by reference in its entirety, disclose genes for the production of IAA (first MOI) from tryptophan (second MOI), and deletions in trpR and tnaA genes to increase the production of IAA, also see other embodiments disclosed herein for further genes and configurations for the production of IAA from various molecules.

WO2016/090343A1 and WO2020/232063A1 (both Synlogic Operating Company, Inc), each of which is incorporated herein by reference in its entirety, disclose methods of converting glutamate (second MOI) to arginine (first MOI) in an eight-step enzymatic process involving N-acetylglutamate synthetase (ArgA), N-acetylglutamate kinase (ArgB), N-acetylglutamate phosphate reductase (ArgC), acetylornithine aminotransferase (ArgD), N-acetylornithinase (Argf), carbamoylphosphate synthase (encoded by carA and carB), ornithine transcarbamylase (ArgF and ArgI), argininosuccinate synthase (ArgG), and argininosuccinate lyase (ArgH). Ornithine acetyltransferase (Arg)) is bifunctional and can be used instead of both N-acetylglutamate synthetase (ArgA) and N-acetylornithinase (ArgE). All of the genes encoding these enzymes are subject to repression by arginine via its interaction with ArgR to form a complex that binds to the regulatory region of each gene and inhibits transcription. Thus, the PMS may encode a nucleic acid inhibitor or peptide molecule which reduces or prevents arginine-mediated repression, for example by preventing binding of arginine (first MOI) to ArgR or by preventing ArgR binding to the regulatory region of each gene (known as an “ARG box”), or by arginine binding to N-acetylglutamate synthetase.

WO2016/210384A1 (Synlogic Operating Company, Inc.), incorporated herein by reference in its entirety, discloses genes for the production of various metabolic or satiety effector molecules, including bile salt hydrolase, n-acyl-phophatidylethanolamine (NAPE), n-acyl-ethanolamines (NAE), ghrelin receptor antagonist, peptide YY3-36, acholecystokinin (CCK), CCK58, CCK33, CCK22, CCK8, bombesin, gastrin releasing peptides (GRP), neuromedin B (P), glucagon, GLP-1, GLP-2, apolipoprotein A-IV, amylin, somatostatin, enterostatin, oxyntomodulin, pancreatic peptides, short-chain fatty acids, butyrate, propionate, acetate, serotonin receptor agonists, nicotinamide adenine dinucleotides (NAD), nicotinamide mononucleotides (NMN), nucleotide riboside (NR), nicotinamide, nicotinic acid (NA), but in particular examples tryptophan, kynurenine, kynurenic acid, indole and IAA, as well as transporters for importing these molecules into the cell.

In an embodiment, the second MOI may be a detrimental metabolite.

WO2016/201380A1, WO2021/188618A1 and WO2021/146394A1 (all Synlogic Operating Company, Inc.), each of which is incorporated herein by reference in its entirety, disclose genes for the reduction of branched chain amino acids, such as leucine, isoleucine, valine (second MOIs), as well as various mutations in exporters to reduce export of the second MOIs, and importers to increase import of the second MOIs into the cell for increased degradation.

Further, WO2020/257610A1 and WO2020/257707A1 (both Synlogic Operating Company, Inc. and Ginko Bioworks, Inc.) each of which is incorporated herein by reference in its entirety, disclose genes for the reduction of leucine (second MOI), converting it into isopentanol (first MOI) and including importers to increase import of the second MOI into the cell for increased degradation.

WO2022/169909A2 (Novome Biotechnologies, Inc.), incorporated herein by reference in its entirety, describes Bacteroides strains which have been engineered to convert CDCA (second MOI) to ursodeoxycholic acid, UDCA (first MOI). Chenodeoxycholic acid, CDCA is detrimental metabolite which is elevated in diseases such as IBS, whereas UDCA is generally considered tolerable for humans. Two enzymes 7α-HSDH from E. coli Nissle 1917 and 7β-HSDH from Colinsella aerofaciens ATCC 25986 are used to convert CDCA into UDCA.

Oxalate accumulation in urine causes hyperoxaluria. Elevated urinary oxalate has been linked to recurrent calcium oxalate kidney stones, kidney damage, and eventually end-stage renal disease. WO2020/123483A1 (Novome Biotechnologies, Inc.), incorporated herein by reference in its entirety, describes various oxalate-metabolising enzymes and proteins (e.g. oxalate decarboxylase (OXDC), EC 4.1.1.2, e.g. from O. formigenes; oxalate decarboxylase; oxalate oxidase (OXO), EC 1.2.3.4, e.g. from Hordeum vulgare, oxalate oxidoreductase (OOR), EC 1.2.7.10, e.g. from Moorea themoacebica, ATCC 39073; oxalate-CoA ligase/oxalyl-CoA synthetase (OXS), EC 6.2.1.8, e.g. from A. thaliana or S. cerevisiae, formyl-CoA:oxalate CoAtransferase (FCOCT), EC 2.8.3.16, e.g. from E. coli; acetyl-CoA:oxalate CoA-transferase (ACOCT), EC 2.8.3.19, e.g. from E. coli, succinyl-CoA:oxalate CoA-transferase (SCOCT), EC 2.8.3.2, e.g. from Cupriavidus oxalaticus, oxalyl-CoA decarboxylase (OXC), EC 4.1.1.8, e.g. from A. thaliana or S cerevisiae; and oxalyl-CoA reductase/glyoxylate:NADP+oxidoreductase (OXR), EC 1.2.1.17, e.g. from Methylobacterium extorquens) for the degradation of oxalate (second MOI) into various products such as formate (first MOI), oxalyl-CoA (first MOI), formyl-CoA (first MOI), or glyoxylate (first MOI).

Importers of oxalate (e.g. oxalate:formate antiporter (OxIT) and exporters of formate may further be included in the transmissible elements.

WO2017/040719A1, WO2021/146397A1 and WO2022/204406A1 (all Synlogic Operating Company, Inc.), and each incorporated herein by reference in their entirety, disclose various genes, such as frc, ScAAE3, YfdE, Oxc for the degradation of oxalate (second MOI), optionally in combination with OxIT importer of oxalate.

WO2021/173808A1 (Synlogic Operating Company, Inc.), incorporated herein by reference in its entirety, discloses genes such as uricase, rasburicase, aegA or ygfT for the degradation of uric acid (second MOI), optionally in combination with an importer of uric acid, such as ygfY, uacT or ygfU.

WO2017/023818A1 (Synlogic Operating Company, Inc.), incorporated herein by reference in its entirety, discloses genes for the degradation of propionate (second MOI) and genes which are importers of propionate.

Dietary methionine (second MOI) is converted to cysteine using several different enzymes, and mutations in one or more of these enzymes leads to homocystinuria. WO2021/163421A1 (Synlogic Operating Company, Inc.), incorporated herein by reference in its entirety, discloses genes for the degradation of methionine using methionine gamma lyase (MGL) or methionine decarboxylase (MDC) from various sources, along with increasing expression of the methionine importer, metNIQ, and decreasing or preventing expression of the methionine exporter yjeH.

In the case of first MOIs which are peptides or proteins, the transmissible element may further comprise a nucleic acid encoding a signal peptide as described herein.

In some embodiments, the second MOI is a molecule that can be metabolized by the donor or recipient bacterium and its conversion to the first MOI allows the donor or recipient bacterium to selectively grow in the presence of the second MOI. Thus, the second MOI may be an exogenously-added compound (e.g. rare carbohydrate) or may be a molecule present only in the local environment (such as in the gut, e.g. lower gastrointestinal tract or upper gastrointestinal tract). The addition of metabolic pathways converting the second MOI to the first MOI give a fitness advantage to the donor or recipient bacterium, and help it to effectively compete against other, native bacteria in the environment. This results in an increase in the relative abundance of the donor or recipient bacterium in the native microbiome.

Thus, in one embodiment, the transmissible element may comprise a metabolic pathway which enables the bacterium to metabolize and selectively grow on a second MOI which is an exogenously-added compound (e.g. a rare carbohydrate), after conversion of the second MOI to a first MOI which can be used as a food or carbon source by the donor or recipient bacterium. In one embodiment, the transmissible element may comprise a metabolic pathway which enables the bacterium to metabolize and selectively grow in the presence of compounds (i.e. second MOIs) which are present only in the local environment (such as in the gut, e.g. lower gastrointestinal tract or upper gastrointestinal tract), after conversion of the second MOI to a first MOI which can be used as a food or carbon source by the donor or recipient bacterium. Thus, in one embodiment, the first MOI is a sugar which can be used by the donor or recipient bacterium as a carbon source and the second MOI is rare carbohydrate. In an embodiment, the second MOI is porphyran, agarose, carrageenan, and any combination thereof and the exogenous nucleic acid of A) encodes porphyranase, glycoside hydrolase, sulfatase, galactosidase, and any combination thereof. In other embodiments, the rare carbohydrate is alginate, fucoidan, laminarin, xylan, ulvan and/or xylan. Genes for the use of rare carbohydrates, and their application in modified bacteria are described, for example, in WO2022/013269 (Danmarks Tekniske Universitet), WO2018/112194A1 (The Board of Trustees of the Leyland Standford Junior University & Novome Biotechnologies, Inc) and WO2023/057598A1 (Eligo Bioscience), each of which is incorporated herein by reference in its entirety, in particular for disclosures therein relating to the identify of rare carbohydrates and genes used for their conversion to sugar molecules that can be used by the recipient bacterium.

For all first MOIs, the transmissible element may further comprise a nucleic acid encoding one or more exporter(s) of the first MOI (e.g. the therapeutic protein molecule or metabolite). In any embodiment herein, the transmissible element may further comprise an exogenous nucleic acid encoding one or more exporter(s) of the first MOI (e.g. the therapeutic protein molecule or metabolite). In a particular embodiment, the nucleic acid encodes an exporter of the first MOI.

For all first MOIs, the transmissible element may further comprise a nucleic acid encoding one or more importer(s) of the second MOI (e.g. a protein molecule or substrate for the production of the first MOI). In any embodiment herein, the transmissible element may further comprise an exogenous nucleic acid encoding one or more importer(s) of the second MOI (e.g. a protein molecule or substrate for the production of the first MOI). In a particular embodiment, the nucleic acid encodes an importer of the second MOI.

Where the first MOI is a metabolite, the transmissible element may further comprise a nucleic acid encoding one or more importer(s) of any further substrate which is converted by one of the protein(s) (G1, G2 . . . GX) for the conversion of the second MOI (B) to the first MOI (A) (e.g. a substrate used in the pathway for the production of the first MOI). In any embodiment herein, the transmissible element may further comprise an exogenous nucleic acid encoding one or more importer(s) of any further substrate which is converted by one of the protein(s) (G1, G2 . . . GX) for the conversion of the second MOI (B) to the first MOI (A) (e.g. a substrate used in the pathway for the production of the first MOI). In a particular embodiment, the nucleic acid encodes an importer of the any further substrate.

In one particular embodiment, the transmissible element comprises an exogenous nucleic acid encoding one or more protein(s) (G1, G2 . . . GX) for the conversion of a second MOI (B) to the first MOT (A) and an exporter of the first MOI. The exogenous nucleic acid may further encode an importer of a further substrate which is converted by one of the protein(s) (G1, G2 . . . GX) for the conversion of the second MOI (B) to the first MOI (A).

In one embodiment, the transmissible element comprises an exogenous nucleic acid encoding one or more protein(s) (G1, G2 . . . GX) for the conversion of a second MOI (B) to the first MOI (A) and an importer of the second MOI. The exogenous nucleic acid may further encode an importer of a further substrate which is converted by one of the protein(s) (G1, G2 . . . GX) for the conversion of the second MOI (B) to the first MOI (A).

In one embodiment, the transmissible element comprises an exogenous nucleic acid encoding one or more protein(s) (G1, G2 . . . GX) for the conversion of a second MOI (B) to the first MOI (A) and an exporter of the first MOI and an importer of the second MOI. The exogenous nucleic acid may further encode an importer of a further substrate which is converted by one of the protein(s) (G1, G2 . . . GX) for the conversion of the second MOI (B) to the first MOI (A).

In one embodiment, the at least one exogenous nucleic acid sequence of A) does not comprise any reporter genes. In one embodiment, the at least one exogenous nucleic acid sequence of A) does not comprise any antibiotic resistance genes.

### Promoters

The at least one exogenous nucleic acid for the production of the first MOI may use a promoter which is endogenous to the transmissible element, for example a promoter which is naturally occurring in the plasmid, conjugative plasmid, phage or phagemid.

In other embodiments, the at least one exogenous nucleic acid for the production of the first MOI may use a promoter which is exogenous to the transmissible element, for example a promoter which is engineered into the plasmid, conjugative plasmid, phage or phagemid. Where the at least one exogenous nucleic acid for the production of the first MOI comprises more than one expressed product (for example more than one of an enzyme, an importer and/or an exporter), each element of the exogenously-added nucleic acid encoding said expressed product of A) may be under the control of a different promoter. In other embodiments, al the expressed proteins from the at least one exogenous nucleic acid for the production of the first MOI are comprised by a single operon under the control of a single promoter. The promoters may be any of the promoters described elsewhere herein.

In other embodiments, the at least one exogenous nucleic acid for the conversion of a second MOI to the first MOI may use a promoter which is exogenous to the transmissible element, for example a promoter which is engineered into the plasmid, conjugative plasmid, phage or phagemid. Where the at least one exogenous nucleic acid for the production of the first MOI comprises more than one expressed product (for example more than one of an enzyme, an importer and/or an exporter), each element of the exogenously-added nucleic acid encoding said expressed product of A) may be under the control of a different promoter.

In other embodiments, where the at least one exogenous nucleic acid for the production of the first MOI comprises more than one expressed protein product (G1, G2 . . . GX) for the conversion of a second MOI to the first MOI, the exogenously-added nucleic acid encoding all of said expressed proteins of A) may be comprised by a single operon under the control of a single promoter. The promoters may be any of the promoters described elsewhere herein.

In one embodiment where the at least one exogenous nucleic acid for the production of the first MOI encodes one or more expressed protein product(s) (G1, G2 . . . GX) for the conversion of a second MOI to the first MOI and an exporter of the first MOI, the exogenously-added nucleic acid encoding all of said expressed proteins and the exporter of the first MOI of A) may be comprised by a single operon under the control of a single promoter. The promoters may be any of the promoters described elsewhere herein.

In one embodiment where the at least one exogenous nucleic acid for the production of the first MOI encodes one or more expressed protein product(s) (G1, G2 . . . GX) for the conversion of a second MOI to the first MOI and an importer of the second MOI, the exogenously-added nucleic acid encoding all of said expressed proteins and the importer of the second MOI of A) may be comprised by a single operon under the control of a single promoter. The promoters may be any of the promoters described elsewhere herein.

In one embodiment where the at least one exogenous nucleic acid for the production of the first MOI encodes one or more expressed protein product(s) (G1, G2 . . . GX) for the conversion of a second MOI to the first MOI and an importer of any further substrate which is converted by one of the expressed protein(s) (G1, G2 . . . GX) for the conversion of the second MOI (B) to the first MOI (A), the exogenously-added nucleic acid encoding all of said expressed proteins and the importer of any further substrate of A) may be comprised by a single operon under the control of a single promoter. The promoters may be any of the promoters described elsewhere herein.

In one embodiment, all of the exogenous nucleic acids involved in the production of the first MOI encodes from a second MOI (i.e. all of the enzyme(s), importer(s) and exporter(s) which are exogenous to the recipient bacterium) may be comprised by a single operon under the control of a single promoter. The promoters may be any of the promoters described elsewhere herein.

The expression of the PMS which encodes a nucleic acid modifier or a peptide molecule may be controlled by a promoter which is endogenous to the transmissible element, for example a promoter which is naturally occurring in the plasmid, conjugative plasmid, phage or phagemid.

In other embodiments, the expression of the PMS which encodes a nucleic acid modifier or a peptide molecule may be controlled by a promoter which is exogenous to the transmissible element, for example a promoter which is engineered into the plasmid, conjugative plasmid, phage or phagemid. Where the nucleic acid modifier comprises more than one expressed product (for example a multi-subunit protein and a nucleic acid sequence, such as a guide RNA), each element of the nucleic acid modifier of B) may be under the control of a different promoter. The promoters may be any of the promoters described elsewhere herein.

In a particular embodiment, where the nucleic acid modifier comprises more than one expressed product (for example a multi-subunit protein and a nucleic acid sequence, such as a guide RNA), each element of the nucleic acid modifier of B) may be comprised by a single operon under the control of a single promoter. The promoters may be any of the promoters described elsewhere herein.

Constitutive promoters may be particularly useful in the transmissible elements described herein, providing sustained and high level production of the nucleic acids of A) and B).

Thus, in one embodiment, the PMS which encodes a nucleic acid modifier or a peptide molecule is under the control of one or more constitutive promoter(s) (e.g. any of the constitutive promoters described herein).

In one embodiment, the components of the at least one exogenous nucleic acid for the production of the first MOI are each or all under the control of one or more constitutive promoter(s) (e.g. any of the constitutive promoters described herein). The components of the at least one exogenous nucleic acid for the production of the first MOI may all be under the control of one constitutive promoter(s) (e.g. any of the constitutive promoters described herein).

In one embodiment, the promoter is a promoter which is based on the sequence of a tac promoter. Tac promoters are based on a combination of promoters from the trp and lac operons, see de Boer, et al., PNAS, 80(1), 21-25, 1983. doi:10.1073/pnas.80.1.21, which is incorporated herein by reference in its entirety. Several tac-based promoters have been reported in the art, see e.g. Zhang et al., Microb. Cell Fact, 16:84, 2017, doi: 10.1186/s12934-017-0700-2, which is incorporated herein by reference in its entirety. In one embodiment, the promoter is a Pc-tga promoter. In one embodiment, the promoter has a nucleotide sequence of Seq ID No:42.

Constitutive promoters and variants are well known in the art and include, but are not limited to, BBa_J23100, a constitutive Escherichia coli cs promoter (e.g. an osmY promoter (International Genetically Engineered Machine (iGEM) Registry of Standard Biological Parts Name BBa_J45992; BBa_J45993)), a constitutive Escherichia coli σ² promoter (e.g. htpG heat shock promoter (BBa_J45504)), a constitutive Escherichia coli σ⁷⁰ promoter (e.g. lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001), M13K07 gene I promoter (BBa_M13101), M13K07 gene II promoter (BBa_M13102), M13K07 gene III promoter (BBa_M13103), M13K07 gene IV promoter (BBa_MM13104), M13K07 gene V promoter (BBa_MM13105), M13K07 gene VI promoter (BBa_M13106), M13K07 gene VIII promoter (BBa_M13108), M13110 (BBa_M13110)), a constitutive Bacillus subtilis σ^{A} promoter (e.g. promoter veg (BBa_K143013), promoter 43 (BBa_K143013), P_{liaG} (BBa_K823000), P_{lepA} (BBa_K823002), P_{veg}(BBa_K823003)), a constitutive Bacillus subtilis σ^{B} promoter (e.g. promoter ctc (BBa_K143010) or promoter gsiB (BBa_K143011)), a Salmonella promoter (e.g. Pspv2 from Salmonella (BBa_K112706), Pspv from Salmonella (BBa_K112707)), a bacteriophage T7 promoter (e.g. T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g. SP6 promoter (BBa_J64998)).

In another embodiment, the promoter is a constitutive Pc promoter having the nucleic acid sequence of SEQ ID No: 102.

One way for measuring the strength of activity is by measuring the Anderson score of any given promoter. The activity of the reporters is measured by the relative fluorescence of the promoter when used in the control plasmid EX-Ptet-S-rbsRFP-P “RFP reporter” (see http://parts.igem.org/Part:BBa_J61002) in strain TG1 grown in LB media to saturation. BBa_J23119 is the “consensus” promoter sequence and the strongest member of the family. The NheI and AvrII restriction sites present within these promoter parts make them a scaffold for furte modification. For more information, see http:parts.igem.org/Part:BBaga23114.

Thus, in one embodiment, the constitutive promoter is a strong constitutive promoter (for example a promoter having an Anderson Score (AS) of AS≥0.4, such as ≥0.5). In another embodiment, the promoter has an Anderson score of between 0.1 and 0.4 or between 0.1 and 0.5.

TABLE 1
Anderson Promoter Collection

| | | | Measured |
|---|---|---|---|
| SEQ ID NO: | Identifier | Sequenceª | Strength^{b} |
| Seq ID No: 42 | BBa J23119 | ttgacagctagctcagtcctaggtataatgctagc | n/a |
| Seq ID No: 44 | BBa J23100 | ttgacggctagctcagtcctaggtacagtgctagc | 1 |
| Seq ID No: 45 | BBa J23101 | tttacagctagctcagtcctaggtattatgctagc | 0.7 |
| Seq ID No: 46 | BBa J23102 | ttgacagctagctcagtcctaggtactgtgctagc | 0.86 |
| Seq ID No: 47 | BBa J23103 | ctgatagctagctcagtcctagggattatgctagc | 0.01 |
| Seq ID No: 48 | BBa J23104 | ttgacagctagctcagtcctaggtattgtgctagc | 0.72 |
| Seq ID No: 49 | BBa J23105 | tttacggctagctcagtcctaggtactatgctagc | 0.24 |
| Seq ID No: 50 | BBa J23106 | tttacggctagctcagtcctaggtatagtgctagc | 0.47 |
| Seq ID No: 51 | BBa J23107 | tttacggctagctcagccctaggtattatgctagc | 0.36 |
| Seq ID No: 52 | BBa J23108 | ctgacagctagctcagtcctaggtataatgctagc | 0.51 |
| Seq ID No: 53 | BBa J23109 | tttacagctagctcagtcctagggactgtgctagc | 0.04 |
| Seq ID No: 54 | BBa J23110 | tttacggctagctcagtcctaggtacaatgctagc | 0.33 |
| Seq ID No: 55 | BBa J23111 | ttgacggctagctcagtcctaggtatagtgctagc | 0.58 |
| Seq ID No: 56 | BBa J23112 | ctgatagctagctcagtcctagggattatgctagc | 0 |
| Seq ID No: 57 | BBa J23113 | ctgatggctagctcagtcctagggattatgctagc | 0.01 |
| Seq ID No: 58 | BBa J23114 | tttatggctagctcagtcctaggtacaatgctagc | 0.1 |
| Seq ID No: 59 | BBa J23115 | tttatagctagctcagcccttggtacaatgctagc | 0.15 |
| Seq ID No: 60 | BBa J23116 | ttgacagctagctcagtcctagggactatgctagc | 0.16 |
| Seq ID No: 61 | BBa J23117 | ttgacagctagctcagtcctagggattgtgctagc | 0.06 |
| Seq ID No: | BBa J23118 | ttgacggctagctcagtcctaggtattgtgctagc | 0.56 |

^{a}also shown in the Anderson Catalog, see http://parts.igem.org/Promoters/Catalog/Anderson
^{b}Strength is the Anderson Score (AS), e.g. a strength of 1 is a AS of 1. Reported activities of the promoters are given as the relative fluorescence of plasmids in strain TG1 grown in LB media to saturation. A suitable plasmid is EX-Ptet-S-rbsRFP-P “RFP reporter” as described at http://parts.igem.org/Part:BBa_J61002; insertion of a promoter element between XbaI and SpeI sites results in a RFP reporter.

In one embodiment, the PMS which encodes a nucleic acid modifier or a peptide molecule is under the control of one or more inducible promoter(s) (e.g. any of the inducible promoters described herein).

In one embodiment, the components of the at least one exogenous nucleic acid for the production of the first MOI are each or all under the control of one or more inducible promoter(s) (e.g. any of the inducible promoters described herein). The components of the at least one exogenous nucleic acid for the production of the first MOI may all be under the control of one inducible promoter(s) (e.g. any of the constitutive promoters described herein).

In some embodiments, it may be desirable to include a promoter which is inducible to produce the first MOI and/or the PMS under only certain conditions. For example, the at least one exogenous nucleic acid sequence to the recipient bacterium for the production of a first molecule of interest (MOI) in the recipient bacterium could be under the control of a promoter which is active only under certain environmental conditions. Alternatively or additionally, the expression of the PMS could be under the control of a promoter which is active only under certain environmental conditions. For example, the inducible promoter is active under environmental conditions which are specific to the gut of a subject. In one embodiment, the inducible promoter is active under environmental conditions which are specific to the upper gastrointestinal tract of a subject (e.g. bile acids). In one embodiment, the inducible promoter is active under environmental conditions which are specific to the lower gastrointestinal tract of a subject (e.g. anaerobic conditions). In one embodiment, the inducible promoter is active under the low oxygen or anaerobic conditions which are specific to gut (e.g. the upper gastrointestinal tract and/or the lower gastrointestinal tract) of a subject.

In another embodiment, the inducible promoter is a temperature sensitive promoter, such as one which is active under physiological temperatures (e.g. approximately 35 to 39° C., for example approximately 36 to 38° C., such as approximately 37° C.). For a discussion on the use of this type of promoter in a kill switch, see https://wyss.harvard.edu/news/kill-switches-for-engineered-microbes-gone-rogue/and the “cryodeath” system which is described in more detail in Stirling et al., Mol. Cell, 68, 686-697.e683, 2017, which is incorporated herein in its entirety.

In another embodiment, the promoter is active only in the presence of certain molecules present in the local physiological environment (such as molecules present only in the gut). These inducible promoters may therefore turn on and off production and secretion of the first MOI and/or the PMS when in the desired location (such as the gut). Such inducible promoters are described herein.

Other promoters of interest are ones which have been designed to be active when the bacterial cell is in a certain state, for example when it is a “stress-phase active”. Such stress-phase active promoters (SPAs) are described in GB2303409.3, which is incorporated herein in its entirety and for its disclosure relating to SPAs, in particular for any of promoters of SEQ ID Nos: 1 to 10 disclosed therein (Seq ID Nos: 68 to 77 in Table 6 hereinbelow) or any promoters in claims 77 to 86 therein.

Thus, in one embodiment the promoter is a promoter selected from a RelB, BolA, Hya, YiaG and a RpoH promoter. The promoter may be a promoter selected from a ReB promoter sequence, σ70; a BoA promoter sequence, σS, σ70; a Hya promoter sequence, σS, σ70; a KaG promoter sequence, σS; a RpoH promoter sequence P1, σ70; a RpoH promoter sequence P2, σS; a RpoH promoter sequence P3, σ24; a RpoH promoter sequence P4, σ70; a RpoH promoter sequence P5, σ70; and a RpoH promoter sequence P6, σ54. The promoter may be a promoter having a nucleotide sequence selected from any one of Seq ID Nos: 68 to 77, or a nucleotides sequence having 90% (or 95%) homology thereto.

### Kill Switches

In one embodiment, the donor (host) bacterium or the transmissible element further comprises a kill switch.

In some embodiments, it may be desirable to include a kill switch in the transmissible element A kill switch is a biocontainment system which is included in the transmissible element and is designed to destroy the transmissible element (e.g. the conjugative plasmid or plasmid) itself only, or the transmissible element and the bacterium comprising the transmissible element (e.g. the recipient bacterium) together, when no longer contained within its desired environment (e.g. within a microbiome, such as a gut microbiome, within a subject). Such means are well-known in the art, and are regulatable, for example by the addition of non-naturally occurring substances (e.g. synthetic amino acids), temperature and the like. Specific examples of promoters and kill switches (in particular, for removal of plasmids from bacteria) are provided in WO2023/012109A2 (SNIPR Biome, ApS), which is incorporated herein by reference in its entirety.

Bacteria comprising kill switches have been engineered for in vitro research purposes, e.g. to limit the spread of a biofuel-producing microorganism outside of a laboratory environment Bacteria engineered for in vivo administration to treat a disease may also be programmed to die at a specific time after the expression and delivery of the first MOI, or after the subject has experienced the therapeutic effect. For example, in some embodiments, the kill switch is activated to remove the transmissible element (e.g. the conjugative plasmid or plasmid) from the bacterium comprising the transmissible element (e.g. the recipient bacterium), or to kill the bacterium comprising the transmissible element (e.g. the recipient bacterium), after a period of time following oxygen level-dependent expression of the first MOI. In some embodiments, the kill switch is activated in a delayed fashion following oxygen level dependent expression of the first MOI. Alternatively, the bacterium comprising the transmissible element (e.g. the recipient bacterium) may be engineered to die after the bacterium has spread outside of a disease site. Specifically, it may be useful to prevent long-term colonization of subjects by the bacterium comprising the transmissible element (e.g. the recipient bacterium), spread of the bacterium comprising the transmissible element (e.g. the recipient bacterium) outside the area of interest (for example, outside the gut) within the subject, or spread of the bacterium comprising the transmissible element (e.g. the recipient bacterium) outside of the subject into the environment (for example, spread to the environment through the stool of the subject).

Kill-switches can be designed such that toxin(s) is/are produced in response to an environmental condition or external signal (e.g. the recipient bacterium is killed in response to an external cue) or, alternatively designed such that a toxin is produced once an environmental condition no longer exists or an external signal is ceased. Examples of such promoters are also described elsewhere herein. The toxin(s) is/are toxic to the bacterium which produces and secretes the first MOI.

The switches that control production of the toxin(s) can be based on, for example, transcriptional activation, translation (riboregulators), or DNA recombination (recombinase-based switches), and can sense environmental stimuli such as anaerobiosis, reactive oxygen species, temperature, bile acids, pH, lactate, caffeine or other biosensors. These switches can be activated by a single environmental factor or may require several activators in AND, OR, NAND and NOR logic configurations to induce cell death. For example, an AND riboregulator switch is activated by tetracycline, isopropyl P-D-I-thiogaiactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death.

Thus, in some embodiments, the bacterium comprising the transmissible element (e.g. the recipient bacterium) is further programmed to die or specifically degrade the transmissible element (e.g. the conjugative plasmid or plasmid) after sensing an exogenous environmental signal, for example, in a low-oxygen environment.

Kill switches can be permissive, such that the recipient bacterium will continue to thrive in its environment, unless a defined condition changes. Cell survival or proliferation may be impeded by the expression of toxins and lysis proteins (e.g. as discussed in Knudsen, et al., Appl. Environ. Microbiol., 57, 85-92, 1991; Callura, et al., Proc. Nati Acad. Sci. USA, 107, 15898-15903, 2010, each of which is incorporated herein by reference in its entirety), or the degradation of essential proteins (e.g. as discussed in Chan, et al., Nat. Chem. Biol., 12, 82-86, 2016 which is incorporated herein by reference in its entirety).

Thus, in one embodiment, the kill switch comprises a toxin gene, expression of which is induced in response to an environmental condition(s) and/or signal(s).

Expression of the toxin gene may be provided by an inducible promoter. Such promoters are well-known to those skilled in the art, and may include oxygen level-dependent promoters (e.g. an FNR [fumarate and nitrate reductase regulator]-inducible promoter, the ANR [anaerobic arginine deiminiase and nitrate reductase regulator]-inducible promoter, and the DNR [dissimilatory nitrate respiration regulator]-inducible promoter).

The inducible promoter may be a promoter which is induced by inflammation or an inflammatory response (e.g. an RNS and/or ROS-inducible promoter).

The inducible promoter may be a temperature-sensitive promoters (e.g. as described elsewhere herein). Temperature sensitive promoters include TlpA, TcI, TetR (and A89D and I193N mutants thereof), LacI (and A241T and G265D mutants thereof), GrpE, HtpG, Lon, RpoH, Clp and DnaK (which are described in more detail in Piraner et al., Nature Chemical Biology, 13, 75-80, 2016, doi:10.1038/nchembio.2233; which is incorporated herein in its entirety).

In one embodiment, the kill switch comprises a toxin gene expression of which is induced by a change in temperature. In one embodiment, the kill switch comprises a toxin gene expression of which is induced by an increase in oxygen levels. In one embodiment, the kill switch comprises a toxin gene expression of which is induced by the addition of a molecule which is not usually present in the local environment, such as the gut (e.g. arabinose, sugar alcohol (e.g. sorbitol), tetracycline, IPTG, rhamnose, and non-naturally occurring amino acids).

The inducible promoter may be a promoter induced by a substance that may or may not be naturally present in the local environment (i.e. is exogenously added to the local environment). Examples include, but are not limited to an arabinose-inducible promoter (e.g. a pBAD promoter), tetracycline-inducible promoters, IPTG-inducible promoters, rhamnose-inducible promoters, xylitol-inducible promoters, sorbitol-inducible promoters, and nutritional-inducible promoters.

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination.

### Bacteria

In any embodiment herein, the donor or recipient bacterium may be a gram-negative bacterium.

In any embodiment herein, the donor or recipient bacterium may be a gram-positive bacterium.

In any embodiment herein, the donor or recipient bacterium is a strain selected from any of the strains in Table 2.

In any embodiment herein, the donor or recipient bacterium may be an E. coli strain. In one embodiment, the E. coli strain is an E. coli strain from phylogroup A. In one embodiment, the E. coli strain is an E. coli strain from phylogroup B1. In one embodiment, the E. coli strain is an E. coli strain from phylogroup E. In one embodiment, the E. coli strain is an E. coli strain which is present in a probiotic product. The probiotic product may be colinfant New Born (e.g. strain A0 34/86). The probiotic product may be symbioflor2 (e.g. strain G1/2, G4/9, G5, G6/7, and G8). The probiotic product may be Mutaflor (e.g. E. coli Nissle).

In any embodiment herein, the donor or recipient bacterium may be a strain belonging to a genera selected from Bifidobaterium, Bacteroides, Lactobacillus, Lacticaseibacillus, Lactiplantibacillus, Levilactobacillus, Legilactobacillus, Limosilactobacillus and Lactococcus.

In any embodiment herein, the donor or recipient bacterium may be a strain belonging to a genera selected from a Bifidobacterium genus or a Bacteroides genus. In one embodiment, the genera is a Bacteroides genus. In one embodiment, the genera is a Bifidobacterium genus.

In any embodiment herein, the donor or recipient bacterium may be a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacills brevis, Ligilactobacillus salivarius, Limosilactobacillus reuteri and Lactococcus lactis.

In any embodiment herein, the donor or recipient bacterium may be a species which is selected from Bifidobacterium longum, Bifidobacterium breve, Bifidobacterium adolescentis, Bacteroides uniformis, Bacteroides vulgatus and Bacteroides thetaiotaomicron.

In any embodiment herein, the donor or recipient bacterium may be a strain belonging to the genus Spirulina.

In any embodiment herein, the donor or recipient bacterium may be a strain belonging to the genus Cyanobacteria.

In one embodiment, the donor (host) bacterium comprising the transmissible elements described herein has been engineered to remove some or all (e.g. all) prophage genes present in the bacterial genome. The donor (host) bacterium may be devoid of some or all (e.g. all) prophage genes.

In one embodiment, the donor (host) bacterium comprising the transmissible elements described herein has been engineered to remove any identified pathogenicity factors (such as hlyA, hlyB, hlyC and/or hlyD or any combination thereof) present in the bacterial genome. The donor (host) bacterium may be devoid of pathogenicity factors (such as hlyA, hlyB, hlyC and/or hlyD or any combination thereof).

### Formulations and Compositions Comprising the Bacteria, Conjugative Plasmids or Host Bacteria

There is provided a pharmaceutical composition comprising a donor (host) bacterium as described herein, and a pharmaceutically acceptable excipient or carrier.

There is provided a pharmaceutical composition comprising a transmissible element as described herein, and a pharmaceutically acceptable excipient or carrier.

The donor (host) bacterium, or transmissible element may be formulated in a pharmaceutical composition comprising a diluent, excipient or carrier. The formulation may be comprised within a medical device (such as an ampoule, a syringe, or an inhaler) or is formulated in a tincture, a capsule or a slow-release formulation. The formulation may be an oral tablet, comprised within a blister pack.

In one embodiment, the pharmaceutical composition comprising donor (host) bacterium, or transmissible element as described herein is formulated for oral or rectal administration. In one embodiment, the pharmaceutical composition is formulated for oral administration. In one embodiment, the pharmaceutical composition is formulated as a capsule or coated tablet.

The formulation comprising the donor (host) bacterium, or transmissible element may be freeze dried prior to encapsulation. In one embodiment, the pharmaceutical composition comprising a donor (host) bacterium, or transmissible element as described herein is a lyophilised formulation. In one embodiment, the pharmaceutical composition comprising a donor (host) bacterium, or transmissible element as described herein is an encapsulated formulation to be released in the lower gut of a subject.

Acceptable carriers, excipients, or stabilizers are non-toxic to subjects at the dosages and concentrations employed, and include buffers such as phosphate, citrate, and other organic acids; antioxidants including ascorbic acid and methionine; low molecular weight (less than about 10 residues) polypeptides; proteins, such as serum albumin, gelatine, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and metal complexes (e.g. Zn-protein complexes). In limited circumstances, due to stability of the vectors, the formulation may include preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol). A skilled formulator is aware of agents which are compatible with the different modes of delivery of the bacteria, conjugative plasmids or host (e.g. donor) bacteria described herein.

The donor (host) bacteria, or transmissible elements can also be formulated in liposomes. Liposomes containing the donor (host) bacteria, or transmissible elements are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545, each of which is incorporated herein by reference in its entirety. Liposomes with enhanced circulation time are disclosed in U.S. Pat. No. 5,013,556, incorporated herein by reference.

Donor (host) bacteria, or transmissible elements described herein can also be entrapped in microcapsule prepared, for example, by coacervation techniques or by interfacial polymerization, for example, hydroxymethylcellulose or gelatin-microcapsule and poly-(methyimethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, Pa.

Sustained-release preparations can also be prepared. Suitable examples of sustained-release preparations include semipermeable matrices of solid hydrophobic polymers containing the antagonist, which matrices are in the form of shaped articles, e.g. films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The formulation may be comprised within a medical device, such as an ampoule, a syringe, or an inhaler.

Suitable dosage amounts for the donor (host) bacteria may range from about 10⁵ to 10¹² bacteria, e.g. approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹² bacteria. The composition may be administered daily, weekly, or monthly. It may be administered multiple times per day (e.g. twice or three times per day).

In some embodiments, the donor (host) bacteria are enterically coated for release into the gut or a particular region of the gut, for example, the small or large intestines. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Uses of the Methods. Recipient Bacteria, Transmissible Elements and Host Bacteria Disclosed Herein

The methods described herein can be carried out ex vivo. The methods described herein can be carried out in vitro. The methods described herein can be carried out in vivo.

There is provided a method of modifying recipient bacteria in the gut of a subject, comprising administering to said subject a donor (host) bacterium, a transmissible element (e.g. a conjugative plasmid, phage or phagemid), or a pharmaceutical composition as described herein. The subject may comprise one or more strains of bacteria which are capable of the transmissible element being transmitted to said one or more strains of bacteria.

There is provided a method of treating or preventing a disease or condition in a subject in need thereof, said method comprising administering to a subject in need thereof a donor (host) bacterium, a transmissible element (e.g. a conjugative plasmid, phage or phagemid), or a pharmaceutical composition as described herein, and wherein the subject comprises one or more strains of bacteria which are capable of the transmissible element being transmitted to said one or more strains of bacteria.

There is provided a donor (host) bacterium, a transmissible element (e.g. a conjugative plasmid, phage or phagemid), or a pharmaceutical formulation as described herein for use in a method to treat or prevent a disease or condition mediated in a subject by a lack of, or insufficient amount of the first MOI. The subject may comprise one or more strains of bacteria which are capable of the transmissible element being transmitted to said one or more strains of bacteria.

There is provided method of producing a first MOI in the gut of a subject, comprising administering to said subject a bacterium, a conjugative plasmid, a host (e.g. donor) bacterium or pharmaceutical formulation as described herein. The subject may comprise one or more strains of bacteria which are capable of the transmissible element being transmitted to said one or more strains of bacteria.

The subject can be a human or animal subject. The subject can be a mammal such as a non-primate (e.g. cows, pigs, horses, cats, dogs, rats, etc.) or a primate (e.g. monkey and human). The subject may be a rodent, mouse or rat. The subject may be a vertebrate, reptile, bird or fish. In particular, the subject is a human.

There is provided a donor (host) bacterium, a transmissible element (e.g. a conjugative plasmid, phage or phagemid), or a pharmaceutical formulation as described herein for use as a medicament or for use in therapy.

There is provided a method of treating a metabolic disease comprising administering to a subject in need thereof a donor (host) bacterium, a transmissible element (e.g. a conjugative plasmid, phage or phagemid), or a pharmaceutical formulation as described herein, wherein the metabolic disease is caused by a deficiency of the first MOI and/or wherein the first MOI is a molecule capable of treating said metabolic disease. The subject may comprise one or more strains of bacteria which are capable of the transmissible element being transmitted to said one or more strains of bacteria.

There is provided a method of treating a cardiovascular metabolic disease comprising administering to a subject in need thereof a donor (host) bacterium, a transmissible element (e.g. a conjugative plasmid, phage or phagemid), or a pharmaceutical formulation as described herein, wherein the cardiovascular metabolic disease is caused by a deficiency of the first MOI and/or wherein the first MOI is a molecule capable of treating said cardiovascular metabolic disease. The subject may comprise one or more strains of bacteria which are capable of the transmissible element being transmitted to said one or more strains of bacteria.

There is provided a method of treating a metabolic disease selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase 1/3 deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome, said method comprising administering to a subject in need thereof a donor (host) bacterium, a transmissible element (e.g. a conjugative plasmid, phage or phagemid), or a pharmaceutical formulation as described herein. The metabolic disease may be caused by a deficiency of the first MOI and/or the first MOI may be a molecule capable of treating said metabolic disease. The subject may comprise one or more strains of bacteria which are capable of the transmissible element being transmitted to said one or more strains of bacteria.

There is provided a method of treating a metabolic disease selected from metabolic syndrome, type 2 diabetes (including complications of type 2 diabetes, e.g. insulin sensitivity in type 2 diabetes), and non-alcoholic fatty liver disease, said method comprising administering to a subject in need thereof a donor (host) bacterium, a transmissible element (e.g. a conjugative plasmid, phage or phagemid), or a pharmaceutical formulation as described herein. The metabolic disease may be caused by a deficiency of the first MOI and/or the first MOI may be a molecule capable of treating said metabolic disease. The subject may comprise one or more strains of bacteria which are capable of the transmissible element being transmitted to said one or more strains of bacteria.

There is provided a donor (host) bacterium, a transmissible element (e.g. a conjugative plasmid, phage or phagemid), or a pharmaceutical formulation as described herein for use in the treatment of a metabolic disease.

There is provided a donor (host) bacterium, a transmissible element (e.g. a conjugative plasmid, phage or phagemid), or a pharmaceutical formulation as described herein for use in the treatment of a cardiovascular metabolic disease.

There is provided a donor (host) bacterium, a transmissible element (e.g. a conjugative plasmid, phage or phagemid), or a pharmaceutical formulation as described herein for use in the treatment of a metabolic disease selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase 1/3 deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome.

There is provided a donor (host) bacterium, a transmissible element (e.g. a conjugative plasmid, phage or phagemid), or a pharmaceutical formulation as described herein for use in the treatment of a metabolic disease selected from metabolic syndrome, type 2 diabetes (including complications of type 2 diabetes, e.g. insulin sensitivity in type 2 diabetes), and non-alcoholic fatty liver disease.

Metabolic Syndrome affects approximately 20-30% of the middle-aged population, and represents an increased risk to cardiovascular disorders, the leading cause of death in the United States. Obesity, dyslipidemia, hypertension, and type 2 diabetes are described as metabolic syndrome. In some embodiments, the bacteria, conjugative plasmids, host (e.g. donor) cells or pharmaceutical compositions described herein are useful in the treatment, prevention and/or management of metabolic syndrome and/or obesity.

Metabolic syndrome is a clustering of at least three of five of the following medical conditions: abdominal (central) obesity, elevated blood pressure, elevated fasting plasma glucose, high serum triglycerides, and low high-density lipoprotein (HDL) levels.

Metabolic diseases are associated with a variety of physiological changes, including but not limited to elevated glucose levels, elevated triglyceride levels, elevated cholesterol levels, insulin resistance, high blood pressure, hypogonadism, subfertility, infertility, abdominal obesity, pro-thrombotic conditions, and pro-inflammatory conditions.

Cardiovascular disease includes coronary artery diseases (CAD) such as angina and myocardial infarction, stroke, hypertensive heart disease, rheumatic heart disease, cardiomyopathy, heart arrhythmia, congenital heart disease, valvular heart disease, carditis, aortic aneurysms, peripheral artery disease, and venous thrombosis. Coronary artery disease, stroke, and peripheral artery disease involve atherosclerosis, caused inter alia by high blood pressure, smoking, diabetes, lack of exercise, obesity, high blood cholesterol, poor diet, and excessive alcohol consumption, and the like.

Obesity is a common, deadly, and costly disease in developed countries which impacts all age groups, race, and gender. Obesity can be classified as an inflammatory disease because it is associated with immune activation and a chronic, low-grade systemic inflammation. Endotoxemia, a process resulting from translocation of endotoxic compounds (lipopolysaccharides [LPS]) of gram-negative intestinal bacteria. In the last decade, it has become evident that insulin resistance and T2DM are characterized by low-grade inflammation. In this respect, LPS trigger a low-grade inflammatory response, and the process of endotoxemia can therefore result in the development of insulin resistance and other metabolic disorders. Other anti-inflammatory ALMs as described herein may also be useful in the treatment of type 2 diabetes.

In certain embodiments, the donor (host) bacteria, transmissible elements (e.g. conjugative plasmids, phage or phagemids), or pharmaceutical compositions as described herein decrease tryptophan levels in the subject, e.g. in the serum and/or in the gut, e.g. for the prevention, treatment, and/or management of obesity.

Metabolic syndrome is an important risk factor for cardiovascular disease incidence and mortality, as well as all-cause mortality. Thus, the detection, prevention, and treatment of the underlying risk factors of the metabolic syndrome are a critical approach to lower the cardiovascular disease incidence in the general population.

The donor (host) bacteria, transmissible elements (e.g. conjugative plasmids, phage or phagemids), or pharmaceutical compositions as described herein can be administered to the subject in one or more doses. It is understood that the precise dosage and duration of treatment is a function of the disease being treated and can be determined empirically using known testing protocols or by extrapolation from in vivo or in vito test data, and taking into account age, weight and sex of the subject. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions.

The disease or condition may be metabolic syndrome or cardiometabolic disease (e.g. selected from obesity, diabetes, insulin resistance and non-alcoholic fatty liver disease). The disease may be inflammatory bowel disease (e.g. selected from Crohn's disease and Ulcerative Colitis). The condition may be irritable bowel syndrome or leaky gut syndrome.

TABLE 2
Example Bacteria
Optionally, the modified bacteria or the bacteria to
which the conjugative plasmid is hosted are/or transferred
(i.e. recipient bacteria) are selected from this Table.

Abiotrophia
Abiotrophia defectiva
Acaricomes
Acaricomes phytoseiuli
Acetitomaculum
Acetitomaculum ruminis
Acetivibrio
Acetivibrio cellulolyticus
Acetivibrio ethanolgignens
Acetivibrio multivorans
Acetoanaerobium
Acetoanaerobium noterae
Acetobacter
Acetobacter aceti
Acetobacter cerevisiae
Acetobacter cibinongensis
Acetobacter estunensis
Acetobacter fabarum
Acetobacter ghanensis
Acetobacter indonesiensis
Acetobacter lovaniensis
Acetobacter malorum
Acetobacter nitrogenifigens
Acetobacter oeni
Acetobacter orientalis
Acetobacter orleanensis
Acetobacter pasteurianus
Acetobacter pornorurn
Acetobacter senegalensis
Acetobacter xylinus
Acetobacterium
Acetobacterium bakii
Acetobacterium carbinolicum
Acetobacterium dehalogenans
Acetobacterium fimetarium
Acetobacterium malicum
Acetobacterium paludosum
Acetobacterium tundrae
Acetobacterium wieringae
Acetobacterium woodii
Acetofilamentum
Acetofilamentum rigidum
Acetohalobium
Acetohalobium arabaticum
Acetomicrobium
Acetomicrobium faecale
Acetomicrobium flavidum
Acetonema
Acetonema longum
Acetothermus
Acetothermus paucivorans
Acholeplasma
Acholeplasma axanthum
Acholeplasma brassicae
Acholeplasma cavigenitalium
Acholeplasma equifetale
Acholeplasma granularum
Acholeplasma hippikon
Acholeplasma laidlawii
Acholeplasma modicum
Acholeplasma morum
Acholeplasma multilocale
Acholeplasma oculi
Acholeplasma palmae
Acholeplasma parvum
Acholeplasma pleciae
Acholeplasma vituli
Achromobacter
Achromobacter denitrificans
Achromobacter insolitus
Achromobacter piechaudii
Achromobacter ruhlandii
Achromobacter spanius
Acidaminobacter
Acidaminobacter hydrogenoformans
Acidaminococcus
Acidaminococcus fermentans
Acidaminococcus intestini
Acidicaldus
Acidicaldus organivorans
Acidimicrobium
Acidimicrobium ferrooxidans
Acidiphilium
Acidiphilium acidophilum
Acidiphilium angustum
Acidiphilium cryptum
Acidiphilium multivorum
Acidiphilium organovorum
Acidiphilium rubrum
Acidisoma
Acidisoma sibiricum
Acidisoma tundrae
Acidisphaera
Acidisphaera rubrifaciens
Acidithiobacillus
Acidithiobacillus albertensis
Acidithiobacillus caldus
Acidithiobacillus ferrooxidans
Acidithiobacillus thiooxidans
Acidobacterium
Acidobacterium capsulatum
Acidocella
Acidocella aminolytica
Acidocella facilis
Acidomonas
Acidomonas methanolica
Acidothermus
Acidothermus cellulolyticus
Acidovorax
Acidovorax anthurii
Acidovorax caeni
Acidovorax cattleyae
Acidovorax citrulli
Acidovorax defluvii
Acidovorax delafieldii
Acidovorax facilis
Acidovorax konjaci
Acidovorax temperans
Acidovorax valerianellae
Acinetobacter
Acinetobacter baumannii
Acinetobacter baylyi
Acinetobacter bouvetii
Acinetobacter calcoaceticus
Acinetobacter gerneri
Acinetobacter haemolyticus
Acinetobacter johnsonii
Acinetobacter junii
Acinetobacter lwoffi
Acinetobacter parvus
Acinetobacter radioresistens
Acinetobacter schindleri
Acinetobacter soli
Acinetobacter tandoil
Acinetobacter tjernbergiae
Acinetobacter towneri
Acinetobacter ursingil
Acinetobacter venetianus
Acrocarpospora
Acrocarpospora corrugata
Acrocarpospora macrocephala
Acrocarpospora pleiomorpha
Actibacter
Actibacter sediminis
Actinoalloteichus
Actinoalloteichus cyanogriseus
Actinoalloteichus hymeniacidonis
Actinoalloteichus spitiensis
Actinobaccillus
Actinobacillus capsulatus
Actinobacillus delphinicola
Actinobacillus hominis
Actinobacillus indolicus
Actinobacillus lignieresii
Actinobacillus minor
Actinobacillus muris
Actinobacillus pleuropneumoniae
Actinobacillus porcinus
Actinobacillus rossii
Actinobacillus scotiae
Actinobacillus seminis
Actinobacillus succinogenes
Actinobaccillus suis
Actinobacillus ureae
Actinobaculum
Actinobaculum massiliense
Actinobaculum schaalii
Actinobaculum suis
Actinomyces urinale
Actinocatenispora
Actinocatenispora rupis
Actinocatenispora thailandica
Actinocatenispora sera
Actinocorallia
Actinocorallia aurantiaca
Actinocorallia aurea
Actinocorallia cavernae
Actinocorallia glomerata
Actinocorallia herbida
Actinocorallia libanotica
Actinocorallia longicatena
Actinomadura
Actinomadura alba
Actinomadura atramentaria
Actinomadura bangladeshensis
Actinomadura catellatispora
Actinomadura chibensis
Actinomadura chokoriensis
Actinomadura citrea
Actinomadura coerulea
Actinomadura echinospora
Actinomadura fibrosa
Actinomadura formosensis
Actinomadura hibisca
Actinomadura kijaniata
Actinomadura atina
Actinomadura livida
Actinomadura luteofluorescens
Actinomadura macra
Actinomadura madurae
Actinomadura oligospora
Actinomadura pelletieri
Actinomadura rubrobrunea
Actinomadura rugatobispora
Actinomadura umbrina
Actinomadura verrucosospora
Actinomadura vinacea
Actinomadura viridilutea
Actinomadura viridis
Actinomadura yumaensis
Actinomyces
Actinomyces bovis
Actinomyces denticolens
Actinomyces europaeus
Actinomyces georgiae
Actinomyces gerencseriae
Actinomyces hordeovulneris
Actinomyces howellii
Actinomyces hyovaginalis
Actinomyces israelii
Actinomyces johnsonii
Actinomyces meyeri
Actinomyces naeslundii
Actinomyces neuii
Actinomyces odontolyticus
Actinomyces oris
Actinomyces radingae
Actinomyces slackii
Actinomyces turicensis
Actinomyces viscosus
Actinoplanes
Actinoplanes auranticolor
Actinoplanes brasiliensis
Actinoplanes consettensis
Actinoplanes deccanensis
Actinoplanes derwentensis
Actinoplanes digitatis
Actinoplanes durhamensis
Actinoplanes ferrugineus
Actinoplanes globisporus
Actinoplanes humidus
Actinoplanes italicus
Actinoplanes liguriensis
Actinoplanes lobatus
Actinoplanes missouriensis
Actinoplanes palleronit
Actinoplanes philippinensis
Actinoplanes rectilineatus
Actinoplanes regularis
Actinoplanes teichomyceticus
Actinoplanes utahensis
Actinopolyspora
Actinopolyspora halophila
Actinopolyspora mortivallis
Actinosynnema
Actinosynnema mirum
Actinotalea
Actinotalea fermentans
Aerococcus
Aerococcus sanguinicola
Aerococcus urinae
Aerococcus urinaeequi
Aerococcus urinaehominis
Aerococcus viridans
Aeromicrobium
Aeromicrobium erythreum
Aeromonas
Aeromonas allosaccharophila
Aeromonas bestiarum
Aeromonas caviae
Aeromonas encheleia
Aeromonas enteropelogenes
Aeromonas eucrenophila
Aeromonas ichthiosmia
Aeromonas jandaei
Aeromonas media
Aeromonas popoffii
Aeromonas sobria
Aeromonas veronii
Agrobacterium
Agrobacterium gelatinovorum
Agrococcus
Agrococcus citreus
Agrococcus jenensis
Agromonas
Agromonas oligotrophica
Agromyces
Agromyces fucosus
Agromyces hippuratus
Agromyces luteolus
Agromyces mediolanus
Agromyces ramosus
Agromyces rhizospherae
Akkermansia
Akkermansia muciniphila
Albidiferax
Albidiferax ferrireducens
Albidovulum
Albidovulum inexpectatum
Alcaligenes
Alcaligenes denitrificans
Alcaligenes faecalis
Alcanivorax
Alcanivorax borkumensis
Alcanivorax jadensis
Algicola
Algicola bacteriolytica
Alicyclobacillus
Alicyclobacillus disulfidooxidans
Alicyclobacillus sendaiensis
Alicyclobacillus vulcanalis
Alishewanella
Alishewanella fetalis
Alkalibacillus
Alkalibacillus haloalkaliphilus
Alkalilimnicola
Alkalilimnicola ehrlichii
Alkaliphilus
Alkaliphilus oremlandii
Alkaliphilus transvaalensis
Allochromatium
Allochromatium vinosum
Allolococcus
Alloiococcus otitis
Allokutzneria
Allokutzneria albata
Altererythrobacter
Altererythrobacter ishigakiensis
Altermonas
Altermonas haloplanktis
Altermonas macleodii
Alysiella
Alysiella crassa
Alysiella filiformis
Aminobacter
Aminobacter aganoensis
Aminobacter aminovorans
Aminobacter niigataensis
Aminobacterium
Aminobacterium mobile
Aminomonas
Aminomonas paucivorans
Ammoniphilus
Ammoniphilus oxalaticus
Ammoniphilus oxalivorans
Amphibacillus
Amphibacillus xylanus
Amphritea
Amphritea balenae
Amphritea japonica
Amycolatopsis
Amycolatopsis alba
Amycolatopsis albidoflavus
Amycolatopsis azurea
Amycolatopsis coloradensis
Amycolatopsis lurida
Amycolatopsis mediterranei
Amycolatopsis rifamycinica
Amycolatopsis rubida
Amycolatopsis sulphurea
Amycolatopsis tolypomycina
Anabaena
Anabaena cylindrica
Anabaena flos-aquae
Anabaena variabilis
Anaeroarcus
Anaeroarcus burkinensis
Anaerobaculum
Anaerobaculum mobile
Anaerobiospirillum
Anaerobiospirillum succiniciproducens
Anaerobiospirillum thomasii
Anaerococcus
Anaerococcus hydrogenalis
Anaerococcus lactolyticus
Anaerococcus prevotii
Anaerococcus tetradius
Anaerococcus vaginalis
Anaerofustis
Anaerofustis stercorihominis
Anaeromusa
Anaeromusa acidaminophila
Anaeromyxobacter
Anaeromyxobacter dehalogenans
Anaerorhabdus
Anaerorhabdus furcosa
Anaerosinus
Anaerosinus glycerini
Anaerovirgula
Anaerovirgula multivorans
Ancalomicroblum
Ancalomicrobium adetum
Ancylobacter
Ancylobacter aquaticus
Aneurinibacillus
Aneurinibacillus aneurinilyticus
Aneurinibacillus migulanus
Aneurinibacillus thermoaerophilus
Angiococcus
Angiococcus disciformis
Angulomicrobium
Angulomicrobium tetraedrale
Anoxybacillus
Anoxybacillus pushchinoensis
Aquabacterium
Aquabacterium commune
Aquabacterium parvum
Aquaspirillum
Aquaspirillum polymorphum
Aquaspirillum putridiconchylium
Aquaspirillum serpens
Aquimarina
Aquimarina latercula
Arcanobacterium
Arcanobacterium haemolyticum
Arcanobacterium pyogenes
Archangium
Archangium gephyra
Arcobacter
Arcobacter butzieri
Arcobacter cryaerophilus
Arcobacter halophilus
Arcobacter nitrofigilis
Arcobacter skirrowii
Arhodomonas
Arhodomonas aquaeolei
Arsenophonus
Arsenophonus nasoniae
Arthrobacter
Arthrobacter agilis
Arthrobacter albus
Arthrobacter aurescens
Arthrobacter chlorophenolicus
Arthrobacter citreus
Arthrobacter crystallopoietes
Arthrobacter cumminsii
Arthrobacter globiformis
Arthrobacter histidinolovorans
Arthrobacter ilicis
Arthrobacter luteus
Arthrobacter methylotrophus
Arthrobacter mysorens
Arthrobacter nicotianae
Arthrobacter nicotinovorans
Arthrobacter oxydans
Arthrobacter pascens
Arthrobacter phenanthrenivorans
Arthrobacter polychromogenes
Atrhrobacter protophormiae
Arthrobacter psychrolactophilus
Arthrobacter ramosus
Arthrobacter sulfonivorans
Arthrobacter sulfureus
Arthrobacter uratoxydans
Arthrobacter ureafaciens
Arthrobacter viscosus
Arthrobacter woluwensis
Asala
Asaia bogorensis
Asanoa
Asanoa ferruginea
Asticcacaulis
Asticcacaulis biprosthecium
Asticcacaulis excentricus
Atopobacter
Atopobacter phocae
Atopobium
Atopobium fossor
Atopobium minutum
Atopobium parvulum
Atopobium rimae
Atopobium vaginae
Aureobacterium
Aureobacterium barkeri
Aurobacterium
Aurobacterium liquefaciens
Avibacterium
Avibacterium avium
Avibacterium gallinarum
Avibacterium paragallinarum
Avibacterium volantium
Azoarcus
Azoarcus indigens
Azoarcus tolulyticus
Azoarcus toluvorans
Azohydromonas
Azohydromonas australica
Azohydromonas lata
Azomonas
Azomonas agilis
Azomonas insignis
Azomonas macrocytogenes
Azorhizobium
Azorhizobium caulinodans
Azorhizophilus
Azorhizophilus paspali
Azospirillum
Azospirillum brasilense
Azospirillum halopraeferens
Azospirillum irakense
Azotobacter
Azotobacter beijerinckii
Azotobacter chroococcum
Azotobacter nigricans
Azotobacter salinestris
Azotobacter vinelandii
Bacteriovorax
Bacteriovorax stolpii
Bacteroides
Bacteroides caccae
Bacteroides coagulans
Bacteroides eggerthii
Bacteroides fragilis
Bacteroides galacturonicus
Bacteroides helcogenes
Bacteroides ovatus
Bacteroides pectinophilus
Bacteroides pyogenes
Bacteroides salyersiae
Bacteroides stercoris
Bacteroides suis
Bacteroides tectus
Bacteroides thetaiotaomicron
Bacteroides uniformis
Bacteroides ureolyticus
Bacteroides vulgatus
Balnearium
Balnearium lithotrophicum
Balneatrix
Balneatrix alpica
Balneola
Balneola vulgaris
Barnesiella
Barnesiella viscericola
Bartonella
Bartonella alsatica
Bartonella bacilliformis
Bartonella clarridgeiae
Bartonella doshiae
Bartonella elizabethae
Bartonella grahamii
Bartonella henselae
Bartonella rochalimae
Bartonella vinsonii
Bavariicoccus
Bavariicoccus selleri
Bdellovibrio
Bdellovibrio bacteriovorus
Bdellovibrio exovorus
Beggiatoa
Beggiatoa alba
Beijerinckla
Beijerinckia derxii
Beijerinckia fluminensis
Beijerinckia indica
Beijerinckia mobilis
Belliella
Belliella baltica
Bellilinea
Bellilinea caldifistulae
Belnapia
Belnapia moabensis
Bergeriella
Bergeriella denitrificans
Beutenbergia
Beutenbergia cavernae
Bibersteinia
Bibersteinia trehalosi
Bifidobacterium
Bifidobacterium adolescentis
Bifidobacterium angulatum
Bifidobacterium animalis
Bifidobacterium asteroides
Bifidobacterium bifidum
Bifidobacterium boum
Bifidobacterium breve
Bifidobacterium catenulatum
Bifidobacterium choerinum
Bifidobacterium coryneforme
Bifidobacterium cuniculi
Bifidobacterium dentium
Bifidobacterium gallicum
Bifidobacterium gallinarum
Bifidobacterium indicum
Bifidobacterium longum
Bifidobacterium
magnumBifidobacterium merycicum
Bifidobacterium minimum
Bifidobacterium pseudocatenulatum
Bifidobacterium pseudolongum
Bifidobacterium pullorum
Bifidobacterium ruminantium
Bifidobacterium saeculare
Bifidobacterium subtile
Bifidobacterium thermophilum
Bilophila
Bilophila wadsworthia
Biostraticola
Biostraticola tofi
Bizionia
Bizionia argentinensis
Blastobacter
Blastobacter capsulatus
Blastobacter denitrificans
Blastococcus
Blastococcus aggregatus
Blastococcus saxobsidens
Blastochloris
Blastochloris viridis
Blastomonas
Blastomonas natatoria
Blastopirellula
Blastopirellula marina
Blautia
Blautia coccoides
Blautia hansenii
Blautia producta
Blautia wexlerae
Bogoriella
Bogoriella caseilytica
Bordetella
Bordetella avium
Bordetella bronchiseptica
Bordetella hinzii
Bordetella holmesii
Bordetella parapertussis
Bordetella pertussis
Bordetella petrii
Bordetella trematum
Borrella
Borrelia afzelii
Borrelia americana
Borrelia burgdorferi
Borrelia carolinensis
Borrelia coriaceae
Borrelia garinii
Borrelia japonica
Bosea
Bosea minatitlanensis
Bosea thiooxidans
Brachybacterium
Brachybacterium alimentarium
Brachybacterium faecium
Brachybacterium paraconglomeratum
Brachybacterium rhamnosum
Brachybacterium tyrofermentans
Brachyspira
Brachyspira alvinipulli
Brachyspira hyodysenteriae
Brachyspira innocens
Brachyspira murdochii
Brachyspira pilosicoli
Bradyrhizobium
Bradyrhizobium canariense
Bradyrhizobium elkanii
Bradyrhizobium japonicum
Bradyrhizobium liaoningense
Brenneria
Brenneria alni
Brenneria nigrifluens
Brenneria quercina
Brenneria quercina
Brenneria salicis
Brevibacillus
Brevibacillus agri
Brevibacillus borstelensis
Brevibacillus brevis
Brevibacillus centrosporus
Brevibacillus choshinensis
Brevibacillus invocatus
Brevibacillus laterosporus
Brevibacillus parabrevis
Brevibacillus reuszeri
Brevibacterium
Brevibacterium abidum
Brevibacterium album
Brevibacterium aurantiacum
Brevibacterium celere
Brevibacterium epidermidis
Brevibacterium frigoritolerans
Brevibacterium halotolerans
Brevibacterium iodinum
Brevibacterium linens
Brevibacterium lyticum
Brevibacterium mcbrellneri
Brevibacterium otitidis
Brevibacterium oxydans
Brevibacterium paucivorans
Brevibacterium stationis
Brevinema
Brevinema andersonii
Brevundimonas
Brevundimonas alba
Brevundimonas aurantiaca
Brevundimonas diminuta
Brevundimonas intermedia
Brevundimonas subvibrioides
Brevundimonas vancanneytii
Brevundimonas variabilis
Brevundimonas vesicularis
Brochothrix
Brochothrix campestris
Brochothrix thermosphacta
Brucella
Brucella canis
Brucella neotomae
Bryobacter
Bryobacter aggregatus
Burkholderia
Burkholderia ambifaria
Burkholderia andropogonis
Burkholderia anthina
Burkholderia caledonica
Burkholderia caryophylli
Burkholderia cenocepacia
Burkholderia cepacia
Burkholderia cocovenenans
Burkholderia dolosa
Burkholderia fungorum
Burkholderia glathei
Burkholderia glumae
Burkholderia graminis
Burkholderia kururiensis
Burkholderia multivorans
Burkholderia phenazinium
Burkholderia plantarii
Burkholderia pyrrocinia
Burkholderia silvatlantica
Burkholderia stabilis
Burkholderia thailandensis
Burkholderia tropica
Burkholderia unamae
Burkholderia vietnamiensis
Buttiauxella
Buttiauxella agrestis
Buttiauxella brennerae
Buttiauxella ferragutiae
Buttiauxella gaviniae
Buttiauxella izardii
Buttiauxella noackiae
Buttiauxella warmboldiae
Butyrivibrio
Butyrivibrio fibrisolvens
Butyrivibrio hungatei
Butyrivibrio proteoclasticus
Bacillus
B. acidiceler
B. acidicola
B. acidiproducens
B. acidocaldarius
B. acidoterrestris
B. aeolius
B. aerius
B. aerophilus
B. agaradhaerens
B. agri
B. aidingensis
B. akibai
B. alcalophilus
B. algicola
B. alginolyticus
B. alkalidiazotrophicus
B. alkalinitrilicus
B. alkalisediminis
B. alkalitelluris
B. altitudinis
B. alveayuensis
B. alvei
B. amyloliquefaciens
B. a. subsp. Amyloliquefaciens
B. a. subsp. Plantarum
B. dipsosauri
B. drentensis
B. edaphicus
B. ehimensis
B. eiseniae
B. enclensis
B. endophyticus
B. endoradicis
B. farraginis
B. fastidiosus
B. fengqiuensis
B. firmus
B. flexus
B. foraminis
B. fordii
B. formosus
B. fortis
B. fumarioli
B. funiculus
B. fusiformis
B. galactophilus
B. galactosidilyticus
B. galliciensis
B. gelatini
B. gibsonii
B. ginsengi
B. ginsengihumi
B. ginsengisoli
B. globisporus (eg, B. g. subsp. Globisporus; or B. g. subsp. Marinus)
B. aminovorans
B. amylolyticus
B. andreesenii
B. aneurinilyticus
B. anthracis
B. aquimaris
B. arenosi
B. arseniciselenatis
B. arsenicus
B. aurantiacus
B. arvi
B. aryabhattai
B. asahii
B. atrophaeus
B. axarquiensis
B. azotofixans
B. azotoformans
B. badius
B. barbaricus
B. bataviensis
B. beijingensis
B. benzoevorans
B. beringensis
B. berkeleyi
B. beveridgei
B. bogoriensis
B. boroniphilus
B. borstelensis
B. brevis Migula
B. butanolivorans
B. canaveralius
B. carboniphilus
B. cecembensis
B. cellulosilyticus
B. centrosporus
B. cereus
B. chagannorensis
B. chitinolyticus
B. chondroitinus
B. choshinensis
B. chungangensis
B. cibi
B. circulans
B. clarkii
B. clausii
B. coagulans
B. coahuilensis
B. cohnii
B. composti
B. curdlanolyticus
B. cycloheptanicus
B. cytotoxicus
B. daliensis
B. decisifrondis
B. decolorationis
B. deserti
B. glucanolyticus
B. gordonae
B. gottheilii
B. graminis
B. halmapalus
B. haloalkaliphilus
B. halochares
B. halodenitrificans
B. halodurans
B. halophilus
B. halosaccharovorans
B. hemicellulosilyticus
B. hemicentrotroti
B. herbersteinensis
B. horikoshii
B. horneckiae
B. horti
B. huizhouensis
B. humi
B. hwajinpoensis
B. idriensis
B. indicus
B. infantis
B. infernus
B. insolitus
B. invictae
B. iranensis
8. isabeliae
B. isronensis
B. jeotgali
B. kaustophilus
B. kobensis
B. kochii
B. kokeshilformis
B. koreensis
B. korlensis
B. kribbensis
B. krulwichiae
B. laevolacticus
B. larvae
B. laterosporus
B. salexigens
B. saliphilus
B. schlegelii
B. sediminis
B. selenatarsenatis
B. selenitireducens
B. seohaeanensis
B. shacheensis
B. shackletonii
B. siamensis
B. silvestris
B. simplex
B. siralis
B. smithii
B. soli
B. solimangrovi
B. solisalsi
B. songklensis
B. sonorensis
B. sphaericus
B. sporothermodurans
B. stearothermophilus
B. stratosphericus
B. subterraneus
B. subtilis (eg, B. s. subsp. Inaquosorum; or B. s.
subsp. Spizizeni; or B. s. subsp. Subtilis)
B. taeanensis
B. tequilensis
B. thermantarcticus
B. thermoaerophilus
B. thermoamylovorans
B. thermocatenulatus
B. thermocloacae
B. thermocopriae
B. thermodenitrificans
B. thermoglucosidasius
B. thermolactis
B. thermoleovorans
B. thermophilus
B. thermoruber
B. thermosphaericus
B. thiaminolyticus
B. thioparans
B. thuringiensis
B. tianshenii
B. trypoxylicola
B. tusciae
B. validus.
B. vallismortis
B. vedderi
B. velezensis
B. vietnamensis
B. vireti
B. vulcani
B. wakoensis
B. weihenstephanensis
B. xiamenensis
B. xiaoxiensis
B. zhanjiangensis
B. peoriae
B. persepolensis
B. persicus
B. pervagus
B. plakortidis
B. pocheonensis
B. polygoni
B. polymyxa
B. popilliae
B. pseudalcalophilus
B. pseudofirmus
B. pseudomycoides
B. psychrodurans
B. psychrophilus
B. psychrosaccharolyticus
B. psychrotolerans
B. pulvifaciens
B. pumilus
B. purgationiresistens
B. pycnus
B. qingdaonensis
B. qingshengii
B. reuszeri
B. rhizosphaerae
B. rigui
B. ruris
B. safensis
B. salarius
B. lautus
B. lehensis
B. lentimorbus
B. lentus
B. licheniformis
B. ligniniphilus
B. litoralis
B. locisalis
B. luciferensis
B. luteolus
B. luteus
B. macauensis
B. macerans
B. macquariensis
B. macyae
B. malacitensis
B. mannanilyticus
B. marisflavi
B. marismortui
B. marmarensis
B. massiliensis
B. megaterium
B. mesonae
B. methanolicus
B. methylotrophicus
B. migulanus
B. mojavensis
B. mucilaginosus
B. muralis
B. murimartini
B. mycoides
B. naganoensis
B. nanhaiensis
B. nanhaiisediminis
B. nealsonii
B. neidei
B. neizhouensis
B. niabensis
B. niacini
B. novalis
B. oceanisediminis
B. odysseyi
B. okhensis
B. okuhidensis
B. oleronius
B. oryzaecorticis
B. oshimensis
B. pabuli
B. pakistanensis
B. pallidus
B. pallidus
B. panacisoli
B. panaciterrae
B. pantothenticus
B. parabrevis
B. paraflexus
B. pasteurii
B. patagoniensis
Caenimonas
Caenimonas koreensis
Caldalkalibacillus
Caldalkalibacillus uzonensis
Caldanaerobacter
Caldanaerobacter subterraneus
Caldanaerobius
Caldanaerobius fijiensis
Caldanaerobius polysaccharolyticus
Caldanaerobius zeae
Caldanaerovirga
Caldanaerovirga acetigignens
Caldicellulosiruptor
Caldicellulosiruptor bescii
Caldicellulosiruptor kristjanssonii
Caldicellulosiruptor owensensis
Campylobacter
Campylobacter coli
Campylobacter concisus
Campylobacter curvus
Campylobacter fetus
Campylobacter gracilis
Campylobacter helveticus
Campylobacter hominis
Campylobacter hyointestinalis
Campylobacter jejuni
Campylobacter lari
Campylobacter mucosalis
Campylobacter rectus
Campylobacter showae
Campylobacter sputorum
Campylobacter upsaliensis
Capnocytophaga
Capnocytophaga canimorsus
Capnocytophaga cynodegmi
Capnocytophaga gingivalis
Capnocytophaga granulosa
Capnocytophaga haemolytica
Capnocytophaga ochracea
Capnocytophaga sputigena
Cardiobacterium
Cardiobacterium hominis
Carnimonas
Carnimonas nigrificans
Carnobacterium
Carnobacterium alterfunditum
Carnobacterium divergens
Carnobacterium funditum
Carnobacterium gallinarum
Carnobacterium maltaromaticum
Carnobacterium mobile
Carnobacterium viridans
Caryophanon
Caryophanon latum
Caryophanon tenue
Catellatospora
Catellatospora citrea
Catellatospora methionotrophica
Catenococcus
Catenococcus thiocycli
Catenuloplanes
Catenuloplanes atrovinosus
Catenuloplanes castaneus
Catenuloplanes crispus
Catenuloplanes indicus
Catenuloplanes japonicus
Catenuloplanes nepalensis
Catenuloplanes niger
Chryseobacterium
Chryseobacterium balustinum
Citrobacter
C. amalonaticus
C. braakii
C. diversus
C. farmeri
C. freundii
C. gillenii
C. koseri
C. murliniae
C. pasteurii
C. rodentium
C. sedlakii
C. werkmanii
C. youngae
Coccochloris
Coccochloris elabens
Corynebacterium
Corynebacterium flavescens
Corynebacterium variabile
Curtobacterium
Curtobacterium albidum
Curtobacterium citreus
Clostridium
Clostridium absonum,
Clostridium aceticum,
Clostridium acetireducens,
Clostridium acetobutylicum,
Clostridium acidisoli,
Clostridium aciditolerans,
Clostridium acidurici,
Clostridium aerotolerans,
Clostridium aestuarii,
Clostridium akagii,
Clostridium aldenense,
Clostridium aldrichii,
Clostridium algidicarni,
Clostridium algidixylanolyticum,
Clostridium algifaecis,
Clostridium algoriphilum,
Clostridium alkalicellulosi,
Clostridium aminophilum,
Clostridium aminovalericum,
Clostridium amygdalinum,
Clostridium amylolyticum,
Clostridium arbusti,
Clostridium arcticum,
Clostridium argentinense,
Clostridium asparagiforme,
Clostridium aurantibutyricum,
Clostridium autoethanogenum,
Clostridium baratii,
Clostridium barkeri,
Clostridium bartlettii,
Clostridium beijerinckii,
Clostridium bifermentans,
Clostridium bolteae,
Clostridium bornimense,
Clostridium botulinum,
Clostridium bowmanii,
Clostridium bryantii,
Clostridium butyricum,
Clostridium cadaveris,
Clostridium caenicola,
Clostridium caminithermale,
Clostridium carboxidivorans,
Clostridium carnis,
Clostridium cavendishii,
Clostridium celatum,
Clostridium celerecrescens,
Clostridium cellobioparum,
Clostridium cellulofermentans,
Clostridium cellulolyticum,
Clostridium cellulosi,
Clostridium cellulovorans,
Clostridium chartatabidum,
Clostridium chauvoei,
Clostridium chromiireducens,
Clostridium citroniae,
Clostridium clariflavum,
Clostridium clostridioforme,
Clostridium coccoides,
Clostridium cochlearium,
Clostridium colletant,
Clostridium colicanis,
Clostridium colinum,
Clostridium collagenovorans,
Clostridium cylindrosporum,
Clostridium difficile,
Clostridium diolis,
Clostridium disporicum,
Clostridium drakei,
Clostridium durum,
Clostridium estertheticum,
Clostridium estertheticum estertheticum,
Clostridium estertheticum laramiense,
Clostridium fallax,
Clostridium felsineum,
Clostridium fervidum,
Clostridium fimetarium,
Clostridium formicaceticum,
Clostridium frigidicarnis,
Clostridium frigoris,
Clostridium ganghwense,
Clostridium gasigenes,
Clostridium ghonii,
Clostridium glycolicum,
Clostridium glycyrrhizinilyticum,
Clostridium grantii,
Clostridium haemolyticum,
Clostridium halophilum,
Clostridium hastiforme,
Clostridium hathewayi,
Clostridium herbivorans,
Clostridium hiranonis,
Clostridium histolyticum,
Clostridium homopropionicum,
Clostridium huakuii,
Clostridium hungatei,
Clostridium hydrogeniformans,
Clostridium hydroxybenzoicum,
Clostridium hylemonae,
Clostridium jejuense,
Clostridium indolis,
Clostridium innocuum,
Clostridium intestinale,
Clostridium irregulare,
Clostridium isatidis,
Clostridium josui,
Clostridium kluyveri,
Clostridium lactatifermentans,
Clostridium lacusfryxellense,
Clostridium laramiense,
Clostridium lavalense,
Clostridium lentocellum,
Clostridium lentoputrescens,
Clostridium leptum,
Clostridium limosum,
Clostridium litorale,
Clostridium lituseburense,
Clostridium ljungdahlii,
Clostridium lortetii,
Clostridium lundense,
Clostridium magnum,
Clostridium malenominatum,
Clostridium mangenotii,
Clostridium mayombei,
Clostridium methoxybenzovorans,
Clostridium methylpentosum,
Clostridium neopropionicum,
Clostridium nexile,
Clostridium nitrophenolicum,
Clostridium novyi,
Clostridium oceanicum,
Clostridium orbiscindens,
Clostridium oroticum,
Clostridium oxalicum,
Clostridium papyrosolvens,
Clostridium paradoxum,
Clostridium paraperfringens (Alias: C. welchii),
Clostridium paraputrificum,
Clostridium pascui,
Clostridium pasteurianum,
Clostridium peptidivorans,
Clostridium perenne,
Clostridium perfringens,
Clostridium pfennigii,
Clostridium phytofermentans,
Clostridium piliforme,
Clostridium polysaccharolyticum,
Clostridium populeti,
Clostridium propionicum,
Clostridium proteoclasticum,
Clostridium proteolyticum,
Clostridium psychrophilum,
Clostridium puniceum,
Clostridium purinilyticum,
Clostridium putrefaciens,
Clostridium putrificum,
Clostridium quercicolum,
Clostridium quinii,
Clostridium ramosum,
Clostridium rectum,
Clostridium roseum,
Clostridium saccharobutylicum,
Clostridium saccharogumia,
Clostridium saccharolyticum,
Clostridium saccharoperbutylacetonicum
Clostridium sardiniense,
Clostridium sartagoforme,
Clostridium scatologenes,
Clostridium schirmacherense,
Clostridium scindens,
Clostridium septicum,
Clostridium sordellii,
Clostridium sphenoides,
Clostridium spiroforme,
Clostridium sporogenes,
Clostridium sporosphaeroides,
Clostridium stercorarium,
Clostridium stercorarium leptospartum,
Clostridium stercorarium stercorarium,
Clostridium stercorarium thermolacticum,
Clostridium sticklandii,
Clostridium straminisolvens,
Clostridium subterminale,
Clostridium sufflavum,
Clostridium sulfidigenes,
Clostridium symbiosum,
Clostridium tagluense,
Clostridium tepidiprofundi,
Clostridium termitidis,
Clostridium tertium,
Clostridium tetani,
Clostridium tetanomorphum,
Clostridium thermaceticum,
Clostridium thermautotrophicum,
Clostridium thermoalcaliphilum,
Clostridium thermobutyricum,
Clostridium thermocellum,
Clostridium thermocopriae,
Clostridium thermohydrosulfuricum,
Clostridium thermolacticum,
Clostridium thermopalmarium,
Clostridium thermopapyrolyticum,
Clostridium thermosaccharolyticum,
Clostridium thermosuccinogenes,
Clostridium thermosulfurigenes,
Clostridium thiosulfatireducens,
Clostridium tyrobutyricum,
Clostridium uliginosum,
Clostridium ultunense,
Clostridium villosum,
Clostridium vincentii,
Clostridium viride,
Clostridium xylanolyticum,
Clostridium xylanovorans
Dactylosporangium
Dactylosporangium aurantiacum
Dactylosporangium fulvum
Dactylosporangium matsuzakiense
Dactylosporangium roseum
Dactylosporangium thailandense
Dactylosporangium vinaceum
Deinococcus
Deinococcus aerius
Deinococcus apachensis
Deinococcus aquaticus
Deinococcus aquatilis
Deinococcus caeni
Deinococcus radiodurans
Deinococcus radiophilus
Delftia
Delftia acidovorans
Desulfovibrio
Desulfovibrio desulfuricans
Diplococcus
Diplococcus pneumoniae
Echinicola
Echinicola pacifica
Echinicola vietnamensis
Enterobacter
E. aerogenes
E. amnigenus
E. agglomerans
E. arachidis
E. asburiae
E. cancerogenous
E. cloacae
E. cowanii
E. dissolvens
E. gergoviae
E. helveticus
E. hormaechei
E. intermedius
Enterobacter kobei
E. ludwigii
E. mori
E. nimipressuralis
E. oryzae
E. pulveris
E. pyrinus
E. radicincitans
E. taylorae
E. turicensis
E. sakazakii Enterobacter soli
Enterococcus
Enterococcus durans
Enterococcus faecalis
Enterococcus faecium
Erwinia
Erwinia hapontici
Escherichia
Escherichia coli
Faecalibacterium
Faecalibacterium prausnitzii
Fangia
Fangia hongkongensis
Fastidiosipila
Fastidiosipila sanguinis
Fusobacterium
Fusobacterium nucleatum
Flavobacterium
Flavobacterium antarcticum
Flavobacterium aquatile
Flavobacterium aquidurense
Flavobacterium balustinum
Flavobacterium croceum
Flavobacterium cucumis
Flavobacterium daejeonense
Flavobacterium defluvii
Flavobacterium degerlachei
Flavobacterium denitrificans
Flavobacterium filum
Flavobacterium flevense
Flavobacterium frigidarium
Flavobacterium mizutaii
Flavobacterium okeanokoites
Gaetbulibacter
Gaetbulibacter saemankumensis
Gallibacterium
Gallibacterium anatis
Gallicola
Gallicola barnesae
Garciella
Garciella nitratireducens
Geobacillus
Geobacillus thermoglucosidasius
Geobacillus stearothermophilus
Geobacter
Geobacter bemidjiensis
Geobacter bremensis
Geobacter chapellei
Geobacter grbiciae
Geobacter hydrogenophilus
Geobacter lovleyi
Geobacter metallireducens
Geobacter pelophilus
Geobacter pickeringii
Geobacter sulfurreducens
Geodermatophilus
Geodermatophilus obscurus
Gluconacetobacter
Gluconacetobacter xylinus
Gordonia
Gordonia rubripertincta
Haemophilus
Haemophilus aegyptius
Haemophilus aphrophilus
Haemophilus felis
Haemophilus gallinarum
Haemophilus haemolyticus
Haemophilus influenzae
Haemophilus paracuniculus
Haemophilus parahaemolyticus
Haemophilus parainfluenzae
Haemophilus paraphrohaemolyticus
Haemophilus parasuis
Haemophilus pittmaniae
Hafnia
Hafnia alvei
Hahella
Hahella ganghwensis
Halalkalibacillus
Halalkalibacillus halophilus
Helicobacter
Helicobacter pylori
Ideonella
Ideonella azotifigens
Idiomarina
Idiomarina abyssalis
Idiomarina baltica
Idiomarina fontislapidosi
Idiomarina loihiensis
Idiomarina ramblicola
Idiomarina seosinensis
Idiomarina zobellii
Ignatzschineria
Ignatzschineria larvae
Ignavigranum
Ignavigranum ruoffiae
Ilumatobacter
Ilumatobacter fluminis
Ilyobacter
Ilyobacter delafieldii
Ilyobacter insuetus
Ilyobacter polytropus
Ilyobacter tartaricus
Janibacter
Janibacter anophelis
Janibacter corallicola
Janibacter limosus
Janibacter melonis
Janibacter terrae
Jannaschia
Jannaschia cystaugens
Jannaschia helgolandensis
Jannaschia pohangensis
Jannaschia rubra
Janthinobacterium
Janthinobacterium agaricidamnosum
Janthinobacterium lividum
Jejuia
Jejuia pallidilutea
Jeotgalibacillus
Jeotgalibacillus alimentarius
Jeotgalicoccus
Jeotgalicoccus halotolerans
Kaistia
Kaistia adipata
Kaistia soli
Kangiella
Kangiella aquimarina
Kangiella koreensis
Kerstersia
Kerstersia gyiorum
Kiloniella
Kiloniella laminariae
Klebsiella
K. granulomatis
K. oxytoca
K. pneumoniae
K. terrigena
K. variicola
Kluyvera
Kluyvera ascorbata
Kocuria
Kocuria roasea
Kocuria varians
Kurthia
Kurthia zopfii
Labedella
Labedella gwakjiensis
Labrenzia
Labrenzia aggregata
Labrenzia alba
Labrenzia alexandrii
Labrenzia marina
Labrys
Labrys methylaminiphilus
Labrys miyagiensis
Labrys monachus
Labrys okinawensis
Labrys portucalensis
Laceyella
Laceyella putida
Lechevalieria
Lechevalieria aerocolonigenes
Listeria
L. aquatica
L. booriae
L. cornellensis
L. fleischmannii
L. floridensis
L. grandensis
L. grayi
L. innocua
Listeria ivanovii
L. marthii
L. monocytogenes
L. newyorkensis
L. riparia
L. rocourtiae
L. seeligeri
L. weihenstephanensis
L. welshimeri
Listonella
Listonella anguillarum
Macrococcus
Macrococcus bovicus
Marinobacter
Marinobacter algicola
Marinobacter bryozoorum
Marinobacter flavimaris
Meiothermus
Meiothermus ruber
Methylophilus
Methylophilus methylotrophus
Microbacterium
Microbacterium ammoniaphilum
Microbacterium arborescens
Microbacterium liquefaciens
Microbacterium oxydans
Micrococcus
Micrococcus luteus
Micrococcus lylae
Moraxella
Moraxella bovis
Moraxella nonliquefaciens
Moraxella osloensis
Nakamurella
Nakamurella mulipartita
Nannocystis
Nannocystis pusilla
Natranaerobius
Natranaerobius thermophilus
Natranaerobius trueperi
Naxibacter
Naxibacter alkalitolerans
Neisseria
Neisseria cinerea
Neisseria denitrificans
Neisseria gonorrhoeae
Neisseria lactamica
Neisseria mucosa
Neisseria sicca
Neisseria subflava
Neptunomonas
Neptunomonas japonica
Nesterenkonia
Nesterenkonia holobia
Nocardia
Nocardia argentinensis
Nocardia corallina
Nocardia otitidiscaviarum
Lactobacillus
L. acetotolerans
L. acidifarinae
L. acidipiscis
L. acidophilus
Lactobacillus agilis
L. algidus
L. alimentarius
L. amylolyticus
L. amylophilus
L. amylotrophicus
L. amylovorus
L. animalis
L. antri
L. apodemi
L. aviarius
L. bifermentans
L. brevis
L. buchneri
L. camelliae
L. casei
L. kitasatonis
L. kunkeei
L. leichmannii
L. lindneri
L. malefermentans
L. catenaformis
L. ceti
L. coleohominis
L. collinoides
L. composti
L. concavus
L. coryniformis
L. crispatus
L. crustorum
L. curvatus
L. delbrueckii subsp. Bulgaricus
L. delbrueckii subsp. Delbrueckii
L. delbrueckii subsp. Lactis
L. dextrinicus
L. diolivorans
L. equi
L. equigenerosi
L. farraginis
L. farciminis
L. fermentum
L. fornicalis
L. fructivorans
L. frumenti
L. mali
L. manihotivorans
L. mindensis
L. mucosae
L. murinus
L. nagelii
L. namurensis
L. nantensis
L. oligofermentans
L. oris
L. panis
L. pantheris
L. parabrevis
L. parabuchneri
L. paracasei
L. paracollinoides
L. parafarraginis
L. homohiochii
L. iners
L. ingluviei
L. intestinalis
L. fuchuensis
L. gallinarum
L. gasseri
L. parakefiri
L. paralimentarius
L. paraplantarum
L. pentosus
L. perolens
L. plantarum
L. pontis
L. protectus
L. psittaci
L. rennini
L. reuteri
L. rhamnosus
L. rimae
L. rogosae
L. rossiae
L. ruminis
L. saerimneri
L. jensenii
L. johnsonii
L. kalixensis
L. kefiranofaciens
L. kefiri
L. aviarius
L. helveticus
L. hilgardii
L. sakei
L. salivarius
L. sanfranciscensis
L. satsumensis
L. secaliphilus
L. sharpeae
L. siliginis
L. spicheri
L. suebicus
L. thailandensis
L. ultunensis
L. vaccinostercus
L. vaginalis
L. versmoldensis
L. vini
L. vitulinus
L. zeae
L. zymae
L. gastricus
L. ghanensis
L. graminis
L. hammesii
L. hamsteri
L. harbinensis
L. hayakitensis
Legionella
Legionella adelaidensis
Legionella anisa
Legionella beliardensis
Legionella birminghamensis
Legionella bozemanae
Legionella brunensis
Legionella busanensis
Legionella cardiaca
Legionella cherrii
Legionella cincinnatiensis
Legionella clemsonensis
Legionella donaldsonii
Legionella drancourtii
Legionella dresdenensis
Legionella drozanskii
Legionella dumoffii
Legionella erythra
Legionella fairfieldensis
Legionella fallonii
Legionella feeleii
Legionella geestiana
Legionella genomospecies
Legionella gormanii
Legionella gratiana
Legionella gresilensis
Legionella hackeliae
Legionella impletisoli
Legionella israelensis
Legionella jamestowniensis
Candidatus Legionella jeonii
Legionella jordanis
Legionella lansingensis
Legionella londiniensis
Legionella longbeachae
Legionella lytica
Legionella maceachernii
Legionella massiliensis
Legionella micdadei
Legionella monrovica
Legionella moravica
Legionella nagasakiensis
Legionella nautarum
Legionella norrlandica
Legionella oakridgensis
Legionella parisiensis
Legionella pittsburghensis
Legionella pneumophila
Legionella quateirensis
Legionella quinlivanii
Legionella rowbothamii
Legionella rubrilucens
Legionella sainthelensi
Legionella santicrucis
Legionella shakespearei
Legionella spiritensis
Legionella steelei
Legionella steigerwaltii
Legionella taurinensis
Legionella tucsonensis
Legionella tunisiensis
Legionella wadsworthii
Legionella waltersii
Legionella worsleiensis
Legionella yabuuchiae
Oceanibulbus
Oceanibulbus indolifex
Oceanicaulis
Oceanicaulis alexandrii
Oceanicola
Oceanicola batsensis
Oceanicola granulosus
Oceanicola nanhaiensis
Oceanimonas
Oceanimonas baumannii
Oceaniserpentilla
Oceaniserpentilla haliotis
Oceanisphaera
Oceanisphaera donghaensis
Oceanisphaera litoralis
Oceanithermus
Oceanithermus desulfurans
Oceanithermus profundus
Oceanobacillus
Oceanobacillus caeni
Oceanospirillum
Oceanospirillum linum
Paenibacillus
Paenibacillus thiaminolyticus
Pantoea
Pantoea agglomerans
Paracoccus
Paracoccus alcaliphilus
Paucimonas
Paucimonas lemoignei
Pectobacterium
Pectobacterium aroidearum
Pectobacterium atrosepticum
Pectobacterium betavasculorum
Pectobacterium cacticida
Pectobacterium carnegieana
Pectobacterium carotovorum
Pectobacterium chrysanthemi
Pectobacterium cypripedii
Pectobacterium rhapontici
Pectobacterium wasabiae
Planococcus
Planococcus citreus
Planomicrobium
Planomicrobium okeanokoites
Plesiomonas
Plesiomonas shigelloides
Proteus
Proteus vulgaris
Prevotella
Prevotella albensis
Prevotella amnii
Prevotella bergensis
Prevotella bivia
Prevotella brevis
Prevotella bryantii
Prevotella buccae
Prevotella buccalis
Prevotella copri
Prevotella dentalis
Prevotella denticola
Prevotella disiens
Prevotella histicola
Prevotella intermedia
Prevotella maculosa
Prevotella marshii
Prevotella melaninogenica
Prevotella micans
Prevotella multiformis
Prevotella nigrescens
Prevotella oralis
Prevotella oris
Prevotella oulorum
Prevotella pallens
Prevotella salivae
Prevotella stercorea
Prevotella tannerae
Prevotella timonensis
Prevotella veroralis
Providencia
Providencia stuartii
Pseudomonas
Pseudomonas aeruginosa
Pseudomonas alcaligenes
Pseudomonas anguillispetica
Pseudomonas fluorescens
Pseudoalteromonas haloplanktis
Pseudomonas mendocina
Pseudomonas pseudoalcaligenes
Pseudomonas putida
Pseudomonas tutzeri
Pseudomonas syringae
Psychrobacter
Psychrobacter faecalis
Psychrobacter phenylpyruvicus
Quadrisphaera
Quadrisphaera granulorum
Quatrionicoccus
Quatrionicoccus australiensis
Quinella
Quinella ovalis
Ralstonia
Ralstonia eutropha
Ralstonia insidiosa
Ralstonia mannitolilytica
Ralstonia pickettii
Ralstonia pseudosolanacearum
Ralstonia syzygii
Ralstonia solanacearum
Ramlibacter
Ramlibacter henchirensis
Ramlibacter tataouinensis
Raoultella
Raoultella ornithinolytica
Raoultella planticola
Raoultella terrigena
Rathayibacter
Rathayibacter caricis
Rathayibacter festucae
Rathayibacter iranicus
Rathayibacter rathayi
Rathayibacter toxicus
Rathayibacter tritici
Rhodobacter
Rhodobacter sphaeroides
Ruegeria
Ruegeria gelatinovorans
Saccharococcus
Saccharococcus thermophilus
Saccharomonospora
Saccharomonospora azurea
Saccharomonospora cyanea
Saccharomonospora viridis
Saccharophagus
Saccharophagus degradans
Saccharopolyspora
Saccharopolyspora erythraea
Saccharopolyspora gregorii
Saccharopolyspora hirsuta
Saccharopolyspora hordei
Saccharopolyspora rectivirgula
Saccharopolyspora spinosa
Saccharopolyspora taberi
Saccharothrix
Saccharothrix australiensis
Saccharothrix coeruleofusca
Saccharothrix espanaensis
Saccharothrix longispora
Saccharothrix mutabilis
Saccharothrix syringae
Saccharothrix tangerinus
Saccharothrix texasensis
Sagittula
Sagittula stellata
Salegentibacter
Salegentibacter salegens
Salimicrobium
Salimicrobium album
Salinibacter
Salinibacter ruber
Salinicoccus
Salinicoccus alkaliphilus
Salinicoccus hispanicus
Salinicoccus roseus
Salinispora
Salinispora arenicola
Salinispora tropica
Salinivibrio
Salinivibrio costicola
Salmonella
Salmonella bongori
Salmonella enterica
Salmonella subterranea
Salmonella typhi
Sanguibacter
Sanguibacter keddieii
Sanguibacter suarezii
Saprospira
Saprospira grandis
Sarcina
Sarcina maxima
Sarcina ventriculi
Sebaldella
Sebaldella termitidis
Serratia
Serratia fonticola
Serratia marcescens
Sphaerotilus
Sphaerotilus natans
Sphingobacterium
Sphingobacterium multivorum
Stenotrophomonas
Stenotrophomonas maltophilia
Streptomyces
Streptomyces achromogenes
Streptomyces cesalbus
Streptomyces cescaepitosus
Streptomyces cesdiastaticus
Streptomyces cesexfoliatus
Streptomyces fimbriatus
Streptomyces fradiae
Streptomyces fulvissimus
Streptomyces griseoruber
Streptomyces griseus
Streptomyces lavendulae
Streptomyces phaeochromogenes
Streptomyces thermodiastaticus
Streptomyces tubercidicus
Tatlockia
Tatlockia maceachernii
Tatlockia micdadei
Tenacibaculum
Tenacibaculum amylolyticum
Tenacibaculum discolor
Tenacibaculum gallaicum
Tenacibaculum lutimaris
Tenacibaculum mesophilum
Tenacibaculum skagerrakense
Tepidanaerobacter
Tepidanaerobacter syntrophicus
Tepidibacter
Tepidibacter formicigenes
Tepidibacter thalassicus
Thermus
Thermus aquaticus
Thermus filiformis
Thermus thermophilus
Staphylococcus
S. arlettae
S. agnetis
S. aureus
S. auricularis
S. capitis
S. caprae
S. carnosus
S. caseolyticus
S. chromogenes
S. cohnii
S. condimenti
S. delphini
S. devriesei
S. epidermidis
S. equorum
S. felis
S. fleurettii
S. gallinarum
S. haemolyticus
S. hominis
S. hyicus
S. intermedius
S. kloosii
S. leei
S. lentus
S. lugdunensis
S. lutrae
S. lyticans
S. massiliensis
S. microti
S. muscae
S. nepalensis
S. pasteuri
S. petrasii
S. pettenkoferi
S. piscifermentans
S. pseudintermedius
S. pseudolugdunensis
S. pulvereri
S. rostri
S. saccharolyticus
S. saprophyticus
S. schleiferi
S. sciuri
S. simiae
S. simulans
S. stepanovicii
S. succinus
S. vitulinus
S. warneri
S. xylosus
Streptococcus
Streptococcus agalactiae
Streptococcus anginosus
Streptococcus bovis
Streptococcus canis
Streptococcus constellatus
Streptococcus downei
Streptococcus dysgalactiae
Streptococcus equines
Streptococcus faecalis
Streptococcus ferus
Streptococcus infantarius
Streptococcus iniae
Streptococcus intermedius
Streptococcus lactarius
Streptococcus milleri
Streptococcus mitis
Streptococcus mutans
Streptococcus oralis
Streptococcus tigurinus
Streptococcus orisratti
Streptococcus parasanguinis
Streptococcus peroris
Streptococcus pneumoniae
Streptococcus pseudopneumoniae
Streptococcus pyogenes
Streptococcus ratti
Streptococcus salivariu
Streptococcus thermophilus
Streptococcus sanguinis
Streptococcus sobrinus
Streptococcus suis
Streptococcus uberis
Streptococcus vestibularis
Streptococcus viridans
Streptococcus zooepidemicus
Uliginosibacterium
Uliginosibacterium gangwonense
Ulvibacter
Ulvibacter litoralis
Umezawaea
Umezawaea angerina
Undibacterium
Undibacterium pigrum
Ureaplasma
Ureaplasma urealyticum
Ureibacillus
Ureibacillus composti
Ureibacillus suwonensis
Ureibacillus terrenus
Ureibacillus thermophilus
Ureibacillus thermosphaericus
Vagococcus
Vagococcus carniphilus
Vagococcus elongatus
Vagococcus fessus
Vagococcus fluvialis
Vagococcus lutrae
Vagococcus salmoninarum
Variovorax
Variovorax boronicumulans
Variovorax dokdonensis
Variovorax paradoxus
Variovorax soli
Veillonella
Veillonella atypica
Veillonella caviae
Veillonella criceti
Veillonella dispar
Veillonella montpellierensis
Veillonella parvula
Veillonella ratti
Veillonella rodentium
Venenivibrio
Venenivibrio stagnispumantis
Verminephrobacter
Verminephrobacter eiseniae
Verrucomicrobium
Verrucomicrobium spinosum
Vibrio
Vibrio aerogenes
Vibrio aestuarianus
Vibrio albensis
Vibrio alginolyticus
Vibrio campbellii
Vibrio cholerae
Vibrio cincinnatiensis
Vibrio coralliilyticus
Vibrio cyclitrophicus
Vibrio diazotrophicus
Vibrio fluvialis
Vibrio furnissii
Vibrio gazogenes
Vibrio halioticoli
Vibrio harveyi
Vibrio ichthyoenteri
Vibrio mediterranei
Vibrio metschnikovii
Vibrio mytili
Vibrio natriegens
Vibrio navarrensis
Vibrio nereis
Vibrio nigripulchritudo
Vibrio ordalii
Vibrio orientalis
Vibrio parahaemolyticus
Vibrio pectenicida
Vibrio penaeicida
Vibrio proteolyticus
Vibrio shilonii
Vibrio splendidus
Vibrio tubiashii
Vibrio vulnificus
Virgibacillus
Virgibacillus halodenitrificans
Virgibacillus pantothenticus
Weissella
Weissella cibaria
Weissella confusa
Weissella halotolerans
Weissella hellenica
Weissella kandleri
Weissella koreensis
Weissella minor
Weissella paramesenteroides
Weissella soli
Weissella thailandensis
Weissella viridescens
Williamsia
Williamsia marianensis
Williamsia maris
Williamsia serinedens
Winogradskyella
Winogradskyella thalassocola
Wolbachia
Wolbachia persica
Wolinella
Wolinella succinogenes
Zobellia
Zobellia galactanivorans
Zobellia uliginosa
Zoogloea
Zoogloea ramigera
Zoogloea resiniphila
Xanthobacter
Xanthobacter agilis
Xanthobacter aminoxidans
Xanthobacter autotrophicus
Xanthobacter flavus
Xanthobacter tagetidis
Xanthobacter viscosus
Xanthomonas
Xanthomonas albilineans
Xanthomonas alfalfae
Xanthomonas arboricola
Xanthomonas axonopodis
Xanthomonas campestris
Xanthomonas citri
Xanthomonas codiaei
Xanthomonas cucurbitae
Xanthomonas euvesicatoria
Xanthomonas fragariae
Xanthomonas fuscans
Xanthomonas gardneri
Xanthomonas hortorum
Xanthomonas hyacinthi
Xanthomonas perforans
Xanthomonas phaseoli
Xanthomonas pisi
Xanthomonas populi
Xanthomonas theicola
Xanthomonas translucens
Xanthomonas vesicatoria
Xylella
Xylella fastidiosa
Xylophilus
Xylophilus ampelinus
Xenophilus
Xenophilus azovorans
Xenorhabdus
Xenorhabdus beddingii
Xenorhabdus bovienii
Xenorhabdus cabanillasii
Xenorhabdus doucetiae
Xenorhabdus griffiniae
Xenorhabdus hominickii
Xenorhabdus koppenhoeferi
Xenorhabdus nematophila
Xenorhabdus poinarii
Xylanibacter
Xylanibacter oryzae
Yangia
Yangia pacifica
Yaniella
Yaniella flava
Yaniella halotolerans
Yeosuana
Yeosuana aromativorans
Yersinia
Yersinia aldovae
Yersinia bercovieri
Yersinia enterocolitica
Yersinia entomophaga
Yersinia frederiksenii
Yersinia intermedia
Yersinia kristensenii
Yersinia mollaretii
Yersinia philomiragia
Yersinia pestis
Yersinia pseudotuberculosis
Yersinia rohdei
Yersinia ruckeri
Yokenella
Yokenella regensburgei
Yonghaparkia
Yonghaparkia alkaliphila
Zavarzinia
Zavarzinia compransoris
Zooshikella
Zooshikella ganghwensis
Zunongwangia
Zunongwangia profunda
Zymobacter
Zymobacter palmae
Zymomonas
Zymomonas mobilis
Zymophilus
Zymophilus paucivorans
Zymophilus raffinosivorans
Zobellella
Zobellella denitrificans
Zobellella taiwanensis
Zeaxanthinibacter
Zeaxanthinibacter enoshimensis
Zhihengliuella
Zhihenglivella halotolerans
Xylanibacterium
Xylanibacterium ulmi

The present invention is described in more detail in the following non-limiting Examples.

### EXAMPLES

### Example 1: The Type-S CRISPRi/Cas System for CRISPRi Applications

### 1.1 Aim

This study evaluated i) the efficiency of the Type-S CRISPR/Cas system to bind to E. coli chromosomal and plasmid targets, acting as a CRISPRi tool, ii) which subunits are essential for DNA binding.

### 1.2 Introduction

We previously demonstrated that the Type-S CRISPR/Cas system drives crRNA-dependent hinderance of plasmid replication (see Example 1.3.2 and 1.3.3) while lacking a DNA nuclease domain. The crRNA-guided DNA binding activity of the Type-S CRISPR/Cas system can be exploited for the development of a CRISPR interference (CRISPRi) tool in E. coli. We expect that it will also be able to be used in other cell types. In this Example, the CRISPRi potential of the Type-S CRISPR/Cas system was evaluated via the targeted downregulation of the transcription, and subsequent translation, of a reporter green fluorescent protein (gfp) gene. The targeted gfp gene was either chromosomally integrated or plasmid-borne. Moreover, this Example revealed which Type-S CRISPR/Cas system subunits are essential for the DNA-binding activity of Type-S CRISPR/Cas system.

### 1.3 Methods and Results

Five variations of the Type-S CRISPR/Cas system were tested for their CRISPRi potential;
i) the wild type (wt) Type-S CRISPR/Cas system
ii) the Cas-S1 deletion mutant (ΔCas-S1) Type-S CRISPR/Cas system
iii) the Cas-S3 deletion mutant (ΔCas-S3) Type-S CRISPR/Cas system
iv) the Cas-S4 deletion mutant (ΔCas-S4) Type-S CRISPR/Cas system
v) the Cas-S1/Cas-S3 double deletion mutant (ΔCas-S1 ACa-S3) Type-S CRISPR/Cas system.

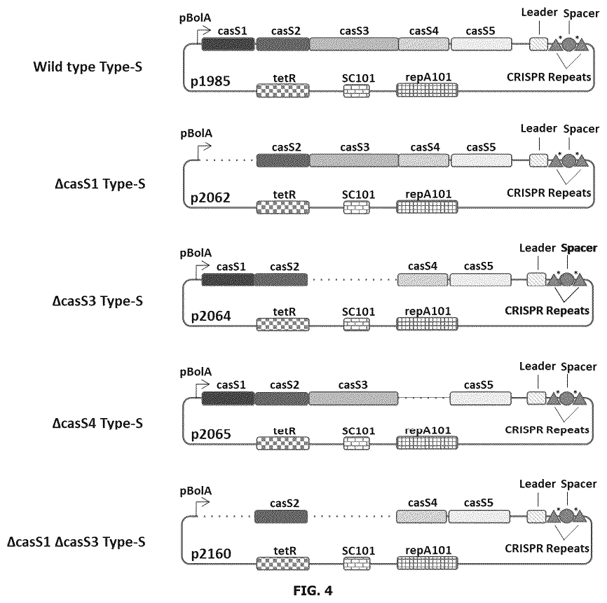

All variants were set under the transcriptional control of the pBolA promoter (SEQ ID No:14) and were cloned together with a non-targeting crRNA expressing module, generating plasmids p1985 (wt), p2062 (ΔCas-S1), p2064 (ΔCas-S3), p2065 (ΔCas-S4) and p2160 (ΔCas-S1ΔCas-S3) respectively (FIG. 4). The 5′ and 3′ end of the spacer of the crRNA module contained recognition sites for the BsmBI-v2 (NEB) restriction enzyme, which facilitated the cloning of the targeting spacers (FIG. 4). All the plasmids contained the repA4101 replication protein gene, the SC101 low copy origin of replication and the teR tetracycline resistance marker gene (FIG. 4).

### 1.4 Chromosomal CRISPRi

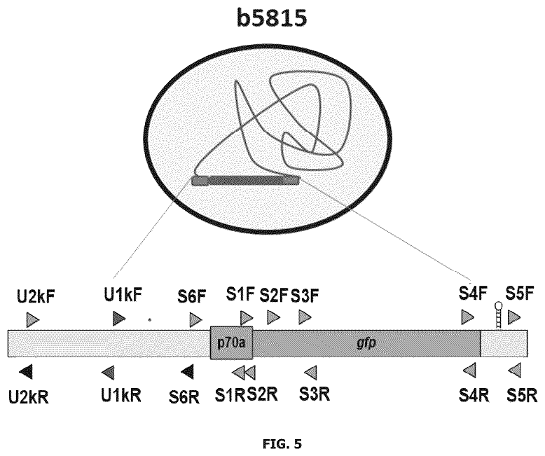

Strains MG1655 and b5815 (gfp+ve strain) were employed for the chromosomal Type-S CRISPR/Cas system CRISPRi assays. b5815 is an E. coli MG1655 derivative, constructed to contain the gfp gene under the control of the p70a promoter (SEQ ID No:15) and positioned in between the ybcB and ybcCgenes (FIG. 5). Sixteen protospacers within or near the gfp gene of the b5815 (gfp+ve) strain were selected and targeted for the chromosomal CRISPRi assays (FIG. 5, Table 6, Seq ID Nos:25 to 41). Eight of the protospacers were located on the gfp coding strand (namely Protospacer U2kF, U1kF, S1F, S2F, S3F, S4F, S5F, S6F) and eight on the non-coding strand (namely Protospacer U2kR, U1kR, SiR, S2R, S3R, S4R, S5R, S6R).

### 1.4.1 Methods

The construction of the plasmids that express the different Type-S CRISPR/Cas system variants and target the prementioned protospacers was performed by digesting the p1985 (wt), p2062 (ΔCas-S1), p2064 (ΔCas-S3), p2065 (ΔCas-S4) and p2160 (ΔCas-S1 ΔCas-S3) plasmids with the BsmBi-v2 (NEB) restriction enzyme and then ligating pre-annealed complementary ssDNA oligos that code for the corresponding spacers into the digested plasmids using T4 DNA ligase (NEB). 5 μL from each ligation reaction were transformed with electroporation into 50 μL of NEB100 electro competent cells (NEB). The cells were subsequently left to recover in SOC medium (500 μL final volume) for 1 hour at 37° C. and shaking at 200 rpm. 100 μL from each recovery were removed and plated on LB agar plates supplemented with 20 μg/mL chloramphenicol. The plates were incubated overnight at 37° C.

The following day, single colonies were randomly selected and used for inoculation of 10 mL LB liquid medium cultures supplemented with 10 μg/mL tetracycline. The cultures were incubated overnight at 37° C. with rigorous shaking (200 rpm).

The following day, glycerol stocks were prepared for each culture and the remaining cultures were used for plasmid isolation with the Qiagen plasmid mini kit. Each plasmid was sequence-verified with NGS. Each of the plasmids was transformed into electrocompetent b5815 (gfp+ve strain) cells. The electrocompetent cells were prepared using the protocol described in (https://barricklab.org/twiki/bin/view/Lab/ProtocolsElectrocompetentCells). The transformation reactions were performed using 40 ng from each plasmid per reaction. The cells were allowed to recover in 500 μL of SOC medium for 1 hour at 37° C. and shaking at 200 rpm. 100 μL from each recovery were plated on LB agar plates supplemented with 10 μg/mL tetracycline. The plates were incubated overnight at 37° C.

The following day, three single colonies were randomly selected from each transformation and they were used for inoculation of 5 mL M9 minimal medium cultures supplemented with glycerol (0.4% w/v) and 10 μg/mL tetracycline. The cultures were incubated overnight at 37° C. with rigorous shaking (200 rpm).

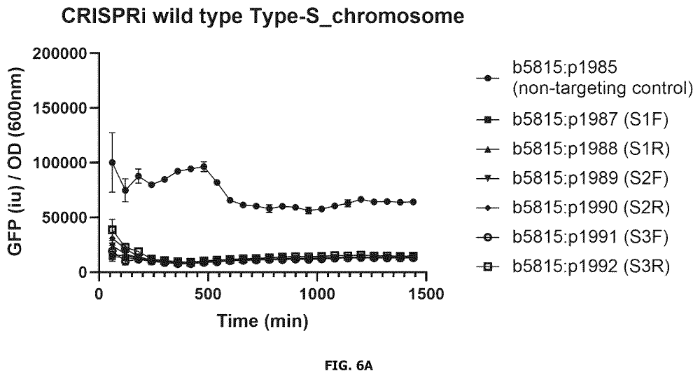

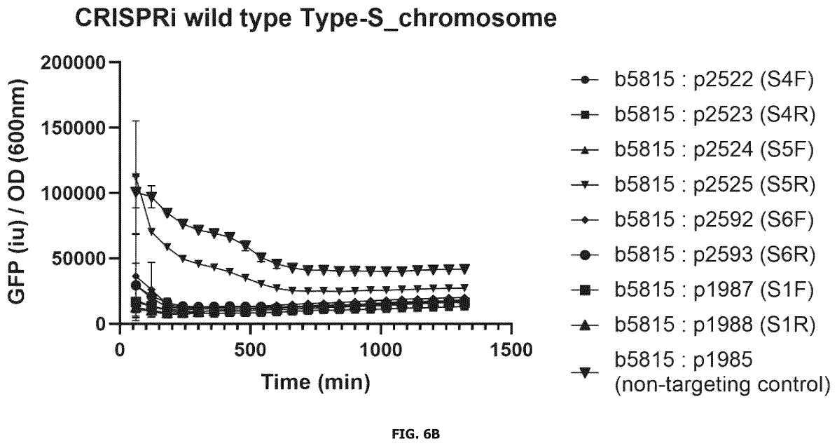

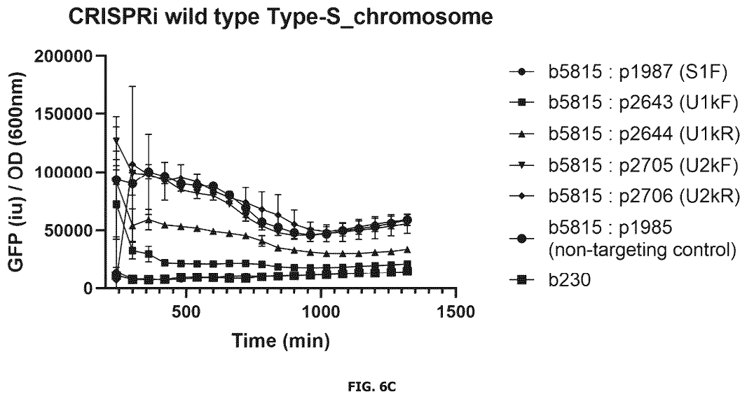

The following day, 2 μL from each culture were transferred to 198 μL of M9 medium supplemented with glycerol (0.4% w/v) and 10 μg/mL tetracycline. The resulting 200 μL cultures were transferred to distinct wells in Thermo Scientific™ Nunc microWell 96-well optical-bottom plates with polymer base. The plates were sealed with Breathe-Easy® sealing membranes (Sigma-Aldrich) and loaded into Synergy H1 microplate readers (Biotek) for incubation at 37° C. with continuous shaking. The optical densities (absorbance at 600 nm) and the green fluorescence emissions (excitation at 485 nm, emission at 516 nm) of the cultures were measured in 10 minute intervals for a 24-hour period. The fluorescence emissions data from each culture and each time point were normalized with the corresponding OD600 nm data. The normalized data from the cultures that belong to biological triplicates were averaged and the corresponding standard deviations were calculated. The final data were plotted in the graphs presented in FIG. 6.

### 1.4.2 Results

The Type-S CRISPR/Cas system downregulated GFP expression from b5815 (gfp+ve strain) to the no-fluorescence control strain (b230) levels, when targeting protospacers at the beginning of the gfp orf and in the p70a promoter region (Protospacers S1F, S1R, S2F, S2R, S3F, S3R) (FIG. 6A) or protospacers at the end of the gfp orf and the 200 bp regions upstream and downstream of gfp (Protospacers S4F, S4R, S5F, S6F, S6R) (FIG. 68), demonstrating extremely efficient and tight CRISPRi performance. The sole exception was the targeting of the S5R protospacer, which downregulated GFP expression by approximately 50%. The GFP expression was also downregulated when the Type-S CRISPR/Cas system targeted protospacers located 1 kb upstream of the gfp orf (Protospacers U1kF, U1kR), whereas GFP expression was completely unaffected for protospacers 2 kb upstream of the gfp orf (U2kF, U2kR) (FIG. 6C).

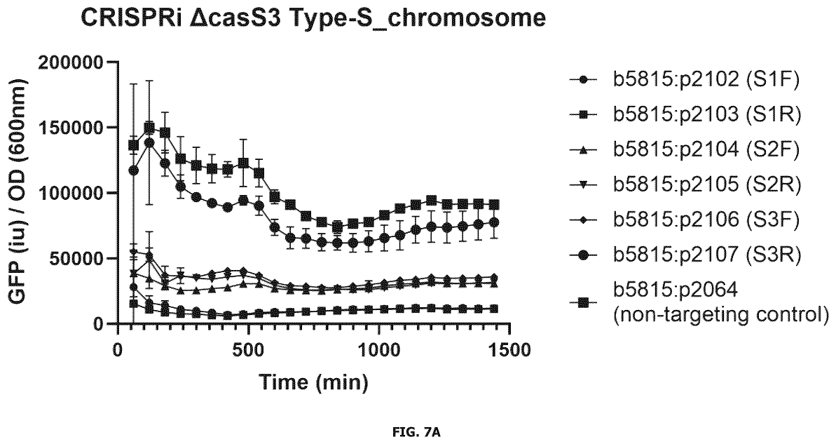

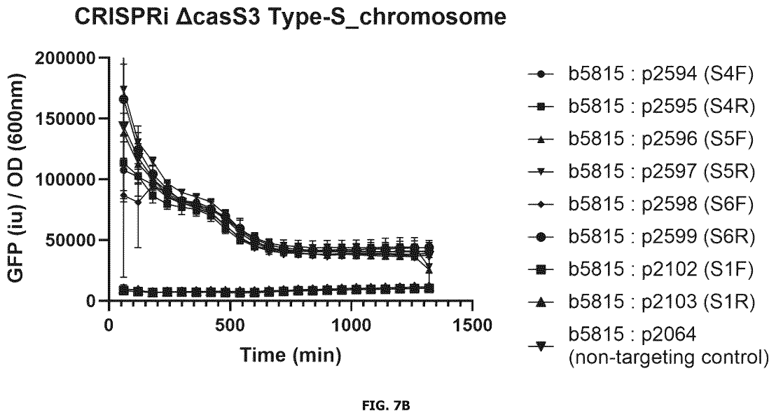

The helicase-deficient ACa9-S3 Type-S CRISPR/Cas system extensively or completely downregulated GFP expression from b5815 (gfp+ve strain) when targeting the promoter p70a region or the forward strand in the gfp orf (Protospacers S1F, S1R, S2F, S2R, S3F), but did not affect the GFP expression when it targeted the reverse strand in the gfp orf (Protospacer S3R) (FIG. 7A). It was here demonstrated that Cas-S3 cannot unwind DNA, and subsequently block transcription of genes, that reside 2 kb away from the targeted protospacer.

To confirm that the Type-S CRISPR/Cas system CRISPRi function is Cas-S3-dependent when targeting protospacers at the very end of, or outside of, a gene's orf, the targeting assays on protospacers S4F, S4R, S5F, S5R, S6F and S6R were repeated employing ΔCas-S3 Type-S CRISPR/Cas system (FIG. 7B). Indeed, the GFP expression of b5815 (gfp+ve strain) was unaffected for the selected targets in the absence of Cas-S3. These results indicate that Cas-S3 is not essential for the formation of the Type-S CRISPR/Cas system RNP complex and its binding to the DNA target.

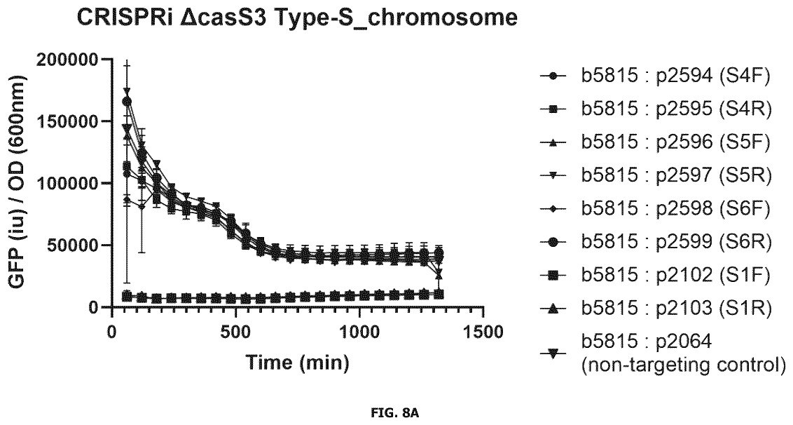

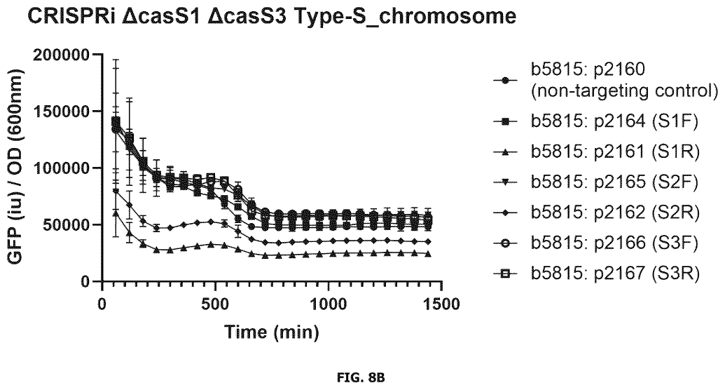

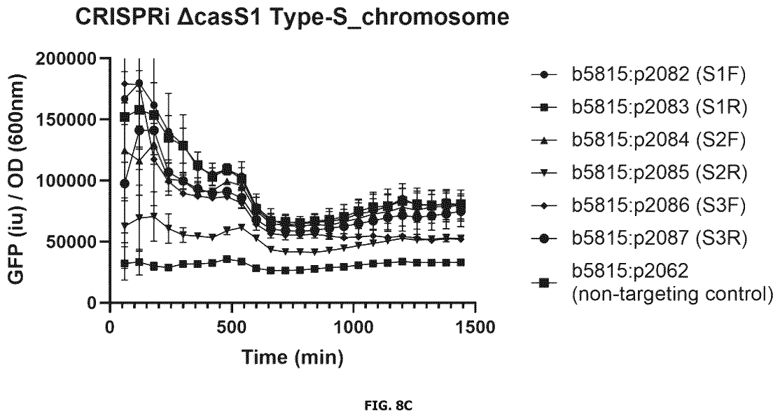

The ΔCas-S4 Type-S CRISPR/Cas system did not affect GFP expression from b5815 (gfp+ve strain), indicating that the subunit is essential for the for the formation of the Type-S CRISPR/Cas system RNP complex and/or its binding to the DNA target (FIG. 8A).

The ΔCas-S1 Type-S CRISPR/Cas system and the ΔCas-S1, ΔCas-S3 Type-S CRISPR/Cas system reduced GFP expression from b5815 (gfp+ve strain) in a similar manner and only when they targeted (completely or partially) the reverse strand of the p70a promoter (FIGS. 8B, 8C). These results indicate that Cas-S1 is not essential for the formation of the Type-S CRISPR/Cas system RNP complex and its binding to the DNA target, albeit it is important for its stability and/or the efficiency of its DNA binding activity. Moreover, these results indicate that Cas-S1 is vital for the loading of Cas-S3 to the complex.

### 1.5 Plasmid CRISPRi

Strain b6386 was employed for the plasmid Type-S CRISPR/Cas system CRISPRi assays. b6386 is an E. coli MG1655 strain transformed with the plasmid p2370 plasmid that contains the gfp gene (under the control of the p70a promoter, SEQ ID No:15), the p15a origin of replication and the onR chloramphenicol resistance marker gene. Only four out of the five Type-S variants tested for chromosomal CRISPRi in Example 4.4 were also tested for plasmid CRISPRi. The wild type Type-S CRISPR/Cas system comprising Cas-S1-Cas-S5 was not tested, due to its proven activity to inhibit plasmid replication (see Example 1.3.2, and 1.3.3). Six protospacers within the p70a promoter region and the gfp gene in b6386 were selected and targeted for the on-plasmid CRISPRi assays, the same as for the chromosomal CRISPRi assays; three of the protospacers were located on the gfp coding strand (namely Protospacer S1F, S2F, S3F) and three on the non-coding strand (namely Protospacer S1R, S2R, S3R) (see FIG. 5).

### 1.5.1 Methods

The construction of the various Type-S expressing plasmids that targeted these protospacers was performed as previously described in Example 4.4.1.

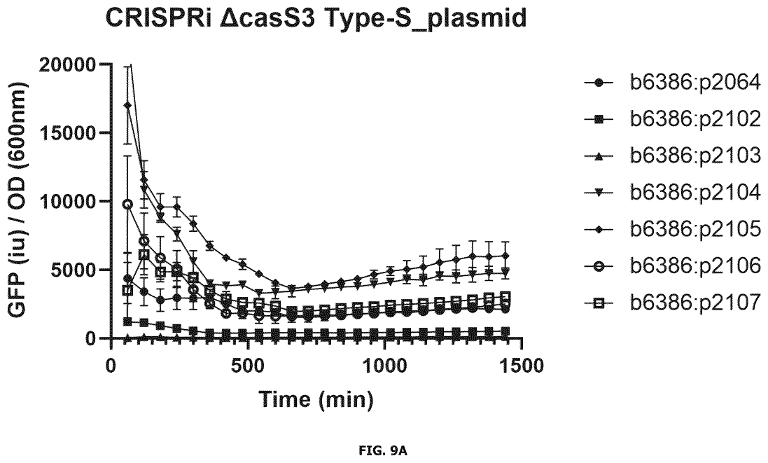

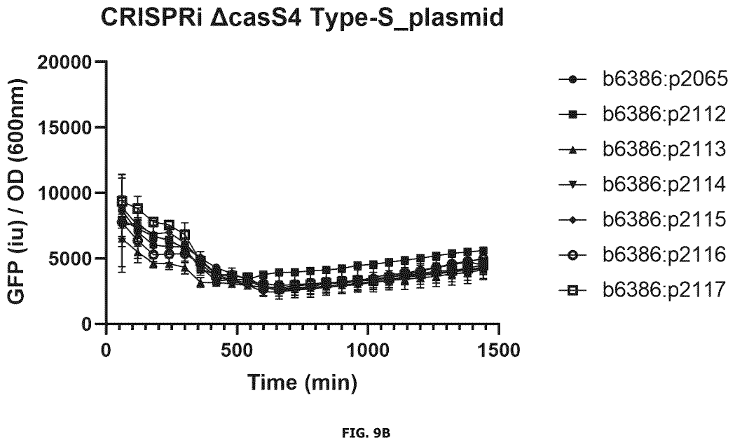

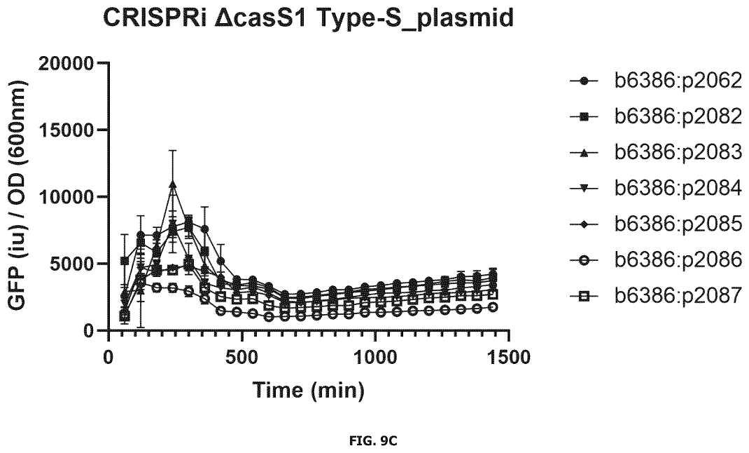

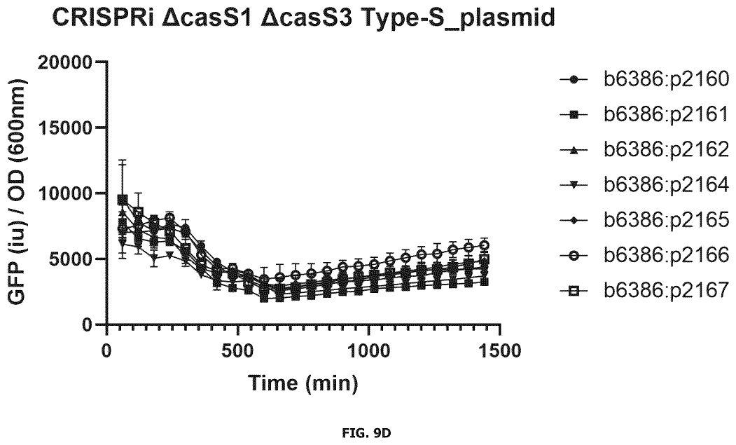

The transformation of the plasmids was performed as described in Example 4.4.1, with the following two changes:
1. 100 μL from each recovery were plated on LB agar plates supplemented with 20 μg/mL chloramphenicol, instead of 10 μg/mL tetracycline
2. 2) The inoculation cultures were supplemented with 20 μg/mL chloramphenicol, instead of 10 μg/mL tetracycline.
3. The final data were plotted in the graphs presented in FIG. 9.

### 1.5.2 Results

ΔCas-S3 Type-S downregulated, completely or efficiently (but not completely), GFP expression from b6386 (gfp+ve strain) only when it targeted protospacers within the promoter p70a region (S1F and S1R), regardless of the orientation of the targeted strand (FIG. 9A). None of the other tested variants downregulated GFP expression (FIGS. 9B, 9C, 9D). This comes in complete contrast to the results obtained by the chromosomal CRISPRi experiments.

### Example 2: Type-S CRISPRi/Cas System in Base Editing Applications

### 2.1 Introduction

The currently developed and reported CRISPR/Cas base-editing tools rely on the single-subunit effector modules of different Class 2 CRISPR/Cas systems. Class 1 CRISPR/Cas systems have multi-subunit effector modules, complicating the development of base editing tools. Consequently, the base editing capacities of Class 1 base-editors, regarding for example editing specificity, efficiency and window, have remained unexplored.

In this Example, the potential of the Type-S CRISPR/Cas system to transform into a flexible base-editing platform was extensively studied. A collection of base editors was designed and constructed by fusing, with a 16 amino acids long XTEN linker (SEQ ID Nos:16 and 17, see Schellenberger et al., Nat. Biotechnol., 27(12), 1186-1190, 2009, doi:10.1038/nbt.1588), the PmCDA1 cytidine deaminase from sea lamprey (SEQ ID Nos:18 and 19, see Nishida et al., Science, 353(6305), aaf8729, 2016, doi:10.1126/science.aaf8729) to either the N- or the C-terminus of the Cas-S1, Cas-S3 or Cas-S4 subunits of the Type-S CRISPR/Cas system. The Uracil DNA glycosylase inhibitor (UGI) protein (SEQ ID Nos:20 and 21, see Mol et al., Cell, 82(5), 701-8, 1995, doi:10.1016/0092-8674(95)90467-0) was fused to the C-terminus of each chimera with a 10 amino acids long linker (SEQ ID Nos:22 and 23). Subsequently, these base-editors were tested for their ability to generate targeted, chromosomal C-to-T modifications.

### 2.2 Experimental Design

### 2.2.1 Overview of Screening Strain and Plasmids:

A gfp gene was inserted into the chromosome of E. coli MG1655 under the transcriptional control of the p70a promoter (SEQ ID No:15). The resulting b5815 (gfp+ve) strain was employed as the test strain for all the base-editing assays. The six protospacer targets for the base-editing assays were selected to have a 5′-AAG-3′ PAM at their 5′ end and to be located within a 249 bp long region, comprised of the last 52 bp of the p70a promoter and the first 197 bp of the gfp sequence. Three of the protospacers were on the template (regarding transcription) strand and three were on the non-template strand (FIG. 5).

Two categories of plasmids were designed and constructed for the base editing assays:

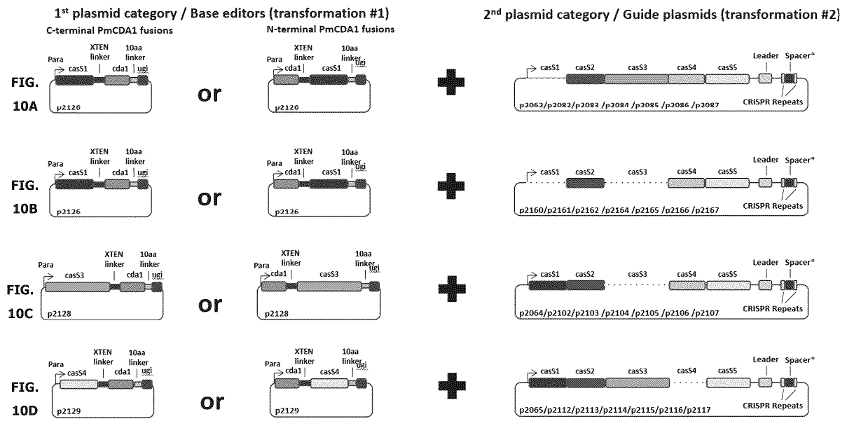

The 1^{st} category contained the ‘base editor’ plasmids responsible for the expression of the pm_cda1 gene which was fused to either the N- or the C-terminus of either the Cas-S1, Cas-S3 or the Cas-S4 gene (see FIG. 10, left hand side). The expression of the chimeras was set under the transcriptional control of the arabinose inducible promoter (p_{BAD}, SEQ ID No:24, see Guzman et al., J. Bacteriol., 177(14), 4121-4130, 1995, doi: 10.1128/jb.177.14.4121-4130.1995). The backbone of the plasmids contained the chloramphenicol resistance marker gene (crm^{R}), for selection on the antibiotic chloramphenicol, and the p15a origin of replication.

All the PCR reactions were performed with Q5 Hi Fi 2X Master Mix (NEB), with appropriately designed primers and PCR templates to construct the plasmids. The PCR fragments were employed for NEBuilder® HiFi DNA Assembly Master Mix reactions. 5 μL from each assembly reaction were transformed with electroporation into 50 μL of NEB10β electro competent cells (NEB). The cells were subsequently left to recover in SOC medium (500 μL final volume) for 1 hour at 37° C. and shaking at 200 rpm. 100 μL from each recovery were removed and plated on LB agar plates supplemented with 20 μg/mL chloramphenicol. The plates were incubated overnight at 37° C.

The following day, single colonies were randomly selected and used for inoculation of 10 mL LB liquid medium cultures supplemented with 20 μg/mL chloramphenicol. The cultures were incubated overnight at 37° C. with rigorous shaking (200 rpm).

The following day, glycerol stocks were prepared for each culture and the remaining cultures were used for plasmid isolation with the Qiagen plasmid mini kit. Each plasmid was sequence verified with NGS.

The 2^{nd} category contained the ‘guide’ plasmids responsible for the expression of i) the crRNA expressing modules (one for each of the non-targeting control, S1F, S1R, S2F, S2R, S3F and S3R) and ii) different versions of the Type-S Cas operon with either Cas-S1, Cas-S3 or Cas-S4 genes being deleted (see FIG. 10, right hand plasmids). The expression of the crRNAs was set under the control of the original leader (promoter) sequence as it was found in the CRISPR locus of the Type-S CRISPR/Cas system. The expression of the operons was set under the transcriptional control of the constitutive promoter of the bolA gene (SEQ ID No:14) present in the E. coli genome (p_{bolA}). The backbone of the plasmids contains the tetracycline resistance marker gene (tet^{R}), for selection on the antibiotic tetracycline, and the pSC101 origin of replication.

The construction methodology for the ‘guide’ plasmids was the same as for the 1^{st} category, except that (i) 10 μg/mL tetracycline was used for plating on LB agar, instead of 20 μg/mL chloramphenicol and (ii) 10 μg/mL tetracycline was used for inoculation of the LB liquid medium, instead of 20 μg/mL chloramphenicol.

Subsequently, the p2062 (non-targeting control, ΔCas-S1), p2064 (non-targeting control, ΔCas-S3), p2065 (non-targeting control, ΔCas-S4) and p2160 (non-targeting control, ΔCas-S1, ΔCas-S3) plasmids were subjected to restriction digestion with BsmBI-v2 (NEB) and the digestion products were gel purified with the Zymoclean Gel DNA Recovery Kit (Zymoresearch). Appropriately designed oligos were annealed and ligated into the digested plasmids with T4 DNA ligase (NEB). 5 μL from each ligation reaction was transformed with electroporation into 50 μL of NEB100 electro competent cells (NEB). The cells were subsequently left to recover in SOC medium (500 μL final volume) for 1 hour at 37° C. and shaking at 200 rpm. 100 μL from each recovery were removed and plated on LB agar plates supplemented with 10 μg/mL tetracycline. The plates were incubated overnight at 37° C.

The following day, single colonies were randomly selected and used for inoculation of 10 mL LB liquid medium cultures supplemented with 10 μg/mL tetracycline. The cultures were incubated overnight at 37° C. with rigorous shaking (200 rpm).

The following day, glycerol stocks were prepared for each culture and the remaining cultures were used for plasmid isolation with the Qiagen plasmid mini kit. Each plasmid was sequence verified with NGS.

### 2.2.2 Base Editing Assays

Each of the 6 ‘base editor’ plasmids having the PmCDA1 cytidine deaminase fused to either the N- or C-terminus of each of the wt Type-S subunits Cas-S1, Cas-S3 and Cas-S4 (see FIG. 10, left hand side) was transformed into electrocompetent b5815 cells. The electrocompetent cells were prepared using the protocol described in (https://barricklab.org/twiki/bin/view/Lab/ProtocolsElectmcompetentCells). The transformation reactions were performed using 40 ng of each plasmid per reaction. The cells were allowed to recover in 500 μL of SOC medium for 1 hour at 37° C. and shaking at 200 rpm. 100 μL from each recovery was plated on LB agar plates supplemented with 20 μg/mL chloramphenicol. The plates were incubated overnight at 37° C.

The following day, single colonies were randomly selected and used for inoculation of 10 mL LB liquid medium cultures supplemented with 20 μg/mL chloramphenicol. The cultures were incubated overnight at 37° C. with rigorous shaking (200 rpm).

The following day, electrocompetent cells were prepared for each one of the six produced b5815 strains with the base-editor plasmids, according to the protocol described in (https://barricklab.org/twiki/bin/view/Lab/ProtocolsElectmcompetentCells). Subsequently, each one of the six strains was electro-transformed with the corresponding group of guide plasmids, as presented on the right side of FIG. 10. These plasmids were responsible for the expression of i) Type-S subunits that are not expressed by the pairing base editor plasmids and ii) targeting and non-targeting crRNAs. The transformation reactions were performed using 40 ng from each plasmid per reaction. The cells were allowed to recover in 500 μL of SOC medium for 1 hour at 37° C. and shaking at 200 rpm. 100 μL from each recovery were plated on LB agar plates supplemented with 20 μg/mL chloramphenicol, 10 μg/mL tetracycline and 0.2% v/w arabinose. The plates were incubated overnight at 37° C.

The following day, single colonies were randomly selected and subjected to colony PCR with appropriately designed genome-specific primers. The PCR products were sequenced by Sanger sequencing and the results were analyzed for the detection of C-to-T modifications- or G-to-A modifications when the reverse strand was targeted and modified.

### 2.3 Results

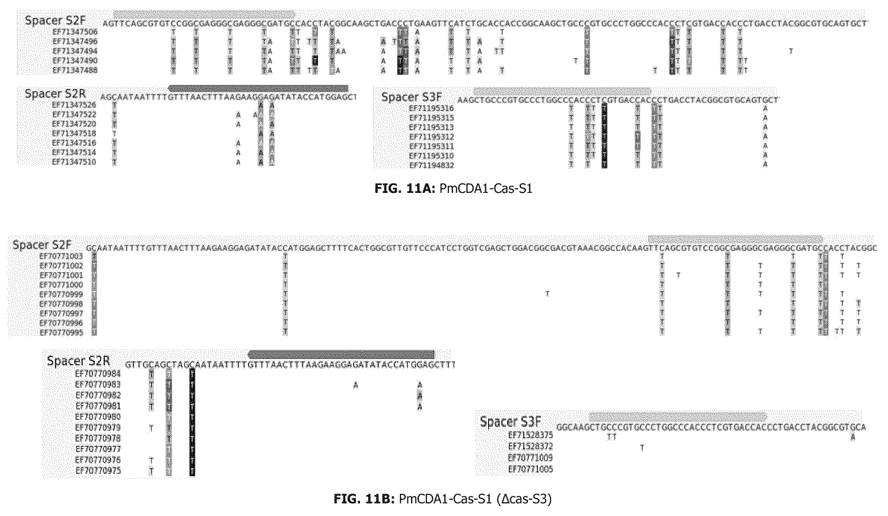

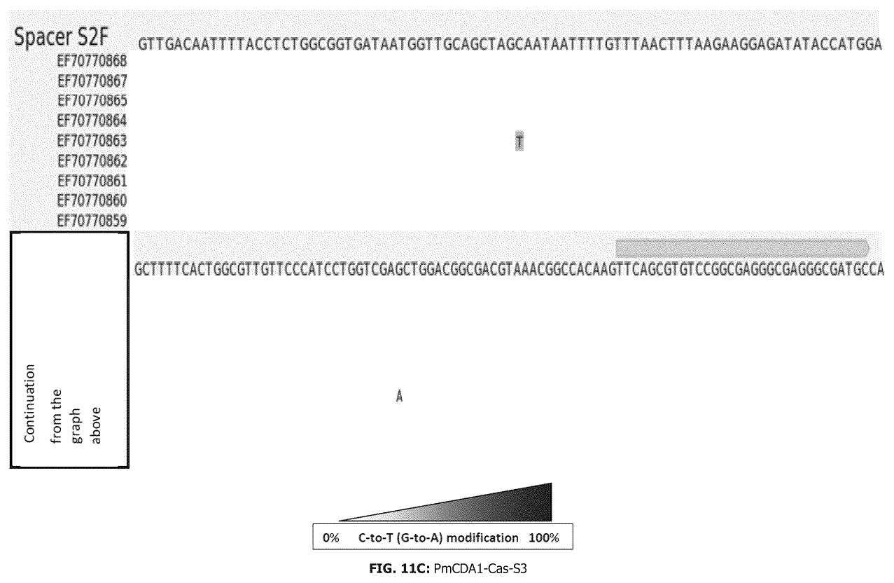

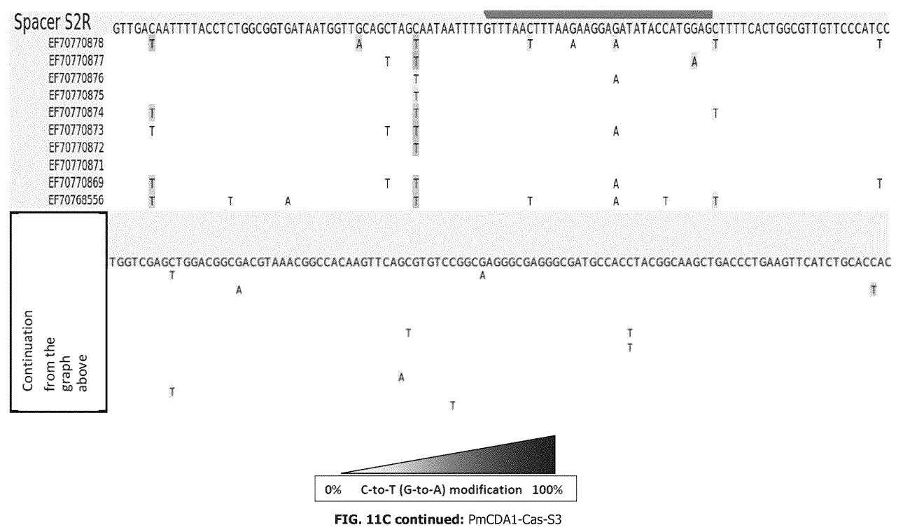

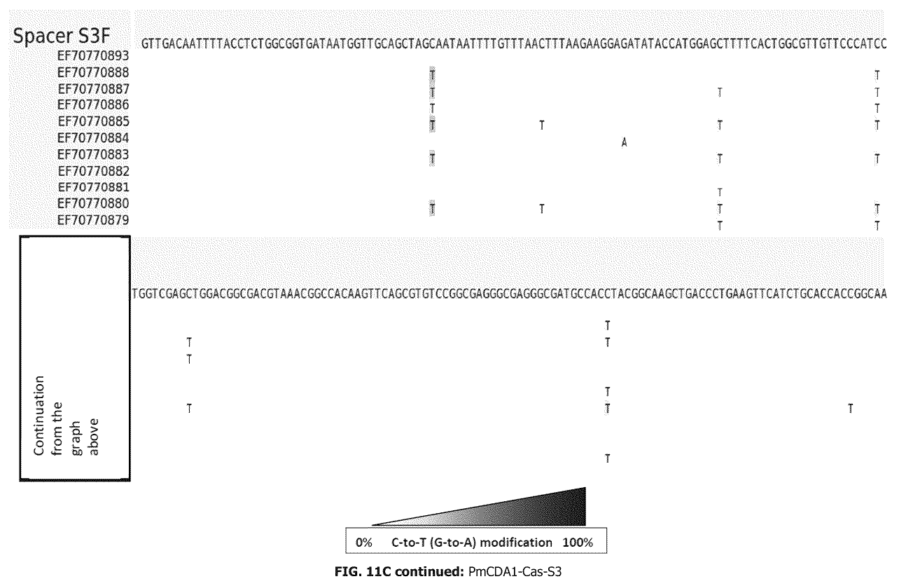

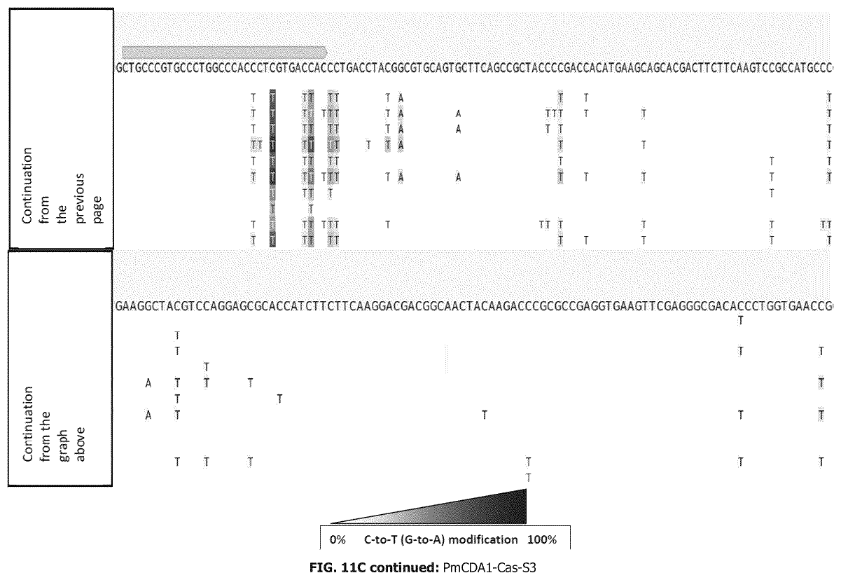

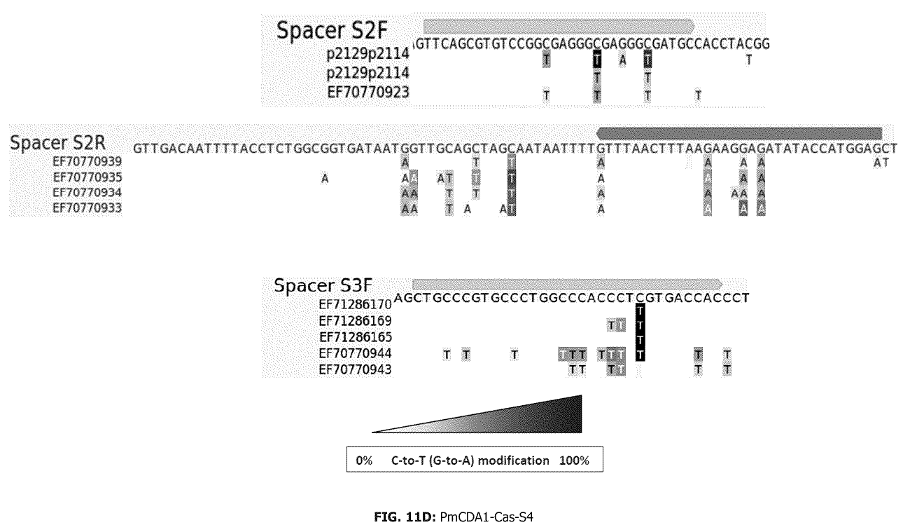

Representative results from the base editing assays are presented in FIG. 11.

Detailed analysis of the Sanger sequencing data from all the base editing assays is provided below:

### 2.3.1 PmCDA1 Linked to the N-Terminus of

Cas-S1 (WT Type-S): no base-editing
Cas-S3 (WT Type-S): single C-to-T mutation in only 1 of the targeted protospacers
Cas-S4 (WT Type-S): single C-to-T mutation in only 1 of the targeted protospacers (same as for Cas-S3-N term CDA)

### 2.3.2 PmCDA1 Linked to the C-Terminus of

### Cas-S1:

Protospacer S1F: No base-editing
Protospacer S1R: No base-editing
Protospacer S2F: Multiple positions base-edited (105 nt editing window) with different levels of efficiency for each position. Not only C-to-T but also a few G-to-A conversions
Protospacer S2R: 4 positions base-edited (29 nt editing window) with different levels of efficiency for each position. Not only G-to-A but also 1 C-to-T conversion
Protospacer S3F: 8 positions base-edited (35 nt editing window) with different levels of efficiency for each position. Not only C-to-T but also 1 G-to-A conversion
Protospacer S3R: few positions base-edited (outside the protospacer region) with low efficiency. Streaking improved efficiency and extended editing window (96 nt). A mixture of C-to-T and G-to-A conversions were detected.

### Cas-S1 (in the Absence of Cas-S3: Type-S_ΔCs-S3):

Protospacer S1F: No base-editing
Protospacer S1R: Single G-to-A modification within the protospacer
Protospacer S2F: Multiple positions were C-toT modified, with different levels of efficiency, within and 7 nt downstream of the protospacer region. 2 additional C-to-T conversions were detected within a 102 nt editing window upstream of the 5′end of the protospacer.
Protospacer S2R: 4 positions base-edited (48 nt editing window) with different levels of efficiency for each position. Only 1 weak G-to-A modification at the 3 PAM proximal position, and 3 efficient C-to-T modifications downstream of the protospacer
Protospacer S3F: multiple very weak modifications
Protospacer S3R: few very weak modifications (mixture of C-to-T and G-to-A conversions) were detected, as well as 1 very efficient C-to-T modification 40 nt downstream of the 3′ end of the protospacer.

### Cas-S3:

Protospacer S1F: No base-editing
Protospacer S1R: Dominant single position was base-edited in the middle of the protospacer and a few more were inefficiently edited. Streaking extended the base editing window (50 nt), while not only G-to-A but also a few C-to-T conversions were detected
Protospacer S2F: No base-editing
Protospacer S2R: Multiple positions were inefficiently base-edited within a wide editing window (up to 208 nt). Not only G-to-A but mostly C-to-T conversions were detected. Streaking improved the efficiency as it resulted in 3 clean conversions (both G-to-A and C-to-T) within a 178 nt window.
Protospacer S3F: Multiple positions were base-edited (388 nt editing window). Conversions within the protospacer region were the cleanest ones. Only 2 positions were detected with G-to-A conversions.
Protospacer S3R: No base-editing detected initially, but the streaking generated multiple G-to-A and C-to-T conversions within a 388 nt editing window.

### Cas-S4:

All targeted protospacers were edited in multiple positions within the protospacer region. Exception was S2R, that was also edited downstream the protospacer region, and S3R that had a single G-to-A mutation at the 2^{nd} PAM distal position of the protospacer.
Protospacers S1F and S2R had mixtures of C-to-T and G-to-A modifications, while the rest of the protospacers had either C-to-T or G-to-A modifications, depending on the direction of the protospacer.

### 2.4 Conclusions

This Example generated the first reported Class I CRISPR/Cas platform for efficient base-editing. The seven developed base editors employed the PmCDA1 cytidine deaminase. Six of the base editors employed the wild type Type-S CRISPR/Cas system and one of the base editors employed the ΔCas-S3 Type-S CRISPR/Cas system.

Fusion of PmCDA1 to the N-terminus of Cas-S1 did not generate any detectable modification.

Fusion of PmCDA1 to the N-terminus of Cas-S4 and Cas-S3 generated only a single detectable, mixed (wild type/mutant genotype) modification for one of the tested spacers. These systems could be used when very specific point mutations are required. But the low number of spacers that can be used restricts the applicability of these systems.

Fusion of PmCDA1 to the C-terminus of Cas-S1, Cas-54 and Cas-S3 generated three base editing systems with very different base editing outcomes (editing windows, editing efficiencies and edited positions) even on the same protospacers. In general, the Cas-S1- and Cas-S3-based base editors had very wide editing windows and can be very valuable in the generation of amino-acid libraries for a single protein or the introduction of stop codons. In the absence of Cas-S3, the editing window of the Cas-S1-based base editor was reduced and a shift in the edited positions was noted. The Cas-S4-based base editor had a narrower base-editing window and was the only base editor that introduced modifications into all the targeted protospacers.

### Example 3: CasS Base Editor in Conjugative Plasmids for Targeting Endogenous Genes of Recipient Bacteria

As is shown in FIG. 12, the conjugative plasmid in this example comprises four exogenous genes, G1, G2, G3, and G4. G1, G2 and G3 are exogenous enzymes which convert an endogenous substrate (i.e. the second MOI) into the first MOI. G4 is an exogenous exporter which is capable of exporting the first MOI out of the donor and recipient cells.

The PMS is comprised of a base editing moiety (PmCDA1) fused to SNIPR's CasS system. The CasS-based base editing system is under the control of a constitutive promoter, PcBE (Seq ID No: 102), see FIG. 12. The spacer included is programmed to introduce a stop codon in an endogenous gene sequence of the recipient bacterium. This stop codon has 3 downstream effects: 1) results in de-repression of the synthesis of an upstream precursor of the second MOI (with reference to FIG. 2, an increase in the synthesis of S); 2) results in depression of the enzyme which converts the upstream precursor to the immediate precursor of the second MOI (with reference to FIG. 2, and increase in production of enzyme E1, which converts S into S1, and S1 is the immediate precursor to the second MOI, B); and 3) derepresses an endogenous importer of the immediate precursor of the second MOI and the second MOI (with reference to FIG. 2, an importer [not shown] of substrate S1, and an importer of the second MOI, B [also not shown]).

### 3.1 Materials and Methods

### 3.1.1 Engineering

A schematic of the engineered conjugative plasmid expressing the MOI pathway and the CasS base-editor system is shown in FIG. 12.

Non conjugative plasmids are constructed using in-Fusion cloning (Takara) according to manufacturers protocol and chromosomal manipulations as well as manipulations of conjugative plasmids are done using scarless lambda red mediated recombineering from plasmid p1910 (Datsenko & Wanner PNAS 97, 6640-45, 2000) (Blank et al. 2011 PLoS ONE)). All primer sequences are listed in Tables 4 and 6 (Seq ID Nos:79 to 101).

Conjugative plasmid p2464 are made by amplifying a chloramphenicol cassette from pBAD33 using oli8116×oli8117 and recombineering it into wt 810 in b3557. This creates strain b6584 containing p2464, which is a control conjugative plasmid containing neither the MOI production pathway, nor the CasS-base editor (see FIG. 12). The strain is full genome sequenced to verify correct modifications.

Conjugative plasmid p2806 is made by amplifying the MOI production pathway, which includes an exporter of the first MOI (G4) and chloramphenicol cassette from p2719 using primers oli8805×oli8743 and recombineering it into wt B10 in b3557. This creates b7830 containing p2806, which comprises only the MOI production pathway, and not the CasS-base editor (see FIG. 12). The strain is full genome sequenced to verify correct modifications.

Conjugative plasmid p3024 is made by amplifying the casS base-editor system and a kanR cassette using primers oli9580×oli9579 from p2970 and recombineering it into p2806 in b230. This creates strain b8286 containing p3024, which comprises both the MOI production pathway (including an exporter of the first MOI as G4), and the CasS-base editor. The strain is full genome sequenced to verify correct modifications (see FIG. 12). CasS1 has the nucleic acid sequence of Seq ID No: 7, CasS2 has the nucleic acid sequence of Seq ID No: 8, CasS3 has the nucleic acid sequence of Seq ID No: 9, CasS4 has the nucleic acid sequence of Seq ID No: 10, which is fused to PmCDA1 with the XTEN linker of Seq ID No: 16, the PmCDA1 has the nucleic acid sequence of Seq ID No: 19, the PmCDA1 is fused to UGI with the linker of Seq ID No: 22, the UGI has the nucleic acid sequence of Seq ID No: 20, and the CasS5 has the nucleic acid sequence of Seq ID No: 11.

Conjugative plasmid p2986 is made by amplifying the casS base-editor system and a kanR cassette using primers oli9581×oli9579 from p2970 and recombineering it into wt 610 in b3557. This creates strain b8150 containing p2986, which comprises only the CasS-base editor, and not the MOI production pathway (see FIG. 12). The strain is full genome sequenced to verify correct modifications.

Strain b8524 (donor strain) is made based on b463 using four rounds of recombineering (Δp3, Δhly, I-tev-clbS at csiR and ΔdapA) with the primers listed in table 4. The strain is full genome sequenced to verify correct modifications.

Strain b5652 (recipient strain) is made based on b463 using one round of recombineering (Δfix) with the primers listed in table 4. The strain is full genome sequenced to verify correct modifications.

TABLE 4
Primers used for engineering

| PCR products | | |
|---|---|---|
| used for | | |
| chromosomal | | |
| engineering | 1. step, ZeoR cassette | 2. step |
| Δp3 | Oli7154 × Oli7156 | Oli7159 × Oli7160 on b463 |
| | on p1884 | |
| Δhly | Oli8199 × Oli8200 | Oli8201 × Oli8197 on b463 |
| | on p1884 | |
| I-tev-clbS | Oli2906 × Oli2907 | Oli8729 × Oli2893, then |
| at csiR | on p1884 | Oli5622 × Oli2893. on b6022 |
| ΔdapA | Oli8822 × Oli8821 | |
| | on b3104 | |
| Δfix | Oli2906 × oli2907 | Oli2892 × oli2893 on |
| | on p1884 | E. coli K12 mg1655 |

### 3.1.2 Conjugation Assay

Donor and recipients are inoculated in LB with appropriate supplements and grown to early exponential phase (OD 0.4). Donor and recipients are spotted for CFU and mixed in the ratio of 5:1. For each, 100 uL of mixed cells is placed in the middle of an LB plate and incubated overnight at 37° C. The cells are scraped of the plate and resuspended in 300 uL LB. 10-fold dilutions are spotted on non-selective LB plates, as well as plates selective for donor and transconjugants. The plates are incubated ON at 37° C. and counted for CFU.

### 3.1.3 Quantification of MOI Production in Bacterial Supernatants

A standard assay for the detection of the desired MOI is used to quantify the amount (μM) of the MOI in the bacterial supernatants. Such assays are well known to those skilled in the art

### 3.2 Results

### 3.2.1 Conjugation Efficiency of Engineered Vectors

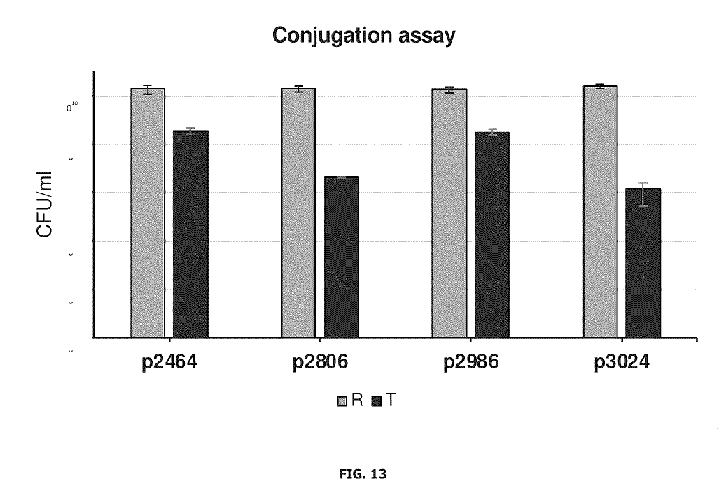

A conjugation assay was performed (see section 3.1.2 for methodology) to evaluate the effect of the MOI pathway and CasS base-editor on the conjugation efficiency. The donor strains were b8524 (SBF G6/7 ΔdapA) with p2464 (wild-type β10), p2806 (810 with MOI pathway), p2986 (β10 with CasS base-editor) or p3024 (β10 with both MOI pathway and CasS base-editor). These plasmids were conjugated to b5652 (recipient strain) following the solid conjugation protocol as described in section 3.1.2, and the number of transconjugants as well as the total number of recipients were counted (see FIG. 13). Plasmids p2806 and p3024 appear to have the lowest levels of transconjugants, indicating that the presence of the MOI pathway possesses a burden to the cell. It does not appear to be due to a size increase as plasmid p2986, carrying only the CasS base-editing system, seems to conjugate as efficiently as the wild-type β10.

### 3.2.2 Effect of CasS-BE on MOI Production

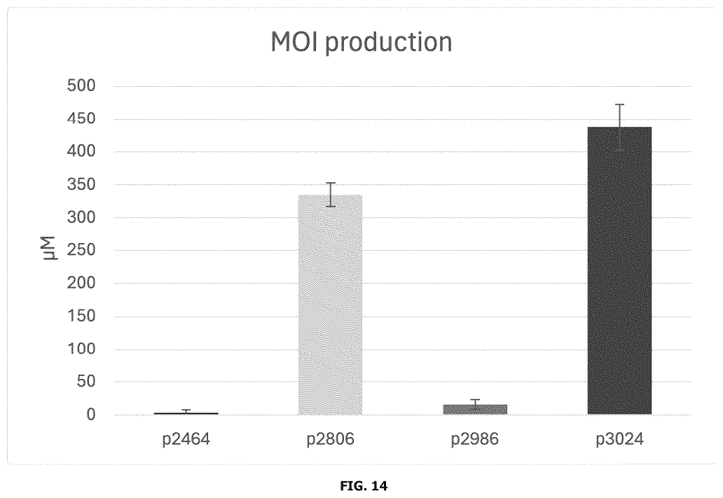

To evaluate the effect of mutations introduced by the base-editing, on the production of our molecule of interest (MOI), we measure MOI levels produced by the obtained transconjugants. The p2986 and p3024 transconjugants chosen for the assay had acquired a stop codon within the protospacer located within the target gene, besides this all have the same genetic background (b5652). High levels of MOI are produced by those receiving a conjugative plasmid carrying the MOI production pathway (p2806 and p3024), yet the introduction of a premature stop codon by p3024 leads to a significant increase in the detected levels of MOI. No MOI is being produced when the pathway is not present in the conjugative plasmid (p2464 and p2986), see FIG. 14.

### 3.2.3 Base Editing Sequence Analysis

To analyse the base-editing effect, transconjugants from all four conjugations were sequenced. As shown in Table 5, those receiving plasmids p2986 and p3024 containing the CasS base-editor picked up premature stop codons in the target gene. However, those receiving p2464 or p2806 did not pick up any mutations. The CasS based base-editing system seems to function well and efficiently when transferred by conjugation.

TABLE 5

| | | Fraction of transconjugants |
|---|---|---|
| Plasmid | Mutations | with premature stop-codons |
| received | Introduced | within protospacer |
| P2464 | Na. | 0 |
| P2806 | Na. | 0 |
| P2986 | G to A | ⅝ |
| P3024 | G to A | ⅝ |

Table 5 showing mutations introduced into the target nucleic acid sequence on the chromosome of recipients of the listed conjugative plasmids. Those that received conjugative plasmids carrying a base-editing system had mutations introduced at the target sequence.

### 3.3 Conclusions

A conjugative plasmid carrying a pathway for the production of a MOI, capable of transferring between strains in the native microbiome, can be made. In order to improve the production in recipients, a CasS based base-editing system was added to this conjugative plasmid. Recipients acquired the desired chromosomal modifications in the target gene. This led to an increase in the MOI production from transconjugants.

TABLE 6
Sequence listing

| Sequence | | |
|---|---|---|
| number | Description | Sequence |
| Seq ID No: 1 | Cas-S1.1 amino | LNHPVESVYSALTSILLPYMGEPVPVQRNCSCCGRAPSEFDGVGFELVNAYRERVVHCRPCQTFFVSAPE |
| | acid sequence | LMGVENPKKPTTGQKFGMWSGVGAVINVEDNSSVLLAPQGVVNKLPEHFFDHVEVITATSGQHLEYLFNT |
| | | ELKFPLIYIQNFGVKTYELVRSLRVSLSADAIYTCADQLLTRQNEVLYMLDLTKAKELHQEIKNYSKKEI |
| | | DIFIRTVTLLAYSRITPEAASNEFKKNNLIPLLLLLPTDPHQRLSILHLLKKV |
| Seq ID No: 2 | Cas-S2.1 amino | MIYYDNAFRLRIRHTSVYQIHQHLDFFMQERKGEKLPYSFKIFPGTGDDSLLLVRTATALELPGEKKREL |
| | acid sequence | ILSEGHEIKFITSMAIFHKGTKSEGRGRRQFAPSEEASYQLALTKLAKAGFKPGQIVVSGPKFVHIVKGN |
| | | AGRGFTLPVFTVQGTAIISNQQEAEVGIVYGVGPKRVFGCGFMHLAGQ |
| Seq ID No: 3 | Cas-S3.1 amino | MNTSAVFESAGLSLRKVQQDYIEAAAGALTQDHKVALISAETGVGKTLGYLVPALLILLKNPEAKFVIAT |
| | acid sequence | NSHALMHQIFRSDRPLLEQIAEQCGIKVTFSRLMGKANYVSLEKVRGLLLMDEFTDLDTVKVLEKLANWS |
| | | KPLVEFEEEYGELPAQITPEMVTYSIWDDIQDIDDIRLNALSANFIVTTHAMVMVDCMCNHRILGDKENM |
| | | YLIIDEADIFVDMLEVWKQRRFNLRELTSAFNEHIPRNGVHVIDQLMNDVTSIAGDLHFCSTPAAVALFD |
| | | NSFNALSKVGREIKNEAARKAFFDCIYSWEMLGLSGGQKGVGVSNKRREPALIAVNPFIGMNVGRYCTQW |
| | | RSALLTSATLSITSTPETGMEWLCKALGLTSDTISIRKIFSPDVYGSMKLTIAGADFPKVFNDPKEQIFS |
| | | GQWLKAVVEQLSCIQGPALVLTASHYETRMIANQLGEVSQPVYIQKAGQALSEIIKQYQEIPGILISAGA |
| | | SVGVSPRGENGEQIFQDLIITRIPFLPPDRMKAESLYGYLKERGYSRTFEAVNRNIYLDNLRKVIRKAKQ |
| | | SIGRGIRSENDTVRIIILDPRFPEPTDLSSKHRSLEHIIPVRFRRAYRSCEILSPAYCEEDIQC |
| Seq ID No: 4 | Cas-S4.1 amino | VLNFKPYRVIMSSLTPVVISGIAPSLDGILYEALSQAIPSNEPGVVLARLKEILLFNDELGVFHASSLRF |
| | acid sequence | GITPEQGIGATTSVRCDYLSPEKLSTAMFSPRIHRGVFTRVLLTGGPTKRRMTTRPAYSAPYLTFDFVGS |
| | | SEAVEILLNHAHVGVGYDFFSAANGEFNNVTILPLDIDTSISNEGMALRPVPVNSGLNGIKGVSPLIPPY |
| | | FVGEKLNIVHPAPVRTQLISSLLRG |
| Seq ID No: 5 | Cas-S5.1 amino | MRTLNFNGKISTLEPLTVTVKNAVSTSGHRLPRNGGFNAAPYFPGTSIRGTLRHAAHKVIVDRVGLNADG |
| | acid sequence | KSSFDLAEHFMLAQGVDINGEAETFAPGEINAGAELRSKNPLISLFGRWGLSGKVGIGNAIPDGDNQWGM |
| | | FGGGARSIMFQRDESLMEFLETDQVDRLERLLEEQAEASVDISQIKTEQDALKKAMKSADKDTKAELQIK |
| | | VRELDEKIQARKDQKQESRESIRRPIDPYEAFITGAELSHRMSIKNATDEEAGLFISALIRFAAEPRFGG |
| | | HANHNCGLVEAHWTVTTWKPGELVPVTLGEIVITPNGVEITGDELFAMVKAFNENQSFDFTAR |
| Seq ID No: 6 | Repeat sequence | GTATTCCCCCCGTGTGGGGGGTTATCGG |
| Seq ID No: 7 | Cas-S1.1 DNA | TTGAATCATCCTGTAGAGTCAGTATATAGCGCTCTGACGAGTATTTTATTGCCCTATATGGGTGAGCCTG |
| | sequence | TGCCTGTGCAACGAAATTGCTCATGCTGCGGCAGAGCGCCCTCAGAGTTCGACGGGGTAGGCTTCGAGCT |
| | | GGTCAACGCCTACAGGGAACGTGTTGTGCATTGCCGTCCTTGCCAGACATTTTTTGTTTCTGCTCCAGAA |
| | | CTGATGGGCGTGGAGAACCCCAAAAAACCAACAACCGGCCAGAAATTTGGCATGTGGTCTGGTGTTGGGG |
| | | CTGTTATTAACGTTGAAGACAATTCTTCGGTTCTTTTAGCCCCTCAGGGAGTGGTCAATAAACTACCTGA |
| | | GCATTTTTTCGATCACGTAGAAGTCATCACGGCCACCAGCGGTCAGCACCTGGAATACCTGTTTAACACC |
| | | GAACTTAAATTCCCGCTGATTTATATTCAAAACTTCGGCGTGAAAACCTACGAGCTTGTTCGCTCACTGG |
| | | GGTCAGTCTGAGTGCCGACGCAATTTATACCTGTGCCGATCAACTACTGACCCGGCAGAACGAAGTCCTT |
| | | TACATGCTGGATTTAACAAAGGCTAAAGAACTCCATCAGGAAATAAAAAACTACTCCAAAAAAGAGATAG |
| | | ACATCTTTATCAGAACAGTAACCCTGCTGGCCTATTCGCGGATAACTCCTGAAGCTGCCTCGAATGAGTT |
| | | TAAGAAAAACAACCTCATTCCGTTACTGCTGCTTCTGCCGACTGATC |
| Seq ID No: 8 | Cas-S2.1 DNA | ATGATTTACTACGATAATGCGTTTCGGTTGCGTATCCGTCACACAAGTGTTTACCAGATCCACCAGCACC |
| | sequence | TGGATTTTTTCATGCAGGAACGTAAGGGAGAGAAGCTGCCGTACTCTTTTAAAATTTTTCCCGGTACCGG |
| | | GGATGATTCGTTACTCCTGGTCCGCACAGCCACAGCTCTGGAGTTGCCAGGGGAGAAAAAAAGAGAGCTT |
| | | ATTCTCTCTGAGGGCCATGAAATCAAGTTCATTACGTCAATGGCCATCTTTCATAAAGGAACAAAGTCAG |
| | | AAGGCCGTGGCCGACGCCAGTTTGCTCCATCCGAAGAGGCTAGTTATCAGTTGGCCCTCACCAAATTAGC |
| | | GAAGGCTGGTTTTAAACCCGGTCAAATCGTCGTCAGTGGCCCGAAATTCGTCCATATCGTCAAAGGAAAT |
| | | GCTGGACGCGGTTTTACGCTACCCGTATTCACGGTACAGGGTACCGCCATAATCAGCAATCAACAGGAAG |
| | | CTGAAGTTGGAATCGTTTATGGCGTAGGCCCTAAACGCGTATTTGGTTGCGGTTTCATGCATCTGGGGGG |
| | | GCAGTAG |
| Seq ID No: 9 | Cas-S3.1 DNA | ATGAATACATCAGCGGTATTTGAGTCTGCCGGATTGTCGTTACGTAAGGTCCAACAGGATTATATCGAGG |
| | sequence | CTGCAGCTGGTGCGCTGACTCAGGATCATAAAGTTGCACTGATTAGTGCTGAAACAGGTGTAGGAAAAAC |
| | | GCTGGGCTATCTGGTACCGGCGCTTCTGATTCTGCTGAAAAACCCAGAGGCAAAATTTGTGATAGCCACG |
| | | AATTCTCATGCCCTGATGCACCAGATATTCAGAAGCGATCGTCCTCTCCTTGAACAGATAGCTGAACAAT |
| | | GCGGCATAAAAGTTACTTTTTCCCGGCTTATGGGGAAAGCAAATTACGTATCCCTGGAGAAAGTACGTGG |
| | | ACTGTTACTCATGGACGAATTTACCGATCTGGATACGGTTAAGGTCCTTGAAAAACTCGCTAACTGGTCA |
| | | AAACCTCTGGTTGAGTTTGAAGAAGAGTATGGTGAACTGCCAGCGCAGATCACGCCAGAGATGGTCACCT |
| | | ACTCCATCTGGGATGATATACAGGACATTGATGATATTCGCCTGAATGCTCTCAGTGCTAACTTTATAGT |
| | | TACGACCCATGCGATGGTAATGGTCGACTGTATGTGTAATCACCGCATCCTCGGTGACAAAGAAAATATG |
| | | TACCTCATTATTGATGAGGCAGACATTTTTGTAGATATGCTTGAGGTCTGGAAACAGCGGCGCTTCAACC |
| | | TCAGAGAGTTGACCAGTGCGTTCAATGAACATATTCCACGTAATGGCGTGCATGTCATCGACCAGTTAAT |
| | | GAACGACGTGACTTCGATAGCGGGCGATCTGCATTTTTGTAGTACGCCTGCAGCGGTTGCCTTGTTTGAC |
| | | AATAGCTTTAACGCCTTATCAAAGGTAGGGGGGAAATAAAAAATGAAGCGGCCCGGAAAGCTTTCTTTGA |
| | | CTGTATCTACAGCTGGGAAATGCTCGGATTGTCCGGGGGGCAAAAGGGTGTAGGCGTTTCCAATAAACGT |
| | | CGTGAACCAGCCCTGATCGCTGTAAACCCGTTTATCGGGATGAATGTGGGCAGATACTGCACTCAATGGC |
| | | GTAGTGCGTTACTGACGTCGGCTACGCTCTCAATTACCAGTACCCCAGAGACCGGAATGGAGTGGTTATG |
| | | TAAAGCTCTGGGGTTAACCAGCGATACGATTTCAATCAGGAAAATATTCTCCCCAGACGTCTATGGCTCA |
| | | ATGAAGCTGACCATCGCCGGTGCTGATTTTCCAAAGGTGTTCAATGACCCGAAAGAACAGATATTTTCAG |
| | | GTCAATGGTTAAAGGCAGTAGTTGAACAATTGTCCTGTATTCAGGGGCCAGCTCTGGTATTAACTGCCAG |
| | | CCATTATGAAACCAGGATGATTGCCAATCAGCTGGGTGAGGTCTCGCAACCAGTTTATATTCAGAAAGCA |
| | | GGTCAGGCCCTGTCCGAAATTATTAAACAGTATCAGGAGATACCAGGGATTCTTATTTCTGCCGGTGCTT |
| | | CTGTTGGTGTGAGCCCACGAGGCGAGAATGGGGAACAGATTTTCCAGGATTTAATCATTACCCGGATACC |
| | | TTTCTTACCTCCGGACAGAATGAAGGCTGAAAGCCTTTATGGGTATCTGAAGGAACGAGGCTATAGTCGT |
| | | ACCTTTGAGGCAGTTAACCGTAACATCTACCTGGATAATTTGCGGAAAGTTATTCGTAAAGCAAAGCAGT |
| | | CTATAGGGGGGGGGATTCGTAGTGAAAATGATACCGTCAGGATAATTATCCTCGATCCGCGTTTCCCGGA |
| | | ACCTACTGACCTTTCGTCTAAACATCGGTCGCTTGAACACATTATTCCCGTTCGATTTCGTCGTGCATAT |
| | | CGTTCGTGTGAAATTTTATCTCCGGCGTATTGTGAAGAGGATATCCAGTG |
| Seq ID No: 10 | Cas-S4.1 DNA | GTGTTAAATTTTAAACCGTATCGGGTCATCATGTCTTCTCTGACACCGGTGGTCATCAGTGGGATAGCCC |
| | sequence | CATCGCTGGACGGCATTCTGTATGAAGCACTGTCTCAGGCGATACCCAGTAACGAGCCAGGAGTCGTATT |
| | | GGCTCGTCTTAAGGAAATACTTCTATTCAACGATGAACTGGGTGTTTTCCATGCTTCATCTCTACGTTTT |
| | | GGCATAACGCCCGAACAGGGGATTGGGGCGACAACAAGCGTACGTTGCGATTATCTCAGTCCCGAAAAAC |
| | | TGAGCACTGCAATGTTCTCTCCTCGTATCCACAGGGGGGTGTTTACGCGTGTCCTCCTGACCGGGGGCCC |
| | | CACGAAGAGAAGGATGACAACCCGTCCAGCTTATTCTGCACCCTATTTAACGTTTGATTTTGTCGGCTCT |
| | | TCTGAGGCTGTAGAAATACTGCTTAACCATGCCCATGTTGGCGTTGGTTATGACTTTTTCTCTGCAGCTA |
| | | ATGGAGAGTTTAACAATGTGACAATTCTTCCTCTCGATATCGACACGAGTATATCCAACGAGGGTATGGC |
| | | ATTGCGTCCGGTACCTGTTAATTCGGGTCTGAATGGTATCAAAGGAGTATCACCACTTATTCCTCCCTAT |
| | | TTCGTTGGTGAAAAGCTAAACATTGTCCACCCAGCACCAGTTCGTACTCAATTGATTTCTTCTTTATTGC |
| | | GAGGCTAA |
| Seq ID No: 11 | Cas-S5.1 DNA | ATGAGAACTTTAAACTTCAACGGTAAAATTTCGACTCTGGAACCACTGACTGTAACAGTAAAAAATGCGG |
| | sequence | TCTCAACTTCCGGCCATCGCTTGCCTCGTAACGGTGGATTCAATGCAGCCCCGTACTTTCCAGGTACCAG |
| | | CATTCGGGGAACTCTTCGTCATGCTGCGCATAAGGTCATTGTCGATCGTGTGGGCCTTAATGCAGACGGA |
| | | AAATCATCCTTCGACCTGGCAGAACATTTCATGCTGGCCCAGGGGGTGGATATCAATGGTGAAGCCGAAA |
| | | CCTTCGCGCCAGGTGAAATCAATGCTGGTGCTGAACTACGTAGCAAAAACCCGTTAATCAGCTTGTTTGG |
| | | TCGTTGGGGATTAAGCGGCAAAGTTGGTATAGGGAATGCTATCCCGGATGGTGATAACCAGTGGGGGATG |
| | | TTTGGTGGTGGTGCCCGCTCAATTATGTTTCAGCGTGACGAAAGTCTGATGGAGTTTCTTGAGACAGACC |
| | | AGGTTGATCGTCTTGAACGCCTGCTCGAGGAACAGGCTGAGGCAAGTGTGGATATCTCTCAGATCAAAAC |
| | | AGAGCAAGATGCACTCAAAAAGGCGATGAAGTCGGCTGATAAAGACACCAAAGCTGAGCTTCAAATCAAA |
| | | GTACGGGAGCTCGATGAAAAAATTCAGGCCCGCAAAGATCAGAAGCAGGAATCTCGCGAGTCAATTCGCC |
| | | GCCCGATTGACCCGTATGAAGCGTTTATCACCGGCGCAGAACTCAGCCATCGCATGAGTATTAAAAATGC |
| | | GACTGATGAGGAAGCAGGGCTTTTCATTTCTGCATTAATCCGCTTTGCAGCCGAACCACGTTTTGGGGGT |
| | | CATGCGAATCATAACTGCGGTCTGGTGGAGGCTCACTGGACAGTTACGACCTGGAAGCCGGGTGAACTGG |
| | | TACCAGTTACACTTGGAGAAATCGTCATCACACCGAATGGTGTTGAGATTACCGGGGACGAGTTGTTTGC |
| | | TATGGTAAAGGCATTCAATGAAAATCAATCTTTTGATTTCACTGCCCGCTAA |
| Seq ID No: 12 | DNA sequence | TTGAATCATCCTGTAGAGTCAGTATATAGCGCTCTGACGAGTATTTTATTGCCCTATATGGGTGAGCCTG |
| | encoding Cas- | TGCCTGTGCAACGAAATTGCTCATGCTGCGGCAGAGCGCCCTCAGAGTTCGACGGGGTAGGCTTCGAGCT |
| | S1.1, 2.1, 3.1, | GGTCAACGCCTACAGGGAACGTGTTGTGCATTGCCGTCCTTGCCAGACATTTTTTGTTTCTGCTCCAGAA |
| | 4.1 and 5.1 | CTGATGGGCGTGGAGAACCCCAAAAAACCAACAACCGGCCAGAAATTTGGCATGTGGTCTGGTGTTGGGG |
| | | CTGTTATTAACGTTGAAGACAATTCTTCGGTTCTTTTAGCCCCTCAGGGAGTGGTCAATAAACTACCTGA |
| | | GCATTTTTTCGATCACGTAGAAGTCATCACGGCCACCAGCGGTCAGCACCTGGAATACCTGTTTAACACC |
| | | GAACTTAAATTCCCGCTGATTTATATTCAAAACTTCGGCGTGAAAACCTACGAGCTTGTTCGCTCACTGC |
| | | GGGTCAGTCTGAGTGCCGACGCAATTTATACCTGTGCCGATCAACTACTGACCCGGCAGAACGAAGTCCT |
| | | TTACATGCTGGATTTAACAAAGGCTAAAGAACTCCATCAGGAAATAAAAAACTACTCCAAAAAAGAGATA |
| | | GACATCTTTATCAGAACAGTAACCCTGCTGGCCTATTCGCGGATAACTCCTGAAGCTGCCTCGAATGAGT |
| | | TTAAGAAAAACAACCTCATTCCGTTACTGCTGCTTCTGCCGACTGATCCCCATCAACGCCTGAGCATTTT |
| | | GCACCTCTTGAAAAAGGTATAACCATGATTTACTACGATAATGCGTTTCGGTTGCGTATCCGTCACACAA |
| | | GTGTTTACCAGATCCACCAGCACCTGGATTTTTTCATGCAGGAACGTAAGGGAGAGAAGCTGCCGTACTC |
| | | TTTTAAAATTTTTCCCGGTACCGGGGATGATTCGTTACTCCTGGTCCGCACAGCCACAGCTCTGGAGTTG |
| | | CCAGGGGAGAAAAAAAGAGAGCTTATTCTCTCTGAGGGCCATGAAATCAAGTTCATTACGTCAATGGCCA |
| | | TCTTTCATAAAGGAACAAAGTCAGAAGGCCGTGGCCGACGCCAGTTTGCTCCATCCGAAGAGGCTAGTTA |
| | | TCAGTTGGCCCTCACCAAATTAGCGAAGGCTGGTTTTAAACCCGGTCAAATCGTCGTCAGTGGCCCGAAA |
| | | TTCGTCCATATCGTCAAAGGAAATGCTGGACGCGGTTTTACGCTACCCGTATTCACGGTACAGGGTACCG |
| | | CCATAATCAGCAATCAACAGGAAGCTGAAGTTGGAATCGTTTATGGCGTAGGCCCTAAACGCGTATTTGG |
| | | TTGCGGTTTCATGCATCTGGCGGGGCAGTAGTCATGAATACATCAGGGGTATTTGAGTCTGCCGGATTGT |
| | | CGTTACGTAAGGTCCAACAGGATTATATCGAGGCTGCAGCTGGTGCGCTGACTCAGGATCATAAAGTTGC |
| | | ACTGATTAGTGCTGAAACAGGTGTAGGAAAAACGCTGGGCTATCTGGTACCGGCGCTTCTGATTCTGCTG |
| | | AAAAACCCAGAGGCAAAATTTGTGATAGCCACGAATTCTCATGCCCTGATGCACCAGATATTCAGAAGCG |
| | | ATCGTCCTCTCCTTGAACAGATAGCTGAACAATGCGGCATAAAAGTTACTTTTTCCCGGCTTATGGGGAA |
| | | AGCAAATTACGTATCCCTGGAGAAAGTACGTGGACTGTTACTCATGGACGAATTTACCGATCTGGATACG |
| | | GTTAAGGTCCTTGAAAAACTCGCTAACTGGTCAAAACCTCTGGTTGAGTTTGAAGAAGAGTATGGTGAAC |
| | | TGCCAGCGCAGATCACGCCAGAGATGGTCACCTACTCCATCTGGGATGATATACAGGACATTGATGATAT |
| | | TCGCCTGAATGCTCTCAGTGCTAACTTTATAGTTACGACCCATGCGATGGTAATGGTCGACTGTATGTGT |
| | | AATCACCGCATCCTCGGTGACAAAGAAAATATGTACCTCATTATTGATGAGGCAGACATTTTTGTAGATA |
| | | TGCTTGAGGTCTGGAAACAGCGGCGCTTCAACCTCAGAGAGTTGACCAGTGCGTTCAATGAACATATTCC |
| | | ACGTAATGGCGTGCATGTCATCGACCAGTTAATGAACGACGTGACTTCGATAGGGGGCGATCTGCATTTT |
| | | TGTAGTACGCCTGCAGCGGTTGCCTTGTTTGACAATAGCTTTAACGCCTTATCAAAGGTAGGGGGGGAAA |
| | | TAAAAAATGAAGCGGCCCGGAAAGCTTTCTTTGACTGTATCTACAGCTGGGAAATGCTCGGATTGTCCGG |
| | | GGGGCAAAAGGGTGTAGGCGTTTCCAATAAACGTCGTGAACCAGCCCTGATCGCTGTAAACCCGTTTATC |
| | | GGGATGAATGTGGGCAGATACTGCACTCAATGGCGTAGTGCGTTACTGACGTCGGCTACGCTCTCAATTA |
| | | CCAGTACCCCAGAGACCGGAATGGAGTGGTTATGTAAAGCTCTGGGGTTAACCAGCGATACGATTTCAAT |
| | | CAGGAAAATATTCTCCCCAGACGTCTATGGCTCAATGAAGCTGACCATCGCCGGTGCTGATTTTCCAAAG |
| | | GTGTTCAATGACCCGAAAGAACAGATATTTTCAGGTCAATGGTTAAAGGCAGTAGTTGAACAATTGTCCT |
| | | GTATTCAGGGGCCAGCTCTGGTATTAACTGCCAGCCATTATGAAACCAGGATGATTGCCAATCAGCTGGG |
| | | TGAGGTCTCGCAACCAGTTTATATTCAGAAAGCAGGTCAGGCCCTGTCCGAAATTATTAAACAGTATCAG |
| | | GAGATACCAGGGATTCTTATTTCTGCCGGTGCTTCTGTTGGTGTGAGCCCACGAGGCGAGAATGGGGAAC |
| | | AGATTTTCCAGGATTTAATCATTACCCGGATACCTTTCTTACCTCCGGACAGAATGAAGGCTGAAAGCCT |
| | | TTATGGGTATCTGAAGGAACGAGGCTATAGTCGTACCTTTGAGGCAGTTAACCGTAACATCTACCTGGAT |
| | | AATTTGCGGAAAGTTATTCGTAAAGCAAAGCAGTCTATAGGGGGGGGGATTCGTAGTGAAAATGATACCG |
| | | TCAGGATAATTATCCTCGATCCGCGTTTCCCGGAACCTACTGACCTTTCGTCTAAACATCGGTCGCTTGA |
| | | ACACATTATTCCCGTTCGATTTCGTCGTGCATATCGTTCGTGTGAAATTTTATCTCCGGCGTATTGTGAA |
| | | GAGGATATCCAGTGTTAAATTTTAAACCGTATCGGGTCATCATGTCTTCTCTGACACCGGTGGTCATCAG |
| | | TGGGATAGCCCCATCGCTGGACGGCATTCTGTATGAAGCACTGTCTCAGGCGATACCCAGTAACGAGCCA |
| | | GGAGTCGTATTGGCTCGTCTTAAGGAAATACTTCTATTCAACGATGAACTGGGTGTTTTCCATGCTTCAT |
| | | CTCTACGTTTTGGCATAACGCCCGAACAGGGGATTGGGGCGACAACAAGCGTACGTTGCGATTATCTCAG |
| | | TCCCGAAAAACTGAGCACTGCAATGTTCTCTCCTCGTATCCACAGGGGGGTGTTTACGCGTGTCCTCCTG |
| | | ACCGGGGGCCCCACGAAGAGAAGGATGACAACCCGTCCAGCTTATTCTGCACCCTATTTAACGTTTGATT |
| | | TTGTCGGCTCTTCTGAGGCTGTAGAAATACTGCTTAACCATGCCCATGTTGGCGTTGGTTATGACTTTTT |
| | | CTCTGCAGCTAATGGAGAGTTTAACAATGTGACAATTCTTCCTCTCGATATCGACACGAGTATATCCAAC |
| | | GAGGGTATGGCATTGCGTCCGGTACCTGTTAATTCGGGTCTGAATGGTATCAAAGGAGTATCACCACTTA |
| | | TTCCTCCCTATTTCGTTGGTGAAAAGCTAAACATTGTCCACCCAGCACCAGTTCGTACTCAATTGATTTC |
| | | TTCTTTATTGCGAGGCTAATTTCATGAGAACTTTAAACTTCAACGGTAAAATTTCGACTCTGGAACCACT |
| | | GACTGTAACAGTAAAAAATGCGGTCTCAACTTCCGGCCATCGCTTGCCTCGTAACGGTGGATTCAATGCA |
| | | GCCCCGTACTTTCCAGGTACCAGCATTCGGGGAACTCTTCGTCATGCTGCGCATAAGGTCATTGTCGATC |
| | | GTGTGGGCCTTAATGCAGACGGAAAATCATCCTTCGACCTGGCAGAACATTTCATGCTGGCCCAGGGGGT |
| | | GGATATCAATGGTGAAGCCGAAACCTTCGCGCCAGGTGAAATCAATGCTGGTGCTGAACTACGTAGCAAA |
| | | AACCCGTTAATCAGCTTGTTTGGTCGTTGGGGATTAAGCGGCAAAGTTGGTATAGGGAATGCTATCCCGG |
| | | ATGGTGATAACCAGTGGGGGATGTTTGGTGGTGGTGCCCGCTCAATTATGTTTCAGCGTGACGAAAGTCT |
| | | GATGGAGTTTCTTGAGACAGACCAGGTTGATCGTCTTGAACGCCTGCTCGAGGAACAGGCTGAGGCAAGT |
| | | GTGGATATCTCTCAGATCAAAACAGAGCAAGATGCACTCAAAAAGGCGATGAAGTCGGCTGATAAAGACA |
| | | CCAAAGCTGAGCTTCAAATCAAAGTACGGGAGCTCGATGAAAAAATTCAGGCCCGCAAAGATCAGAAGCA |
| | | GGAATCTCGCGAGTCAATTCGCCGCCCGATTGACCCGTATGAAGCGTTTATCACCGGCGCAGAACTCAGC |
| | | CATCGCATGAGTATTAAAAATGCGACTGATGAGGAAGCAGGGCTTTTCATTTCTGCATTAATCCGCTTTG |
| | | CAGCCGAACCACGTTTTGGCGGTCATGCGAATCATAACTGCGGTCTGGTGGAGGCTCACTGGACAGTTAC |
| | | GACCTGGAAGCCGGGTGAACTGGTACCAGTTACACTTGGAGAAATCGTCATCACACCGAATGGTGTTGAG |
| | | ATTACCGGGGACGAGTTGTTTGCTATGGTAAAGGCATTCAATGAAAATCAATCTTTTGATTTCACTGCCC |
| | | GCTAACTCCAAAAGCTGGTGGATTTTAGTGGCGCTATTTAATATTTTATAATCAACCGGTTATTTTTAGA |
| | | GTATTCCCCCCGTGTGCGGGGGTTATCGGTGGGGCGTTGTTGAATAAATAGATATTATGCTGCGGGCCTA |
| | | CCAAAACGTCGGTCTTTCCCAACGCAAAATCAGTAGGTTAAGGTTAAAGTCGGGAGCTACTCACAATAGT |
| | | GACAAGCATCTTTGACTAAAGCAGGCGCGCCAAAACAGCATGATTCCCTATTATTCAACAAAGCCTATCG |
| | | GTGGTGCTCTCAACCGTCACCCGCTGGCTGGAAGTATTCCCCCCGTGTGCGGGGGTTATCGGTCGTGTTG |
| | | TCCACGGTTACCCGCTGGCTGGAAGTATTCCCCCCGCATGCGGGGGTTATCGGTTACCAATGGGGAAAAA |
| | | TCTTCATTTGTAAATGTATTCCCCCCGCATGCGGGGGTTATCGGAACATCAGTGGAAATCCACTGCGGCG |
| | | TATTCTCCCCGCATGCGGGGGTTATCGGCGAAAACGGCAACCTTCATAAAAACGTCTTTTGTATTCCCCC |
| | | CGTGTGCGGGGGTTATCGGCCGAGATTGAGTAAAGCAAAGTAACGGGGGTGGTATTCCCCCCGCATGCGG |
| | | GGGTTATCGGCAGGTTATACTGGCAAAACGTCGATG |
| Seq ID No: 13 | PAM | AAG |
| Seq ID No: 14 | pBolA promoter | aacctaaatatttgttgttaagctgcaatggaaacggtaaaagcggctagtatttaaaggGatggatgac |
| | | atctcagcgttgtcg |
| Seq ID No: 15 | P70a promoter | CCTGATGCGTGAACGTGACGGACGTAACCACCGCGACATGTGTGTGCTGTTCCGCTGGGCATGCTGAGCT |
| | | AACACCGTGCGTGTTGACAATTTTACCTCTGGCGGTGATAATGGTTGCAGCTAGCAATAATTTTGTTTAA |
| | | CTTTAAGAAGGAGATATACC |
| Seq ID No: 16 | XTEN linker | agcggcagcgagactcccgggacctcagagtccgccacacccgaaagt |
| | (nucleotide) | |
| Seq ID No: 17 | XTEN linker | SGSETPGTSESATPES |
| | (amino acid) | |
| Seq ID No: 18 | PmCDA1 cytidine | ATGACCGACGCTGAGTACGTGAGAATCCATGAGAAGTTGGACATCTACACGTTTAAGAAACAGTTTTTCA |
| | deaminase | ACAACAAAAAATCCGTGTCGCATAGATGCTACGTTCTCTTTGAATTAAAACGACGGGGTGAACGTAGAGC |
| | (nucleotide) | GTGTTTTTGGGGCTATGCTGTGAATAAACCACAGAGCGGGACAGAACGTGGCATTCACGCCGAAATCTTT |
| | | AGCATTAGAAAAGTCGAAGAATACCTGCGCGACAACCCCGGACAATTCACGATAAATTGGTACTCATCCT |
| | | GGAGTCCTTGTGCAGATTGCGCTGAAAAGATCTTAGAATGGTATAACCAGGAGCTGCGGGGGAACGGCCA |
| | | CACTTTGAAAATCTGGGCTTGCAAACTCTATTACGAGAAAAATGCGAGGAATCAAATTGGGCTGTGGAAT |
| | | CTCAGAGATAACGGGGTTGGGTTGAATGTAATGGTAAGTGAACACTACCAATGTTGCAGGAAAATATTCA |
| | | TCCAATCGTCGCACAATCAATTGAATGAGAATAGATGGCTTGAGAAGACTTTGAAGCGAGCTGAAAAACG |
| | | ACGGAGCGAGTTGTCCATTATGATTCAGGTAAAAATACTCCACACCACTAAGAGTCCTGCTGTT |
| Seq ID No: 19 | PmCDA1 cytidine | MTDAEYVRIHEKLDIYTFKKQFFNNKKSVSHRCYVLFELKRRGERRACFWGYAVNKPQSGTERGIHAEIF |
| | deaminase (amino | SIRKVEEYLRDNPGQFTINWYSSWSPCADCAEKILEWYNQELRGNGHTLKIWACKLYYEKNARNQIGLWN |
| | acid) | LRDNGVGLNVMVSEHYQCCRKIFIQSSHNQLNENRWLEKTLKRAEKRRSELSIMIQVKILHTTKSPAV |
| Seq ID No: 20 | Uracil DNA | ATGACCAACCTTTCCGACATCATAGAGAAGGAAACAGGCAAACAGTTGGTCATCCAAGAGTCGATACTCA |
| | glycosylase | TGCTTCCTGAAGAAGTTGAGGAGGTCATTGGGAATAAGCCGGAAAGTGACATTCTCGTACACACTGCGTA |
| | inhibitor (UGI) | TGATGAGAGCACCGATGAGAACGTGATGCTGCTCACGTCAGATGCCCCAGAGTACAAACCCTGGGCTCTG |
| | protein | GTGATTCAGGACTCTAATGGAGAGAACAAGATCAAGATGCTATAA |
| | (nucleotide) | |
| Seq ID No: 21 | Uracil DNA | MTNLSDIIEKETGKQLVIQESILMLPEEVEEVIGNKPESDILVHTAYDESTDENVMLLTSDAPEYKPWAL |
| | glycosylase | VIQDSNGENKIKML |
| | inhibitor (UGI) | |
| | protein (amino | |
| | acid) | |
| Seq ID No: 22 | Linker | TCTGGTGGATCTGGAGGTTCTGGTGGATCT |
| | (nucleotide) | |
| Seq ID No: 23 | Linker (amino | SGGSGGSGGS |
| | acid) | |
| Seq ID No: 24 | pBAD promoter | gaccaaagccatgacaaaaacgcgtaacaaaagtgtctataatcacggcagaaaagtccacattgattat |
| | (nucleotide) | ttgcacggcgtcacactttgctatgccatagcatttttatccataagattagcggatcctacctgacgct |
| | | ttttatcgcaactctctactgtttctccatacccgtttttttgggaattcgagctctaaggaggttataa |
| | | aaa |
| Seq ID No: 25 | Non-target | GGAGACGTCATGTCAAGCTCATAGGCGTCTCG |
| | Protospacer | |
| | sequence | |
| Seq ID No: 26 | U1kF protospacer | ACATACGCTTCTTGCTGAGTACGTGAGTTCAC |
| | sequence | |
| Seq ID No: 27 | U2kF protospacer | CAGATTGCTGGTCAGGAGCATATTGATCCGCT |
| | sequence | |
| Seq ID No: 28 | S1F protospacer | AAGGAGATATACCATGGAGCTTTTCACTGGCG |
| | sequence | |
| Seq ID No: 29 | S2F protospacer | TTCAGCGTGTCCGGCGAGGGCGAGGGCGATGC |
| | sequence | |
| Seq ID No: 30 | S3F protospacer | CTGCCCGTGCCCTGGCCCACCCTCGTGACCAC |
| | sequence | |
| Seq ID No: 31 | S4F protospacer | GAGAAGCGCGATCACATGGTCCTGCTGGAGTT |
| | sequence | |
| Seq ID No: 32 | S5F protospacer | TACCAGATGGCATTGCGCCATCTGGCAGAGTG |
| | sequence | |
| Seq ID No: 33 | S6F protospacer | AAAGTCAGGTTGCGCAGGCAGGGTAATCGGGG |
| | sequence | |
| Seq ID No: 34 | U1kR protospacer | TCACGTACTCAGCAAGAAGCGTATGTGTTTGC |
| | sequence | |
| Seq ID No: 35 | U2KR protospacer | ACCTCTGTCAGCTCGCTGACATTCCCCGCCAG |
| | sequence | |
| Seq ID No: 36 | S1R protospacer | TTAAACAAAATTATTGCTAGCTGCAACCATTA |
| | sequence | |
| Seq ID No: 37 | S2R protospacer | CTCCATGGTATATCTCCTTCTTAAAGTTAAAC |
| | sequence | |
| Seq ID No: 38 | S3R protospacer | CACTGCACGCCGTAGGTCAGGGTGGTCACGAG |
| | sequence | |
| Seq ID No: 39 | S4R protospacer | TCCAGCAGGACCATGTGATCGCGCTTCTCGTT |
| | sequence | |
| Seq ID No: 40 | S5R protospacer | CACTCTGCCAGATGGCGCAATGCCATCTGGTA |
| | sequence | |
| Seq ID No: 41 | S6R protospacer | CTGACTTTGTTGTCGGCCCGGCGGGCACTCAA |
| | sequence | |
| Seq ID No: 42 | Pc-tga promoter | aattcagcgggttcgtgcgaactgttgacaattaatactcggctcgtataatgtgtggaaggctagcagg |
| | | aggaattca |
| Seq ID No: 43 | BBa_J23119 | ttgacagctagctcagtcctaggtataatgctagc |
| Seq ID No: | BBa_J23100 | ttgacggctagctcagtcctaggtacagtgctagc |
| 441 | | |
| Seq ID No: 45 | BBa_J23101 | tttacagctagctcagtcctaggtattatgctagc |
| Seq ID No: 46 | BBa_J23102 | ttgacagctagctcagtcctaggtactctcctagc |
| Seq ID No: 47 | BBa_J23103 | ctgatagctagctcagtcctagggattatgctagc |
| Seq ID No: 48 | BBa_J23104 | ttgacagctagctcagtcctaggtattgtgctagc |
| Seq ID No: 49 | BBa_J23105 | tttacggctagctcagtcctaggtactatgctagc |
| Seq ID No: 50 | BBa_J23106 | tttacggctagctcagtcctaggtatagtgctagc |
| Seq ID No: 51 | BBa_J23107 | tttacggctagctcagccctaggtattatgctagc |
| Seq ID No: 52 | BBa_J23108 | ctgacagctagctcagtcctaggtataatgctagc |
| Seq ID No: 53 | BBa_J23109 | tttacagctagctcagtcctagggactgtgctagc |
| Seq ID No: 54 | BBa_J23110 | tttacggctagctcagtcctaggtacaatgctagc |
| Seq ID No: 55 | BBa_J23111 | ttgacggctagctcagtcctaggtatagtgctagc |
| Seq ID No: 56 | BBa_J23112 | ctgatagctagctcagtcctagggattatgctagc |
| Seq ID No: 57 | BBa_J23113 | ctgatggctagctcagtcctagggattatgctagc |
| Seq ID No: 58 | BBa_J23114 | tttatggctagctcagtcctaggtacaatgctagc |
| Seq ID No: 59 | BBa_J23115 | tttatagctagctcagcccttggtacaatgctagc |
| Seq ID No: 60 | BBa_J23116 | ttgacagctagctcagtcctagggactatgctagc |
| Seq ID No: 61 | BBa_J23117 | ttgacagctagctcagtcctagggattgtgctagc |
| Seq ID No: 62 | BBa_J23118 | ttgacggctagctcagtcctaggtattgtgctagc |
| Seq ID No: 63 | Pc-aga promoter | aattcagcgggttcgtgcgaactgtagacaattaatactcggctcgtataatgtgtggaaggctagcagg |
| | | aggaattca |
| Seq ID No: 64 | aspC (E. coli) | atgtttgagaacattaccgccgctcctgccgacccgattctgggcctggccgatctgtttcgtgccgatg |
| | | aacgtcccggcaaaattaacctcgggattggtgtctataaagatgagacgggcaaaaccccggtactgac |
| | | cagcgtgaaaaaggctgaacagtatctgctcgaaaatgaaaccaccaaaaattacctcggcattgacggc |
| | | atccctgaatttggtcgctgcactcaggaactgctgtttggtaaaggtagcgccctgatcaatgacaaac |
| | | gtgctcgcacggcacagactccggggggcactggcgcactacgcgtggctgccgatttcctggcaaaaaa |
| | | taccagcgttaagcgtgtgtgggtgagcaacccaagctggccgaaccataagagcgtctttaactctgca |
| | | ggtctggaagttcgtgaatacgcttattatgatgcggaaaatcacactcttgacttcgatgcactgatta |
| | | acagcctgaatgaagctcaggctggcgacgtagtgctgttccatggctgctgccataacccaaccggtat |
| | | cgaccctacgctggaacaatggcaaacactggcacaactctccgttgagaaaggctggttaccgctgttt |
| | | gacttcgcttaccagggttttgcccgtggtctggaagaagatgctgaaggactgcgcgctttcgcggcta |
| | | tgcataaagagctgattgttgccagttcctactctaaaaactttggcctgtacaacgagcgtgttggcgc |
| | | ttgtactctggttgctgccgacagtgaaaccgttgatcgcgcattcagccaaatgaaagcggcgattcgc |
| | | gctaactactctaacccaccagcacacggcgcttctgttgttgccaccatcctgagcaacgatgcgttac |
| | | gtgcgatttgggaacaagagctgactgatatgcgccagcgtattcagcgtatgcgtcagttgttcgtcaa |
| | | tacgctgcaggaaaaaggcgcaaaccgcgacttcagctttatcatcaaacagaacggcatgttctccttc |
| | | agtggcctgacaaaagaacaagtgctgcgtctgcgcgaagagtttggcgtatatgcggttgcttctggtc |
| | | gcgtaaatgtggccgggatgacaccagataacatggctccgctgtgcgaagcgattgtggcagtgctgt |
| | | aa |
| Seq ID No: 65 | taa1 (Arabidopsis | atggttaagttagagaatagtcgcaaacctgagaaaatctcaaataagaatattcctatgagcgacttcg |
| | thaliana) | tcgtgaatttagaccacggtgatccgacagcgtacgaagagtactggcgcaaaatgggagaccgctgtac |
| | | cgtgaccattcgcggttgtgacttgatgagttatttttctgatatgacgaatctgtgttggttcttggag |
| | | ccggaactggaggatgcgattaaggatttacacggtgtcgttggcaatgcagctacggaggaccgctata |
| | | tcgtggtgggaactgggtctacgcagttatgccaggctgcggtccacgcattgtcttctcttgcgcgtag |
| | | tcaaccagtttccgttgtggctgcggcaccattttacagcacgtacgtggaggagaccacctatgttcgc |
| | | agtgggatgtacaagtgggagggagacgcgtgggggtttgataagaaaggtccttatattgaacttgtga |
| | | catcaccgaataacccggacgggacgatccgcgagaccgtggttaatcgcccggatgatgatgaagcaaa |
| | | ggtaatccacgacttcgcttactattggccccattatacacctattacgcgtcgccaggatcacgacatc |
| | | atgctttttacctttagcaaaatcacggggcacgctggtagtcgtattggatgggcgctggtgaaggata |
| | | aagaggtcgctaagaaaatggttgagtatattatcgtgaacagtatcggcgtttcgaaagaatcgcaggt |
| | | gcgtacagcgaaaattttgaatgtcctgaaggaaacgtgtaaatctgaatccgaatccgagaacttcttc |
| | | aaatatggtcgtgagatgatgaagaatcgctgggagaagctgcgtgaagtagtcaaggagtcagacgcct |
| | | tcacattgcccaaatatcctgaggctttttgtaattatttcgggaagagtttagaatcatacccggcgtt |
| | | tgcctggctggggacaaaggaagagaccgacttggtatcggaattgcgtcgtcacaaggtaatgtcgcgt |
| | | gcaggcgagcgttgcggttcagataagaagcatgttcgcgtatcaatgctgtcccgcgaggatgttttca |
| | | atgtcttcttagagcgcttagccaacatgaaattgatcaaatctatcgatttgtaa |
| Seq ID No: 66 | ipdC (Pantoea | atgtcgacttttacagtaggtgactatttattaactcgcttacaagagatcggggtaaagcatttatttg |
| | agglomerans) | gagtacctggtgactataacttacaatttcttgaccgtgtgatcgctcatcccgctatttcatgggtagg |
| | | ctgtgccaacgagttgaacgcagcctatgcggctgatggatatgcacgttgcaatggggccggtgctctt |
| | | cttacgaccttcggggttggcgagctttccgcgatcaacggcatcgctggttcttatgccgagtatctgc |
| | | cggtcattcatattgtcggggcaccagctacgcaggcgcagttgcaaggggactgcgtgcatcattcttt |
| | | aggagatggggacttccagcattttatccgcatggcgactgaagtatcggctgccacggctcaacttaac |
| | | gccgacaacgccactgccgaaatcgaccgtgtaatcgtaagtgccctgcaagcacgccgtcccggatatc |
| | | tgtctctggctgtggatgtggcagccatggaagttcaaccaccagctcaaccattgaacagccaccaaag |
| | | ctctagtccggcagcccgtcgcgccttctcagatgctgccgagcgtttattggcgccagcacaacgcgtc |
| | | agtctgcttgcggattttttagccctgcgttggcagcaacaatcagccttagctgcattgcgcgaacaat |
| | | gcgccatcccgtgcgcttcgctgttaatggggaagggagtcttggatgagcagcagcccgggtatgttgg |
| | | tacttatgctggcgaggcgtctgcgggccaggtttgtgaacagattgaacaagtagacgtcgcaatttgt |
| | | gtcggggtccgttttacggacaccatcaccgccggatttacacagcagtttaatcccgagcgcctgatcg |
| | | acttgcagccacttagcgctagtgtcggcggagaacgctttgccccactttcaatggcagacgctctttc |
| | | cgaacttttgccgcttttcgagcgctatgggcaacagtggcaaccaggggctacgccacctgcagcacaa |
| | | ccggctgaacccgccgccgtcatttcccaacaagcgttttggcaagcaatgcaagccttcttacagccgg |
| | | gcgatcttattttggccgaacaaggtactgcggcatttggggctgccgcgcttcgtctgccaagccaggc |
| | | ccagttagtagtacaacctctttgggggtctattggatacaccctgccagcagcttttggtgcgcaaacg |
| | | gctgaccctgatcgtcgcgtgatcctgatcattggagatggttcagcgcaattgactatccaagagttag |
| | | gaagcatgctgcgcgaccaacaacgcttgatccttttcctgctgaacaatgagggctatactgttgaacg |
| | | tgcgattcacggtcctacgcagcgttataatgacattgcccagtggaattggaccgctctgcctcacgca |
| | | ctttctttgcaaggccaggcacaatcatggcgtatctcggagacggtgcaattagaggaggtgatggctc |
| | | gtttgtccgaacccaagtggctgtcattggtcgaggtcgtgatgcagaaggaggacctgcctcctttatt |
| | | acgcaaagtaacagcttgtcttaaccagcgtaatggctgctaa |
| Seq ID No: 67 | iad1 (Ustilago | atgccaactctcaacctcgatctgcctaacggcatcaagtcgaccatccaggctgacttgttcatcaaca |
| | mayadis) | acaagtttgtgcccgctcttgacggcaagacttttgcgaccatcaacccttccaccggtaaggagattgg |
| | | tcaggtcgccgaggcttccgccaaggacgttgatctcgctgtcaaggctgcgcgcgaggcattcgagact |
| | | acttggggcgagaacacacccggtgacgctcgtggcagacttctgatcaagctcgccgagctggtcgagg |
| | | ccaacattgacgagcttgctgccatcgagtcgctcgacaacggcaaggccttctcgatcgctaaaagctt |
| | | tgacgtcgctgccgtcgctgccaacttgcgatactatggtggctgggccgacaagaaccacggcaaggtc |
| | | atggaggtcgacaccaagcgcctcaactacacgcgccacgagcccatcggcgtgtgtggtcagatcatcc |
| | | cttggaacttccccttgctcatgttcgcctggaagctcggccctgcgctcgccacaggtaacacgatcgt |
| | | cctcaaaaccgccgagcagacaccactgtcagccatcaagatctctgagcttatcgtcgagcccgccttc |
| | | ccgcctggtgtggtcaacgtcatttccggtttcggtcccgtcgccggtgccgccatctcacagcacatgg |
| | | acattgacaagatcgccttcaccggctcaacactggtgggccgcaacattatgaaggctgctgcttcgac |
| | | caacctgaagaaggtgacgctcgagctcggcggcaagtcgcccaacattatctttaaggacgccgacctg |
| | | gaccaggggttcgatggtcggcgttcggtatcatgttcaatcacggacaatgctgctgtgcgggctcgcg |
| | | tgtctatgtggaggaatcgatctatgacgctttcatggagaagatgactgcccactgcaaggcgctccag |
| | | gtgggtgaccccttttccgccaacacgttccaaggtcctcaggtttcgcagctccagtacgaccgtatca |
| | | tggagtacatcgagtccggcaagaaggacgctaaccttgcgctcggtggtgtgcgcaagggtaacgaagg |
| | | ctactttatcgagcctaccatcttcaccgacgttcctcacgatgccaagattgccaaggaggagatcttc |
| | | ggccctgtcgtggtcgtctccaagttcaaagatgaaaaggacctcattcgcatcgccaacgactcgatct |
| | | acggcctcgctgccgctgtcttcagccgcgacatcagccgtgccatcgagactgcccacaagctcaaggc |
| | | cggcaccgtctgggtcaactgctacaaccagctcatccctcaggttccgttcggcgattacaaggcgtcc |
| | | ggtatcggtcgagagctcggcgagtatgcgctcagcaactacacaaacatcaaggcggtgcatgtgaacc |
| | | tcagccagcctgcccctatctaa |
| Seq ID No: 68 | RelB promoter | cgactactttctgacttccttcgtgacttgccctaagcatgttgtagtgcgatacttgtaAtgacatttg |
| | sequence, σ70 | taattacaagaggtg |
| Seq ID No: 69 | BolA promoter | aacctaaatatttgttgttaagctgcaatggaaacggtaaaagcggctagtatttaaaggGatggatgac |
| | sequence, σS, σ70 | atctcagcgttgtcg |
| Seq ID No: 70 | Hya promoter | aaaagataaatccacacagtttgtattgttttgtgcaaaagtttcactacgctttattaaCaatactttc |
| | sequence, σS, σ70 | tggcgacgtgcgcca |
| Seq ID No: 71 | YiaG promoter | agccagagcatgccctgacttcaccccgctgtgtctgcttttcccgactattcttaatgaGcttcgatgc |
| | sequence, σS | aattcacgatcccgc |
| Seq ID No: 72 | RpoH promoter | acggtacaacatttacgccactttacgcctgaataataaaagcgtgttatactctttcccTgcaatgggt |
| | sequence P1, σ70 | tccgtagcagggaaa |
| Seq ID No: 73 | RpoH promoter | acggtacaacatttacgccactttacgcctgaataataaaagcgtgttatactctttcccTgcaatgggt |
| | sequence P2, σS | tccgtagcagggaaa |
| Seq ID No: 74 | RpoH promoter | tgcgtaatttattcacaagcttgcattgaacttgtggataaaatcacggtctgataaaacAgtgaatgat |
| | sequence P3, σ24 | aacctcgttgctctt |
| Seq ID No: 75 | RpoH promoter | tttattcacaagcttgcattgaacttgtggataaaatcacggtctgataaaacagtgaatGataacctcg |
| | sequence P4, σ70 | ttgctcttaagctct |
| Seq ID No: 76 | RpoH promoter | tgcattgaacttgtggataaaatcacggtctgataaaacagtgaatgataacctcgttgcTcttaagctc |
| | sequence P5, σ70 | tggcacagttgttgc |
| Seq ID No: 77 | RpoH promoter | aacagtgaatgataacctcgttgctcttaagctctggcacagttgttgctaccactgaagCgccagaaga |
| | sequence P6, σ54 | tatcgattgagagga |
| Seq ID No: 78 | hypC2 sequence | ATGCAGAATAAACCTACACCTGAAGAAGTAAAGAATGCGCGGGTTGCGGCAGGTCTTACTCTTAAAGAAG |
| | | CTGCTGATATTTTTGGTTATCAACTGAATTCCTGGCAGATGAAAGAAAGTGCAGGTAAGGCCAGTCGTTC |
| | | TTTATCTATTGGTGAATATCAGTATTTATTGTTATTAGCAAATATGCATCCGTCTTACAGGCTGGTAAAA |
| | | AAATAA |
| Seq ID No: 79 | oli9579 | gacaataaataatgatcatccccagaaatcatgtggagcacaaccaacaactgacgaaaaatactgaaga |
| | | gtttgtagaaacgcaaaaag |
| Seq ID No: 80 | oli9580 | caaaaaatacgcccggtagtgatcttatttcattatggtgaaagttcgaacctcttacgtgccgatcatt |
| | | agaaaaactcatcgagcatc |
| Seq ID No: 81 | oli9581 | cggacttagtgaacagggttttacagtctgattgacagatatggtcaccagaaaatatttgtattccttt |
| | | agaaaaactcatcgagcatc |
| Seq ID No: 82 | oli8805 | cgcagacaataaataatgatcatccccagaaatcatgtggagcacaaccaacaactgacgaaaaatactg |
| | | atgatcggcacgtaagagg |
| Seq ID No: 83 | oli8743 | accccggacttagtgaacagggttttacagtctgattgacagatatggtcaccagaaaatatttgtattc |
| | | ctaattcagcgggttcgtgc |
| Seq ID No: 84 | Oli7154 | tacggctgcatgcccgaggcagacagcctcaagcacccgcagctattctacagtaaaaactcgcgcttct |
| | | ctagactatattacccctgtt |
| Seq ID No: 85 | Oli7156 | agccgtacggaagtttcccccgacaccatgatggcggttagttgcgtcagaaatagttacgtatgcaact |
| | | aatcagtcctgctcctcggc |
| Seq ID No: 86 | Oli7159 | ccgcacgtaagaggctaacc |
| Seq ID No: 87 | Oli7160 | ggaagtttcccccgacaccatgatggcggttagttgcgtcagaaatagttacgtatgcaactaaaagcgc |
| | | gagtttttactgtagaatag |
| Seq ID No: 88 | oli8199 | gtggtttgccacaaaacagtgcagtcacacatgacaggagaagatatgagccgggtaccgcggctctgag |
| | | actcagtcctgctcctcggc |
| Seq ID No: 89 | oli8200 | ggtgcaggaaataaaaataatcatttctttatatgatctttttatcaatggagatagtatcatttatact |
| | | ctagactatattaccctgtt |
| Seq ID No: 90 | oli8201 | cctgtatttttctgctcaatgg |
| Seq ID No: 91 | oli8197 | aaacagtgcagtcacacatgacaggagaagatatgagccgggtaccgcggctctgagactataaatgata |
| | | ctatctccattgataaaaag |
| Seq ID No: 92 | oli2906 | gtgatctccaccggcttattagcgatagggattatctgtaccgtgccaattatggctcgcccttagttcc |
| | | tattccgaagttcct |
| Seq ID No: 93 | oli2907 | gtcgaaaatatcgagcagctcctgctctgacatagacgccacccgataccctttttgattcaccaccgcc |
| | | ctgccactcatcgcagt |
| Seq ID No: 94 | oli8729 | attcagcgggttcgtgcgagctgttgacaattaatactcggctcgtataatgtgtggaaggctagcagga |
| | | ggtgggctagcgaattcgag |
| Seq ID No: 95 | oli2893 | ttaattgccagccatcgcct |
| Seq ID No: 96 | oli5622 | ccggcttattagcgatagggattatctgtaccgtgccaattatggctcgctggaaaaagctggtattgtg |
| | | gcaaaaaacacccgttcataatacgcgctgaattcagcgggttcgtgcg |
| Seq ID No: 97 | oli8821 | gtgatcaccagataatgttgc |
| Seq ID No: 98 | oli8822 | gcttcatcaccactgacc |
| Seq ID No: 99 | Oli8116 | ccggtatcgggttaaagagtgggaaaaagttgccggtgttcctgttgcagaagtattttgactcacttcc |
| | | ctgttaagtatcttcct |
| Seq ID No: | Oli8117 | aaaagaaaaagccggttcagagaaaaccggctgacggattatcgtctaacagttgatagctaccattacg |
| 100 | | ccccgccctgccactca |
| Seq ID No: | oli2892 | gcgtcaaggatgctctactc |
| 101 | | |
| Seq ID No: | PcBE promoter | aacctaaatatttgttgttaagctgcaatggaaacggtaaaagcggctagtatttaaagggatggatgac |
| 102 | (constitutive) | atctcagcgttgtcgggattaacaatataagctgaccttcaagtattgaattgggataatgtgtggaatt |
| | | gtgaa |
| Seq ID No: | Repeat sequence | GTATTCCCCCCGCATGCGGGGGTTATCGG |
| 103 | | |

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

Concepts relating to conjugative plasmids for the production of a first MOI which is indole-3-acetic Recent evidence has linked indole-3-acetic acid (IAA, a gut microbiota-derived metabolite from dietary tryptophan), with resistance to liver damage and steatosis in mice (Ji et al., Nutrient 11:2062, 2019; Li et al, Frontiers in Pharmacology, 12:769501, 2021), and improved epithelial barrier function in mice (Laurans et al., Nature Medicine, 24:1113, 2018) and piglets (Liang et al, Frontiers in Microbiology, 9:1736, 2018). In humans, decreased levels of tryptophan and IAA, and increased levels of kynurenine, have been observed in the fecal samples from patients with obesity and T2D compared to healthy subjects, and similarly a change in tryptophan metabolism towards more kynurenine and less IAA in people with obesity and T2D (Laurans et al., 2018, supr, Natividad et al., Cell Metabolism, 28:737, 2018).

In vitro data demonstrated that IAA induces activation of the aryl hydrocarbon receptor (AHR) in intestinal immune cells, increasing the production of anti-inflammatory interleukins like IL-17 and IL-22, which are important for antimicrobial immunity and mucosal barrier integrity (Laurans et al, 2018, supra). Moreover, IAA has been shown to attenuate lipogenesis in hepatocytes induced by cytokine and free fatty acids (Krishnan et al., Cell Reports, 23:1099, 2018). Ji et al., supra showed that activation of AHR by IAA alleviates high-fat diet (HFD)-induced hepatotoxicity in mice.

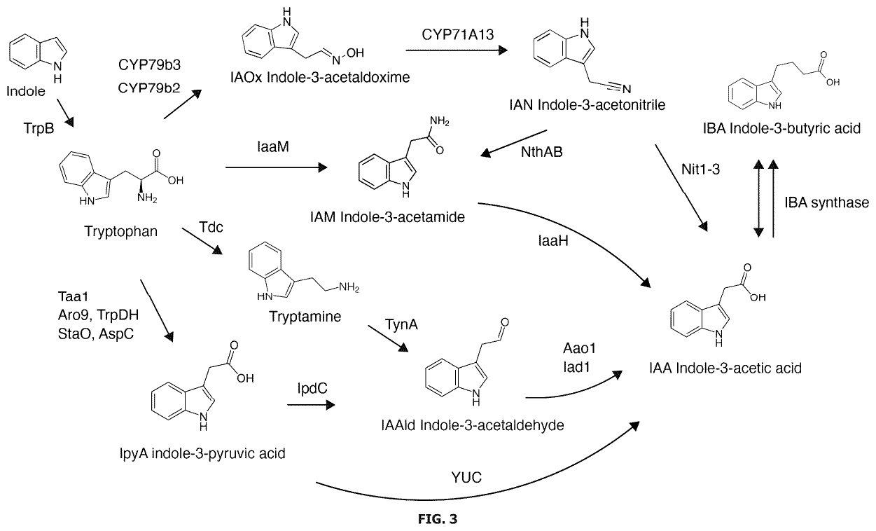

There are various pathways for the production of IAA, which are shown schematically in FIG. 3. Each step of the pathway is governed by one or more heterologous genes. Any of the genes of the pathways (or part pathways, if it is desirable to start production of IAA from an intermediate molecule) shown in FIG. 3 could be inserted into a bacterium or into a plasmid (e.g. into a conjugative plasmid) described herein to produce IAA.

Thus, IAA is a therapeutically useful molecules and there remains a need for efficient in sVi production of IAA. Thus, there is provided the following concepts:

Concept 1. A transmissible element (e.g. a plasmid, conjugative plasmid, phage or phagemid, e.g. as described elsewhere herein) for transmission to a recipient bacterium (e.g. as described elsewhere herein) or a bacterium (e.g. as described elsewhere herein) comprising said transmissible element, wherein the transmissible element comprises:
A) at least one exogenous nucleic acid sequence to the recipient bacterium comprising one or more heterologous gene(s) for the biosynthesis of IAA, and a heterologous gene encoding an exporter which is capable of exporting IAA out of the recipient bacterium; and
B) a Pathway Modulation System (PMS), e.g. as described elsewhere herein, which encodes a nucleic acid modifier or a peptide molecule to modulate a target endogenous nucleic acid or protein for the production of IAA in the recipient bacterium.
   Concept 2. The transmissible element or bacterium according to concept 1, wherein the one or more heterologous gene(s) for the biosynthesis of IAA are selected from:
A. a gene for the conversion of tryptophan to tryptamine (optionally a gene which is tryptophan decarboxylase (tdc)), a gene for the conversion of tryptamine to indole-3-acetaldehyde (IAAld) (optionally a gene which is monoamine oxidase (tynA)), a gene for the conversion of IAAld to IAA (optionally a gene selected from indole-3-acetaldehyde dehydrogenase (iad1) and Indole-3-acetaldehyde oxidase (aao1));
B. a gene for the conversion of indole to tryptophan (optionally a gene which is #p), a gene for the conversion of tryptophan to tryptamine (optionally a gene which is tryptophan decarboxylase (tdc)), a gene for the conversion of tryptamine to indole-3-acetaldehyde (IAAld) (optionally a gene which is monoamine oxidase (tynA)), a gene for the conversion of IAAld to IAA (optionally a gene selected from indole-3-acetaldehyde dehydrogenase (WI) and Indole-3-acetaldehyde oxidase (aao1));
C. a gene for the conversion of tryptophan to indole-3-pyruvic acid (IPyA) (optionally a gene selected from L-tryptophan oxidase (staO), L-tryptophan aminotransferase (am9), aspartate aminotransferase (aspC), L-tryptophan-pyruvate aminotransferase (taa1) and tryptophan dehydrogenase (trpDH)), a gene for the conversion of IPyA to IAAld (optionally a gene which is indole-3-pyruvate decarboxylase (ipdC)), a gene for the conversion of IAAId to IAA (optionally a gene selected from iad1 and aao1);
D. a gene for the conversion of indole to tryptophan (optionally a gene which is trpB), a gene for the conversion of tryptophan to indole-3-pyruvic acid (IPyA) (optionally a gene selected from L-tryptophan oxidase (staO), L-tryptophan aminotransferase (aro9), aspartate aminotransferase (aspC), L-tryptophan-pyruvate aminotransferase (taa1) and tryptophan dehydrogenase (trpDH)), a gene for the conversion of IPyA to IAAld (optionally a gene which is indole-3-pyruvate decarboxylase (ipdC)), a gene for the conversion of IAAld to IAA (optionally a gene selected from iad1 and aao1);
E. a gene for the conversion of tryptophan to indole-3-pyruvic acid (IPyA) (optionally a gene selected from staO, aro9, aspC taa1 and trpDH), a gene for the conversion of IPyA to IAA (optionally a gene which is an indole-3-pyruvate monooxygenase (e.g. selected from YUC genes, yuc2, and yuc6));
F. a gene for the conversion of indole to tryptophan (optionally a gene which is trpB), a gene for the conversion of tryptophan to indole-3-pyruvic acid (IPyA) (optionally a gene selected from staO, aro9, aspC, taa1 and trpDH), a gene for the conversion of IPyA to IAA (optionally a gene which is an indole-3-pyruvate monooxygenase (e.g. selected from YUC genes, yuc2, and yuc6));
G. a gene for the conversion of tryptophan to indole-3-acetamide (IAM) (optionally a gene which is tryptophan 2-monooxygenase (iaaM)), a gene for the conversion of IAM to IAA (optionally a gene which is indoleacetamide hydrolase (iaaH));
H. a gene for the conversion of indole to tryptophan (optionally a gene which is trpB), a gene for the conversion of tryptophan to indole-3-acetamide (IAM) (optionally a gene which is tryptophan 2-monooxygenase (iaaM)), a gene for the conversion of IAM to IAA (optionally a gene which is indoleacetamide hydrolase (iaaH));
I. a gene for the conversion of tryptophan to indole-3-acetaldoximine (IAOx) (optionally a gene selected from tryptophan N-monooxygenase (CYP79B2) and tryptophan N-monooxygenase (CYP79B3)), a gene for the conversion of IAOx to indole-3-acetonitrile (IAN) (optionally a gene which is indoleacetaldoxime dehydratase (CYP71A13)), a gene for the conversion of IAN to IAA (optionally a gene which is a nitrilase (e.g. selected from nit1, nit2 and nit3);
J. a gene for the conversion of indole to tryptophan (optionally a gene which is trpB), a gene for the conversion of tryptophan to indole-3-acetaldoxime (IAOx) (optionally a gene selected from tryptophan N-monooxygenase (CYP79B2) and tryptophan N-monooxygenase (CYP7983)), a gene for the conversion of IAOx to indole-3-acetonitrile (IAN) (optionally a gene which is indoleacetaldoxime dehydratase (CYP71A13)), a gene for the conversion of IAN to IAA (optionally a gene which is a nitrilase (e.g. selected from nit1, nit2 and nit3);
K. a gene for the conversion of tryptophan to indole-3-acetaldoximine (IAOx) (optionally a gene selected from CYP7982 and CYP79B3), a gene for the conversion of IAOx to indole-3-acetonitrile (IAN) (optionally a gene which is CYP71A13), a gene for the conversion of IAN to IAM (optionally a gene which is nitrile hydratase (nthAB)), a gene for the conversion of IAM to IAA (optionally a gene which is iaaH);
L. a gene for the conversion of indole to tryptophan (optionally a gene which is trpB), a gene for the conversion of tryptophan to indole-3-acetaldoxime (IAOx) (optionally a gene selected from CYP79B2 and CYP79B3), a gene for the conversion of IAOx to indole-3-acetonitrile (IAN) (optionally a gene which is CYP71A13), a gene for the conversion of IAN to IAM (optionally a gene which is nitrile hydratase (nthAB), and a gene for the conversion of TAM to IAA (optionally a gene which is iaaf);
M. any combination of pathways A to L; and
N. any part of pathways A to L.

A gene for the conversion of tryptophan to tryptamine includes but is not limited to tdc. Tryptophan decarboxylase (tdc) may be from the genus Catharanthus, e.g. Catharanthus roseus or from the genus Clostridium, e.g. Clostridium sporogenes.

A gene for the conversion of tryptamine to IAAld includes but is not limited to tynA. Monoamine oxidase (tynA) may be from the genus Escherichia, e.g. E. coli.

Genes for the conversion of IAAld to IAA include but are not limited to iad1 and aao1. Indole-3-acetaldehyde dehydrogenase (iad1) may be from the genus Ustilago, e.g. Ustilago maydis. Indole-3-acetaldehyde oxidase (aao1) may be from the genus Arabidopsis, e.g. Arabidopsis thaliana. In one embodiment, the iad1 comprises the nucleotide sequence of Seq ID No: 67. In one embodiment, the gene has at least 80% identity (such as 85%, 90% or 95%, in particular 90° % identity) to Seq ID No: 67, and converts IAAId to IAA.

A gene for the conversion of indole to tryptophan includes, but is not limited to trpB. Tryptophan synthase β-subunit (rpB) may be from the genus Escherichia, e.g E. coli. trpB includes paralogs TrpB1 and TrpB2.

Genes for the conversion of tryptophan to IPyA include, but are not limited to staO, aro9, aspC, taa1 and trpDH. L-tryptophan oxidase (staO) may be from the genus Streptomycese, e.g. streptomyces sp. 7P-A0274. L-tryptophan aminotransferase (am9), may be from S. cerevisae. Aspartate aminotransferase (asp) may be from E. coli. L-tryptophan-pyruvate aminotransferase (taa1) may be from the genus Arabidopsis, e.g. Arabidopsis thaliana. Tryptophan dehydrogenase (trpDH) may be from the genus Nostoc, e.g. a strain of Nostoc punctiforme, such as Nostoc punctiforme NIES-2108. In one embodiment, the aspC comprises the nucleotide sequence of Seq ID No: 64 In one embodiment, the gene has at least 80% identity (such as 85%, 90% or 95%, in particular 90% identity) to Seq ID No: 64, and converts tryptophan to IPyA. In one embodiment, the taa1 comprises the nucleotide sequence of Error!Reference source not found. In one embodiment, the gene has at least 80% identity (such as 85%, 90% or 95%, in particular 90% identity) to Seq ID No: 65, and converts tryptophan to IPyA.

trpDH may be disadvantageous, due to its reported instability, see Matsui et al., J. Biotechnol., 196-197:27-32, 2015, doi: 10.1016/j.jbiotec.2015.01.010, which is incorporated herein in its entirety. Even at low temperatures (4° C.), trpDH (EC1.4.1.19) showed >80% reduction in activity after 24 hours. Depending on the desired application, aspC may be less attractive, due to its range of substrates/catalytic reactions. aspC has been identified as a multifunctional enzyme, that catalyses the synthesis of aspartate, phenylalanine, tyrosine and other compounds via a transamination reaction. In contrast, whilst taa1 may catalyse reactions with tryptophan, phenylalanine and tyrosine, taa1 has a much higher affinity for tryptophan than its other substrate, and thus is likely to provide higher levels of conversion of tryptophan to IPyA than aspC

A gene for the conversion of IPyA to IAAld includes, but is not limited to IpdC. Indole-3-pyruvate decarboxylase (IpdC) may be from the genus Enterobacter, e.g. Enterobacter cloacae or from the genus Pantoea, e.g. Pantoea agglomerans, in particular Pantoea agglomerans. In one embodiment, the IpdC comprises the nucleotide sequence of Seq ID No: 66. In one embodiment, the gene has at least 80% identity (such as 85%, 90% or 95%, in particular 90% identity) to Seq ID No: 66, and converts IPyA to IAAld.

A gene for the conversion of IPyA to IAA includes, but is not limited to YUC genes. Indole-3-pyruvate monooxygenase (YUC) genes may be from the genus Arabidopsis, e.g. Arabidopsis thaliana. YUC genes may also be from the genus Escherichia, e.g. E. coli, such as YUC2 and YUC6.

A gene for the conversion of tryptophan to IAM includes, but is not limited to iaaM. Tryptophan 2-monooxygenase (iaaM) may be from the genus Pseudomonas, e.g. Pseudomaonas savastanoi.

A gene for the conversion of IAM to IAA includes, but is not limited to iaaH. Indoleacetamide hydrolase (iaaH) may be from the genus Pseudomonas, e.g. Pseudomonas savastanoi.

Genes for the conversion of tryptophan to IAOx include, but are not limited CYP79B2 and CYP7983. Tryptophan N-monooxygenase (CYP7982) may be from the genus Arabidopsis, e.g. Arabidopsis thaliana. Tryptophan N-monooxygenase (CYP79B3) may be from the genus Arabidopsi, e.g. Arabidopsis thaliana.

A gene for the conversion of IAOx to IAN includes but is not limited to CYP71A13. Indoleacetaldoxime dehydratase (CYP71413) may be from the genus Arabidopsis, e.g. Arabidopsis thaliana. The enzyme is described in more detail in Nafisi et al., The Plant Cell, 19(6), 2039-2052, 2007, which is incorporated herein in its entirety.

Genes for the conversion of IAN to IAA include but are not limited to nit1, nit2 and nit3. Nitrilases may be from the genus Arabidopsis, e.g. Arabidopsis thaliana.

A gene for the conversion of IAN to IAM includes but it not limited to nbAB. Nitrile hydratase (nthA) may be from the genus Arabidopsis, e.g. Arabidopsis thaliana. nthAB may be from a Pseudomonas strain, e.g. Pseudomonas sp Strain UW4. The enzyme is described in more detail in Duca et al., Applied and Environmental Microbiology 80(15), 2014, doi:10.1128/AEM.00649-14 which is incorporated herein in its entirety.

As will be apparent to those skilled in the art, the above list may not encompass all known genes which catalyse the stated reaction. Further genes may be identified, for example, through literature searching, using known databases (such as GenBank, Kegg, EMBL or protein databases such as Uniprot and the like), either through keyword searching, putative annotations and/or sequence homology to a known enzyme (protein and/or DNA sequence). A list of databases can be found here: https://en.wikipedia.org/wiki/List-of_biological_databases.

Concept 3. The transmissible element or bacterium according to concept 2, wherein the one or more heterologous gene(s) for the biosynthesis of IAA comprise:
i. Indole-3-pyruvate decarboxylase (ipdC);
ii. Tryptophan-pyruvate aminotransferase 1 (taa1); and
iii. Indole-3-acetaldehyde dehydrogenase (iad1).
   Concept 4. The transmissible element or bacterium according to claim 3, wherein;
i. the ipdC is from a Pantoea species, e.g. from Pantoea agglomerans, optionally having the nucleic acid sequence of SEQ ID No:41; and/or
ii. the taa1 is from an Arabidopsis species, e.g. from Arabidopsis thaliana, optionally having the nucleic acid sequence of SEQ ID No:40; and/or
iii. the iad1 is from an Ustilago species, e.g. from Ustilago maydis, optionally having the nucleic acid sequence of SEQ ID No:42.

In one embodiment, heterologous gene(s) for the biosynthesis of IAA comprise:
i. Indole-3-pyruvate decarboxylase (ipdC);
ii. Tryptophan-pyruvate aminotransferase 1 (taa1); and
iii. Indole-3-acetaldehyde dehydrogenase (iad1).

In one embodiment, heterologous gene(s) for the biosynthesis of IAA comprise:
i. Indole-3-pyruvate decarboxylase (ipdC) from a Pantoea species;
ii. Tryptophan-pyruvate aminotransferase 1 (taa1) from an Arabidopsis species; and
iii. Indole-3-acetaldehyde dehydrogenase (iad1) from an Ustilago species.

In one embodiment, heterologous gene(s) for the biosynthesis of IAA comprise:
i. Indole-3-pyruvate decarboxylase (pdC) from Pantoea agglomerans,
ii. Tryptophan-pyruvate aminotransferase 1 (taa1) from Arabidopsis thaliana; and
iii. Indole-3-acetaldehyde dehydrogenase (iad1) from Ustilago maydis.
   Concept 5. The transmissible element or bacterium according to any preceding concept, wherein the one or more heterologous genes for the biosynthesis of IAA are each or all under the control of one or more constitutive promoter(s), e.g. as described elsewhere herein.
   Concept 6. A transmissible element (e.g. a plasmid, conjugative plasmid, phage or phagemid, e.g. as described elsewhere herein) for the biosynthesis of indole-3-acetic acid (IAA) and for transmission to a recipient bacterium (e.g. as described elsewhere herein), or a bacterium (e.g. as described elsewhere herein) comprising said transmissible element, wherein the transmissible element comprises:
A) at least one exogenous nucleic acid sequence to the recipient bacterium comprising
i. Indole-3-pyruvate decarboxylase (ipdC) from a Pantoea species, e.g. from Pantoea agglomerans, optionally having the nucleic acid sequence of SEQ ID No:41;
ii. Tryptophan-pyruvate aminotransferase 1 (taa1) from an Arabidopsis species, e.g. from Arabidopsis thaliana, optionally having the nucleic acid sequence of SEQ ID No:40; and
iii. Indole-3-acetaldehyde dehydrogenase (iad1) from an Ustilago species, e.g. from Ustilago maydis, optionally having the nucleic acid sequence of SEQ ID No:42; and
B) a Pathway Modulation System (PMS), e.g. as described elsewhere herein, which encodes a nucleic acid modifier to modulate a target endogenous nucleic acid or protein for the production of IAA in the recipient bacterium, and
wherein heterologous genes i. to iii. are each or all under the control of one or more constitutive promoter(s).
   Concept 7. The transmissible element or bacterium according to concept 6, wherein the at least one exogenous nucleic acid sequence of A) further comprises a heterologous gene encoding an exporter which is capable of exporting IAA out of the recipient bacterium.
   Concept 8. The transmissible element or bacterium according to any preceding concept, wherein the heterologous gene encoding an exporter which is capable of exporting IAA is under the control of a constitutive promoter.
   Concept 9. The transmissible element or bacterium according to any preceding concept, wherein the one or more heterologous gene(s) for the biosynthesis of IAA are comprised within an operon under the control of a single constitutive promoter.
   Concept 10. The transmissible element or bacterium according to concept 9, wherein the operon comprises the following genes in downstream order:
i. Indole-3-pyruvate decarboxylase (ipdC), optionally from a Pantoea species, e.g. from Pantoea agglomerans, further optionally having the nucleic acid sequence of SEQ ID No:41;
ii. Tryptophan-pyruvate aminotransferase 1 (taa1), optionally from an Arabidopsis species, e.g. from Arabidopsis thaliana, further optionally having the nucleic acid sequence of SEQ ID No:40; and
iii. Indole-3-acetaldehyde dehydrogenase (iad1), optionally from an Ustilago species, e.g. from Ustilago maydis, further optionally having the nucleic acid sequence of SEQ ID No:42.
   Concept 11. The transmissible element or bacterium according to concept 9 or concept 10, wherein the one or more heterologous gene(s) for the biosynthesis of IAA and the heterologous gene encoding an exporter which is capable of exporting IAA are comprised within an operon under the control of a single constitutive promoter.
   Concept 12. The transmissible element or bacterium according to any one of concepts 5 to 11, wherein the constitutive promoter is a strong constitutive promoter having an Anderson score >0.4 (e.g. >0.5), e.g. a tac promoter, for example a Pc-tga promoter having the sequence of SEQ ID No: 1.
   Concept 13. The transmissible element or bacterium according to any one of concepts 5 to 11, wherein the promoter is a promoter selected from a RelB, BolA, Hya, YiaG and a RpoH promoter, such as a promoter selected from a RelB promoter sequence, σ70; a BolA promoter sequence, σS, σ70; a Hya promoter sequence, σS, σ70; a YiaG promoter sequence, σS; a RpoH promoter sequence P1, σ70; a RpoH promoter sequence P2, σS; a RpoH promoter sequence P3, σ24; a RpoH promoter sequence P4, σ70; a RpoH promoter sequence P5, σ70; and a RpoH promoter sequence P6, σ54, in particular a promoter having a nucleotide sequence selected from any one of Seq ID Nos: 60 to 69, or a nucleotides sequence having 90% (or 95%) homology thereto.
   Concept 14. The transmissible element or bacterium according to any one of concepts 11 to 13, wherein the one or more heterologous gene(s) for the biosynthesis of IAA and the heterologous gene encoding an exporter which is capable of exporting IAA comprised within the operon comprise the following genes in downstream order:
i. Indole-3-pyruvate decarboxylase (ipdC), optionally from a Pantoea species, e.g. from Pantoea agglomerans, further optionally having the nucleic acid sequence of SEQ ID No:41;
ii. Tryptophan-pyruvate aminotransferase 1 (taa1), optionally from an Arabidopsis species, e.g. from Arabidopsis thaliana, further optionally having the nucleic acid sequence of SEQ ID No:40; and
iii. Indole-3-acetaldehyde dehydrogenase (iad1), optionally an Ustilago species, e.g. from Ustilago maydis, further optionally having the nucleic acid sequence of SEQ ID No:42; and
iv. the heterologous gene encoding an exporter of IAA.

In any embodiment or concept herein, further genes may be included in the transmissible element to increase the production of tryptophan.

Thus, in any embodiment, the transmissible element may comprise one or more heterologous gene(s) for the biosynthesis of tryptophan from chorismate ((3R,4R)-3-[(1-carboxyvinyl)oxy]-4-hydroxycyclohexa-1,5-diene-1-carboxylic acid).

In any embodiment, or concept the transmissible element may comprise one or more heterologous gene(s) for the biosynthesis of tryptophan from anthranilic acid.

In one embodiment, the one or more heterologous gene(s) for the biosynthesis of tryptophan comprise (or consists of) a gene for the conversion of anthranilic acid to N-(5-phosphoribosyl)-anthranilate (optionally a gene which is phosphoribosyl transferase (trp)), a gene for the conversion of N-(5-phosphoribosyl)-anthranilate to 1-(o-carboxyphenylamino-1-deoxyribulose-5-phosphate (optionally a gene which is (trpF)), a gene for the conversion of 1-(o-carboxyphynylamino-1-deoxyribulose-5-phosphate to indole-3-glycerol phosphate (optionally a gene which is InGP synthase (trpC), and a gene for the conversion of ndole-3-glycerol phosphate to indole (optionally a gene which is tryptophan synthase (trpA)).

In any embodiment or concept, the transmissible element may comprise one or more heterologous gene(s) for the biosynthesis of tryptophan from N-(5-phosphoribosyl)-anthranilate.

In one embodiment, the one or more heterologous gene(s) for the biosynthesis of tryptophan comprise (or consists of) a gene for the conversion of N-(5-phosphoribosyl)-anthranilate to 1-(o-carboxyphynylamino-1-deoxyribulose-5-phosphate (optionally a gene which is (tp)), a gene for the conversion of 1-(o-carboxyphynylamino-1-deoxyribulose-5-phosphate to indole-3-glycerol phosphate (optionally a gene which is InGP synthase (trpC)), and a gene for the conversion of indole-3-glycerol phosphate to indole (optionally a gene which is tryptophan synthase (trpA)).

In any embodiment or concept, the transmissible element may comprise one or more heterologous gene(s) for the biosynthesis of tryptophan from 1-(o-carboxyphynylamino-1-deoxyribulose-5-phosphate.

In one embodiment, the one or more heterologous gene(s) for the biosynthesis of tryptophan comprise (or consists of) a gene for the conversion of 1-(o-carboxyphynylamino-1-deoxyribulose-5-phosphate to indole-3-glycerol phosphate (optionally a gene which is InGP synthase (trpC)), and a gene for the conversion of indole-3-glycerol phosphate to indole (optionally a gene which is tryptophan synthase (trpA)).

In any embodiment or concept, the transmissible element may comprise one or more heterologous gene(s) for the biosynthesis of tryptophan from indole-3-glycerol phosphate.

In one embodiment, the one or more heterologous gene(s) for the biosynthesis of tryptophan comprise (or consists of) a gene for the conversion of indole-3-glycerol phosphate to indole (optionally a gene which is tryptophan synthase (trpA)).

Concept 15. The transmissible element or bacterium according to any preceding concept, wherein the nucleic acid modifier (as described elsewhere herein) or peptide molecule reduces or prevents biologic activity of an expressed tryptophan transcriptional repressor (TrpR) protein in the recipient bacterium.

The trpR gene, when transcribed, forms a homodimer, which binds to tryptophan. Upon tryptophan binding, TrpR binds to the DNA of the promoter of the trp operon and sterically blocks RNA polymerase from binding and initiating transcription. Thus, the mutation in endogenous trpR results in increased transcription of the trp operon. The effect is threefold: (i) to de-repress synthesis of chorismate (thus increasing the amount of chorismate in the cell), (ii) to de-repress bpEDC4, which converts chorismate to indole (thus increasing the amount of indole in the cell), and (iii) to de-repress the mtr proton symporter of tryptophan and indole into the cell (thus increasing the amount of tryptophan and indole in the cell).

In one embodiment or concept, the PMS reduces or prevents (e.g. prevents) biologic activity of endogenously expressed tryptophan transcriptional repressor (TrpR) in the recipient bacterium. In one embodiment, the PMS introduces a modification (e.g. a deletion, or substitution) in endogenous tryptophan transcriptional repressor (trpR) which reduces or prevents (e.g. prevents) biologic activity of endogenously expressed tryptophan transcriptional repressor (TrpR) in the recipient bacterium. In one embodiment, the PMS deletes endogenous tryptophan transcriptional repressor (trpR) in the recipient bacterium. The benefits of preventing the biological activity of TrpR is explained elsewhere herein.

In any embodiment or concept, the modification in endogenous trpR is a deletion of one or more nucleotides in an endogenous trpR gene which prevents or reduces (e.g. prevents) transcription or expression of trpR. In another embodiment, the PMS introduces a modification in an endogenous trpR gene, which results in TrpR no longer being able to form a homodimer (e.g. the modification is a deletion or mutation of one or more amino acids in the dimerization interface, which prevents dimerization of TrpR). In another embodiment, the PMS introduces a modification in an endogenous trpR gene, which results in TrpR no longer being able to bind to tryptophan (e.g. the modification is a deletion or mutation of one or more amino acids in the tryptophan binding site, which prevents the conformational change in the TrpR dimer upon tryptophan binding). Methods for identification of such sites are described elsewhere herein.

The crystal structure of TrpR has been solved (see for example, Schevitz et al, Nature, 317(6040), 782-786, 198, doi: 10.1038/317782a0, incorporated herein by reference in its entirety) and several studies on its dimerization and effect of mutations have been published (see, for example, Pal et al., Structure, 25, 867-877, 2017, http://dx.doi.org/10.1016/j.str.2017.04.015 and Sprenger et al., Acta Crystallogr. F. Struct. Biol. Commun., 77(Pt 7), 215-225, 2021, doi: 10.1107/S2053230X21006142, both incorporated herein by reference in their entirety). Using this information and similar publications, a skilled person can identify potential sites for modification (e.g. deletion or substitution).

In another embodiment or concept, the PMS reduces or prevents (e.g. prevents) transcription or expression of a target gene (e.g. tnaA and/or tnaC, or trpR in the recipient bacterium). In one embodiment, the PMS introduces a modification in a target gene (e.g. tnaA and/or tnaC, or trpR) which reduces or prevents (e.g. prevents) expression of the target in the recipient bacterium. In another embodiment, the PMS deletes a target gene (e.g. tnaA and/or tnaC, or trpR in the recipient bacterium).

Concept 16. The transmissible element or bacterium according to any preceding concept, wherein the nucleic acid modifier (as described elsewhere herein) targets and reduces expression (or prevents expression) of an endogenous tryptophan transcriptional repressor (trpR) gene in the recipient bacterium.

Thus, in one embodiment or concept, the PMS targets and reduces expression (or prevents expression) of an endogenous tryptophan transcriptional repressor (trpR) gene in the recipient bacterium. In another embodiment, the PMS expresses a molecule which results in downregulation or prevention of expression of trpR, for example a molecule which blocks binding of the molecular machinery which is necessary for gene transcription, such as blocking binding of DNA or RNA polymerases (e.g. via CRISPRi) in the recipient bacterium. In another embodiment, the PMS is designed to introduce a mutation (e.g. using a base editor as described elsewhere herein) in trpR or its signal sequence which prevents expression of the trpR gene in the recipient bacterium. For example, the PMS may introduce a stop codon (e.g. using a base editor as described elsewhere herein) in the signal sequence or early in the transcribed nucleic acid, preventing expression of trpR in the recipient bacterium.

In any embodiment or concept, the PMS introduces a deletion of one or more nucleotides in an endogenous trpR gene which prevents or reduces (e.g. prevents) transcription or expression of trpR.

Concept 17. The transmissible element or bacterium according to any preceding concept, wherein the nucleic acid modifier (as described elsewhere herein) reduces or prevents enzymatic activity of endogenously expressed tryptophanase (TnaA) in the recipient bacterium.

The TnaA protein is responsible for conversion of tryptophan into indole. When active in the bacteria described herein, TnaA activity reduces the amount of tryptophan which is available to be used in other pathways for the production of IAA (see FIG. 3). Thus, inactivation of the tnaA gene will increase the amount of tryptophan which is available to be used in the biosynthesis of IAA.

In one embodiment or concept, the PMS expresses a molecule (e.g. a peptide) which inhibits enzymatic activity of a protein target (e.g. TnaA).

In one embodiment or concept, the PMS reduces or prevents enzymatic activity of endogenously expressed TnaA in the recipient bacterium. In one embodiment, the PMS expresses an inhibitor molecule which blocks the enzymatic activity of TnaA, thereby preventing or reducing catalytic activity of the endogenous tryptophanase (TnaA) in the recipient bacterium.

Concept 18. The transmissible element or bacterium according to any preceding concept, wherein the nucleic acid modifier (as described elsewhere herein) reduces expression (or prevents expression) of an endogenous tryptophanase (tnaA) gene in the recipient bacterium (for example by introducing a point mutation which introduces a stop codon in the tnaA gene).

In one embodiment or concept, the PMS targets and reduces expression (or prevents expression) of an endogenous tryptophanase (InaA) gene in the recipient bacterium. In one embodiment, the PMS targets and reduces expression (or prevents expression) of a TnaC peptide in the recipient bacterium. In another embodiment, the PMS expresses a molecule which results in downregulation or prevention of expression of endogenous tnaA and/or tnaC, for example a molecule which blocks binding of the molecular machinery which is necessary for gene transcription, such as blocking binding of DNA or RNA polymerases (e.g. via CRISPRi) in the recipient bacterium.

Concept 19. The transmissible element or bacterium according to any preceding concept, wherein the PMS reduces or prevents activity of an endogenously expressed MaCleader sequence in the recipient bacterium.
Concept 20. The transmissible element or bacterium according to any preceding concept, wherein the PMS reduces expression (or prevents expression) of an endogenous MaC leader sequence in the recipient bacterium (for example by introducing a point mutation which introduces a stop codon in the tnaC leader sequence).

In one embodiment or concept, the PMS may modify the target nucleic acid sequence to result in a deletion of tnaCA, which, in addition to the effect of increasing the amount of tryptophan available by reducing its conversion into indole, further increases the amount of tryptophan in the cell through deletion of the tnaC leader peptide and the intergenic region found immediately upstream of tnaA. Bacterial cells may include genes (tnaB) which encode an importer of tryptophan, known as tnaB, located just downstream of tnaA. Deletion of the leader sequence including the TnaC peptide removes the tryptophan dependent regulation of the TnaB importer of tryptophan, thereby further increasing the amount of tryptophan available for the biosynthesis of ALMs.

Thus, in another embodiment or concept, the PMS introduces a mutation (e.g. using a base editor as described elsewhere herein) in tnaA or its leader sequence which reduces or prevents (e.g. prevents) expression of tnaA and/or tuaC in the recipient bacterium. For example, the PMS may introduce a missense mutation or stop codon (e.g. using a base editor as described elsewhere herein) in tnaC, the leader sequence or in tnaA, reducing or preventing (e.g. preventing) expression of tnaA and/or tnaC in the recipient bacterium.

Concept 21. The transmissible element or bacterium according to any preceding concept wherein the PMS increases enzymatic activity of endogenously expressed tryptophan-specific permease (tnaB) in the recipient bacterium.
Concept 22. The transmissible element or bacterium according to any preceding concept wherein the PMS increases expression of an endogenous tryptophan-specific permease (tnaB) gene in the recipient bacterium (for example by introducing a point mutation which introduces a stop codon in the leader sequence of the tnaC gene).
Concept 23. The transmissible element or bacterium according to any preceding concept, wherein the transmissible element or recipient bacterium does not comprise any exogenous genes for the biosynthesis of tryptophan, for example genes encoding one or more genes selected from trpA, trpB, trpC, tbrD and trpE, for example all of trpA, trpC, trpD and trpE.

In an alternative, in any embodiment or concept described herein, the transmissible element or bacterium, does not comprise any heterologous genes for the biosynthesis of tryptophan.

Thus, in any embodiment described herein, the transmissible element or bacterium, does not comprise genes encoding one or more genes selected from trpA, trpB, trpC, trpD and trpE. In one embodiment, the transmissible element or bacterium, does not comprise genes encoding trpA, trpC, and trpD. In one embodiment, the transmissible element or bacterium, does not comprise genes encoding trpA, trpC, and trpE. In one embodiment, the transmissible element or bacterium, does not comprise genes encoding trpC, trpD, and trpE.

In any embodiment described herein, transmissible element or bacterium, does not comprise the genes consisting of trpA, trpB, trpC, trpD and trpE.

In any embodiment described herein, transmissible element or bacterium, does not comprise the genes consisting of trpA, trpC, trpD and trpE.

A gene for the conversion of chorismate to anthranilic acid includes, but is not limited to trpE. Anthranilate synthase (trpE) may be from the genus Escherichia, e.g. E. coli.

A gene for the conversion of anthranilic acid to N-(5-phosphoribosyl)-anthranilate includes, but is not limited to trpD. Phosphoribosyl transferase (trpD) may be from the genus Escherichia, e.g. E. coli.

A gene for the conversion of N-(5-phosphoribosyl)-anthranilate to 1-(o-carboxyphynylamino-1-deoxyribulose-5-phosphate includes, but is not limited to trpC. InGP synthase (trpC)) may be from the genus Escherichia, e.g. E. coli.

A gene for the conversion of 1-(o-carboxyphynylamino-1-deoxyribulose-5-phosphate to indole-3-glycerol phosphate includes, but is not limited to ip4. Tryptophan synthase (trpA)) may be from the genus Escherichia, e.g. E. coli.

Concept 24. The transmissible element or bacterium according to any one of Arrangements 15 to 32, wherein the transmissible element or bacterium comprises no other exogenous genes other than:
I. the PMS;
II. the at least one exogenous gene for the biosynthesis of IAA; and
III. optionally the one or more exogenous genes encoding one or more exporters which is/are capable of exporting IAA;
IV. optionally one or more kill switch(es)

In any embodiment described herein, the transmissible element or bacterium comprises no other heterologous genes other than the one or more heterologous gene(s) for the biosynthesis of IAA and the PMS.

In any embodiment described herein, the transmissible element or bacterium comprises no other heterologous genes other than the one or more heterologous gene(s) for the biosynthesis of IAA, any kill switch genes and the PMS.

In any embodiment described herein, the transmissible element or bacterium comprises no other heterologous genes other than the one or more heterologous gene(s) for the biosynthesis of IAA, the PMS and the heterologous gene encoding an exporter which is capable of exporting IAA.

In any embodiment, the transmissible element or bacterium comprises no other heterologous genes other than the one or more heterologous gene(s) for the biosynthesis of IAA, the heterologous gene encoding an exporter which is capable of exporting IAA, any kill switch genes and the PMS.

In any of the embodiments herein, the one or more genes for the biosynthesis of IAA are each or all under the control of one or more promoter(s) as described elsewhere herein. In one embodiment, the one or more promoter(s) is one or more constitutive promoter(s) (e.g. any of the constitutive promoters described elsewhere herein).

In any of the embodiments herein, the one or more genes for the biosynthesis of IAA are comprised within an operon under the control of a single promoter as described elsewhere herein. In one embodiment, the promoter is a constitutive promoter (e.g. any of the constitutive promoters described elsewhere herein).

In any of the embodiments herein, the gene encoding an exporter which is capable of exporting IAA) out of the recipient bacterium is under the control of a promoter as described elsewhere herein. In one embodiment, the promoter is a constitutive promoter (e.g. any of the constitutive promoters described elsewhere herein).

In any of the embodiments herein, the one or more genes for the biosynthesis of IAA and the gene encoding an exporter which is capable of exporting IAA out of the recipient bacterium are each or all under the control of one or more promoter(s) as described elsewhere herein. In one embodiment, the promoter is a constitutive promoter (e.g. any of the constitutive promoters described elsewhere herein).

In any of the embodiments herein, the one or more genes for the biosynthesis of IAA and the gene encoding an exporter which is capable of exporting IAA out of the recipient bacterium are all under the control of a single promoter as described elsewhere herein. In one embodiment, the promoter is a constitutive promoter (e.g. any of the constitutive promoters described elsewhere herein).

In any of the embodiments herein, the one or more genes for the biosynthesis of IAA and the gene encoding an exporter which is capable of exporting IAA out of the recipient bacterium are comprised within an operon under the control of a single promoter as described elsewhere herein. In one embodiment, the promoter is a constitutive promoter (e.g. any of the constitutive promoters described elsewhere herein).

### Arrangements

Arrangement 1. A transmissible element (e.g. a plasmid, conjugative plasmid, phage or phagemid) for transmission to a recipient bacterium, or a bacterium comprising said transmissible element, wherein the transmissible element comprises:
A) at least one exogenous nucleic acid sequence to the recipient bacterium for the production of a first molecule of interest (MOI) in the recipient bacterium; and
B) a Pathway Modulation System (PMS) which encodes a nucleic acid modifier or a peptide molecule to modulate a target endogenous nucleic acid or protein for the production or consumption (or degradation) of the first MOI in the recipient bacterium.

Arrangement 2. The transmissible element or bacterium according to Arrangement 1 for use as medicament.

Arrangement 3. The transmissible element or bacterium according to Arrangement 1 or Arrangement 2, wherein the exogenous nucleic acid (G1) of A) encodes the first MOI (A).

Arrangement 4. The transmissible element or bacterium according to any preceding Arrangement, wherein the MOI is a therapeutic molecule, such as a peptide molecule (e.g. an antibody fragment, a hormone such as GLP-1, an interleukin, a cytokine or an enzyme) or a small molecule (e.g. a metabolite such as L-DOPA, indole-3-acetic acid, butyrate etc).

Arrangement 5. The transmissible element or bacterium according to any preceding Arrangement, wherein the exogenous nucleic acid of A) encodes one or more (e.g. one) exporter(s) of the first MOI from the recipient bacterium.

Arrangement 6. The transmissible element or bacterium according to any preceding Arrangement, wherein the exogenous nucleic acid of A) further encodes a sequence encoding a signal peptide for the secretion of the first MOI to the periplasm of the recipient bacterium, to the cell surface of the recipient bacterium, or to the extracellular space outside of the recipient bacterium.

Arrangement 7. The transmissible element or bacterium according to any preceding Arrangement, wherein the exogenous nucleic acid of A) encodes, in 5′ to 3′ direction, a promoter, a sequence encoding a signal peptide, at least one nucleic acid for the expression of the first MOI and optionally a further nucleic acid sequence encoding one or more (e.g. one) exporter(s) of the first MOI from the recipient bacterium.

Arrangement 8. The transmissible element or bacterium according to Arrangement 1 or Arrangement 2, wherein the exogenous nucleic acid of A) encodes one or more protein(s) (G1, G2 . . . GX) for the conversion of a second MOI (B) to the first MOI (A).

Arrangement 9. The transmissible element or bacterium according to Arrangement 8, wherein the exogenous nucleic acid of A) encoding one or more protein(s) (G1, G2 . . . GX) is comprised by one or more operons (e.g. by one operon).

Arrangement 10. The transmissible element or bacterium according to Arrangement 9, wherein the one or more operons are under the control of a constitutive promoter.

Arrangement 11. The transmissible element or bacterium according to any one of Arrangements 8 to 10, wherein the first MOI is selected from a therapeutic molecule (e.g. a peptide molecule) and a beneficial or therapeutic metabolite, in particular a beneficial or therapeutic metabolite, or alternatively, the second MOI is a detrimental metabolite.

Arrangement 12. The transmissible element or bacterium according to any one of Arrangements 9 to 11, wherein the exogenous nucleic acid of A) further encodes one or more (e.g. one) exporter(s) of the first MOI, and/or one or more (e.g. one) importer(s) of the second MOT, and/or one or more (e.g. one) importer(s) of any further substrate which is converted by one of the protein(s) (G1, G2 . . . GX) for the conversion of the second MOI (B) to the first MOI (A).

Arrangement 13. The transmissible element or bacterium according to Arrangement 11 or Arrangement 12, wherein the first MOI is a sugar which can be used by the recipient bacterium as a carbon source and the second MOI is rare carbohydrate,
optionally wherein the rare carbohydrate is selected from the group consisting of porphyran, agarose, carrageenan, and any combination thereof and the exogenous nucleic acid of A) encodes one or more protein(s) selected from the group consisting of porphyranase, glycoside hydrolase, sulfatase, galactosidase, and any combination thereof.

Arrangement 14. The transmissible element or bacterium according to Arrangement 13, wherein the exogenous nucleic acid of A) further encodes an importer for the importation of the rare carbohydrate.

Arrangement 15. The transmissible element or bacterium according to any one of Arrangements 8 to 12, wherein the first MOI is indole-3-acetic acid (IAA), and wherein the exogenous nucleic acid of A) encodes at least one exogenous gene for the biosynthesis of IAA.

Arrangement 16. The transmissible element or bacterium according to Arrangement 15, wherein the at least one exogenous gene for the biosynthesis of IAA is selected from:
a) a gene for the conversion of tryptophan to tryptamine (optionally a gene which is tryptophan decarboxylase (tdc)), a gene for the conversion of tryptamine to indole-3-acetaldehyde (IAAld) (optionally a gene which is monoamine oxidase (tynA)), a gene for the conversion of IAAld to IAA (optionally a gene selected from indole-3-acetaldehyde dehydrogenase (iad1) and Indole-3-acetaldehyde oxidase (aao1)), and the second MOI is tryptophan;
b) a gene for the conversion of indole to tryptophan (optionally a gene which is trpB), a gene for the conversion of tryptophan to tryptamine (optionally a gene which is tryptophan decarboxylase (tdc)), a gene for the conversion of tryptamine to indole-3-acetaldehyde (IAAld) (optionally a gene which is monoamine oxidase (tynA)), a gene for the conversion of IAAld to IAA (optionally a gene selected from indole-3-acetaldehyde dehydrogenase (iad1) and Indole-3-acetaldehyde oxidase (aao1)), and the second MOI is indole;
c) a gene for the conversion of tryptophan to indole-3-pyruvic acid (IPyA) (optionally a gene selected from L-tryptophan oxidase (staO), L-tryptophan aminotransferase (aro9), aspartate aminotransferase (aspC), L-tryptophan-pyruvate aminotransferase (taa1) and tryptophan dehydrogenase (tpDH)), a gene for the conversion of IPyA to IAAld (optionally a gene which is indole-3-pyruvate decarboxylase (ipdC)), a gene for the conversion of IAAld to IAA (optionally a gene selected from iad1 and aao1), and the second MOI is tryptophan;
d) a gene for the conversion of indole to tryptophan (optionally a gene which is trpB), a gene for the conversion of tryptophan to indole-3-pyruvic acid (IPyA) (optionally a gene selected from L-tryptophan oxidase (staO), L-tryptophan aminotransferase (aro9), aspartate aminotransferase (aspC), L-tryptophan-pyruvate aminotransferase (taa1) and tryptophan dehydrogenase (trpDH)), a gene for the conversion of IPyA to IAAld (optionally a gene which is indole-3-pyruvate decarboxylase (ipdC)), a gene for the conversion of IAAld to IAA (optionally a gene selected from iad1 and aao1), and the second MOI is indole;
e) a gene for the conversion of tryptophan to indole-3-pyruvic acid (IPyA) (optionally a gene selected from staO, aro9, aspC, taa1 and trpDH), a gene for the conversion of IPyA to IAA (optionally a gene which is an indole-3-pyruvate monooxygenase (e.g. selected from YUC genes, yuc2, and yuc6)), and the second MOI is tryptophan;
f) a gene for the conversion of indole to tryptophan (optionally a gene which is trpB), a gene for the conversion of tryptophan to indole-3-pyruvic acid (IPyA) (optionally a gene selected from staO, aro9, aspC taa1 and trpDH, a gene for the conversion of IPyA to IAA (optionally a gene which is an indole-3-pyruvate monooxygenase (e.g. selected from YUC genes, yuc2, and yuc6)), and the second MOI is indole;
g) a gene for the conversion of tryptophan to indole-3-acetamide (IAM) (optionally a gene which is tryptophan 2-monooxygenase (iaaM)), a gene for the conversion of LAM to IAA (optionally a gene which is indoleacetamide hydrolase (iaaH)), and the second MOI is tryptophan;
h) a gene for the conversion of indole to tryptophan (optionally a gene which is trpB), a gene for the conversion of tryptophan to indole-3-acetamide (IAM) (optionally a gene which is tryptophan 2-monooxygenase (iaaM)), a gene for the conversion of LAM to IAA (optionally a gene which is indoleacetamide hydrolase (iaaH)), and the second MOT is indole;
i) a gene for the conversion of tryptophan to indole-3-acetaldoxime (IAOx) (optionally a gene selected from tryptophan N-monooxygenase (CYP79B2) and tryptophan N-monooxygenase (CYP79B3)), a gene for the conversion of IAOx to indole-3-acetonitrile (IAN) (optionally a gene which is indoleacetaldoxime dehydratase (CYP71A13)), a gene for the conversion of IAN to IAA (optionally a gene which is a nitrilase (e.g. selected from nit1, nit2 and nit3), and the second MOI is tryptophan;
j) a gene for the conversion of indole to tryptophan (optionally a gene which is trpB), a gene for the conversion of tryptophan to indole-3-acetaldoximine (IAOx) (optionally a gene selected from tryptophan N-monooxygenase (CYP79B2) and tryptophan N-monooxygenase (CYP79B3)), a gene for the conversion of IAOx to indole-3-acetonitrile (IAN) (optionally a gene which is indoleacetaldoxime dehydratase (CYP71A13)), a gene for the conversion of IAN to IAA (optionally a gene which is a nitrilase (e.g. selected from nit1, nit2 and n3), and the second MOT is indole;
k) a gene for the conversion of tryptophan to indole-3-acetaldoxime (IAOx) (optionally a gene selected from CYP79B2and CYP79B3), a gene for the conversion of IAOx to indole-3-acetonitrile (IAN) (optionally a gene which is CYP71A13), a gene for the conversion of IAN to LAM (optionally a gene which is nitrile hydratase (nthAB)), a gene for the conversion of LAM to IAA (optionally a gene which is iaaH), and the second MOT is tryptophan;
l) a gene for the conversion of indole to tryptophan (optionally a gene which is trpB), a gene for the conversion of tryptophan to indole-3-acetaldoxime (IAOx) (optionally a gene selected from CYP79B2 and CYP79B3), a gene for the conversion of IAOx to indole-3-acetonitrile (IAN) (optionally a gene which is CYP71A13), a gene for the conversion of IAN to LAM (optionally a gene which is nitrile hydratase (nthAB)), and a gene for the conversion of LAM to IAA (optionally a gene which is iaaH), and the second MOT is indole;
m) any combination of pathways A to L; and
n) any part of pathways A to L which produce IAA as the first MOI.

Arrangement 17. The transmissible element or bacterium according to Arrangement 16, wherein the at least one exogenous gene for the biosynthesis of IAA comprises:
i. Indole-3-pyruvate decarboxylase (ipdC);
ii. Tryptophan-pyruvate aminotransferase 1 (taa1); and
iii. Indole-3-acetaldehyde dehydrogenase (iad1).

Arrangement 18. The transmissible element or bacterium according to Arrangement 17, wherein:
i. the ipdC is from a Pantoea species, e.g. from Pantoea agglomerans; and/or
ii. the taa1 is from an Arabidopsis species, e.g. from Arabidopsis thaliana; and/or
iii. the iad1 is from an Ustilago species, e.g. from Ustilago maydis.

Arrangement 19. The transmissible element or bacterium according to Arrangement 18, wherein:
i. the ipdC is from a Pantoea species, e.g. from Pantoea agglomerans;
ii. the taa1 is from an Arabidopsis species, e.g. from Arabidopsis thaliana; and
iii. the iad1 is from an Ustilago species, e.g. from Ustilago maydis,
wherein genes i. to iii. are each or all under the control of one or more constitutive promoter(s).

Arrangement 20. The transmissible element or bacterium according to any one of Arrangements 15 to 19, wherein the transmissible element further comprises one or more (e.g. one) exogenous genes encoding one or more (e.g. one) exporters which is/are capable of exporting IAA out of the recipient bacterium, optionally under the control of a constitutive promoter.

Arrangement 21. The transmissible element or bacterium according to Arrangement 20, wherein the one or more exogenous genes encoding one or more exporters which is/are capable of exporting IAA encode an auxin efflux protein, for example an exporter selected from:
1. an auxin efflux carrier (AEC) family protein transporter (for example an AEC family protein from a bacterial species);
2. a PIN family protein transporter (for example a PIN family protein transporter from a plant species); and
3. an ABC family protein transporter, such as an ABCD subfamily protein transporter or an ABCB subfamily protein transporter, e.g. an ABCB-PGP sub-family protein transporter (for example where the ABC family protein transporter is from a plant species).

Arrangement 22. The transmissible element or bacterium according to Arrangement 21, wherein the exporter is an AEC family protein transporter.

Arrangement 23. The transmissible element or bacterium according to Arrangement 21 or Arrangement 22, wherein the auxin efflux protein is from a Pantoea species, e.g. Pantoea agglomerans.

Arrangement 24. The transmissible element or bacterium according to any one of Arrangements 20 to 23, wherein the one or more exogenous genes encoding the one or more exporters comprises the nucleotide sequence of SEQ ID No:2.

Arrangement 25. The transmissible element or bacterium according to Arrangement 21, wherein the exporter which is capable of exporting IAA is a PIN transporter (e.g. PIN2 or PIN7), optionally wherein the PIN transporter is from a plant species (e.g. from an Arabidopsis species, e.g. from Arabidopsis thaliana), or wherein the exogenous gene encoding the exporter comprises the nucleotide sequence of SEQ ID No:3.

Arrangement 26. The transmissible element or bacterium according to any one of Arrangements 20 to 25, wherein the at least one exogenous gene for the biosynthesis of IAA and the one or more exogenous genes encoding one or more exporters which is/are capable of exporting IAA are comprised within a single operon under the control of a single constitutive promoter.

Arrangement 27. The transmissible element or bacterium according to Arrangement 26, wherein the operon comprise the following genes in 5′ to 3′ direction:
i. Indole-3-pyruvate decarboxylase (ipdC), optionally from a Pantoea species, e.g. from Pantoea agglomerans;
ii. Tryptophan-pyruvate aminotransferase 1 (taa1), optionally from an Arabidopsis species, e.g. from Arabidopsis thaliana; and
iii. Indole-3-acetaldehyde dehydrogenase (iad1), optionally an Ustilago species, e.g. from Ustilago maydis; and
iv. the one or more exogenous genes encoding one or more exporters of IAA.

Arrangement 28. The transmissible element or bacterium according to any one of Arrangements 15 to 27, wherein the PMS (i) reduces or prevents biologic activity of endogenously expressed tryptophan transcriptional repressor (TrpR) in the recipient bacterium, or (ii) targets and reduces expression (or prevents expression) of an endogenous tryptophan transcriptional repressor (trpR) gene in the recipient bacterium.

Arrangement 29. The transmissible element or bacterium according to any one of Arrangements 15 to 28, wherein the PMS (i) reduces or prevents enzymatic activity of endogenously expressed tryptophanase (TnaA) in the recipient bacterium, or (ii) reduces expression (or prevents expression) of an endogenous tryptophanase (tnaA) gene in the recipient bacterium (for example by introducing a point mutation which introduces a stop codon in the tnaA gene).

Arrangement 30. The transmissible element or bacterium according to any one of Arrangements 15 to 29, wherein the PMS (i) reduces or prevents activity of an endogenously expressed tnaC leader sequence in the recipient bacterium, or (ii) reduces expression (or prevents expression) of an endogenous tnaC leader sequence in the recipient bacterium (for example by introducing a point mutation which introduces a stop codon in the tnaC leader sequence).

Arrangement 31. The transmissible element or bacterium according to any one of Arrangements 15 to 30, wherein the PMS (i) increases enzymatic activity of endogenously expressed tryptophan-specific permease (tnaB) in the recipient bacterium, or (ii) increases expression of an endogenous tryptophan-specific permease (tnaB) gene in the recipient bacterium (for example by introducing a point mutation which introduces a stop codon in the leader sequence of the tnaC gene)

Arrangement 32. The transmissible element or bacterium according to any one of Arrangements 15 to 31, wherein the transmissible element or recipient bacterium does not comprise any exogenous genes for the biosynthesis of tryptophan, for example genes encoding one or more genes selected from trpA, trpB, trpC, trpD and trpE, for example all of trpA, trpC, trpD and trpE.

Arrangement 33. The transmissible element or bacterium according to any one of Arrangements 15 to 32, wherein the transmissible element or bacterium comprises no other exogenous genes other than:
I. the PMS;
II. the at least one exogenous gene for the biosynthesis of IAA; and
III. optionally the one or more exogenous genes encoding one or more exporters which is/are capable of exporting IAA;
IV. optionally one or more kill switch(es).

Arrangement 34. The transmissible element or bacterium according to any preceding Arrangement, wherein the nucleic acid modifier expressed by the PMS of B) reduces expression (or prevents expression) of the target endogenous nucleic acid, and wherein the reduction in expression of the target endogenous nucleic acid (i) increases or maintains the production of the first MOI, or (ii) reduces consumption or degradation of the first MOI.

Arrangement 35. The transmissible element or bacterium according to Arrangement 34, wherein the target endogenous nucleic acid encodes an enzyme (D1) which degrades the first MOI (A), and the reduction of expression of the enzyme increases or maintains (e.g. increases) production of the first MOI by reducing consumption or degradation of the first MOI.

Arrangement 36. The transmissible element or bacterium according to Arrangement 34, wherein:
the exogenous nucleic acid of A) encodes one or more protein(s) (G1, G2 . . . GX) for the conversion of a second MOI (B) to the first MOI (A), and
wherein the recipient bacterium comprises an endogenous pathway for the conversion of one or more substrates (S, S1, S2 . . . etc) into the second MOI (B), and
wherein the target endogenous nucleic acid encodes:
(a) an enzyme (E3) in the endogenous pathway which converts the second MOI (B) into a substrate (S1) which is earlier in the endogenous pathway,
   and the reduction of expression of the enzyme (E3) increases or maintains (e.g. increases) production of the first MOI by reducing degradation of said second MOI (B) to said substrate (S1) in the recipient bacterium; or
(b) an enzyme (E4) in the endogenous pathway which converts a first substrate (S1) used in the production of the second MOI (B) into a second substrate (M1) which is not used in the production of said second MOI,
   and the reduction of expression of the enzyme (E4) increases or maintains (e.g. increases) production of the first MOI by increasing the amount of said first substrate (S1) in the recipient bacterium; or
(c) an enzyme (E5) in the endogenous pathway which converts a first substrate (S1) of the endogenous pathway into a second substrate (S) which is earlier in the endogenous pathway,
   and the reduction of expression of the enzyme (E5) increases or maintains (e.g. increases) production of the first MOI by reducing degradation of said first substrate (S1) in the recipient bacterium; or
(d) an exporter (EXP2) of a substrate in the endogenous pathway (S1),
   and the reduction of expression of the exporter (EXP2) increases or maintains (e.g. increases) production of the first MOI by increasing the amount of said substrate (S1) in the recipient bacterium.

Arrangement 37. The transmissible element or bacterium according to Arrangement 34, wherein:
the exogenous nucleic acid of A) encodes one or more protein(s) (G1, G2 . . . GX) for the conversion of a second MOI (B) to the first MOI (A); and
wherein the recipient bacterium comprises:
   a first endogenous pathway comprising at least one enzyme for the conversion of one or more substrates (S, S1, S2 . . . etc) into the second MOI (B), and
   a second endogenous pathway comprising at least one enzyme (E1) which is common to the first and second endogenous pathways,
and the at least one common enzyme (E1) can convert a first substrate of the first endogenous pathway (S) and a second, different substrate of the second endogenous pathway (X); and
wherein the target endogenous nucleic acid encodes:
(a) an importer (IMP2) which imports the second, different substrate of the second endogenous pathway (X),
   and the reduction of expression of the importer (IMP2) increases or maintains (e.g. increases) production of the first MOI by increasing the availability and/or activity of said common enzyme (E1) to convert said first substrate (S) in the first endogenous pathway in the recipient bacterium; or
(b) an enzyme which is part of the second endogenous pathway and is for the production of the second, different substrate of the second endogenous pathway (Q, for example is an endogenous enzyme (P1) which is part of the second endogenous pathway and is for the direct production of the second, different substrate of the second endogenous pathway (X),
   and the reduction of expression of the enzyme (P1) increases or maintains (e.g. increases) production of the first MOI by reducing the amount of the second, different substrate of the second endogenous pathway (X), thereby increasing the availability and/or activity of said common enzyme (E1) to convert said first substrate (S) in the first endogenous pathway in the recipient bacterium.

Arrangement 38. The transmissible element or bacterium according to any one of Arrangements 34 to 37, wherein the nucleic acid modifier expressed by the PMS of B) reduces expression (or prevents expression) of the target endogenous nucleic acid by (i) introducing a modification to one or more nucleotides in the endogenous DNA (e.g. comprised by the chromosome or a plasmid) of the recipient bacterium, for example by one or more point mutations in one or more codons of the target endogenous nucleic acid, or (ii) producing a nucleic acid inhibitor molecule, for example a small regulatory RNA (sRNA).

Arrangement 39. The transmissible element or bacterium according to Arrangement 38(i), wherein:
a. the target endogenous nucleic acid encodes a protein (such as an enzyme, importer or exporter) and the modification introduces a stop codon in the target endogenous nucleic acid, thereby preventing expression of said protein, or truncating expression such that any expressed protein is non-functional; or
b. the target endogenous nucleic acid comprises a promoter to control expression of a protein expressed from the target endogenous nucleic acid (such as an enzyme, importer or exporter) and the modification modifies the promoter, thereby reducing or preventing expression of said protein; or
c. the target endogenous nucleic acid comprises a ribosome binding site or a translational start site to control expression of a protein expressed from the target endogenous nucleic acid (such as an enzyme, importer or exporter) and the modification modifies the ribosome binding site or the translational start site, thereby reducing or preventing expression of said protein.

Arrangement 40. The transmissible element or bacterium according to Arrangement 38(ii), wherein:
a. the target endogenous nucleic acid encodes a protein (such as an enzyme, importer or exporter) and the nucleic acid inhibitor molecule is a small regulatory RNA (sRNA) which binds to the transcribed mRNA encoding the said protein, thereby preventing expression of said protein, or truncating expression such that any expressed protein is non-functional; or
b. the nucleic acid inhibitor molecule is a small regulatory RNA (sRNA) which binds to and causes degradation an mRNA molecule expressed from the target endogenous nucleic acid encoding the protein (e.g. a or the enzyme, or a or the importer, or a or the exporter), thereby reducing or preventing the translation of said target endogenous protein.

Arrangement 41. The transmissible element or bacterium according to any one of Arrangements 1 to 33, wherein the nucleic acid modifier expressed by the PMS of B) modifies the target endogenous nucleic acid to introduce a mutation in a protein expressed from said target endogenous nucleic acid, which mutation reduces the activity or function of said expressed protein, and wherein the reduction in activity or function of the expressed protein (i) increases or maintains the production of the first MOI, or (ii) reduces consumption or degradation of the first MOI.

Arrangement 42. The transmissible element or bacterium according to Arrangement 41, wherein the expressed protein is an enzyme (D1) which degrades the first MOI (A), and the mutation in the enzyme reduces the activity or function of said enzyme and increases or maintains production of the first MOI by reducing consumption or degradation of the first MOI.

Arrangement 43. The transmissible element or bacterium according to Arrangement 41, wherein:
the exogenous nucleic acid of A) encodes one or more protein(s) (G1, G2 . . . GX) for the conversion of a second MOI (B) to the first MOI (A), and
wherein the recipient bacterium comprises an endogenous pathway for the conversion of one or more substrates (S, S1, S2 . . . etc) into the second MOI (B), and
wherein the expressed protein is:
(a) an enzyme (E3) in the endogenous pathway which converts the second MOI (B) into a substrate (S1) which is earlier in the endogenous pathway,
   and the mutation in the enzyme (E3) reduces the activity or function of said enzyme, and increases or maintains (e.g. increases) production of the first MOI by reducing degradation of said second MOI (B) to said substrate (S1) in the recipient bacterium; or
(b) an enzyme (E4) in the endogenous pathway which converts a first substrate (S1) used in the production of the second MOI (B) into a second substrate (M1) which is not used in the production of said second MOI,
   and the mutation in the enzyme (E4) reduces the activity or function of said enzyme, and increases or maintains (e.g. increases) production of the first MOI by increasing the amount of said first substrate (S1) in the recipient bacterium; or
(c) an enzyme (E5) in the endogenous pathway which converts a first substrate (S1) of the endogenous pathway into a second substrate (S) which is earlier in the endogenous pathway,
   and the mutation in the enzyme (E5) reduces the activity or function of said enzyme, and increases or maintains (e.g. increases) production of the first MOI by reducing degradation of said first substrate (S1) in the recipient bacterium; or
(d) an exporter (EXP2) of a substrate in the endogenous pathway (S1),
   and the mutation in the exporter (EXP2) reduces the activity or function of said exporter, and increases or maintains (e.g. increases) production of the first MOI by increasing the amount of the substrate (S1) in the recipient bacterium.

Arrangement 44. The transmissible element or bacterium according to Arrangement 41, wherein:
the exogenous nucleic acid of A) encodes one or more protein(s) (G1, G2 . . . GX) for the conversion of a second MOI (B) to the first MOI (A); and
wherein the recipient bacterium comprises:
   a first endogenous pathway comprising at least one enzyme for the conversion of one or more substrates (S, S1, S2 . . . etc) into the second MOI (B), and
   a second endogenous pathway comprising at least one enzyme (E1) which is common to the first and second endogenous pathways,
and the at least one common enzyme (E1) can convert a first substrate of the first endogenous pathway (S) and a second, different substrate of the second endogenous pathway (X); and
wherein the expressed protein is:
(a) an importer (IMP2) which imports the second, different substrate of the second endogenous pathway (X),
   and the mutation in the importer (IMP2) reduces the activity or function of said importer, and increases or maintains (e.g. increases) production of the first MOI by increasing the availability and/or activity of the common enzyme (E1) to convert said first substrate (S) in the first endogenous pathway in the recipient bacterium; or
(b) an enzyme which is part of the second endogenous pathway and is for the production of the second, different substrate of the second endogenous pathway (X), for example is an endogenous enzyme (P1) which is part of the second endogenous pathway and is for the direct production of the second, different substrate of the second endogenous pathway (X),
   and the mutation in the enzyme (P1) reduces the activity or function of said enzyme, and increases or maintains (e.g. increases) production of the first MOI by reducing the amount of the second, different substrate of the second endogenous pathway (X), thereby increasing the availability and/or activity of said common enzyme (E1) to convert said first substrate (S) in the first endogenous pathway in the recipient bacterium.

Arrangement 45. The transmissible element or bacterium according to any one of Arrangements 41 to 44, wherein the nucleic acid modifier expressed by the PMS of B) introduces a mutation in the expressed protein by targeted modification of one or more nucleotides in the endogenous DNA (e.g. comprised by the chromosome or a plasmid) of the recipient bacterium, for example by one or more point mutations in one or more codons of the target endogenous nucleic acid.

Arrangement 46. The transmissible element or bacterium according to Arrangement 45, wherein:
a. the modification modifies the active site of the expressed enzyme, thereby reducing or preventing the conversion of a substrate of said expressed enzyme (e.g. by changing the kinetics of binding of the substrate, or preventing binding of the substrate);
b. the modification modifies the channel of the expressed importer or exporter, thereby reducing or preventing the movement of a substrate through said channel;
c. the modification prevents the correct folding of the expressed protein, thereby preventing the binding of any substrate, or preventing the expressed protein from performing its function (e.g. transport of a substrate where the expressed protein is an importer or exporter, or performing a catalytic reaction where the expressed protein is an enzyme);
d. the modification prevents the correct assembly of a muti-unit protein which comprises the expressed protein as a sub-unit, thereby preventing the multi-unit protein from performing its function (e.g. transport of a substrate where the multi-unit protein is an importer or exporter, or performing a catalytic reaction where the multi-unit protein is an enzyme).

Arrangement 47. The transmissible element or bacterium according to any one of Arrangements 1 to 33, wherein the nucleic acid modifier expressed by the PMS of B) increases expression of the target endogenous nucleic acid, and wherein the increase in expression of the target endogenous nucleic acid increases or maintains the production of the first MOI.

Arrangement 48. The transmissible element or bacterium according to Arrangement 47, wherein the target endogenous nucleic acid encodes an exporter (EXP1) which is capable of exporting the first MOI (A) to the periplasm of the recipient bacterium, to the cell surface of the recipient bacterium, or to the extracellular space outside of the recipient bacterium, and
the increase of expression of the exporter increases or maintains (e.g. increases) production of the first MOI by increasing export of said first MOI to the periplasm of the recipient bacterium, to the cell surface of the recipient bacterium, or to the extracellular space outside of the recipient bacterium.

Arrangement 49. The transmissible element or bacterium according to Arrangement 47, wherein:
the exogenous nucleic acid of A) encodes one or more protein(s) (G1, G2 . . . GX) for the conversion of a second MOI (B) to the first MOI (A), and
wherein the recipient bacterium comprises an endogenous pathway for the conversion of one or more substrates (S, S1, S2 . . . etc) into the second MOI (B), and
wherein the target endogenous nucleic acid encodes:
(a) an enzyme (E1) in the endogenous pathway which converts a first substrate (S) into a second substrate (S1) in the endogenous pathway,
   and the increase of expression of the enzyme increases or maintains (e.g. increases) production of the first MOI by increasing the amount of said second substrate (S1) in the recipient bacterium; or
(b) an enzyme (E2) in the endogenous pathway which converts a first substrate (S) into the second MOI (B) in the endogenous pathway,
   and the increase of expression of the enzyme increases or maintains (e.g. increases) production of the first MOI by increasing the amount of said second MOI (B) in the recipient bacterium; or
(c) an importer (IMP1) in the endogenous pathway which imports a first substrate (S) into the recipient bacterium for use in the endogenous pathway,
   and the increase of expression of the importer (IMP1) increases or maintains (e.g. increases) production of the first MOI by increasing the amount of said first substrate (S) in the recipient bacterium.

Arrangement 50. The transmissible element or bacterium according to Arrangement 47, wherein:
the exogenous nucleic acid of A) encodes one or more protein(s) (G1, G2 . . . GX) for the conversion of a second MOI (B) to the first MOI (A); and
wherein the recipient bacterium comprises:
   a first endogenous pathway comprising at least one enzyme for the conversion of one or more substrates (S, S1, S2 . . . etc) into the second MOI (B), and
   a second endogenous pathway, which is a branch of the first endogenous pathway and comprises at least one substrate is a substrate (S1) which is common to the first and second endogenous pathways; and
wherein the target endogenous nucleic acid encodes an enzyme (E6) which converts a second substrate of the second endogenous pathway (S2) to the common substrate (S1),
and the increase in expression of the enzyme (E6) increases or maintains (e.g. increases) production of the first MOI by increasing the amount of the common substrate (S1) in the recipient bacterium.

Arrangement 51. The transmissible element or bacterium according to any one of Arrangements 47 to 50, wherein the nucleic acid modifier expressed by the PMS of B) increases expression of the target endogenous nucleic acid by (i) introducing a modification to one or more nucleotides in the endogenous DNA (e.g. comprised by the chromosome or a plasmid) of the recipient bacterium or (ii) producing a nucleic acid activator molecule, for example a small regulatory RNA (sRNA).

Arrangement 52. The transmissible element or bacterium according to Arrangement 51(i), wherein:
a. the target endogenous nucleic acid encodes a gene encoding a protein (e.g. a or the enzyme, or a or the importer, or a or the exporter), and the modification modifies a repressor of said gene, thereby reducing or preventing expression of the repressor, which de-represses expression of said gene and increases expression of the target endogenous nucleic acid; or
b. the target endogenous nucleic acid encodes a gene encoding a protein (e.g. a or the enzyme, or a or the importer, or a or the exporter) preceded by a leader sequence, and the modification modifies nucleic acid encoding the leader sequence in said gene, thereby increasing expression of said protein; or
c. the target endogenous nucleic acid encodes a gene encoding a protein (e.g. a or the enzyme, or a or the importer, or a or the exporter) and the modification modifies the promoter of said gene, thereby increasing expression of said protein, for example by introducing a mutation which increases the strength of the promoter of the target gene; or
d. the target endogenous nucleic acid comprises a ribosome binding site or a translational start site to control expression of a protein expressed from the target endogenous nucleic acid (such as an enzyme, importer or exporter) and the modification modifies the ribosome binding site or the translational start site, thereby increasing expression of said protein, for example by introducing one or more mutations to optimise the sequence of the ribosome binding site or the translational start site.

Arrangement 53. The transmissible element or bacterium according to Arrangement 51(ii), wherein:
a. the target endogenous nucleic acid encodes a protein (e.g. a or the enzyme, or a or the importer, or a or the exporter), and the nucleic acid activator molecule binds a repressor of said gene, thereby reducing or preventing expression of the repressor, which de-represses expression of said gene and increases expression of the target endogenous nucleic acid; or
b. the target endogenous nucleic acid encodes a translation activator mRNA and the nucleic acid activator molecule is a small regulatory RNA (sRNA) which binds the mRNA and activates translation of said translation activator.

Arrangement 54. The transmissible element or bacterium according to any one of Arrangements 1 to 33, wherein the nucleic acid modifier expressed by the PMS of B) modifies the target endogenous nucleic acid to introduce a mutation in a protein expressed from said target endogenous nucleic acid, which mutation increases the activity or function of said expressed protein, and wherein the increase in activity or function of the expressed protein increases or maintains the production of the first MOI.

Arrangement 55. The transmissible element or bacterium according to Arrangement 54, wherein the expressed protein is an exporter (EXP1) which is capable of exporting the first MOI (A) to the periplasm of the recipient bacterium, to the cell surface of the recipient bacterium, or to the extracellular space outside of the recipient bacterium,
and the mutation in the exporter increases the activity or function of said exporter, and increases or maintains (e.g. increases) production of the first MOI by increasing export of said first MOI to the periplasm of the recipient bacterium, to the cell surface of the recipient bacterium, or to the extracellular space outside of the recipient bacterium.

Arrangement 56. The transmissible element or bacterium according to Arrangement 54, wherein:
the exogenous nucleic acid of A) encodes one or more protein(s) (G1, G2 . . . GX) for the conversion of a second MOI (B) to the first MOI (A), and
wherein the recipient bacterium comprises an endogenous pathway for the conversion of one or more substrates (S, S1, S2 . . . etc) into the second MOI (B), and
wherein the expressed protein is:
(a) an enzyme (E1) in the endogenous pathway which converts a first substrate (S) into a second substrate (S1) in the endogenous pathway,
   and the mutation in the enzyme increases the activity or function of said enzyme, and increases or maintains (e.g. increases) production of the first MOI by increasing the amount of said second substrate (S1) in the recipient bacterium; or
(b) an enzyme (E2) in the endogenous pathway which converts a first substrate (S) into the second MOI (B) in the endogenous pathway,
   and the mutation in the enzyme increases the activity or function of said enzyme, and increases or maintains (e.g. increases) production of the first MOI by increasing the amount of said second MOI (B) in the recipient bacterium; or
(c) an importer (IMP1) in the endogenous pathway which imports a first substrate (S) into the recipient bacterium for use in the endogenous pathway,
   and the mutation in the importer (IMP1) increases the activity or function of said importer, and increases or maintains (e.g. increases) production of the first MOI by increasing the amount of said first substrate (S) in the recipient bacterium.

Arrangement 57. The transmissible element or bacterium according to Arrangement 54, wherein:
the exogenous nucleic acid of A) encodes one or more protein(s) (G1, G2 . . . GX) for the conversion of a second MOI (B) to the first MOI (A); and
wherein the recipient bacterium comprises:
   a first endogenous pathway comprising at least one enzyme for the conversion of one or more substrates (S, S1, S2 . . . etc) into the second MOI (B), and
   a second endogenous pathway comprising at least one enzyme (E1) which is common to the first and second endogenous pathways,
and the at least one common enzyme (E1) can convert a first substrate of the first endogenous pathway (S) and a second, different substrate of the second endogenous pathway (X); and
wherein the expressed protein is an enzyme (E6) which converts a second substrate of the second endogenous pathway (S2) to the common substrate (S1),
and the mutation in the enzyme increases the activity or function of said enzyme, and increases or maintains (e.g. increases) production of the first MOI by increasing the amount of the common substrate (S1) in the recipient bacterium.

Arrangement 58. The transmissible element or bacterium according to any one of Arrangements 54 to 57, wherein the nucleic acid modifier expressed by the PMS of B) introduces a mutation in the expressed protein by targeted modification of one or more nucleotides in the endogenous DNA (e.g. comprised by the chromosome or a plasmid) of the recipient bacterium, for example by one or more point mutations in one or more codons of the target endogenous nucleic acid.

Arrangement 59. The transmissible element or bacterium according to Arrangement 58, wherein:
a. the modification modifies the active site of the expressed enzyme, thereby increasing the conversion of a substrate of said expressed enzyme (e.g. by changing the kinetics of binding of the substrate);
b. the modification modifies the channel of the expressed importer or exporter, thereby increasing the movement of a substrate through said channel.

Arrangement 60. The transmissible element or bacterium according to any of Arrangements 1 to 33, wherein the peptide molecule expressed by the PMS of B) is an inhibitor to reduce or prevent (e.g. block) activity or function of the target endogenous protein in the recipient bacterium, wherein the reduction or prevention in the activity or function of said protein (i) increases or maintains the production of the first MOI, or (ii) prevents the consumption of the first MOI.

Arrangement 61. The transmissible element or bacterium according to Arrangement 60, wherein the target endogenous protein is an enzyme (D1) which degrades the first MOI (A), and the reduction or prevention of activity or function of said protein increases or maintains (e.g. increases) production of the first MOI by reducing consumption or degradation of the first MOI.

Arrangement 62. The transmissible element or bacterium according to Arrangement 60, wherein:
the exogenous nucleic acid of A) encodes one or more protein(s) (G1, G2 . . . GX) for the conversion of a second MOI (B) to the first MOI (A), and
wherein the recipient bacterium comprises an endogenous pathway for the conversion of one or more substrates (S, S1, S2 . . . etc) into the second MOI (B), and
wherein the target endogenous protein is:
(a) an enzyme (E3) in the endogenous pathway which converts the second MOI (B) into a substrate (S1) which is earlier in the endogenous pathway,
   and the reduction or prevention of the activity or function of said enzyme (E3) increases or maintains (e.g. increases) production of the first MOI by reducing degradation of said second MOI (B) to said substrate (S1) in the recipient bacterium; or
(b) an enzyme (E4) in the endogenous pathway which converts a first substrate (S1) used in the production of the second MOI (B) into a second substrate (M1) which is not used in the production of the second MOI,
   and the reduction or prevention of the activity or function of said enzyme (E4) increases or maintains (e.g. increases) production of the first MOI by increasing the amount of said first substrate (S1) in the recipient bacterium; or
(c) an enzyme (E5) in the endogenous pathway which converts a first substrate (S1) of the endogenous pathway into a second substrate (S) which is earlier in the endogenous pathway,
   and the reduction or prevention of the activity or function of said enzyme (E5) increases or maintains (e.g. increases) production of the first MOI by reducing degradation of said first substrate (S1) in the recipient bacterium; or
(d) an exporter (EXP2) of a substrate in the endogenous pathway (S1),
   and the reduction or prevention of the activity or function of said exporter (EXP2) increases or maintains (e.g. increases) production of the first MOI by increasing the amount of said substrate (S1) in the recipient bacterium.

Arrangement 63. The transmissible element or bacterium according to Arrangement 60, wherein:
the exogenous nucleic acid of A) encodes one or more protein(s) (G1, G2 . . . GX) for the conversion of a second MOI (B) to the first MOI (A); and
wherein the recipient bacterium comprises:
   a first endogenous pathway comprising at least one enzyme for the conversion of one or more substrates (S, S1, S2 . . . etc) into the second MOI (B), and
   a second endogenous pathway comprising at least one enzyme (E1) which is common to the first and second endogenous pathways,
and the at least one common enzyme (E1) can convert a first substrate of the first endogenous pathway (S) and a second, different substrate of the second endogenous pathway (X); and
wherein the target endogenous protein is:
(a) an importer (IMP2) which imports the second, different substrate of the second endogenous pathway (X),
   and the reduction or prevention of the activity or function of said importer (IMP2) increases or maintains (e.g. increases) production of the first MOI by increasing the availability and/or activity of said common enzyme (E1) to convert said first substrate (S) in the first endogenous pathway in the recipient bacterium; or
(b) an enzyme which is part of the second endogenous pathway and is for the production of the second, different substrate of the second endogenous pathway (X), for example is an endogenous enzyme (P1) which is part of the second endogenous pathway and is for the direct production of the second, different substrate of the second endogenous pathway (X),
   and the reduction or prevention of the activity or function of the enzyme (P1) increases or maintains (e.g. increases) production of the first MOI by increasing the availability and/or activity of said common enzyme (E1) to convert said first substrate (S) in the first endogenous pathway in the recipient bacterium.

Arrangement 64. The transmissible element or bacterium according to any one of Arrangements 60 to 63, wherein the peptide molecule inhibitor:
a. binds to the active site of the target endogenous enzyme but cannot be converted by the enzyme, thereby preventing a new substrate from being bound and converted by said enzyme; or
b. binds in a channel of the target endogenous importer or exporter, but does not get transported through the channel of said importer or exporter, thereby preventing transport of any other substrate through said channel; or
c. binds to the target endogenous protein and changes the conformation of said protein, thereby reducing or preventing the endogenous function of said target endogenous protein (such as preventing correct folding of said protein or correct assembly of said protein as part of a multi-unit protein).

Arrangement 65. The transmissible element or bacterium according to any one of Arrangements 1 to 64, wherein the exogenous nucleic acid of A):
a) does not comprise any reporter genes; and/or
b) does not comprise any antibiotic resistance genes.

Arrangement 66. The transmissible element or bacterium according to any preceding Arrangement, wherein the PMS:
a) does not modulate the target endogenous nucleic add in the recipient bacterium which is a virulence gene, antibiotic resistance gene, or an essential gene; and/or
b) does not modulate the target endogenous nucleic add in the recipient bacterium which is a toxin gene; and/or
c) does not modulate the target endogenous nucleic acid in the recipient bacterium by introducing a double stranded break in DNA or RNA comprised by the recipient bacterium; and/or
d) does not modulate the target endogenous nucleic acid in the recipient bacterium by removing a (second) plasmid comprised by the recipient bacterium, wherein the (second) plasmid is not the transmissible element; and/or
e) does not modulate the target endogenous nucleic acid in the recipient bacterium by killing the recipient bacterium.

Arrangement 67. The transmissible element or bacterium according to any preceding Arrangement, wherein the transmissible element:
a) is capable of being introduced into the recipient bacterium without heat stock; and/or
b) is capable of being introduced into a recipient bacterium without electroporation; and/or
c) is capable of being introduced into a recipient bacterium without in vitro transformation techniques, including chemical induction (e.g. using calcium).

Arrangement 68. The transmissible element or bacterium according to any preceding Arrangement, wherein the PMS is under the control of an inducible promoter.

Arrangement 69. The transmissible element or bacterium according to any preceding Arrangement, wherein the promoter is a promoter selected from a RelB, BolA Hya, YiaG and a RpoH promoter, such as a promoter selected from a RelB promoter sequence, σ70; a BolA promoter sequence, σS, σ70; a Hya promoter sequence, σS, σ70; a ViaG promoter sequence, σS; a RpoH promoter sequence P1, σ70; a RpoH promoter sequence P2, σS; a RpoHpromoter sequence P3, σ24; a RpoH promoter sequence P4, σ70; a RpoH promoter sequence P5, σ70; and a RpoH promoter sequence P6, σ54, in particular a promoter having a nucleotide sequence selected from any one of Seq ID Nos: 68 to 77, or a nucleotides sequence having 90% (or 95%) homology thereto.

Arrangement 70. The transmissible element or bacterium according to any one of Arrangements 1 to 67, wherein the PMS is under the control of a constitutive promoter, for example where the constitutive promoter is a strong constitutive promoter having an Anderson score >0.4 (e.g. >0.5), e.g. a tac promoter, for example a Pc-tga promoter having the sequence of SEQ ID No: 1, or a Pc promoter have the sequence of SEQ ID No: 102.

Arrangement 71. The transmissible element or bacterium according to any preceding Arrangement, wherein the PMS encodes a nucleic acid modifier selected from the group consisting of:
a) a base editor;
b) a prime editor;
c) an RNAi system;
d) a CRISPRi system; and
e) a nuclease (e.g. a CRISPR/Cas system, a TALEN or a zinc finger).

Arrangement 72. The transmissible element or bacterium according to any preceding Arrangement, wherein the nucleic acid modifier is a base editor.

Arrangement 73. The transmissible element according to Arrangement 72, wherein the base editor further comprises a modified nuclease that is modified to be unable to perform DNA double strand breaks, while retaining its DNA binding capacity and is fused to a domain to perform base editing.

Arrangement 74. The transmissible element or bacterium according to Arrangement 72 or Arrangement 73, wherein the base editor is selected from:
A. a Cytosine Base Editor (CBE) which converts C:G into T:A;
B. an Adenine Base Editor (ABE) which converts A:T into G:C;
C. a Cytosine Guanine Base Editors (CGBE) which converts C:G into G:C;
D. a Cytosine Adenine Base Editors (CABEs) which converts C:G into A:T;
E. an Adenine Cytosine Base Editor (ACBE) which converts A:T into C:G;
F. an Adenine Thymine Base Editor (ATBE) which converts A:T into T:A; or
G. a Thymine Adenine Base Editor (TABE) which converts T:A into A:T.

Arrangement 75. The transmissible element or bacterium according to Arrangement 74, wherein the base editor is selected from the group consisting of: POBEC1, rAPOBEC1, APOBEC1 mutant or evolved version (evoAPOBEC1), APOBEC homologs (APOBEC3A (eA3A), Anc689), Cytidine deaminase 1 (CDA1), evoCDA 1, FERNY, evoFERNY, BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Cas12a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRYCBE, SpG-CBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3 and AncBE4.

Arrangement 76. The transmissible element or bacterium according to any one of Arrangements 73 to 75, wherein the PMS encodes a dCas9 (dead-Cas9) or nCas9 (nickase Cas9) fused to a cytosine or adenosine deaminase domain and a guide RNA or crRNA.

Arrangement 77. The transmissible element or bacterium according to any one of Arrangements 72 to 75, wherein the PMS encodes a fusion protein comprising a polypeptide (Px), wherein Px
I. comprises an amino acid sequence that is at least 90% identical to a sequence selected from SEQ ID Nos:1-5, in particular SEQ ID No:4; and
II. is fused to a base editor selected from an adenine base editor (ABE, e.g. an adenosine deaminase), a cytosine base editor (CBE, e.g. a cytidine deaminase or an apolipoprotein B mRNA-editing complex (APOBEC) family deaminase), a cytosine guanine base editor (CGBE), a cytosine adenine base editor (CABE), an adenine cytosine base editor (ACBE), an adenine thymine base editor (ATBE), a thymine adenine base editor (TABE), and a uracil DNA glycosylase inhibitor (UGI) protein, in particular a CBE, such as a PmCDA1 cytidine deaminase (optionally in combination with a UGI protein); and
the PMS further encodes a guide RNA or crRNA.

Arrangement 78. The transmissible element or bacterium according to Arrangement 77, wherein the base editor is a sea lamprey base editor, for example wherein the base editor is a PmCDA1 cytidine deaminase having the amino acid sequence encoded by SEQ ID No:19.

Arrangement 79. The transmissible element or bacterium according to Arrangement 77 or Arrangement 78, wherein the base editor is fused to the C-terminus of the amino acid sequence of part I, optionally wherein the base editor (e.g. PmCDA1 cytidine deaminase) is fused via a peptide linker to the amino acid sequence, for example a linker of from 10 to 20 amino acids (e.g. about 16 amino acids), such as a linker having the amino acid sequence of SEQ ID No:17.

Arrangement 80. The transmissible element or bacterium according to Arrangement 79 wherein a UGI protein is fused to the base editor, optionally wherein the UGI protein has an amino acid sequence of SEQ ID No:21.

Arrangement 81. The transmissible element or bacterium according to Arrangement 80, wherein the UGI protein is fused to the base editor via a peptide linker, for example a linker of from about 8 to 12 amino acids (e.g. about 10 amino acids), such as a linker having the amino acid sequence of SEQ ID No:23 and optionally wherein the linker is fused to the C-terminus of the base editor, and the UGI protein is fused to the C-terminus of the linker.

Arrangement 82. The transmissible element or bacterium according to any one of Arrangements 77 to 81, wherein the fusion protein is in the form of a fusion protein complex which comprises at least 3 further polypeptides,
wherein the three further polypeptides each have an amino acid sequence that is at least 90% identical to a sequence selected from the sequences of SEQ ID No:1, SEQ ID No:2, SEQ ID No:4 and SEQ ID No:5,
and wherein the fusion protein complex comprises amino acid sequences that are at least 90% identical to each of the sequences of SEQ ID No:1, SEQ ID No:2, SEQ ID No:4 and SEQ ID No:5.

Arrangement 83. The transmissible element or bacterium according to Arrangement 82, wherein the protein complex comprises at least 4 further polypeptides, and wherein the fusion protein complex comprises amino acid sequences that are at least 90% identical to each of the sequences of SEQ ID No:1, SEQ ID No:2, SEQ ID No:3, SEQ ID No:4 and SEQ ID No:5.

Arrangement 84. The transmissible element or bacterium according to any one of Arrangements 81 to 83, wherein the base editor of II is fused to the C-terminus of the amino acid sequence of part I.

Arrangement 85. The transmissible element or bacterium according to Arrangement 71, wherein the nucleic acid modifier is a prime editor.

Arrangement 86. The transmissible element or bacterium according to Arrangement 85, wherein the PMS encodes a Cas9 (dead-Cas9) or nCas9 (nickase Cas9) fused to a reverse transcriptase domain and a pegRNA (prime editing guide RNA) which directs the prime editor to the target endogenous nucleic acid to modify said target nucleic acid and modulate the production or consumption of the first MOI.

Arrangement 87. The transmissible element or bacterium according to Arrangement 85 or Arrangement 86, wherein the prime editor is selected from the group consisting of: PE1, PE1-M1, PE1-M2, PE1-M3, PE1-M6, PE1-M15, PE1-M3inv, PE2, PE3 and PE3b.

Arrangement 88. The transmissible element or bacterium according to Arrangement 71, wherein the nucleic acid modifier is an RNAi system.

Arrangement 89. The transmissible element or bacterium according to Arrangement 88, wherein the RNAi system is a small regulatory RNA (sRNA), for example an artificial sRNA.

Arrangement 90. The transmissible element or bacterium according to Arrangement 71, wherein the nucleic acid modifier is a CRISPRi system.

Arrangement 91. The transmissible element or bacterium according to Arrangement 90, wherein the CRISPRi system encodes a Cas9 (dead-Cas9) fused to one or more transcriptional repressor(s) and a gRNA or crRNA which directs the CRISPRi system to the target endogenous nucleic acid to modify said target nucleic acid and modulate the production or consumption of the first MOI.

Arrangement 92. The transmissible element or bacterium according to Arrangement 91, wherein the CRISPRi system encodes a system selected from: dCas9-KRAB, dCas9-KRAB-MeCP2 and dCas9-SALL1-SDS3.

Arrangement 93. The transmissible element or bacterium according to Arrangement 90, wherein the CRISPRi system comprises:
a) a nucleotide sequence that encodes a polypeptide which comprises an amino acid sequence that is at least 90% identical to SEQ ID NO:2;
b) a nucleotide sequence that encodes a polypeptide which comprises an amino acid sequence that is at least 90% identical to SEQ ID NO:4; and
c) a nucleotide sequence that encodes a polypeptide which comprises an amino acid sequence that is at least 90% identical to SEQ ID NO:5; and
d) a guide RNA or crRNA.

Arrangement 94. The transmissible element or bacterium according to Arrangement 93, wherein the CRISPRi system further comprises a nucleotide sequence that encodes a polypeptide which comprises an amino acid sequence that is at least 90% identical to SEQ ID NO:1.

Arrangement 95. The transmissible element or bacterium according to Arrangement 93 or Arrangement 94, wherein the CRISPRi system further comprises a nucleotide sequence that encodes a polypeptide which comprises an amino acid sequence that is at least 90% identical to SEQ ID NO:3.

Arrangement 96. The transmissible element or bacterium according to Arrangement 71, wherein the nucleic acid modifier is a nuclease and optionally a guide RNA or crRNA.

Arrangement 97. The transmissible element or bacterium according to Arrangement 96, wherein the nuclease cannot introduce double stranded breaks in the target endogenous nucleic acid, and the gRNA or crRNA directs the nuclease to the target endogenous nucleic acid to modify said target nucleic acid and modulate the production or consumption of the first MOI.

Arrangement 98. The transmissible element or bacterium according to Arrangement 96 or Arrangement 97, wherein the nuclease is selected from a meganuclease, a zinc finger, a TALEN or a restriction enzyme which has been modified to prevent cutting of DNA or RNA.

Arrangement 99. The transmissible element or bacterium according to any preceding Arrangement, wherein the bacterium or the transmissible element further comprises a kill switch.

Arrangement 100. The bacterium according to any preceding Arrangement, wherein the bacterium is a gram negative bacterium.

Arrangement 101. The bacterium according to any preceding Arrangement, wherein the bacterium is a strain selected from any of the strains in Table 3.

Arrangement 102. The bacterium according to any preceding Arrangement, wherein the bacterium is an E. coli strain, for example an E. coli from phylogroup A, B1 and/or E, or an E. coli strain which is present in a probiotic product, such as colinfant New Born (e.g. strain A0 34/86) or symbioflor2 (e.g. strain G1/2, G4/9, G5, G6/7, and G8), or Mutaflor (e.g. E. coli Nissle).

Arrangement 103. The bacterium according to Arrangement 101, wherein the bacterium is a strain belonging to a genera selected from Bifidobacterium, Bacteroides, Lactobacillus, Lacticaseibacillus, Lactiplantibacillus, Levilactobacillus, Ligilactobacillus, Limosilactobacillus and Lactococcus, (e.g. a species which is selected from Bifidobacterium longum, Bifidobacterium breve, Bifidobacterium adolescentis, Bacteroides uniformis, Bacteroides vulgatus, Bacteroides thetaiotaomicron, Lactobacillus gasseri, Lacticaseibacillus paracasei, Lactiplantibacillus plantarum, Levilactobacillus breve, Ligilactobacillus salivarius, Limosilactobacillus reuteri and Lactococcus lactis), in particular a strain belonging to a Bifidobacterium genus or a Bacteroides genus (e.g. a species which is selected from Bifidobacterium longum, Bifidobacterium breve, Bifidobacterium adolescents, Bacteroides uniform, Bacteroides vulgatus and Bacteroides thetaiotaomicron).

Arrangement 104. The transmissible element or bacterium according to any preceding Arrangement, wherein the transmissible element is a conjugative plasmid.

Arrangement 105. The bacterium according to any one of Arrangements 1 to 103, wherein the bacterium is a recipient bacterium which comprises a transmissible element which is a conjugative plasmid.

Arrangement 106. The bacterium according to any one of Arrangements 1 to 103, wherein the bacterium is a donor bacterium which comprises a transmissible element which is a conjugative plasmid.

Arrangement 107. The transmissible element according to any one of Arrangements 1 to 103, wherein the transmissible element is selected from a phage or a phagemid (e.g. a phagemid which is packaged in a phage particle).

Arrangement 108. The bacterium according to any one of Arrangements 1 to 103, wherein the bacterium is a recipient bacterium which comprises a transmissible element which is a plasmid which has been introduced by a phage or packaged phagemid (i.e. a phagemid which is packaged in a phage particle).

Arrangement 109. The bacterium according to any one of Arrangements 1 to 103, wherein the bacterium is a recipient bacterium which comprises a transmissible element which is a prophage which has been introduced by a phage.

Arrangement 110. The bacterium according to any one of Arrangements 1 to 103, wherein the bacterium is a host bacterium comprising a transmissible element which is a phage or phagemid which is used for the production of said phage or of a packaged phagemid (i.e. a phagemid which is packaged in a phage particle).

Arrangement 111. A pharmaceutical composition comprising (i) a donor bacterium as defined in Arrangement 106, or (ii) a transmissible element as defined in Arrangement 107, and a pharmaceutically acceptable excipient or carrier.

Arrangement 112. A pharmaceutical composition according to Arrangement 111, which is formulated for oral or rectal administration, preferably oral administration, for example formulated as a capsule or coated tablet.

Arrangement 113. A pharmaceutical composition according to Arrangement 111 or Arrangement 112, which is a lyophilised formulation or is an encapsulated formulation to be released in the lower gut of a subject.

Arrangement 114. A method of modifying recipient bacteria in the gut of a subject, comprising administering to said subject (i) a donor bacterium as defined in Arrangement 106, or (ii) a transmissible element as defined in Arrangement 107 or (iii) a pharmaceutical composition as defined in any one of Arrangements 111 to 113.

Arrangement 115. A method of treating or preventing a disease or condition in a subject in need thereof, said method comprising administering to a subject in need thereof (i) a donor bacterium as defined in Arrangement 106, or (ii) a transmissible element as defined in Arrangement 107 or (iii) a pharmaceutical composition as defined in any one of Arrangements 111 to 113, and wherein the subject comprises one or more strains of bacteria which are capable of the transmissible element being transmitted to said one or more strains of bacteria.

Arrangement 116. A method of treating a metabolic disease, such as a cardiovascular metabolic disease, optionally selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase 1/3 deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome (in particular selected from metabolic syndrome, type 2 diabetes (including complications of type 2 diabetes, e.g. insulin sensitivity in type 2 diabetes), and non-alcoholic fatty liver disease), said method comprising administering to a subject in need thereof (i) a donor bacterium as defined in Arrangement 106, or (ii) a transmissible element as defined in Arrangement 107 or (iii) a pharmaceutical composition as defined in any one of Arrangements 111 to 113.

Arrangement 117. The (i) donor bacterium as defined in Arrangement 106, or (ii) transmissible element as defined in Arrangement 107 or (iii) pharmaceutical composition as defined in any one of Arrangements 111 to 113, for use in the treatment of a metabolic disease, such as a cardiovascular metabolic disease, optionally selected from leaky gut, type 1 diabetes, type 2 diabetes (including complications of type 1 and type 2 diabetes, e.g. insulin sensitivity in type 2 diabetes), metabolic syndrome, Bardet-Biedel syndrome, Prader-Willi syndrome, non-alcoholic fatty liver disease, tuberous sclerosis; Albright hereditary osteodystrophy; brain-derived neurotrophic factor (BDNF) deficiency, Single-minded 1 (SIM1) deficiency, leptin deficiency, leptin receptor deficiency, pro-opiomelanocortin (POMC) defects, proprotein convertase subtilisin/kexin type 1 (PCSK1) deficiency, Src homology 2B1 (SH2B1) deficiency, pro-hormone convertase 1/3 deficiency, melanocortin-4-receptor (MC4R) deficiency, Wilms tumor, aniridia, genitourinary anomalies, and mental retardation (WAGR) syndrome, pseudohypoparathyroidism type 1A, Fragile X syndrome, Borjeson-Forsmann-Lehmann syndrome, Alstrom syndrome, Cohen syndrome, and ulnar-mammary syndrome (in particular selected from metabolic syndrome, type 2 diabetes (including complications of type 2 diabetes, e.g. insulin sensitivity in type 2 diabetes), and non-alcoholic fatty liver disease).

1. A transmissible element for transmission to a recipient bacterium, comprising:
A) at least one nucleic acid that is exogenous to the recipient bacterium for the production of a first molecule of interest (MOI) in the recipient bacterium; and
B) a Pathway Modulation System (PMS) which encodes a nucleic acid modifier to modulate a target endogenous nucleic acid or protein for the production, consumption, or degradation of the first MOI in the recipient bacterium, wherein the nucleic acid modifier comprises:
i) a base editor;
ii) a prime editor;
iii) an RNAi system;
iv) a CRISPRi system; or
v) a nuclease,
wherein the transmissible element comprises a conjugative plasmid, phage or phagemid.

2-86. (canceled)

87. The transmissible element of claim 1, wherein the transmissible element comprises a conjugative plasmid.

88. The transmissible element of claim 1, wherein the nucleic acid modifier of B) is a nuclease, and wherein the nuclease is a Cas nuclease, a TALEN or a zinc finger.

89. The transmissible element of claim 1, wherein the first MOI is a therapeutic peptide molecule or a metabolite.

90. The transmissible element of claim 89, wherein the first MOI is a metabolite is selected from the group consisting of L-DOPA, indole-3-acetic acid, and butyrate.

91. The transmissible element of claim 1, wherein the nucleic acid of A) encodes one or more exporters of the first MOI from the recipient bacterium.

92. The transmissible element of claim 1, wherein the nucleic acid of A) comprises, in 5′ to 3′ direction, a promoter, a sequence encoding a signal peptide, and at least one nucleic acid for the expression of the first MOI.

93. The transmissible element of claim 1, wherein the nucleic acid of A) encodes one or more proteins for the conversion of a second MOI to the first MOI.

94. The transmissible element of claim 93, wherein the nucleic acid of A) encoding one or more proteins is comprised by one or more operons.

95. The transmissible element of claim 94, wherein the one or more operons are under the control of a constitutive promoter.

96. The transmissible element of claim 95, wherein the nucleic acid of A) further encodes:
a) one or more importers of the second MOI,
b) one or more importers of a further substrate which is converted by one of the proteins for the conversion of the second MOI to the first MOI, or
c) one or more importers of the second MOI and one or more importers of a further substrate which is converted by one of the proteins for the conversion of the second MOI to the first MOI.

97. The transmissible element of claim 1, wherein the nucleic acid modifier of B) reduces expression of the target endogenous nucleic acid or protein, and wherein the reduction in expression of the target endogenous nucleic acid or protein (i) increases or maintains the production of the first MOI.

98. The transmissible element of claim 1, wherein the nucleic acid modifier reduces expression of the target endogenous nucleic acid or protein, and wherein the reduction in expression of the target endogenous nucleic acid or protein reduces consumption or degradation of the first MOI.

99. The transmissible element of claim 1, wherein the nucleic acid modifier comprises a base editor.

100. The transmissible element of claim 1, wherein the nucleic acid modifier is a fusion protein comprising a polypeptide (Px) comprising a Cas nuclease fused to a base editor.

101. The transmissible element of claim 100, wherein Px comprises
I. an amino acid sequence that is at least 90% identical to a sequence selected from SEQ ID Nos:1-5; and is fused to
II. a base editor; and
wherein the PMS further comprises a nucleic acid sequence encoding a guide RNA or crRNA.

102. A method for increasing production of a first molecule of interest (MOI) in a recipient bacterium, comprising introducing a transmissible element into the recipient bacterium, wherein the transmissible element comprises:
A) at least one nucleic acid that is exogenous to the recipient bacterium for the production of a first molecule of interest (MOI) in the recipient bacterium; and
B) a Pathway Modulation System (PMS) which encodes a nucleic acid modifier to modulate a target endogenous nucleic acid or protein for the production, consumption, or degradation of the first MOI in the recipient bacterium, wherein the nucleic acid modifier comprises:
i) a base editor;
ii) a prime editor;
iii) an RNAi system;
iv) a CRISPRi system; or
v) a nuclease,
wherein the transmissible element comprises a conjugative plasmid, phage or phagemid.

103. A donor bacterium comprising the transmissible element of claim 1.

104. A pharmaceutical composition comprising a) the donor bacterium of claim 103 or transmissible element of claim 1; and b) a pharmaceutically acceptable excipient or carrier.

105. A method of treating or preventing a disease or condition in a subject in need thereof, the method comprising administering to the subject the donor bacterium of claim 103 or the subject the transmissible element of claim 1.


SEQUENCE LISTING (103 sequences)

SEQ ID NO: 1 - protein, 263 aa, synthetic construct
LNHPVESVYSALTSILLPYMGEPVPVQRNCSCCGRAPSEFDGVGFELVNAYRERVVHCRPCQTFFVSAPELMGVENPKKPTTGQKFGMWSGVGAVINVEDNSSVLLAPQGVVNKLPEHFFDHVEVITATSGQHLEYLFNTELKFPLIYIQNFGVKTYELVRSLRVSLSADAIYTCADQLLTRQNEVLYMLDLTKAKELHQEIKNYSKKEIDIFIRTVTLLAYSRITPEAASNEFKKNNLIPLLLLLPTDPHQRLSILHLLKKV

SEQ ID NO: 2 - protein, 188 aa, synthetic construct
MIYYDNAFRLRIRHTSVYQIHQHLDFFMQERKGEKLPYSFKIFPGTGDDSLLLVRTATALELPGEKKRELILSEGHEIKFITSMAIFHKGTKSEGRGRRQFAPSEEASYQLALTKLAKAGFKPGQIVVSGPKFVHIVKGNAGRGFTLPVFTVQGTAIISNQQEAEVGIVYGVGPKRVFGCGFMHLAGQ

SEQ ID NO: 3 - protein, 624 aa, synthetic construct
MNTSAVFESAGLSLRKVQQDYIEAAAGALTQDHKVALISAETGVGKTLGYLVPALLILLKNPEAKFVIATNSHALMHQIFRSDRPLLEQIAEQCGIKVTFSRLMGKANYVSLEKVRGLLLMDEFTDLDTVKVLEKLANWSKPLVEFEEEYGELPAQITPEMVTYSIWDDIQDIDDIRLNALSANFIVTTHAMVMVDCMCNHRILGDKENMYLIIDEADIFVDMLEVWKQRRFNLRELTSAFNEHIPRNGVHVIDQLMNDVTSIAGDLHFCSTPAAVALFDNSFNALSKVGREIKNEAARKAFFDCIYSWEMLGLSGGQKGVGVSNKRREPALIAVNPFIGMNVGRYCTQWRSALLTSATLSITSTPETGMEWLCKALGLTSDTISIRKIFSPDVYGSMKLTIAGADFPKVFNDPKEQIFSGQWLKAVVEQLSCIQGPALVLTASHYETRMIANQLGEVSQPVYIQKAGQALSEIIKQYQEIPGILISAGASVGVSPRGENGEQIFQDLIITRIPFLPPDRMKAESLYGYLKERGYSRTFEAVNRNIYLDNLRKVIRKAKQSIGRGIRSENDTVRIIILDPRFPEPTDLSSKHRSLEHIIPVRFRRAYRSCEILSPAYCEEDIQC

SEQ ID NO: 4 - protein, 235 aa, synthetic construct
VLNFKPYRVIMSSLTPVVISGIAPSLDGILYEALSQAIPSNEPGVVLARLKEILLFNDELGVFHASSLRFGITPEQGIGATTSVRCDYLSPEKLSTAMFSPRIHRGVFTRVLLTGGPTKRRMTTRPAYSAPYLTFDFVGSSEAVEILLNHAHVGVGYDFFSAANGEFNNVTILPLDIDTSISNEGMALRPVPVNSGLNGIKGVSPLIPPYFVGEKLNIVHPAPVRTQLISSLLRG

SEQ ID NO: 5 - protein, 343 aa, synthetic construct
MRTLNFNGKISTLEPLTVTVKNAVSTSGHRLPRNGGFNAAPYFPGTSIRGTLRHAAHKVIVDRVGLNADGKSSFDLAEHFMLAQGVDINGEAETFAPGEINAGAELRSKNPLISLFGRWGLSGKVGIGNAIPDGDNQWGMFGGGARSIMFQRDESLMEFLETDQVDRLERLLEEQAEASVDISQIKTEQDALKKAMKSADKDTKAELQIKVRELDEKIQARKDQKQESRESIRRPIDPYEAFITGAELSHRMSIKNATDEEAGLFISALIRFAAEPRFGGHANHNCGLVEAHWTVTTWKPGELVPVTLGEIVITPNGVEITGDELFAMVKAFNENQSFDFTAR

SEQ ID NO: 6 - DNA, 29 bp, synthetic construct
  source 1..29: mol_type = other DNA
gtattccccccgtgtgcgggggttatcgg

SEQ ID NO: 7 - DNA, 748 bp, synthetic construct
  source 1..748: mol_type = other DNA
ttgaatcatcctgtagagtcagtatatagcgctctgacgagtattttattgccctatatgggtgagcctgtgcctgtgcaacgaaattgctcatgctgcggcagagcgccctcagagttcgacggggtaggcttcgagctggtcaacgcctacagggaacgtgttgtgcattgccgtccttgccagacattttttgtttctgctccagaactgatgggcgtggagaaccccaaaaaaccaacaaccggccagaaatttggcatgtggtctggtgttggggctgttattaacgttgaagacaattcttcggttcttttagcccctcagggagtggtcaataaactacctgagcattttttcgatcacgtagaagtcatcacggccaccagcggtcagcacctggaatacctgtttaacaccgaacttaaattcccgctgatttatattcaaaacttcggcgtgaaaacctacgagcttgttcgctcactgcgggtcagtctgagtgccgacgcaatttatacctgtgccgatcaactactgacccggcagaacgaagtcctttacatgctggatttaacaaaggctaaagaactccatcaggaaataaaaaactactccaaaaaagagatagacatctttatcagaacagtaaccctgctggcctattcgcggataactcctgaagctgcctcgaatgagtttaagaaaaacaacctcattccgttactgctgcttctgccgactgatc

SEQ ID NO: 8 - DNA, 567 bp, synthetic construct
  source 1..567: mol_type = other DNA
atgatttactacgataatgcgtttcggttgcgtatccgtcacacaagtgtttaccagatccaccagcacctggattttttcatgcaggaacgtaagggagagaagctgccgtactcttttaaaatttttcccggtaccggggatgattcgttactcctggtccgcacagccacagctctggagttgccaggggagaaaaaaagagagcttattctctctgagggccatgaaatcaagttcattacgtcaatggccatctttcataaaggaacaaagtcagaaggccgtggccgacgccagtttgctccatccgaagaggctagttatcagttggccctcaccaaattagcgaaggctggttttaaacccggtcaaatcgtcgtcagtggcccgaaattcgtccatatcgtcaaaggaaatgctggacgcggttttacgctacccgtattcacggtacagggtaccgccataatcagcaatcaacaggaagctgaagttggaatcgtttatggcgtaggccctaaacgcgtatttggttgcggtttcatgcatctggcggggcagtag

SEQ ID NO: 9 - DNA, 1871 bp, synthetic construct
  source 1..1871: mol_type = other DNA
atgaatacatcagcggtatttgagtctgccggattgtcgttacgtaaggtccaacaggattatatcgaggctgcagctggtgcgctgactcaggatcataaagttgcactgattagtgctgaaacaggtgtaggaaaaacgctgggctatctggtaccggcgcttctgattctgctgaaaaacccagaggcaaaatttgtgatagccacgaattctcatgccctgatgcaccagatattcagaagcgatcgtcctctccttgaacagatagctgaacaatgcggcataaaagttactttttcccggcttatggggaaagcaaattacgtatccctggagaaagtacgtggactgttactcatggacgaatttaccgatctggatacggttaaggtccttgaaaaactcgctaactggtcaaaacctctggttgagtttgaagaagagtatggtgaactgccagcgcagatcacgccagagatggtcacctactccatctgggatgatatacaggacattgatgatattcgcctgaatgctctcagtgctaactttatagttacgacccatgcgatggtaatggtcgactgtatgtgtaatcaccgcatcctcggtgacaaagaaaatatgtacctcattattgatgaggcagacatttttgtagatatgcttgaggtctggaaacagcggcgcttcaacctcagagagttgaccagtgcgttcaatgaacatattccacgtaatggcgtgcatgtcatcgaccagttaatgaacgacgtgacttcgatagcgggcgatctgcatttttgtagtacgcctgcagcggttgccttgtttgacaatagctttaacgccttatcaaaggtagggcgggaaataaaaaatgaagcggcccggaaagctttctttgactgtatctacagctgggaaatgctcggattgtccggggggcaaaagggtgtaggcgtttccaataaacgtcgtgaaccagccctgatcgctgtaaacccgtttatcgggatgaatgtgggcagatactgcactcaatggcgtagtgcgttactgacgtcggctacgctctcaattaccagtaccccagagaccggaatggagtggttatgtaaagctctggggttaaccagcgatacgatttcaatcaggaaaatattctccccagacgtctatggctcaatgaagctgaccatcgccggtgctgattttccaaaggtgttcaatgacccgaaagaacagatattttcaggtcaatggttaaaggcagtagttgaacaattgtcctgtattcaggggccagctctggtattaactgccagccattatgaaaccaggatgattgccaatcagctgggtgaggtctcgcaaccagtttatattcagaaagcaggtcaggccctgtccgaaattattaaacagtatcaggagataccagggattcttatttctgccggtgcttctgttggtgtgagcccacgaggcgagaatggggaacagattttccaggatttaatcattacccggatacctttcttacctccggacagaatgaaggctgaaagcctttatgggtatctgaaggaacgaggctatagtcgtacctttgaggcagttaaccgtaacatctacctggataatttgcggaaagttattcgtaaagcaaagcagtctatagggcgggggattcgtagtgaaaatgataccgtcaggataattatcctcgatccgcgtttcccggaacctactgacctttcgtctaaacatcggtcgcttgaacacattattcccgttcgatttcgtcgtgcatatcgttcgtgtgaaattttatctccggcgtattgtgaagaggatatccagtg

SEQ ID NO: 10 - DNA, 708 bp, synthetic construct
  source 1..708: mol_type = other DNA
gtgttaaattttaaaccgtatcgggtcatcatgtcttctctgacaccggtggtcatcagtgggatagccccatcgctggacggcattctgtatgaagcactgtctcaggcgatacccagtaacgagccaggagtcgtattggctcgtcttaaggaaatacttctattcaacgatgaactgggtgttttccatgcttcatctctacgttttggcataacgcccgaacaggggattggggcgacaacaagcgtacgttgcgattatctcagtcccgaaaaactgagcactgcaatgttctctcctcgtatccacaggggggtgtttacgcgtgtcctcctgaccgggggccccacgaagagaaggatgacaacccgtccagcttattctgcaccctatttaacgtttgattttgtcggctcttctgaggctgtagaaatactgcttaaccatgcccatgttggcgttggttatgactttttctctgcagctaatggagagtttaacaatgtgacaattcttcctctcgatatcgacacgagtatatccaacgagggtatggcattgcgtccggtacctgttaattcgggtctgaatggtatcaaaggagtatcaccacttattcctccctatttcgttggtgaaaagctaaacattgtccacccagcaccagttcgtactcaattgatttcttctttattgcgaggctaa

SEQ ID NO: 11 - DNA, 1032 bp, synthetic construct
  source 1..1032: mol_type = other DNA
atgagaactttaaacttcaacggtaaaatttcgactctggaaccactgactgtaacagtaaaaaatgcggtctcaacttccggccatcgcttgcctcgtaacggtggattcaatgcagccccgtactttccaggtaccagcattcggggaactcttcgtcatgctgcgcataaggtcattgtcgatcgtgtgggccttaatgcagacggaaaatcatccttcgacctggcagaacatttcatgctggcccagggggtggatatcaatggtgaagccgaaaccttcgcgccaggtgaaatcaatgctggtgctgaactacgtagcaaaaacccgttaatcagcttgtttggtcgttggggattaagcggcaaagttggtatagggaatgctatcccggatggtgataaccagtgggggatgtttggtggtggtgcccgctcaattatgtttcagcgtgacgaaagtctgatggagtttcttgagacagaccaggttgatcgtcttgaacgcctgctcgaggaacaggctgaggcaagtgtggatatctctcagatcaaaacagagcaagatgcactcaaaaaggcgatgaagtcggctgataaagacaccaaagctgagcttcaaatcaaagtacgggagctcgatgaaaaaattcaggcccgcaaagatcagaagcaggaatctcgcgagtcaattcgccgcccgattgacccgtatgaagcgtttatcaccggcgcagaactcagccatcgcatgagtattaaaaatgcgactgatgaggaagcagggcttttcatttctgcattaatccgctttgcagccgaaccacgttttggcggtcatgcgaatcataactgcggtctggtggaggctcactggacagttacgacctggaagccgggtgaactggtaccagttacacttggagaaatcgtcatcacaccgaatggtgttgagattaccggggacgagttgtttgctatggtaaaggcattcaatgaaaatcaatcttttgatttcactgcccgctaa

SEQ ID NO: 12 - DNA, 5636 bp, synthetic construct
  source 1..5636: mol_type = other DNA
ttgaatcatcctgtagagtcagtatatagcgctctgacgagtattttattgccctatatgggtgagcctgtgcctgtgcaacgaaattgctcatgctgcggcagagcgccctcagagttcgacggggtaggcttcgagctggtcaacgcctacagggaacgtgttgtgcattgccgtccttgccagacattttttgtttctgctccagaactgatgggcgtggagaaccccaaaaaaccaacaaccggccagaaatttggcatgtggtctggtgttggggctgttattaacgttgaagacaattcttcggttcttttagcccctcagggagtggtcaataaactacctgagcattttttcgatcacgtagaagtcatcacggccaccagcggtcagcacctggaatacctgtttaacaccgaacttaaattcccgctgatttatattcaaaacttcggcgtgaaaacctacgagcttgttcgctcactgcgggtcagtctgagtgccgacgcaatttatacctgtgccgatcaactactgacccggcagaacgaagtcctttacatgctggatttaacaaaggctaaagaactccatcaggaaataaaaaactactccaaaaaagagatagacatctttatcagaacagtaaccctgctggcctattcgcggataactcctgaagctgcctcgaatgagtttaagaaaaacaacctcattccgttactgctgcttctgccgactgatccccatcaacgcctgagcattttgcacctcttgaaaaaggtataaccatgatttactacgataatgcgtttcggttgcgtatccgtcacacaagtgtttaccagatccaccagcacctggattttttcatgcaggaacgtaagggagagaagctgccgtactcttttaaaatttttcccggtaccggggatgattcgttactcctggtccgcacagccacagctctggagttgccaggggagaaaaaaagagagcttattctctctgagggccatgaaatcaagttcattacgtcaatggccatctttcataaaggaacaaagtcagaaggccgtggccgacgccagtttgctccatccgaagaggctagttatcagttggccctcaccaaattagcgaaggctggttttaaacccggtcaaatcgtcgtcagtggcccgaaattcgtccatatcgtcaaaggaaatgctggacgcggttttacgctacccgtattcacggtacagggtaccgccataatcagcaatcaacaggaagctgaagttggaatcgtttatggcgtaggccctaaacgcgtatttggttgcggtttcatgcatctggcggggcagtagtcatgaatacatcagcggtatttgagtctgccggattgtcgttacgtaaggtccaacaggattatatcgaggctgcagctggtgcgctgactcaggatcataaagttgcactgattagtgctgaaacaggtgtaggaaaaacgctgggctatctggtaccggcgcttctgattctgctgaaaaacccagaggcaaaatttgtgatagccacgaattctcatgccctgatgcaccagatattcagaagcgatcgtcctctccttgaacagatagctgaacaatgcggcataaaagttactttttcccggcttatggggaaagcaaattacgtatccctggagaaagtacgtggactgttactcatggacgaatttaccgatctggatacggttaaggtccttgaaaaactcgctaactggtcaaaacctctggttgagtttgaagaagagtatggtgaactgccagcgcagatcacgccagagatggtcacctactccatctgggatgatatacaggacattgatgatattcgcctgaatgctctcagtgctaactttatagttacgacccatgcgatggtaatggtcgactgtatgtgtaatcaccgcatcctcggtgacaaagaaaatatgtacctcattattgatgaggcagacatttttgtagatatgcttgaggtctggaaacagcggcgcttcaacctcagagagttgaccagtgcgttcaatgaacatattccacgtaatggcgtgcatgtcatcgaccagttaatgaacgacgtgacttcgatagcgggcgatctgcatttttgtagtacgcctgcagcggttgccttgtttgacaatagctttaacgccttatcaaaggtagggcgggaaataaaaaatgaagcggcccggaaagctttctttgactgtatctacagctgggaaatgctcggattgtccggggggcaaaagggtgtaggcgtttccaataaacgtcgtgaaccagccctgatcgctgtaaacccgtttatcgggatgaatgtgggcagatactgcactcaatggcgtagtgcgttactgacgtcggctacgctctcaattaccagtaccccagagaccggaatggagtggttatgtaaagctctggggttaaccagcgatacgatttcaatcaggaaaatattctccccagacgtctatggctcaatgaagctgaccatcgccggtgctgattttccaaaggtgttcaatgacccgaaagaacagatattttcaggtcaatggttaaaggcagtagttgaacaattgtcctgtattcaggggccagctctggtattaactgccagccattatgaaaccaggatgattgccaatcagctgggtgaggtctcgcaaccagtttatattcagaaagcaggtcaggccctgtccgaaattattaaacagtatcaggagataccagggattcttatttctgccggtgcttctgttggtgtgagcccacgaggcgagaatggggaacagattttccaggatttaatcattacccggatacctttcttacctccggacagaatgaaggctgaaagcctttatgggtatctgaaggaacgaggctatagtcgtacctttgaggcagttaaccgtaacatctacctggataatttgcggaaagttattcgtaaagcaaagcagtctatagggcgggggattcgtagtgaaaatgataccgtcaggataattatcctcgatccgcgtttcccggaacctactgacctttcgtctaaacatcggtcgcttgaacacattattcccgttcgatttcgtcgtgcatatcgttcgtgtgaaattttatctccggcgtattgtgaagaggatatccagtgttaaattttaaaccgtatcgggtcatcatgtcttctctgacaccggtggtcatcagtgggatagccccatcgctggacggcattctgtatgaagcactgtctcaggcgatacccagtaacgagccaggagtcgtattggctcgtcttaaggaaatacttctattcaacgatgaactgggtgttttccatgcttcatctctacgttttggcataacgcccgaacaggggattggggcgacaacaagcgtacgttgcgattatctcagtcccgaaaaactgagcactgcaatgttctctcctcgtatccacaggggggtgtttacgcgtgtcctcctgaccgggggccccacgaagagaaggatgacaacccgtccagcttattctgcaccctatttaacgtttgattttgtcggctcttctgaggctgtagaaatactgcttaaccatgcccatgttggcgttggttatgactttttctctgcagctaatggagagtttaacaatgtgacaattcttcctctcgatatcgacacgagtatatccaacgagggtatggcattgcgtccggtacctgttaattcgggtctgaatggtatcaaaggagtatcaccacttattcctccctatttcgttggtgaaaagctaaacattgtccacccagcaccagttcgtactcaattgatttcttctttattgcgaggctaatttcatgagaactttaaacttcaacggtaaaatttcgactctggaaccactgactgtaacagtaaaaaatgcggtctcaacttccggccatcgcttgcctcgtaacggtggattcaatgcagccccgtactttccaggtaccagcattcggggaactcttcgtcatgctgcgcataaggtcattgtcgatcgtgtgggccttaatgcagacggaaaatcatccttcgacctggcagaacatttcatgctggcccagggggtggatatcaatggtgaagccgaaaccttcgcgccaggtgaaatcaatgctggtgctgaactacgtagcaaaaacccgttaatcagcttgtttggtcgttggggattaagcggcaaagttggtatagggaatgctatcccggatggtgataaccagtgggggatgtttggtggtggtgcccgctcaattatgtttcagcgtgacgaaagtctgatggagtttcttgagacagaccaggttgatcgtcttgaacgcctgctcgaggaacaggctgaggcaagtgtggatatctctcagatcaaaacagagcaagatgcactcaaaaaggcgatgaagtcggctgataaagacaccaaagctgagcttcaaatcaaagtacgggagctcgatgaaaaaattcaggcccgcaaagatcagaagcaggaatctcgcgagtcaattcgccgcccgattgacccgtatgaagcgtttatcaccggcgcagaactcagccatcgcatgagtattaaaaatgcgactgatgaggaagcagggcttttcatttctgcattaatccgctttgcagccgaaccacgttttggcggtcatgcgaatcataactgcggtctggtggaggctcactggacagttacgacctggaagccgggtgaactggtaccagttacacttggagaaatcgtcatcacaccgaatggtgttgagattaccggggacgagttgtttgctatggtaaaggcattcaatgaaaatcaatcttttgatttcactgcccgctaactccaaaagctggtggattttagtggcgctatttaatattttataatcaaccggttatttttagagtattccccccgtgtgcgggggttatcggtggggcgttgttgaataaatagatattatgctgcgggcctaccaaaacgtcggtctttcccaacgcaaaatcagtaggttaaggttaaagtcgggagctactcacaatagtgacaagcatctttgactaaagcaggcgcgccaaaacagcatgattccctattattcaacaaagcctatcggtggtgctctcaaccgtcacccgctggctggaagtattccccccgtgtgcgggggttatcggtcgtgttgtccacggttacccgctggctggaagtattccccccgcatgcgggggttatcggttaccaatggggaaaaatcttcatttgtaaatgtattccccccgcatgcgggggttatcggaacatcagtggaaatccactgcggcgtattctccccgcatgcgggggttatcggcgaaaacggcaaccttcataaaaacgtcttttgtattccccccgtgtgcgggggttatcggccgagattgagtaaagcaaagtaacggcggtggtattccccccgcatgcgggggttatcggcaggttatactggcaaaacgtcgatg

SEQ ID NO: 13 - skipped

SEQ ID NO: 14 - DNA, 85 bp, Escherichia coli
  source 1..85: mol_type = other DNA
aacctaaatatttgttgttaagctgcaatggaaacggtaaaagcggctagtatttaaagggatggatgacatctcagcgttgtcg

SEQ ID NO: 15 - DNA, 160 bp, synthetic construct
  source 1..160: mol_type = other DNA
cctgatgcgtgaacgtgacggacgtaaccaccgcgacatgtgtgtgctgttccgctgggcatgctgagctaacaccgtgcgtgttgacaattttacctctggcggtgataatggttgcagctagcaataattttgtttaactttaagaaggagatatacc

SEQ ID NO: 16 - DNA, 48 bp, synthetic construct
  source 1..48: mol_type = other DNA
agcggcagcgagactcccgggacctcagagtccgccacacccgaaagt

SEQ ID NO: 17 - protein, 16 aa, synthetic construct
SGSETPGTSESATPES

SEQ ID NO: 18 - DNA, 624 bp, Petromyzon marinus
  source 1..624: mol_type = genomic DNA
atgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaatctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtt

SEQ ID NO: 19 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 20 - DNA, 255 bp, Homo sapiens
  source 1..255: mol_type = genomic DNA
atgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgctataa

SEQ ID NO: 21 - protein, 84 aa, Homo sapiens
MTNLSDIIEKETGKQLVIQESILMLPEEVEEVIGNKPESDILVHTAYDESTDENVMLLTSDAPEYKPWALVIQDSNGENKIKML

SEQ ID NO: 22 - DNA, 30 bp, synthetic construct
  source 1..30: mol_type = other DNA
tctggtggatctggaggttctggtggatct

SEQ ID NO: 23 - protein, 10 aa, synthetic construct
SGGSGGSGGS

SEQ ID NO: 24 - DNA, 213 bp, synthetic construct
  source 1..213: mol_type = other DNA
gaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaa

SEQ ID NO: 25 - DNA, 32 bp, synthetic construct
  source 1..32: mol_type = other DNA
ggagacgtcatgtcaagctcataggcgtctcg

SEQ ID NO: 26 - DNA, 32 bp, synthetic construct
  source 1..32: mol_type = other DNA
acatacgcttcttgctgagtacgtgagttcac

SEQ ID NO: 27 - DNA, 32 bp, synthetic construct
  source 1..32: mol_type = other DNA
cagattgctggtcaggagcatattgatccgct

SEQ ID NO: 28 - DNA, 32 bp, synthetic construct
  source 1..32: mol_type = other DNA
aaggagatataccatggagcttttcactggcg

SEQ ID NO: 29 - DNA, 32 bp, synthetic construct
  source 1..32: mol_type = other DNA
ttcagcgtgtccggcgagggcgagggcgatgc

SEQ ID NO: 30 - DNA, 32 bp, synthetic construct
  source 1..32: mol_type = other DNA
ctgcccgtgccctggcccaccctcgtgaccac

SEQ ID NO: 31 - DNA, 32 bp, synthetic construct
  source 1..32: mol_type = other DNA
gagaagcgcgatcacatggtcctgctggagtt

SEQ ID NO: 32 - DNA, 32 bp, synthetic construct
  source 1..32: mol_type = other DNA
taccagatggcattgcgccatctggcagagtg

SEQ ID NO: 33 - DNA, 32 bp, synthetic construct
  source 1..32: mol_type = other DNA
aaagtcaggttgcgcaggcagggtaatcgggg

SEQ ID NO: 34 - DNA, 32 bp, synthetic construct
  source 1..32: mol_type = other DNA
tcacgtactcagcaagaagcgtatgtgtttgc

SEQ ID NO: 35 - DNA, 32 bp, synthetic construct
  source 1..32: mol_type = other DNA
acctctgtcagctcgctgacattccccgccag

SEQ ID NO: 36 - DNA, 32 bp, synthetic construct
  source 1..32: mol_type = other DNA
ttaaacaaaattattgctagctgcaaccatta

SEQ ID NO: 37 - DNA, 32 bp, synthetic construct
  source 1..32: mol_type = other DNA
ctccatggtatatctccttcttaaagttaaac

SEQ ID NO: 38 - DNA, 32 bp, synthetic construct
  source 1..32: mol_type = other DNA
cactgcacgccgtaggtcagggtggtcacgag

SEQ ID NO: 39 - DNA, 32 bp, synthetic construct
  source 1..32: mol_type = other DNA
tccagcaggaccatgtgatcgcgcttctcgtt

SEQ ID NO: 40 - DNA, 32 bp, synthetic construct
  source 1..32: mol_type = other DNA
cactctgccagatggcgcaatgccatctggta

SEQ ID NO: 41 - DNA, 32 bp, synthetic construct
  source 1..32: mol_type = other DNA
ctgactttgttgtcggcccggcgggcactcaa

SEQ ID NO: 42 - DNA, 79 bp, synthetic construct
  source 1..79: mol_type = other DNA
aattcagcgggttcgtgcgaactgttgacaattaatactcggctcgtataatgtgtggaaggctagcaggaggaattca

SEQ ID NO: 43 - DNA, 35 bp, synthetic construct
  source 1..35: mol_type = other DNA
ttgacagctagctcagtcctaggtataatgctagc

SEQ ID NO: 44 - DNA, 35 bp, synthetic construct
  source 1..35: mol_type = other DNA
ttgacggctagctcagtcctaggtacagtgctagc

SEQ ID NO: 45 - DNA, 35 bp, synthetic construct
  source 1..35: mol_type = other DNA
tttacagctagctcagtcctaggtattatgctagc

SEQ ID NO: 46 - DNA, 35 bp, synthetic construct
  source 1..35: mol_type = other DNA
ttgacagctagctcagtcctaggtactgtgctagc

SEQ ID NO: 47 - DNA, 35 bp, synthetic construct
  source 1..35: mol_type = other DNA
ctgatagctagctcagtcctagggattatgctagc

SEQ ID NO: 48 - DNA, 35 bp, synthetic construct
  source 1..35: mol_type = other DNA
ttgacagctagctcagtcctaggtattgtgctagc

SEQ ID NO: 49 - DNA, 35 bp, synthetic construct
  source 1..35: mol_type = other DNA
tttacggctagctcagtcctaggtactatgctagc

SEQ ID NO: 50 - DNA, 35 bp, synthetic construct
  source 1..35: mol_type = other DNA
tttacggctagctcagtcctaggtatagtgctagc

SEQ ID NO: 51 - DNA, 35 bp, synthetic construct
  source 1..35: mol_type = other DNA
tttacggctagctcagccctaggtattatgctagc

SEQ ID NO: 52 - DNA, 35 bp, synthetic construct
  source 1..35: mol_type = other DNA
ctgacagctagctcagtcctaggtataatgctagc

SEQ ID NO: 53 - DNA, 35 bp, synthetic construct
  source 1..35: mol_type = other DNA
tttacagctagctcagtcctagggactgtgctagc

SEQ ID NO: 54 - DNA, 35 bp, synthetic construct
  source 1..35: mol_type = other DNA
tttacggctagctcagtcctaggtacaatgctagc

SEQ ID NO: 55 - DNA, 35 bp, synthetic construct
  source 1..35: mol_type = other DNA
ttgacggctagctcagtcctaggtatagtgctagc

SEQ ID NO: 56 - DNA, 35 bp, synthetic construct; identical to SEQ ID NO: 47
  source 1..35: mol_type = other DNA

SEQ ID NO: 57 - DNA, 35 bp, synthetic construct
  source 1..35: mol_type = other DNA
ctgatggctagctcagtcctagggattatgctagc

SEQ ID NO: 58 - DNA, 35 bp, synthetic construct
  source 1..35: mol_type = other DNA
tttatggctagctcagtcctaggtacaatgctagc

SEQ ID NO: 59 - DNA, 35 bp, synthetic construct
  source 1..35: mol_type = other DNA
tttatagctagctcagcccttggtacaatgctagc

SEQ ID NO: 60 - DNA, 35 bp, synthetic construct
  source 1..35: mol_type = other DNA
ttgacagctagctcagtcctagggactatgctagc

SEQ ID NO: 61 - DNA, 35 bp, synthetic construct
  source 1..35: mol_type = other DNA
ttgacagctagctcagtcctagggattgtgctagc

SEQ ID NO: 62 - DNA, 35 bp, synthetic construct
  source 1..35: mol_type = other DNA
ttgacggctagctcagtcctaggtattgtgctagc

SEQ ID NO: 63 - DNA, 79 bp, synthetic construct
  source 1..79: mol_type = other DNA
aattcagcgggttcgtgcgaactgtagacaattaatactcggctcgtataatgtgtggaaggctagcaggaggaattca

SEQ ID NO: 64 - DNA, 1191 bp, Escherichia coli
  source 1..1191: mol_type = genomic DNA
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 65 - DNA, 1176 bp, Arabidopsis thaliana
  source 1..1176: mol_type = genomic DNA
atggttaagttagagaatagtcgcaaacctgagaaaatctcaaataagaatattcctatgagcgacttcgtcgtgaatttagaccacggtgatccgacagcgtacgaagagtactggcgcaaaatgggagaccgctgtaccgtgaccattcgcggttgtgacttgatgagttatttttctgatatgacgaatctgtgttggttcttggagccggaactggaggatgcgattaaggatttacacggtgtcgttggcaatgcagctacggaggaccgctatatcgtggtgggaactgggtctacgcagttatgccaggctgcggtccacgcattgtcttctcttgcgcgtagtcaaccagtttccgttgtggctgcggcaccattttacagcacgtacgtggaggagaccacctatgttcgcagtgggatgtacaagtgggagggagacgcgtgggggtttgataagaaaggtccttatattgaacttgtgacatcaccgaataacccggacgggacgatccgcgagaccgtggttaatcgcccggatgatgatgaagcaaaggtaatccacgacttcgcttactattggccccattatacacctattacgcgtcgccaggatcacgacatcatgctttttacctttagcaaaatcacggggcacgctggtagtcgtattggatgggcgctggtgaaggataaagaggtcgctaagaaaatggttgagtatattatcgtgaacagtatcggcgtttcgaaagaatcgcaggtgcgtacagcgaaaattttgaatgtcctgaaggaaacgtgtaaatctgaatccgaatccgagaacttcttcaaatatggtcgtgagatgatgaagaatcgctgggagaagctgcgtgaagtagtcaaggagtcagacgccttcacattgcccaaatatcctgaggctttttgtaattatttcgggaagagtttagaatcatacccggcgtttgcctggctggggacaaaggaagagaccgacttggtatcggaattgcgtcgtcacaaggtaatgtcgcgtgcaggcgagcgttgcggttcagataagaagcatgttcgcgtatcaatgctgtcccgcgaggatgttttcaatgtcttcttagagcgcttagccaacatgaaattgatcaaatctatcgatttgtaa

SEQ ID NO: 66 - DNA, 1653 bp, Pantoea agglomerans
  source 1..1653: mol_type = genomic DNA
atgtcgacttttacagtaggtgactatttattaactcgcttacaagagatcggggtaaagcatttatttggagtacctggtgactataacttacaatttcttgaccgtgtgatcgctcatcccgctatttcatgggtaggctgtgccaacgagttgaacgcagcctatgcggctgatggatatgcacgttgcaatggggccggtgctcttcttacgaccttcggggttggcgagctttccgcgatcaacggcatcgctggttcttatgccgagtatctgccggtcattcatattgtcggggcaccagctacgcaggcgcagttgcaaggggactgcgtgcatcattctttaggagatggggacttccagcattttatccgcatggcgactgaagtatcggctgccacggctcaacttaacgccgacaacgccactgccgaaatcgaccgtgtaatcgtaagtgccctgcaagcacgccgtcccggatatctgtctctggctgtggatgtggcagccatggaagttcaaccaccagctcaaccattgaacagccaccaaagctctagtccggcagcccgtcgcgccttctcagatgctgccgagcgtttattggcgccagcacaacgcgtcagtctgcttgcggattttttagccctgcgttggcagcaacaatcagccttagctgcattgcgcgaacaatgcgccatcccgtgcgcttcgctgttaatggggaagggagtcttggatgagcagcagcccgggtatgttggtacttatgctggcgaggcgtctgcgggccaggtttgtgaacagattgaacaagtagacgtcgcaatttgtgtcggggtccgttttacggacaccatcaccgccggatttacacagcagtttaatcccgagcgcctgatcgacttgcagccacttagcgctagtgtcggcggagaacgctttgccccactttcaatggcagacgctctttccgaacttttgccgcttttcgagcgctatgggcaacagtggcaaccaggggctacgccacctgcagcacaaccggctgaacccgccgccgtcatttcccaacaagcgttttggcaagcaatgcaagccttcttacagccgggcgatcttattttggccgaacaaggtactgcggcatttggggctgccgcgcttcgtctgccaagccaggcccagttagtagtacaacctctttgggggtctattggatacaccctgccagcagcttttggtgcgcaaacggctgaccctgatcgtcgcgtgatcctgatcattggagatggttcagcgcaattgactatccaagagttaggaagcatgctgcgcgaccaacaacgcttgatccttttcctgctgaacaatgagggctatactgttgaacgtgcgattcacggtgctacgcagcgttataatgacattgcccagtggaattggaccgctctgcctcacgcactttctttgcaaggccaggcacaatcatggcgtatctcggagacggtgcaattagaggaggtgatggctcgtttgtccgaacccaagtggctgtcattggtcgaggtcgtgatgcagaaggaggacctgcctcctttattacgcaaagtaacagcttgtcttaaccagcgtaatggctgctaa

SEQ ID NO: 67 - DNA, 1494 bp, Ustilago maydis
  source 1..1494: mol_type = genomic DNA
atgccaactctcaacctcgatctgcctaacggcatcaagtcgaccatccaggctgacttgttcatcaacaacaagtttgtgcccgctcttgacggcaagacttttgcgaccatcaacccttccaccggtaaggagattggtcaggtcgccgaggcttccgccaaggacgttgatctcgctgtcaaggctgcgcgcgaggcattcgagactacttggggcgagaacacacccggtgacgctcgtggcagacttctgatcaagctcgccgagctggtcgaggccaacattgacgagcttgctgccatcgagtcgctcgacaacggcaaggccttctcgatcgctaaaagctttgacgtcgctgccgtcgctgccaacttgcgatactatggtggctgggccgacaagaaccacggcaaggtcatggaggtcgacaccaagcgcctcaactacacgcgccacgagcccatcggcgtgtgtggtcagatcatcccttggaacttccccttgctcatgttcgcctggaagctcggccctgcgctcgccacaggtaacacgatcgtcctcaaaaccgccgagcagacaccactgtcagccatcaagatgtgtgagcttatcgtcgaggccggcttcccgcctggtgtggtcaacgtcatttccggtttcggtcccgtcgccggtgccgccatctcacagcacatggacattgacaagatcgccttcaccggctcaacactggtgggccgcaacattatgaaggctgctgcttcgaccaacctgaagaaggtgacgctcgagctcggcggcaagtcgcccaacattatctttaaggacgccgacctggaccaggcggttcgatggtcggcgttcggtatcatgttcaatcacggacaatgctgctgtgcgggctcgcgtgtctatgtggaggaatcgatctatgacgctttcatggagaagatgactgcccactgcaaggcgctccaggtgggtgaccccttttccgccaacacgttccaaggtcctcaggtttcgcagctccagtacgaccgtatcatggagtacatcgagtccggcaagaaggacgctaaccttgcgctcggtggtgtgcgcaagggtaacgaaggctactttatcgagcctaccatcttcaccgacgttcctcacgatgccaagattgccaaggaggagatcttcggccctgtcgtggtcgtctccaagttcaaagatgaaaaggacctcattcgcatcgccaacgactcgatctacggcctcgctgccgctgtcttcagccgcgacatcagccgtgccatcgagactgcccacaagctcaaggccggcaccgtctgggtcaactgctacaaccagctcatccctcaggttccgttcggcggttacaaggcgtccggtatcggtcgagagctcggcgagtatgcgctcagcaactacacaaacatcaaggcggtgcatgtgaacctcagccagcctgcccctatctaa

SEQ ID NO: 68 - DNA, 85 bp, Escherichia coli
  source 1..85: mol_type = other DNA
cgactactttctgacttccttcgtgacttgccctaagcatgttgtagtgcgatacttgtaatgacatttgtaattacaagaggtg

SEQ ID NO: 69 - DNA, 85 bp, Escherichia coli; identical to SEQ ID NO: 14
  source 1..85: mol_type = other DNA

SEQ ID NO: 70 - DNA, 85 bp, Escherichia coli
  source 1..85: mol_type = other DNA
aaaagataaatccacacagtttgtattgttttgtgcaaaagtttcactacgctttattaacaatactttctggcgacgtgcgcca

SEQ ID NO: 71 - DNA, 85 bp, Escherichia coli
  source 1..85: mol_type = other DNA
agccagagcatgccctgacttcaccccgctgtgtctgcttttcccgactattcttaatgagcttcgatgcaattcacgatcccgc

SEQ ID NO: 72 - DNA, 85 bp, Escherichia coli
  source 1..85: mol_type = other DNA
acggtacaacatttacgccactttacgcctgaataataaaagcgtgttatactctttccctgcaatgggttccgtagcagggaaa

SEQ ID NO: 73 - DNA, 85 bp, Escherichia coli; identical to SEQ ID NO: 72
  source 1..85: mol_type = other DNA

SEQ ID NO: 74 - DNA, 85 bp, Escherichia coli
  source 1..85: mol_type = other DNA
tgcgtaatttattcacaagcttgcattgaacttgtggataaaatcacggtctgataaaacagtgaatgataacctcgttgctctt

SEQ ID NO: 75 - DNA, 85 bp, Escherichia coli
  source 1..85: mol_type = other DNA
tttattcacaagcttgcattgaacttgtggataaaatcacggtctgataaaacagtgaatgataacctcgttgctcttaagctct

SEQ ID NO: 76 - DNA, 85 bp, Escherichia coli
  source 1..85: mol_type = other DNA
tgcattgaacttgtggataaaatcacggtctgataaaacagtgaatgataacctcgttgctcttaagctctggcacagttgttgc

SEQ ID NO: 77 - DNA, 85 bp, Escherichia coli
  source 1..85: mol_type = other DNA
aacagtgaatgataacctcgttgctcttaagctctggcacagttgttgctaccactgaagcgccagaagatatcgattgagagga

SEQ ID NO: 78 - DNA, 216 bp, Escherichia coli
  source 1..216: mol_type = other DNA
atgcagaataaacctacacctgaagaagtaaagaatgcgcgggttgcggcaggtcttactcttaaagaagctgctgatatttttggttatcaactgaattcctggcagatgaaagaaagtgcaggtaaggccagtcgttctttatctattggtgaatatcagtatttattgttattagcaaatatgcatccgtcttacaggctggtaaaaaaataa

SEQ ID NO: 79 - DNA, 90 bp, synthetic construct
  source 1..90: mol_type = other DNA
gacaataaataatgatcatccccagaaatcatgtggagcacaaccaacaactgacgaaaaatactgaagagtttgtagaaacgcaaaaag

SEQ ID NO: 80 - DNA, 90 bp, synthetic construct
  source 1..90: mol_type = other DNA
caaaaaatacgcccggtagtgatcttatttcattatggtgaaagttggaacctcttacgtgccgatcattagaaaaactcatcgagcatc

SEQ ID NO: 81 - DNA, 90 bp, synthetic construct
  source 1..90: mol_type = other DNA
cggacttagtgaacagggttttacagtctgattgacagatatggtcaccagaaaatatttgtattcctttagaaaaactcatcgagcatc

SEQ ID NO: 82 - DNA, 89 bp, synthetic construct
  source 1..89: mol_type = other DNA
cgcagacaataaataatgatcatccccagaaatcatgtggagcacaaccaacaactgacgaaaaatactgatgatcggcacgtaagagg

SEQ ID NO: 83 - DNA, 90 bp, synthetic construct
  source 1..90: mol_type = other DNA
accccggacttagtgaacagggttttacagtctgattgacagatatggtcaccagaaaatatttgtattcctaattcagcgggttcgtgc

SEQ ID NO: 84 - DNA, 90 bp, synthetic construct
  source 1..90: mol_type = other DNA
tacggctgcatgcccgaggcagacagcctcaagcacccgcagctattctacagtaaaaactcgcgcttctctagactatattaccctgtt

SEQ ID NO: 85 - DNA, 90 bp, synthetic construct
  source 1..90: mol_type = other DNA
agcggtacggaagtttcccccgacaccatgatggcggttagttgcgtcagaaatagttacgtatgcaactaatcagtcctgctcctcggc

SEQ ID NO: 86 - DNA, 20 bp, synthetic construct
  source 1..20: mol_type = other DNA
cggcacgtaagaggctaacc

SEQ ID NO: 87 - DNA, 90 bp, synthetic construct
  source 1..90: mol_type = other DNA
ggaagtttcccccgacaccatgatggcggttagttgcgtcagaaatagttacgtatgcaactaaaagcgcgagtttttactgtagaatag

SEQ ID NO: 88 - DNA, 90 bp, synthetic construct
  source 1..90: mol_type = other DNA
gtggtttgccacaaaacagtgcagtcacacatgacaggagaagatatgagccgggtaccgcggctctgagactcagtcctgctcctcggc

SEQ ID NO: 89 - DNA, 90 bp, synthetic construct
  source 1..90: mol_type = other DNA
ggtgcaggaaataaaaataatcatttctttatatgatctttttatcaatggagatagtatcatttatactctagactatattaccctgtt

SEQ ID NO: 90 - DNA, 22 bp, synthetic construct
  source 1..22: mol_type = other DNA
cctgtatttttctgctcaatgg

SEQ ID NO: 91 - DNA, 90 bp, synthetic construct
  source 1..90: mol_type = other DNA
aaacagtgcagtcacacatgacaggagaagatatgagccgggtaccgcggctctgagactataaatgatactatctccattgataaaaag

SEQ ID NO: 92 - DNA, 85 bp, synthetic construct
  source 1..85: mol_type = other DNA
gtgatctccaccggcttattagcgatagggattatctgtaccgtgccaattatggctcgcccttagttcctattccgaagttcct

SEQ ID NO: 93 - DNA, 87 bp, synthetic construct
  source 1..87: mol_type = other DNA
gtcgaaaatatcgagcagctcctgctctgacatagacgccacccgataccctttttgattcaccaccgccctgccactcatcgcagt

SEQ ID NO: 94 - DNA, 90 bp, synthetic construct
  source 1..90: mol_type = other DNA
attcagcgggttcgtgcgagctgttgacaattaatactcggctcgtataatgtgtggaaggctagcaggaggtgggctagcgaattcgag

SEQ ID NO: 95 - DNA, 20 bp, synthetic construct
  source 1..20: mol_type = other DNA
ttaattgccagccatcgcct

SEQ ID NO: 96 - DNA, 119 bp, synthetic construct
  source 1..119: mol_type = other DNA
ccggcttattagcgatagggattatctgtaccgtgccaattatggctcgctggaaaaagctggtattgtggcaaaaaacacccgttcataatacgcgctgaattcagcgggttcgtgcg

SEQ ID NO: 97 - DNA, 21 bp, synthetic construct
  source 1..21: mol_type = other DNA
gtgatcaccagataatgttgc

SEQ ID NO: 98 - DNA, 18 bp, synthetic construct
  source 1..18: mol_type = other DNA
gcttcatcaccactgacc

SEQ ID NO: 99 - DNA, 87 bp, synthetic construct
  source 1..87: mol_type = other DNA
ccggtatcgggttaaagagtgggaaaaagttgccggtgttcctgttgcagaagtattttgactcacttccctgttaagtatcttcct

SEQ ID NO: 100 - DNA, 87 bp, synthetic construct
  source 1..87: mol_type = other DNA
aaaagaaaaagccggttcagagaaaaccggctgacggattatcgtctaacagttgatagctaccattacgccccgccctgccactca

SEQ ID NO: 101 - DNA, 20 bp, synthetic construct
  source 1..20: mol_type = other DNA
gcgtcaaggatgctctactc

SEQ ID NO: 102 - DNA, 145 bp, synthetic construct
  source 1..145: mol_type = other DNA
aacctaaatatttgttgttaagctgcaatggaaacggtaaaagcggctagtatttaaagggatggatgacatctcagcgttgtcgggattaacaatataagctgaccttcaagtattgaattgggataatgtgtggaattgtgaa

SEQ ID NO: 103 - DNA, 29 bp, synthetic construct
  source 1..29: mol_type = other DNA
gtattccccccgcatgcgggggttatcgg